SEQUENCE LISTING (8 sequences)

SEQ ID NO: 1 - DNA, 10723 bp, synthetic construct
  source 1..10723: mol_type = other DNA; note = chimeric dengue seroytpe 2/1
agttgttagtctacgtggaccgacaaagacagattctttgagggagctaagctcaatgtagttctaacagttttttaattagagagcagatctctgatgaataaccaacggaaaaaggcgaaaaacacgcctttcaatatgctgaaacgcgagagaaaccgcgtgtcgactgtgcaacagctgacaaagagattctcacttggaatgctgcagggacgaggaccattaaaactgttcatggccctggtggcgttccttcgtttcctaacaatcccaccaacagcagggatattgaagagatggggaacaattaaaaaatcaaaagctattaatgttttgagagggttcaggaaagagattggaaggatgctgaacatcttgaataggagacgcagatctgcaggcatgatcattatgctgattccaacagtgatggcgttccatttaaccacgcgtgggggagagccgcatatgatagttagcaagcaggaaagaggaaagtcacttttgttcaagacctctgcaggtgtcaacatgtgcaccctcattgcgatggatttgggagagttgtgtgaggacacgatgacctacaaatgcccccggatcactgaggcggaaccagatgacgttgactgttggtgcaatgccacggacacatgggtgacctatggaacgtgctctcaaactggcgaacaccgacgagacaaacgttccgtcgcattggccccacacgtggggcttggcctagaaacaagagccgaaacgtggatgtcctctgaaggtgcttggaaacagatacaaaaagtagagacttgggctctgagacatccaggattcacggtgatagccctttttctagcacatgccataggaacatccatcacccagaaagggatcattttcattttgctgatgctggtaacaccatctatggccatgcgatgcgtgggaataggcaacagagacttcgtggaaggactgtcaggagcaacatgggtggatgtggtactggagcatggaagttgcgtcaccaccatggcaaaaaacaaaccaacactggacattgaactcttgaagacggaggtcacaaaccctgcagttctgcgtaaattgtgcattgaagctaaaatatcaaacaccaccaccgattcgagatgtccaacacaaggagaagccacactggtggaagaacaagacgcgaactttgtgtgccgacgaacgttcgtggacagaggctggggcaatggctgtgggctattcggaaaaggtagtctaataacgtgtgccaagtttaagtgtgtgacaaaactagaaggaaagatagttcaatatgaaaacctaaaatattcagtgatagtcaccgtccacactggagatcagcaccaggtgggaaatgagactacagaacatggaacaactgcaaccataacacctcaagctcctacgtcggaaatacagctgaccgactacggaacccttacattagattgttcacctaggacagggctagattttaacgagatggtgttgctgacaatgaaagaaagatcatggcttgtccacaaacaatggttcctagacttaccactgccttggacctctggggcttcaacatcccaagagacttggaacagacaagatttactggtcacatttaagacagctcatgcaaagaagcaggaagtagtcgtactaggatcacaagaaggagcaatgcacactgcgctgactggagcgacagaaatccaaacgtcaggaacgacaacaattttcgcaggacacctaaaatgcagactaaaaatggacaaactaactttaaaagggatgtcatatgtgatgtgcacaggctcattcaagttagagaaagaagtggctgagacccagcatggaactgttctggtgcaggttaaatatgaaggaacagacgcaccatgcaagattcccttttcgacccaagatgagaaaggagcaacccagaatgggagattaataacagccaaccccatagtcactgacaaagaaaaaccagtcaatattgaggcagaaccaccctttggtgagagctacatcgtggtaggagcaggtgaaaaagctttgaaactaagctggttcaagaaaggaagcagcatagggaaaatgtttgaagcaactgcccgaggagcacgaaggatggccattctgggagacaccgcatgggacttcggttctataggaggagtgttcacgtctatgggaaaactggtacaccaggtttttggaactgcatatggagttttgtttagcggagtttcttggaccatgaaaataggaatagggattctgctgacatggctaggattaaattcaaggaacacgtccctttcgatgatgtgcatcgcagccggcattgtgacactgtatttgggagtcatggtgcaggccgatagtggttgcgttgtgagctggaaaaacaaagaactgaaatgtggcagtgggattttcatcacagacaacgtgcacacatggacagaacaatacaagttccaaccagaatccccttcaaaactagcttcagctatccagaaagcccatgaagaggacatttgtggaatccgctcagtaacaagactggagaatctgatgtggaaacaaataacaccagaattgaatcacattctatcagaaaatgaggtgaagttaactattatgacaggagacatcaaaggaatcatgcaggcaggaaaacgatctctgcggcctcagcccactgagctgaagtattcatggaaaacatggggcaaagcaaaaatgctctctacagagtctcataaccagacctttctcattgatggccccgaaacagcagaatgccccaacacaaatagagcttggaattcgttggaagttgaagactatggctttggagtattcaccaccaatatatggctaaaattgaaagaaaaacaggatgtattctgcgactcaaaactcatgtcagcggccataaaagacaacagagccgtccatgccgatatgggttattggatagaaagtgcactcaatgacacatggaagatagagaaagcctctttcattgaagttaaaaactgccactggccaaaatcacacaccctctggagcaatggagtgctagaaagtgagatgataattccaaagaatctcgctggaccagtgtctcaacacaactatagaccaggctaccatacacaaataacaggaccatggcatctaggtaagcttgagatggactttgatttctgtgatggaacaacagtggtagtgactgaggactgcggaaatagaggaccctctttgagaacaaccactgcctctggaaaactcataacagaatggtgctgccgatcttgcacattaccaccgctaagatacagaggtgaggatgggtgctggtacgggatggaaatcagaccattgaaggagaaagaagagaatttggtcaactccttggtcacagctggacatgggcaggtcgacaacttttcactaggagtcttgggaatggcattgttcctggaggaaatgcttaggacccgagtaggaacgaaacatgcaatactactagttgcagtttcttttgtgacattgatcacagggaacatgtcctttagagacctgggaagagtgatggttatggtaggcgccactatgacggatgacataggtatgggcgtgacttatcttgccctactagcagccttcaaagtcagaccaacttttgcagctggactactcttgagaaagctgacctccaaggaattgatgatgactactataggaattgtactcctctcccagagcaccctaccagagaccattcttgagttgactgatgcgttagccttaggcatgatggtcctcaaaatggtgagaaatatggaaaagtatcaattggcagtgactatcatggctatcttgtgcgtcccaaacgcagtgatattacaaaacgcatggaaagtgagttgcacaatattggcagtggtgtccgtttccccactgttcttaacatcctcacagcaaaaaacagattggataccattagcattgacgatcaaaggtctcaatccaacagctatttttctaacaaccctctcaagaaccagcaagaaaaggagctggccattaaatgaggctatcatggcagtcgggatggtgagcattttagccagttctctcctaaaaaatgatattcccatgacaggaccattagtggctggagggctcctcactgtgtgctacgtgctcactggacgatcggccgatttggaactggagagagcagccgatgtcaaatgggaagaccaggcagagatatcaggaagcagtccaatcctgtcaataacaatatcagaagatggtagcatgtcgataaaaaatgaagaggaagatcaaacactgaccatactcattagaacaggattgctggtgatctcaggactttttcctgtatcaataccaatcacggcagcagcatggtacctgtgggaagtgaagaaacaacgggccggagtattgtgggatgttccttcacccccacccatgggaaaggctgaactggaagatggagcctatagaattaagcaaaaagggattcttggatattcccagatcggagccggagtttacaaagaaggaacattccatacaatgtggcatgtcacacgtggcgctgttctaatgcataaaggaaagaggattgaaccatcatgggcggacgtcaagaaagacctaatatcatatggaggaggctggaagttagaaggagaatggaaggaaggagaagaagtccaggtattggcactggagcctggaaaaaatccaagagccgtccaaacgaaacctggtcttttcaaaaccaacgccggaacaataggtgctgtatctctggacttttctcctggaacgtcaggatctccaattatcgacaaaaaaggaaaagttgtgggtctttatggtaatggtgttgttacaaggagtggagcatatgtgagtgctatagcccagactgaaaaaagcattgaagacaacccagagatcgaagatgacattttccgaaagagaagactgaccatcatggacctccacccaggagcgggaaagacgaagagataccttccggccatagtcagagaagctataaaacggggtttgagaacattaatcttggcccccactagagttgtggcagctgaaatggaggaagcccttagaggacttccaataagataccagaccccagccatcagagctgtgcacaccgggcgggagattgtggacctaatgtgtcatgccacatttaccatgaggctgctatcaccagttagagtgccaaactacaacctgattatcatggacgaagcccatttcacagacccagcaagtatagcagctagaggatacatctcaactcgagtggagatgggtgaggcagctgggatttttatgacagccactcccccgggaagcagagacccatttcctcagagcaatgcaccaatcatagatgaagaaagagaaatccctgaacgctcgtggaattccggacatgaatgggtcacggattttaaagggaagactgtttggttcgttccaagtataaaagcaggaaatgatatagcagcttgcctgaggaaaaatggaaagaaagtgatacaactcagtaggaagacctttgattctgagtatgtcaagactagaaccaatgattgggacttcgtggttacaactgacatttcagaaatgggtgccaatttcaaggctgagagggttatagaccccagacgctgcatgaaaccagtcatactaacagatggtgaagagcgggtgattctggcaggacctatgccagtgacccactctagtgcagcacaaagaagagggagaataggaagaaatccaaaaaatgagaatgaccagtacatatacatgggggaacctctggaaaatgatgaagactgtgcacactggaaagaagctaaaatgctcctagataacatcaacacgccagaaggaatcattcctagcatgttcgaaccagagcgtgaaaaggtggatgccattgatggcgaataccgcttgagaggagaagcaaggaaaacctttgtagacttaatgagaagaggagacctaccagtctggttggcctacagagtggcagctgaaggcatcaactacgcagacagaaggtggtgttttgatggagtcaagaacaaccaaatcctagaagaaaacgtggaagttgaaatctggacaaaagaaggggaaaggaagaaattgaaacccagatggttggatgctaggatctattctgacccactggcgctaaaagaatttaaggaatttgcagccggaagaaagtctctgaccctgaacctaatcacagaaatgggtaggctcccaaccttcatgactcagaaggcaagagacgcactggacaacttagcagtgctgcacacggctgaggcaggtggaagggcgtacaaccatgctctcagtgaactgccggagaccctggagacattgcttttactgacacttctggctacagtcacgggagggatctttttattcttgatgagcgcaaggggcatagggaagatgaccctgggaatgtgctgcataatcacggctagcatcctcctatggtacgcacaaatacagccacactggatagcagcttcaataatactggagttttttctcatagttttgcttattccagaacctgaaaaacagagaacaccccaagacaaccaactgacctacgttgtcatagccatcctcacagtggtggccgcaaccatggcaaacgagatgggtttcctagaaaaaacgaagaaagatctcggattgggaagcattgcaacccagcaacccgagagcaacatcctggacatagatctacgtcctgcatcagcatggacgctgtatgccgtggccacaacatttgttacaccaatgttgagacatagcattgaaaattcctcagtgaatgtgtccctaacagctatagccaaccaagccacagtgttaatgggtctcgggaaaggatggccattgtcaaagatggacatcggagttccccttctcgccattggatgctactcacaagtcaaccccataactctcacagcagctcttttcttattggtagcacattatgccatcatagggccaggactccaagcaaaagcaaccagagaagctcagaaaagagcagcggcgggcatcatgaaaaacccaactgtcgatggaataacagtgattgacctagatccaataccttatgatccgaagtttgaaaagcagttgggacaagtaatgctcctagtcctctgcgtgactcaagtattgatgatgaggactacatgggctctgtgtgaggctttaaccttagctaccgggcccatctccacattgtgggaaggaaatccagggaggttttggaacactaccattgcggtgtcaatggctaacatttttagagggagttacttggccggagctggacttctcttttctattatgaagaacacaaccaacacaagaaggggaactggcaacataggagagacgcttggagagaaatggaaaagccgattgaacgcattgggaaaaagtgaattccagatctacaagaaaagtggaatccaggaagtggatagaaccttagcaaaagaaggcattaaaagaggagaaacggaccatcacgctgtgtcgcgaggctcagcaaaactgagatggttcgttgagagaaacatggtcacaccagaagggaaagtagtggacctcggttgtggcagaggaggctggtcatactattgtggaggactaaagaatgtaagagaagtcaaaggcctaacaaaaggaggaccaggacacgaagaacccatccccatgtcaacatatgggtggaatctagtgcgtcttcaaagtggagttgacgttttcttcatcccgccagaaaagtgtgacacattattgtgtgacataggggagtcatcaccaaatcccacagtggaagcaggacgaacactcagagtccttaacttagtagaaaattggttgaacaacaacactcaattttgcataaaggttctcaacccatatatgccctcagtcatagaaaaaatggaagcactacaaaggaaatatggaggagccttagtgaggaatccactctcacgaaactccacacatgagatgtactgggtatccaatgcttccgggaacatagtgtcatcagtgaacatgatttcaaggatgttgatcaacagatttacaatgagatacaagaaagccacttacgagccggatgttgacctcggaagcggaacccgtaacatcgggattgaaagtgagataccaaacctagatataattgggaaaagaatagaaaaaataaagcaagagcatgaaacatcatggcactatgaccaagaccacccatacaaaacgtgggcataccatggtagctatgaaacaaaacagactggatcagcatcatccatggtcaacggagtggtcaggctgctgacaaaaccttgggacgtcgtccccatggtgacacagatggcaatgacagacacgactccatttggacaacagcgcgtttttaaagagaaagtggacacgagaacccaagaaccgaaagaaggcacgaagaaactaatgaaaataacagcagagtggctttggaaagaattagggaagaaaaagacacccaggatgtgcaccagagaagaattcacaagaaaggtgagaagcaatgcagccttgggggccatattcactgatgagaacaagtggaagtcggcacgtgaggctgttgaagatagtaggttttgggagctggttgacaaggaaaggaatctccatcttgaaggaaagtgtgaaacatgtgtgtacaacatgatgggaaaaagagagaagaagctaggggaattcggcaaggcaaaaggcagcagagccatatggtacatgtggcttggagcacgcttcttagagtttgaagccctaggattcttaaatgaagatcactggttctccagagagaactccctgagtggagtggaaggagaagggctgcacaagctaggttacattctaagagacgtgagcaagaaagagggaggagcaatgtatgccgatgacaccgcaggatgggatacaagaatcacactagaagacctaaaaaatgaagaaatggtaacaaaccacatggaaggagaacacaagaaactagccgaggccattttcaaactaacgtaccaaaacaaggtggtgcgtgtgcaaagaccaacaccaagaggcacagtaatggacatcatatcgagaagagaccaaagaggtagtggacaagttggcacctatggactcaatactttcaccaatatggaagcccaactaatcagacagatggagggagaaggagtctttaaaagcattcagcacctaacaatcacagaagaaatcgctgtgcaaaactggttagcaagagtggggcgcgaaaggttatcaagaatggccatcagtggagatgattgtgttgtgaaacctttagatgacaggttcgcaagcgctttaacagctctaaatgacatgggaaagattaggaaagacatacaacaatgggaaccttcaagaggatggaatgattggacacaagtgcccttctgttcacaccatttccatgagttaatcatgaaagacggtcgcgtactcgttgttccatgtagaaaccaagatgaactgattggcagagcccgaatctcccaaggagcagggtggtctttgcgggagacggcctgtttggggaagtcttacgcccaaatgtggagcttgatgtacttccacagacgcgacctcaggctggcggcaaatgctatttgctcggcagtaccatcacattgggttccaacaagtcgaacaacctggtccatacatgctaaacatgaatggatgacaacggaagacatgctgacagtctggaacagggtgtggattcaagaaaacccatggatggaagacaaaactccagtggaatcatgggaggaaatcccatacttggggaaaagagaagaccaatggtgcggctcattgattgggttaacaagcagggccacctgggcaaagaacatccaagcagcaataaatcaagttagatcccttataggcaatgaagaatacacagattacatgccatccatgaaaagattcagaagagaagaggaagaagcaggagttctgtggtagaaagcaaaactaacatgaaacaaggctagaagtcaggtcggattaagccatagtacggaaaaaactatgctacctgtgagccccgtccaaggacgttaaaagaagtcaggccatcataaatgccatagcttgagtaaactatgcagcctgtagctccacctgagaaggtgtaaaaaatccgggaggccacaaaccatggaagctgtacgcatggcgtagtggactagcggttagaggagacccctcccttacaaatcgcagcaacaatgggggcccaaggcgagatgaagctgtagtctcgctggaaggactagaggttagaggagacccccccgaaacaaaaaacagcatattgacgctgggaaagaccagagatcctgctgtctcctcagcatcattccaggcacagaacgccagaaaatggaatggtgctgttgaatcaacaggttct

SEQ ID NO: 2 - protein, 3391 aa, synthetic construct
  source 1..3391: note = chimeric dengue seroytpe 2/1
MNNQRKKAKNTPFNMLKRERNRVSTVQQLTKRFSLGMLQGRGPLKLFMALVAFLRFLTIPPTAGILKRWGTIKKSKAINVLRGFRKEIGRMLNILNRRRRSAGMIIMLIPTVMAFHLTTRGGEPHMIVSKQERGKSLLFKTSAGVNMCTLIAMDLGELCEDTMTYKCPRITEAEPDDVDCWCNATDTWVTYGTCSQTGEHRRDKRSVALAPHVGLGLETRAETWMSSEGAWKQIQKVETWALRHPGFTVIALFLAHAIGTSITQKGIIFILLMLVTPSMAMRCVGIGNRDFVEGLSGATWVDVVLEHGSCVTTMAKNKPTLDIELLKTEVTNPAVLRKLCIEAKISNTTTDSRCPTQGEATLVEEQDANFVCRRTFVDRGWGNGCGLFGKGSLITCAKFKCVTKLEGKIVQYENLKYSVIVTVHTGDQHQVGNETTEHGTTATITPQAPTSEIQLTDYGTLTLDCSPRTGLDFNEMVLLTMKERSWLVHKQWFLDLPLPWTSGASTSQETWNRQDLLVTFKTAHAKKQEVVVLGSQEGAMHTALTGATEIQTSGTTTIFAGHLKCRLKMDKLTLKGMSYVMCTGSFKLEKEVAETQHGTVLVQVKYEGTDAPCKIPFSTQDEKGATQNGRLITANPIVTDKEKPVNIEAEPPFGESYIVVGAGEKALKLSWFKKGSSIGKMFEATARGARRMAILGDTAWDFGSIGGVFTSMGKLVHQVFGTAYGVLFSGVSWTMKIGIGILLTWLGLNSRNTSLSMMCIAAGIVTLYLGVMVQADSGCVVSWKNKELKCGSGIFITDNVHTWTEQYKFQPESPSKLASAIQKAHEEDICGIRSVTRLENLMWKQITPELNHILSENEVKLTIMTGDIKGIMQAGKRSLRPQPTELKYSWKTWGKAKMLSTESHNQTFLIDGPETAECPNTNRAWNSLEVEDYGFGVFTTNIWLKLKEKQDVFCDSKLMSAAIKDNRAVHADMGYWIESALNDTWKIEKASFIEVKNCHWPKSHTLWSNGVLESEMIIPKNLAGPVSQHNYRPGYHTQITGPWHLGKLEMDFDFCDGTTVVVTEDCGNRGPSLRTTTASGKLITEWCCRSCTLPPLRYRGEDGCWYGMEIRPLKEKEENLVNSLVTAGHGQVDNFSLGVLGMALFLEEMLRTRVGTKHAILLVAVSFVTLITGNMSFRDLGRVMVMVGATMTDDIGMGVTYLALLAAFKVRPTFAAGLLLRKLTSKELMMTTIGIVLLSQSTLPETILELTDALALGMMVLKMVRNMEKYQLAVTIMAILCVPNAVILQNAWKVSCTILAVVSVSPLFLTSSQQKTDWIPLALTIKGLNPTAIFLTTLSRTSKKRSWPLNEAIMAVGMVSILASSLLKNDIPMTGPLVAGGLLTVCYVLTGRSADLELERAADVKWEDQAEISGSSPILSITISEDGSMSIKNEEEDQTLTILIRTGLLVISGLFPVSIPITAAAWYLWEVKKQRAGVLWDVPSPPPMGKAELEDGAYRIKQKGILGYSQIGAGVYKEGTFHTMWHVTRGAVLMHKGKRIEPSWADVKKDLISYGGGWKLEGEWKEGEEVQVLALEPGKNPRAVQTKPGLFKTNAGTIGAVSLDFSPGTSGSPIIDKKGKVVGLYGNGVVTRSGAYVSAIAQTEKSIEDNPEIEDDIFRKRRLTIMDLHPGAGKTKRYLPAIVREAIKRGLRTLILAPTRVVAAEMEEALRGLPIRYQTPAIRAVHTGREIVDLMCHATFTMRLLSPVRVPNYNLIIMDEAHFTDPASIAARGYISTRVEMGEAAGIFMTATPPGSRDPFPQSNAPIIDEEREIPERSWNSGHEWVTDFKGKTVWFVPSIKAGNDIAACLRKNGKKVIQLSRKTFDSEYVKTRTNDWDFVVTTDISEMGANFKAERVIDPRRCMKPVILTDGEERVILAGPMPVTHSSAAQRRGRIGRNPKNENDQYIYMGEPLENDEDCAHWKEAKMLLDNINTPEGIIPSMFEPEREKVDAIDGEYRLRGEARKTFVDLMRRGDLPVWLAYRVAAEGINYADRRWCFDGVKNNQILEENVEVEIWTKEGERKKLKPRWLDARIYSDPLALKEFKEFAAGRKSLTLNLITEMGRLPTFMTQKARDALDNLAVLHTAEAGGRAYNHALSELPETLETLLLLTLLATVTGGIFLFLMSARGIGKMTLGMCCIITASILLWYAQIQPHWIAASIILEFFLIVLLIPEPEKQRTPQDNQLTYVVIAILTVVAATMANEMGFLEKTKKDLGLGSIATQQPESNILDIDLRPASAWTLYAVATTFVTPMLRHSIENSSVNVSLTAIANQATVLMGLGKGWPLSKMDIGVPLLAIGCYSQVNPITLTAALFLLVAHYAIIGPGLQAKATREAQKRAAAGIMKNPTVDGITVIDLDPIPYDPKFEKQLGQVMLLVLCVTQVLMMRTTWALCEALTLATGPISTLWEGNPGRFWNTTIAVSMANIFRGSYLAGAGLLFSIMKNTTNTRRGTGNIGETLGEKWKSRLNALGKSEFQIYKKSGIQEVDRTLAKEGIKRGETDHHAVSRGSAKLRWFVERNMVTPEGKVVDLGCGRGGWSYYCGGLKNVREVKGLTKGGPGHEEPIPMSTYGWNLVRLQSGVDVFFIPPEKCDTLLCDIGESSPNPTVEAGRTLRVLNLVENWLNNNTQFCIKVLNPYMPSVIEKMEALQRKYGGALVRNPLSRNSTHEMYWVSNASGNIVSSVNMISRMLINRFTMRYKKATYEPDVDLGSGTRNIGIESEIPNLDIIGKRIEKIKQEHETSWHYDQDHPYKTWAYHGSYETKQTGSASSMVNGVVRLLTKPWDVVPMVTQMAMTDTTPFGQQRVFKEKVDTRTQEPKEGTKKLMKITAEWLWKELGKKKTPRMCTREEFTRKVRSNAALGAIFTDENKWKSAREAVEDSRFWELVDKERNLHLEGKCETCVYNMMGKREKKLGEFGKAKGSRAIWYMWLGARFLEFEALGFLNEDHWFSRENSLSGVEGEGLHKLGYILRDVSKKEGGAMYADDTAGWDTRITLEDLKNEEMVTNHMEGEHKKLAEAIFKLTYQNKVVRVQRPTPRGTVMDIISRRDQRGSGQVGTYGLNTFTNMEAQLIRQMEGEGVFKSIQHLTITEEIAVQNWLARVGRERLSRMAISGDDCVVKPLDDRFASALTALNDMGKIRKDIQQWEPSRGWNDWTQVPFCSHHFHELIMKDGRVLVVPCRNQDELIGRARISQGAGWSLRETACLGKSYAQMWSLMYFHRRDLRLAANAICSAVPSHWVPTSRTTWSIHAKHEWMTTEDMLTVWNRVWIQENPWMEDKTPVESWEEIPYLGKREDQWCGSLIGLTSRATWAKNIQAAINQVRSLIGNEEYTDYMPSMKRFRREEEEAGVLW

SEQ ID NO: 3 - DNA, 10723 bp, synthetic construct
  source 1..10723: mol_type = other DNA; note = dengue serotype 2
agttgttagtctacgtggaccgacaaagacagattctttgagggagctaagctcaatgtagttctaacagttttttaattagagagcagatctctgatgaataaccaacggaaaaaggcgaaaaacacgcctttcaatatgctgaaacgcgagagaaaccgcgtgtcgactgtgcaacagctgacaaagagattctcacttggaatgctgcagggacgaggaccattaaaactgttcatggccctggtggcgttccttcgtttcctaacaatcccaccaacagcagggatattgaagagatggggaacaattaaaaaatcaaaagctattaatgttttgagagggttcaggaaagagattggaaggatgctgaacatcttgaataggagacgcagatctgcaggcatgatcattatgctgattccaacagtgatggcgttccatttaaccacacgtaacggagaaccacacatgatcgtcagcagacaagagaaagggaaaagtcttctgtttaaaacagaggttggcgtgaacatgtgtaccctcatggccatggaccttggtgaattgtgtgaagacacaatcacgtacgagtgtccccttctcaggcagaatgagccagaagacatagactgttggtgcaactctacgtccacgtgggtaacttatgggacgtgtaccaccatgggagaacatagaagagaaaaaagatcagtggcactcgttccacatgtgggaatgggactggagacacgaactgaaacatggatgtcatcagaaggggcctggaaacatgtccagagaattgaaacttggatcttgagacatccaggcttcaccatgatggcagcaatcctggcatacaccataggaacgacacatttccaaagagccctgatcttcatcttactgacagctgtcactccttcaatgacaatgcgttgcataggaatgtcaaatagagactttgtggaaggggtttcaggaggaagctgggttgacatagtcttagaacatggaagctgtgtgacgacgatggcaaaaaacaaaccaacattggattttgaactgataaaaacagaagccaaacagcctgccaccctaaggaagtactgtatagaggcaaagctaaccaacacaacaacagaatctcgctgcccaacacaaggggaacccagcctaaatgaagagcaggacaaaaggttcgtctgcaaacactccatggtagacagaggatggggaaatggatgtggactatttggaaagggaggcattgtgacctgtgctatgttcagatgcaaaaagaacatggaaggaaaagttgtgcaaccagaaaacttggaatacaccattgtgataacacctcactcaggggaagagcatgcagtcggaaatgacacaggaaaacatggcaaggaaatcaaaataacaccacagagttccatcacagaagcagaattgacaggttatggcactgtcacaatggagtgctctccaagaacgggcctcgacttcaatgagatggtgttgctgcagatggaaaataaagcttggctggtgcacaggcaatggttcctagacctgccgttaccatggttgcccggagcggacacacaagggtcaaattggatacagaaagagacattggtcactttcaaaaatccccatgcgaagaaacaggatgttgttgttttaggatcccaagaaggggccatgcacacagcacttacaggggccacagaaatccaaatgtcatcaggaaacttactcttcacaggacatctcaagtgcaggctgagaatggacaagctacagctcaaaggaatgtcatactctatgtgcacaggaaagtttaaagttgtgaaggaaatagcagaaacacaacatggaacaatagttatcagagtgcaatatgaaggggacggctctccatgcaagatcccttttgagataatggatttggaaaaaagacatgtcttaggtcgcctgattacagtcaacccaattgtgacagaaaaagatagcccagtcaacatagaagcagaacctccatttggagacagctacatcatcataggagtagagccgggacaactgaagctcaactggtttaagaaaggaagttctatcggccaaatgtttgagacaacaatgaggggggcgaagagaatggccattttaggtgacacagcctgggattttggatccttgggaggagtgtttacatctataggaaaggctctccaccaagtctttggagcaatctatggagctgccttcagtggggtttcatggactatgaaaatcctcataggagtcattatcacatggataggaatgaattcacgcagcacctcactgtctgtgacactagtattggtgggaattgtgacactgtatttgggagtcatggtgcaggccgatagtggttgcgttgtgagctggaaaaacaaagaactgaaatgtggcagtgggattttcatcacagacaacgtgcacacatggacagaacaatacaagttccaaccagaatccccttcaaaactagcttcagctatccagaaagcccatgaagaggacatttgtggaatccgctcagtaacaagactggagaatctgatgtggaaacaaataacaccagaattgaatcacattctatcagaaaatgaggtgaagttaactattatgacaggagacatcaaaggaatcatgcaggcaggaaaacgatctctgcggcctcagcccactgagctgaagtattcatggaaaacatggggcaaagcaaaaatgctctctacagagtctcataaccagacctttctcattgatggccccgaaacagcagaatgccccaacacaaatagagcttggaattcgttggaagttgaagactatggctttggagtattcaccaccaatatatggctaaaattgaaagaaaaacaggatgtattctgcgactcaaaactcatgtcagcggccataaaagacaacagagccgtccatgccgatatgggttattggatagaaagtgcactcaatgacacatggaagatagagaaagcctctttcattgaagttaaaaactgccactggccaaaatcacacaccctctggagcaatggagtgctagaaagtgagatgataattccaaagaatctcgctggaccagtgtctcaacacaactatagaccaggctaccatacacaaataacaggaccatggcatctaggtaagcttgagatggactttgatttctgtgatggaacaacagtggtagtgactgaggactgcggaaatagaggaccctctttgagaacaaccactgcctctggaaaactcataacagaatggtgctgccgatcttgcacattaccaccgctaagatacagaggtgaggatgggtgctggtacgggatggaaatcagaccattgaaggagaaagaagagaatttggtcaactccttggtcacagctggacatgggcaggtcgacaacttttcactaggagtcttgggaatggcattgttcctggaggaaatgcttaggacccgagtaggaacgaaacatgcaatactactagttgcagtttcttttgtgacattgatcacagggaacatgtcctttagagacctgggaagagtgatggttatggtaggcgccactatgacggatgacataggtatgggcgtgacttatcttgccctactagcagccttcaaagtcagaccaacttttgcagctggactactcttgagaaagctgacctccaaggaattgatgatgactactataggaattgtactcctctcccagagcaccataccagagaccattcttgagttgactgatgcgttagccttaggcatgatggtcctcaaaatggtgagaaatatggaaaagtatcaattggcagtgactatcatggctatcttgtgcgtcccaaacgcagtgatattacaaaacgcatggaaagtgagttgcacaatattggcagtggtgtccgtttccccactgttcttaacatcctcacagcaaaaaacagattggataccattagcattgacgatcaaaggtctcaatccaacagctatttttctaacaaccctctcaagaaccagcaagaaaaggagctggccattaaatgaggctatcatggcagtcgggatggtgagcattttagccagttctctcctaaaaaatgatattcccatgacaggaccattagtggctggagggctcctcactgtgtgctacgtgctcactggacgatcggccgatttggaactggagagagcagccgatgtcaaatgggaagaccaggcagagatatcaggaagcagtccaatcctgtcaataacaatatcagaagatggtagcatgtcgataaaaaatgaagaggaagaacaaacactgaccatactcattagaacaggattgctggtgatctcaggactttttcctgtatcaataccaatcacggcagcagcatggtacctgtgggaagtgaagaaacaacgggccggagtattgtgggatgttccttcacccccacccatgggaaaggctgaactggaagatggagcctatagaattaagcaaaaagggattcttggatattcccagatcggagccggagtttacaaagaaggaacattccatacaatgtggcatgtcacacgtggcgctgttctaatgcataaaggaaagaggattgaaccatcatgggcggacgtcaagaaagacctaatatcatatggaggaggctggaagttagaaggagaatggaaggaaggagaagaagtccaggtattggcactggagcctggaaaaaatccaagagccgtccaaacgaaacctggtcttttcaaaaccaacgccggaacaataggtgctgtatctctggacttttctcctggaacgtcaggatctccaattatcgacaaaaaaggaaaagttgtgggtctttatggtaatggtgttgttacaaggagtggagcatatgtgagtgctatagcccagactgaaaaaagcattgaagacaacccagagatcgaagatgacattttccgaaagagaagactgaccatcatggacctccacccaggagcgggaaagacgaagagataccttccggccatagtcagagaagctataaaacggggtttgagaacattaatcttggcccccactagagttgtggcagctgaaatggaggaagcccttagaggacttccaataagataccagaccccagccatcagagctgtgcacaccgggcgggagattgtggacctaatgtgtcatgccacatttaccatgaggctgctatcaccagttagagtgccaaactacaacctgattatcatggacgaagcccatttcacagacccagcaagtatagcagctagaggatacatctcaactcgagtggagatgggtgaggcagctgggatttttatgacagccactcccccgggaagcagagacccatttcctcagagcaatgcaccaatcatagatgaagaaagagaaatccctgaacgctcgtggaattccggacatgaatgggtcacggattttaaagggaagactgtttggttcgttccaagtataaaagcaggaaatgatatagcagcttgcctgaggaaaaatggaaagaaagtgatacaactcagtaggaagacctttgattctgagtatgtcaagactagaaccaatgattgggacttcgtggttacaactgacatttcagaaatgggtgccaatttcaaggctgagagggttatagaccccagacgctgcatgaaaccagtcatactaacagatggtgaagagcgggtgattctggcaggacctatgccagtgacccactctagtgcagcacaaagaagagggagaataggaagaaatccaaaaaatgagaatgaccagtacatatacatgggggaacctctggaaaatgatgaagactgtgcacactggaaagaagctaaaatgctcctagataacatcaacacgccagaaggaatcattcctagcatgttcgaaccagagcgtgaaaaggtggatgccattgatggcgaataccgcttgagaggagaagcaaggaaaacctttgtagacttaatgagaagaggagacctaccagtctggttggcctacagagtggcagctgaaggcatcaactacgcagacagaaggtggtgttttgatggagtcaagaacaaccaaatcctagaagaaaacgtggaagttgaaatctggacaaaagaaggggaaaggaagaaattgaaacccagatggttggatgctaggatctattctgacccactggcgctaaaagaatttaaggaatttgcagccggaagaaagtctctgaccctgaacctaatcacagaaatgggtaggctcccaaccttcatgactcagaaggcaagagacgcactggacaacttagcagtgctgcacacggctgaggcaggtggaagggcgtacaaccatgctctcagtgaactgccggagaccctggagacattgcttttactgacacttctggctacagtcacgggagggatctttttattcttgatgagcgcaaggggcatagggaagatgaccctgggaatgtgctgcataatcacggctagcatcctcctatggtacgcacaaatacagccacactggatagcagcttcaataatactggagttttttctcatagttttgcttattccagaacctgaaaaacagagaacaccccaagacaaccaactgacctacgttgtcatagccatcctcacagtggtggccgcaaccatggcaaacgagatgggtttcctagaaaaaacgaagaaagatctcggattgggaagcattgcaacccagcaacccgagagcaacatcctggacatagatctacgtcctgcatcagcatggacgctgtatgccgtggccacaacatttgttacaccaatgttgagacatagcattgaaaattcctcagtgaatgtgtccctaacagctatagccaaccaagccacagtgttaatgggtctcgggaaaggatggccattgtcaaagatggacatcggagttccccttctcgccattggatgctactcacaagtcaaccccataactctcacagcagctcttttcttattggtagcacattatgccatcatagggccaggactccaagcaaaagcaaccagagaagctcagaaaagagcagcggcgggcatcatgaaaaacccaactgtcgatggaataacagtgattgacctagatccaataccttatgatccaaagtttgaaaagcagttgggacaagtaatgctcctagtcctctgcgtgactcaagtattgatgatgaggactacatgggctctgtgtgaggctttaaccttagctaccgggcccatctccacattgtgggaaggaaatccagggaggttttggaacactaccattgcggtgtcaatggctaacatttttagagggagttacttggccggagctggacttctcttttctattatgaagaacacaaccaacacaagaaggggaactggcaacataggagagacgcttggagagaaatggaaaagccgattgaacgcattgggaaaaagtgaattccagatctacaagaaaagtggaatccaggaagtggatagaaccttagcaaaagaaggcattaaaagaggagaaacggaccatcacgctgtgtcgcgaggctcagcaaaactgagatggttcgttgagagaaacatggtcacaccagaagggaaagtagtggacctcggttgtggcagaggaggctggtcatactattgtggaggactaaagaatgtaagagaagtcaaaggcctaacaaaaggaggaccaggacacgaagaacccatccccatgtcaacatatgggtggaatctagtgcgtcttcaaagtggagttgacgttttcttcatcccgccagaaaagtgtgacacattattgtgtgacataggggagtcatcaccaaatcccacagtggaagcaggacgaacactcagagtccttaacttagtagaaaattggttgaacaacaacactcaattttgcataaaggttctcaacccatatatgccctcagtcatagaaaaaatggaagcactacaaaggaaatatggaggagccttagtgaggaatccactctcacgaaactccacacatgagatgtactgggtatccaatgcttccgggaacatagtgtcatcagtgaacatgatttcaaggatgttgatcaacagatttacaatgagatacaagaaagccacttacgagccggatgttgacctcggaagcggaacccgtaacatcgggattgaaagtgagataccaaacctagatataattgggaaaagaatagaaaaaataaagcaagagcatgaaacatcatggcactatgaccaagaccacccatacaaaacgtgggcataccatggtagctatgaaacaaaacagactggatcagcatcatccatggtcaacggagtggtcaggctgctgacaaaaccttgggacgtcgtccccatggtgacacagatggcaatgacagacacgactccatttggacaacagcgcgtttttaaagagaaagtggacacgagaacccaagaaccgaaagaaggcacgaagaaactaatgaaaataacagcagagtggctttggaaagaattagggaagaaaaagacacccaggatgtgcaccagagaagaattcacaagaaaggtgagaagcaatgcagccttgggggccgtattcactgatgagaacaagtggaagtcggcacgtgaggctgttgaagatagtaggttttgggagctggttgacaaggaaaggaatctccatcttgaaggaaagtgtgaaacatgtgtgtacaacatgatgggaaaaagagagaagaagctaggggaattcggcaaggcaaaaggcagcagagccatatggtacatgtggcttggagcacgcttcttagagtttgaagccctaggattcttaaatgaagatcactggttctccagagagaactccctgagtggagtggaaggagaagggctgcacaagctaggttacattctaagagacgtgagcaagaaagagggaggagcaatgtatgccgatgacaccgcaggatgggatacaagaatcacactagaagacctaaaaaatgaagaaatggtaacaaaccacatggaaggagaacacaagaaactagccgaggccattttcaaactaacgtaccaaaacaaggtggtgcgtgtgcaaagaccaacaccaagaggcacagtaatggacatcatatcgagaagagaccaaagaggtagtggacaagttggcacctatggactcaatactttcaccaatatggaagcccaactaatcagacagatggagggagaaggagtctttaaaagcattcagcacctaacaatcacagaagaaatcgctgtgcaaaactggttagcaagagtggggcgcgaaaggttatcaagaatggccatcagtggagatgattgtgttgtgaaacctttagatgacaggttcgcaagcgctttaacagctctaaatgacatgggaaagattaggaaagacatacaacaatgggaaccttcaagaggatggaatgattggacacaagtgcccttctgttcacaccatttccatgagttaatcatgaaagacggtcgcgtactcgttgttccatgtagaaaccaagatgaactgattggcagagcccgaatctcccaaggagcagggtggtctttgcgggagacggcctgtttggggaagtcttacgcccaaatgtggagcttgatgtacttccacagacgcgacctcaggctggcggcaaatgctatttgctcggcagtaccatcacattgggttccaacaagtcgaacaacctggtccatacatgctaaacatgaatggatgacaacggaagacatgctgacagtctggaacagggtgtggattcaagaaaacccatggatggaagacaaaactccagtggaatcatgggaggaaatcccatacttggggaaaagagaagaccaatggtgcggctcattgattgggttaacaagcagggccacctgggcaaagaacatccaagcagcaataaatcaagttagatcccttataggcaatgaagaatacacagattacatgccatccatgaaaagattcagaagagaagaggaagaagcaggagttctgtggtagaaagcaaaactaacatgaaacaaggctagaagtcaggtcggattaagccatagtacggaaaaaactatgctacctgtgagccccgtccaaggacgttaaaagaagtcaggccatcataaatgccatagcttgagtaaactatgcagcctgtagctccacctgagaaggtgtaaaaaatccgggaggccacaaaccatggaagctgtacgcatggcgtagtggactagcggttagaggagacccctcccttacaaatcgcagcaacaatgggggcccaaggcgagatgaagctgtagtctcgctggaaggactagaggttagaggagacccccccgaaacaaaaaacagcatattgacgctgggaaagaccagagatcctgctgtctcctcagcatcattccaggcacagaacgccagaaaatggaatggtgctgttgaatcaacaggttct

SEQ ID NO: 4 - protein, 3391 aa, synthetic construct
  source 1..3391: note = dengue seroytpe 2
MNNQRKKAKNTPFNMLKRERNRVSTVQQLTKRFSLGMLQGRGPLKLFMALVAFLRFLTIPPTAGILKRWGTIKKSKAINVLRGFRKEIGRMLNILNRRRRSAGMIIMLIPTVMAFHLTTRNGEPHMIVSRQEKGKSLLFKTEVGVNMCTLMAMDLGELCEDTITYECPLLRQNEPEDIDCWCNSTSTWVTYGTCTTMGEHRREKRSVALVPHVGMGLETRTETWMSSEGAWKHVQRIETWILRHPGFTMMAAILAYTIGTTHFQRALIFILLTAVTPSMTMRCIGMSNRDFVEGVSGGSWVDIVLEHGSCVTTMAKNKPTLDFELIKTEAKQPATLRKYCIEAKLTNTTTESRCPTQGEPSLNEEQDKRFVCKHSMVDRGWGNGCGLFGKGGIVTCAMFRCKKNMEGKVVQPENLEYTIVITPHSGEEHAVGNDTGKHGKEIKITPQSSITEAELTGYGTVTMECSPRTGLDFNEMVLLQMENKAWLVHRQWFLDLPLPWLPGADTQGSNWIQKETLVTFKNPHAKKQDVVVLGSQEGAMHTALTGATEIQMSSGNLLFTGHLKCRLRMDKLQLKGMSYSMCTGKFKVVKEIAETQHGTIVIRVQYEGDGSPCKIPFEIMDLEKRHVLGRLITVNPIVTEKDSPVNIEAEPPFGDSYIIIGVEPGQLKLNWFKKGSSIGQMFETTMRGAKRMAILGDTAWDFGSLGGVFTSIGKALHQVFGAIYGAAFSGVSWTMKILIGVIITWIGMNSRSTSLSVTLVLVGIVTLYLGVMVQADSGCVVSWKNKELKCGSGIFITDNVHTWTEQYKFQPESPSKLASAIQKAHEEDICGIRSVTRLENLMWKQITPELNHILSENEVKLTIMTGDIKGIMQAGKRSLRPQPTELKYSWKTWGKAKMLSTESHNQTFLIDGPETAECPNTNRAWNSLEVEDYGFGVFTTNIWLKLKEKQDVFCDSKLMSAAIKDNRAVHADMGYWIESALNDTWKIEKASFIEVKNCHWPKSHTLWSNGVLESEMIIPKNLAGPVSQHNYRPGYHTQITGPWHLGKLEMDFDFCDGTTVVVTEDCGNRGPSLRTTTASGKLITEWCCRSCTLPPLRYRGEDGCWYGMEIRPLKEKEENLVNSLVTAGHGQVDNFSLGVLGMALFLEEMLRTRVGTKHAILLVAVSFVTLITGNMSFRDLGRVMVMVGATMTDDIGMGVTYLALLAAFKVRPTFAAGLLLRKLTSKELMMTTIGIVLLSQSTIPETILELTDALALGMMVLKMVRNMEKYQLAVTIMAILCVPNAVILQNAWKVSCTILAVVSVSPLFLTSSQQKTDWIPLALTIKGLNPTAIFLTTLSRTSKKRSWPLNEAIMAVGMVSILASSLLKNDIPMTGPLVAGGLLTVCYVLTGRSADLELERAADVKWEDQAEISGSSPILSITISEDGSMSIKNEEEEQTLTILIRTGLLVISGLFPVSIPITAAAWYLWEVKKQRAGVLWDVPSPPPMGKAELEDGAYRIKQKGILGYSQIGAGVYKEGTFHTMWHVTRGAVLMHKGKRIEPSWADVKKDLISYGGGWKLEGEWKEGEEVQVLALEPGKNPRAVQTKPGLFKTNAGTIGAVSLDFSPGTSGSPIIDKKGKVVGLYGNGVVTRSGAYVSAIAQTEKSIEDNPEIEDDIFRKRRLTIMDLHPGAGKTKRYLPAIVREAIKRGLRTLILAPTRVVAAEMEEALRGLPIRYQTPAIRAVHTGREIVDLMCHATFTMRLLSPVRVPNYNLIIMDEAHFTDPASIAARGYISTRVEMGEAAGIFMTATPPGSRDPFPQSNAPIIDEEREIPERSWNSGHEWVTDFKGKTVWFVPSIKAGNDIAACLRKNGKKVIQLSRKTFDSEYVKTRTNDWDFVVTTDISEMGANFKAERVIDPRRCMKPVILTDGEERVILAGPMPVTHSSAAQRRGRIGRNPKNENDQYIYMGEPLENDEDCAHWKEAKMLLDNINTPEGIIPSMFEPEREKVDAIDGEYRLRGEARKTFVDLMRRGDLPVWLAYRVAAEGINYADRRWCFDGVKNNQILEENVEVEIWTKEGERKKLKPRWLDARIYSDPLALKEFKEFAAGRKSLTLNLITEMGRLPTFMTQKARDALDNLAVLHTAEAGGRAYNHALSELPETLETLLLLTLLATVTGGIFLFLMSARGIGKMTLGMCCIITASILLWYAQIQPHWIAASIILEFFLIVLLIPEPEKQRTPQDNQLTYVVIAILTVVAATMANEMGFLEKTKKDLGLGSIATQQPESNILDIDLRPASAWTLYAVATTFVTPMLRHSIENSSVNVSLTAIANQATVLMGLGKGWPLSKMDIGVPLLAIGCYSQVNPITLTAALFLLVAHYAIIGPGLQAKATREAQKRAAAGIMKNPTVDGITVIDLDPIPYDPKFEKQLGQVMLLVLCVTQVLMMRTTWALCEALTLATGPISTLWEGNPGRFWNTTIAVSMANIFRGSYLAGAGLLFSIMKNTTNTRRGTGNIGETLGEKWKSRLNALGKSEFQIYKKSGIQEVDRTLAKEGIKRGETDHHAVSRGSAKLRWFVERNMVTPEGKVVDLGCGRGGWSYYCGGLKNVREVKGLTKGGPGHEEPIPMSTYGWNLVRLQSGVDVFFIPPEKCDTLLCDIGESSPNPTVEAGRTLRVLNLVENWLNNNTQFCIKVLNPYMPSVIEKMEALQRKYGGALVRNPLSRNSTHEMYWVSNASGNIVSSVNMISRMLINRFTMRYKKATYEPDVDLGSGTRNIGIESEIPNLDIIGKRIEKIKQEHETSWHYDQDHPYKTWAYHGSYETKQTGSASSMVNGVVRLLTKPWDVVPMVTQMAMTDTTPFGQQRVFKEKVDTRTQEPKEGTKKLMKITAEWLWKELGKKKTPRMCTREEFTRKVRSNAALGAVFTDENKWKSAREAVEDSRFWELVDKERNLHLEGKCETCVYNMMGKREKKLGEFGKAKGSRAIWYMWLGARFLEFEALGFLNEDHWFSRENSLSGVEGEGLHKLGYILRDVSKKEGGAMYADDTAGWDTRITLEDLKNEEMVTNHMEGEHKKLAEAIFKLTYQNKVVRVQRPTPRGTVMDIISRRDQRGSGQVGTYGLNTFTNMEAQLIRQMEGEGVFKSIQHLTITEEIAVQNWLARVGRERLSRMAISGDDCVVKPLDDRFASALTALNDMGKIRKDIQQWEPSRGWNDWTQVPFCSHHFHELIMKDGRVLVVPCRNQDELIGRARISQGAGWSLRETACLGKSYAQMWSLMYFHRRDLRLAANAICSAVPSHWVPTSRTTWSIHAKHEWMTTEDMLTVWNRVWIQENPWMEDKTPVESWEEIPYLGKREDQWCGSLIGLTSRATWAKNIQAAINQVRSLIGNEEYTDYMPSMKRFRREEEEAGVLW

SEQ ID NO: 5 - DNA, 10717 bp, synthetic construct
  source 1..10717: mol_type = other DNA; note = chimeric dengue serotype 2/3
agttgttagtctacgtggaccgacaaagacagattctttgagggagctaagctcaatgtagttctaacagttttttaattagagagcagatctctgatgaataaccaacggaaaaaggcgaaaaacacgcctttcaatatgctgaaacgcgagagaaaccgcgtgtcgactgtgcaacagctgacaaagagattctcacttggaatgctgcagggacgaggaccattaaaactgttcatggccctggtggcgttccttcgtttcctaacaatcccaccaacagcagggatattgaagagatggggaacaattaaaaaatcaaaagctattaatgttttgagagggttcaggaaagagattggaaggatgctgaacatcttgaataggagacgcagatctgcaggcatgatcattatgctgattccaacagtgatggcgttccatttaaccacgcgtgatggagagccgcgcatgattgtggggaagaatgaaagaggaaaatccctacttttcaagacagcctctggaatcaacatgtgcacactcatagctatggatctgggagagatgtgtgatgacacggtcacttacaaatgcccccacattaccgaagtggagcctgaagacattgactgctggtgcaaccttacatcgacatgggtgacttatggaacatgcaatcaagctggagagcatagacgcgataagagatcagtggcgttagctccccatgttggcatgggactggacacacgcactcaaacctggatgtcggctgaaggagcttggagacaagtcgagaaggtagagacatgggcccttaggcacccagggtttaccatactagccctatttcttgcccattacataggcacttccttgacccagaaagtggttatttttatactattaatgctggttaccccatccatgacaatgagatgtgtaggagtaggaaacagagattttgtggaaggcctatcgggagctacgtgggttgacgtggtgctcgagcacggtgggtgtgtgactaccatggctaagaacaagcccacgctggacatagagcttcagaagaccgaggccacccaactggcgaccctaaggaagctatgcattgagggaaaaattaccaacataacaaccgactcaagatgtcccacccaaggggaagcgattttacctgaggagcaggaccagaactacgtgtgtaagcatacatacgtggacagaggctggggaaacggttgtggtttgtttggcaagggaagcttggtgacatgcgcgaaatttcaatgtttagaatcaatagagggaaaagtggtgcaacatgagaacctcaaatacaccgtcatcatcacagtgcacacaggagaccaacaccaggtgggaaatgaaacgcagggagtcacggctgagataacaccccaggcatcaaccgctgaagccattttacctgaatatggaaccctcgggctagaatgctcaccacggacaggtttggatttcaatgaaatgatctcattgacaatgaagaacaaagcatggatggtacatagacaatggttctttgacttacccctaccatggacatcaggagcttcagcagaaacaccaacttggaacaggaaagagcttcttgtgacatttaaaaatgcacatgcaaaaaagcaagaagtagttgttcttggatcacaagagggagcaatgcatacagcactgacaggagctacagagatccaaacctcaggaggcacaagtatctttgcggggcacttaaaatgtagactcaagatggacaaattggaactcaaggggatgagctatgcaatgtgcttgagtagctttgtgttgaagaaagaagtctccgaaacgcagcatgggacaatactcattaaggttgagtacaaaggggaagatgcaccctgcaagattcctttctccacggaggatggacaaggaaaagctctcaatggcagactgatcacagccaatccagtggtgaccaagaaggaggagcctgtcaacattgaggctgaacctccttttggagaaagtaacatagtaattggaattggagacaaagccctgaaaatcaactggtacaagaagggaagctcgattgggaagatgttcgaggccactgccagaggtgcaaggcgcatggccatcttgggagacacagcctgggactttggatcagtgggtggtgttttgaattcattagggaaaatggtccaccaaatatttgggagtgcttacacagccctatttggtggagtctcctggatgatgaaaattggaataggtgtcctcttaacctggatagggttgaactcaaaaaatacttctatgtcattttcatgcatcgcggccggcattgtgacactgtatttgggagtcatggtgcaggccgatagtggttgcgttgtgagctggaaaaacaaagaactgaaatgtggcagtgggattttcatcacagacaacgtgcacacatggacagaacaatacaagttccaaccagaatccccttcaaaactagcttcagctatccagaaagcccatgaagaggacatttgtggaatccgctcagtaacaagactggagaatctgatgtggaaacaaataacaccagaattgaatcacattctatcagaaaatgaggtgaagttaactattatgacaggagacatcaaaggaatcatgcaggcaggaaaacgatctctgcggcctcagcccactgagctgaagtattcatggaaaacatggggcaaagcaaaaatgctctctacagagtctcataaccagacctttctcattgatggccccgaaacagcagaatgccccaacacaaatagagcttggaattcgttggaagttgaagactatggctttggagtattcaccaccaatatatggctaaaattgaaagaaaaacaggatgtattctgcgactcaaaactcatgtcagcggccataaaagacaacagagccgtccatgccgatatgggttattggatagaaagtgcactcaatgacacatggaagatagagaaagcctctttcattgaagttaaaaactgccactggccaaaatcacacaccctctggagcaatggagtgctagaaagtgagatgataattccaaagaatctcgctggaccagtgtctcaacacaactatagaccaggctaccatacacaaataacaggaccatggcatctaggtaagcttgagatggactttgatttctgtgatggaacaacagtggtagtgactgaggactgcggaaatagaggaccctctttgagaacaaccactgcctctggaaaactcataacagaatggtgctgccgatcttgcacattaccaccgctaagatacagaggtgaggatgggtgctggtacgggatggaaatcagaccattgaaggagaaagaagagaatttggtcaactccttggtcacagctggacatgggcaggtcgacaacttttcactaggagtcttgggaatggcattgttcctggaggaaatgcttaggacccgagtaggaacgaaacatgcaatactactagttgcagtttcttttgtgacattgatcacagggaacatgtcctttagagacctgggaagagtgatggttatggtaggcgccactatgacggatgacataggtatgggcgtgacttatcttgccctactagcagccttcaaagtcagaccaacttttgcagctggactactcttgagaaagctgacctccaaggaattgatgatgactactataggaattgtactcctctcccagagcaccataccagagaccattcttgagttgactgatgcgttagccttaggcatgatggtcctcaaaatggtgagaaatatggaaaagtatcaattggcagtgactatcatggctatcttgtgcgtcccaaacgcagtgatattacaaaacgcatggaaagtgagttgcacaatattggcagtggtgtccgtttccccactgttcttaacatcctcacagcaaaaaacagattggataccattagcattgacgatcaaaggtctcaatccaacagctatttttctaacaaccctctcaagaaccagcaagaaaaggagctggccattaaatgaggctatcatggcagtcgggatggtgagcattttagccagttctctcctaaaaaatgatattcccatgacaggaccattagtggctggagggctcctcactgtgtgctacgtgctcactggacgatcggccgatttggaactggagagagcagccgatgtcaaatgggaagaccaggcagagatatcaggaagcagtccaatcctgtcaataacaatatcagaagatggtagcatgtcgataaaaaatgaagaggaagaacaaacactgaccatactcattagaacaggattgctggtgatctcaggactttttcctgtatcaataccaatcacggcagcagcatggtacctgtgggaagtgaagaaacaacgggccggagtattgtgggatgttccttcacccccacccatgggaaaggctgaactggaagatggagcctatagaattaagcaaaaagggattcttggatattcccagatcggagccggagtttacaaagaaggaacattccatacaatgtggcatgtcacacgtggcgctgttctaatgcataaaggaaagaggattgaaccatcatgggcggacgtcaagaaagacctaatatcatatggaggaggctggaagttagaaggagaatggaaggaaggagaagaagtccaggtattggcactggagcctggaaaaaatccaagagccgtccaaacgaaacctggtcttttcaaaaccaacgccggaacaataggtgctgtatctctggacttttctcctggaacgtcaggatctccaattatcgacaaaaaaggaaaagttgtgggtctttatggtaatggtgttgttacaaggagtggagcatatgtgagtgctatagcccagactgaaaaaagcattgaagacaacccagagatcgaagatgacattttccgaaagagaagactgaccatcatggacctccacccaggagcgggaaagacgaagagataccttccggccatagtcagagaagctataaaacggggtttgagaacattaatcttggcccccactagagttgtggcagctgaaatggaggaagcccttagaggacttccaataagataccagaccccagccatcagagctgtgcacaccgggcgggagattgtggacctaatgtgtcatgccacatttaccatgaggctgctatcaccagttagagtgccaaactacaacctgattatcatggacgaagcccatttcacagacccagcaagtatagcagctagaggatacatctcaactcgagtggagatgggtgaggcagctgggatttttatgacagccactcccccgggaagcagagacccatttcctcagagcaatgcaccaatcatagatgaagaaagagaaatccctgaacgctcgtggaattccggacatgaatgggtcacggattttaaagggaagactgtttggttcgttccaagtataaaagcaggaaatgatatagcagcttgcctgaggaaaaatggaaagaaagtgatacaactcagtaggaagacctttgattctgagtatgtcaagactagaaccaatgattgggacttcgtggttacaactgacatttcagaaatgggtgccaatttcaaggctgagagggttatagaccccagacgctgcatgaaaccagtcatactaacagatggtgaagagcgggtgattctggcaggacctatgccagtgacccactctagtgcagcacaaagaagagggagaataggaagaaatccaaaaaatgagaatgaccagtacatatacatgggggaacctctggaaaatgatgaagactgtgcacactggaaagaagctaaaatgctcctagataacatcaacacgccagaaggaatcattcctagcatgttcgaaccagagcgtgaaaaggtggatgccattgatggcgaataccgcttgagaggagaagcaaggaaaacctttgtagacttaatgagaagaggagacctaccagtctggttggcctacagagtggcagctgaaggcatcaactacgcagacagaaggtggtgttttgatggagtcaagaacaaccaaatcctagaagaaaacgtggaagttgaaatctggacaaaagaaggggaaaggaagaaattgaaacccagatggttggatgctaggatctattctgacccactggcgctaaaagaatttaaggaatttgcagccggaagaaagtctctgaccctgaacctaatcacagaaatgggtaggctcccaaccttcatgactcagaaggcaagagacgcactggacaacttagcagtgctgcacacggctgaggcaggtggaagggcgtacaaccatgctctcagtgaactgccggagaccctggagacattgcttttactgacacttctggctacagtcacgggagggatctttttattcttgatgagcgcaaggggcatagggaagatgaccctgggaatgtgctgcataatcacggctagcatcctcctatggtacgcacaaatacagccacactggatagcagcttcaataatactggagttttttctcatagttttgcttattccagaacctgaaaaacagagaacaccccaagacaaccaactgacctacgttgtcatagccatcctcacagtggtggccgcaaccatggcaaacgagatgggtttcctagaaaaaacgaagaaagatctcggattgggaagcattgcaacccagcaacccgagagcaacatcctggacatagatctacgtcctgcatcagcatggacgctgtatgccgtggccacaacatttgttacaccaatgttgagacatagcattgaaaattcctcagtgaatgtgtccctaacagctatagccaaccaagccacagtgttaatgggtctcgggaaaggatggccattgtcaaagatggacatcggagttccccttctcgccattggatgctactcacaagtcaaccccataactctcacagcagctcttttcttattggtagcacattatgccatcatagggccaggactccaagcaaaagcaaccagagaagctcagaaaagagcagcggcgggcatcatgaaaaacccaactgtcgatggaataacagtgattgacctagatccaataccttatgatccaaagtttgaaaagcagttgggacaagtaatgctcctagtcctctgcgtgactcaagtattgatgatgaggactacatgggctctgtgtgaggctttaaccttagctaccgggcccatctccacattgtgggaaggaaatccagggaggttttggaacactaccattgcggtgtcaatggctaacatttttagagggagttacttggccggagctggacttctcttttctattatgaagaacacaaccaacacaagaaggggaactggcaacataggagagacgcttggagagaaatggaaaagccgattgaacgcgttgggaaaaagtgaattccagatctacaagaaaagtggaatccaggaagtggatagaaccttagcaaaagaaggcattaaaagaggagaaacggaccatcacgctgtgtcgcgaggctcagcaaaactgagatggttcgttgagagaaacatggtcacaccagaagggaaagtagtggacctcggttgtggcagaggaggctggtcatactattgtggaggactaaagaatgtaagagaagtcaaaggcctaacaaaaggaggaccaggacacgaagaacccatccccatgtcaacatatgggtggaatctagtgcgtcttcaaagtggagttgacgttttcttcatcccgccagaaaagtgtgacacattattgtgtgacataggggagtcatcaccaaatcccacagtggaagcaggacgaacactcagagtccttaacttagtagaaaattggttgaacaacaacactcaattttgcataaaggttctcaacccatatatgccctcagtcatagaaaaaatggaagcactacaaaggaaatatggaggagccttagtgaggaatccactctcacgaaactccacacatgagatgtactgggtatccaatgcttccgggaacatagtgtcatcagtgaacatgatttcaaggatgttgatcaacagatttacaatgagatacaagaaagccacttacgagccggatgttgacctcggaagcggaacccgtaacatcgggattgaaagtgagataccaaacctagatataattgggaaaagaatagaaaaaataaagcaagagcatgaaacatcatggcactatgaccaagaccacccatacaaaacgtgggcataccatggtagctatgaaacaaaacagactggatcagcatcatccatggtcaacggagtggtcaggctgctgacaaaaccttgggacgtcgtccccatggtgacacagatggcaatgacagacacgactccatttggacaacagcgcgtttttaaagagaaagtggacacgagaacccaagaaccgaaagaaggcacgaagaaactaatgaaaataacagcagagtggctttggaaagaattagggaagaaaaagacacccaggatgtgcaccagagaagaattcacaagaaaggtgagaagcaatgcagccttgggggccatattcactgatgagaacaagtggaagtcggcacgtgaggctgttgaagatagtaggttttgggagctggttgacaaggaaaggaatctccatcttgaaggaaagtgtgaaacatgtgtgtacaacatgatgggaaaaagagagaagaagctaggggaattcggcaaggcaaaaggcagcagagccatatggtacatgtggcttggagcacgcttcttagagtttgaagccctaggattcttaaatgaagatcactggttctccagagagaactccctgagtggagtggaaggagaagggctgcacaagctaggttacattctaagagacgtgagcaagaaagagggaggagcaatgtatgccgatgacaccgcaggatgggatacaagaatcacactagaagacctaaaaaatgaagaaatggtaacaaaccacatggaaggagaacacaagaaactagccgaggccattttcaaactaacgtaccaaaacaaggtggtgcgtgtgcaaagaccaacaccaagaggcacagtaatggacatcatatcgagaagagaccaaagaggtagtggacaagttggcacctatggactcaatactttcaccaatatggaagcccaactaatcagacagatggagggagaaggagtctttaaaagcattcagcacctaacaatcacagaagaaatcgctgtgcaaaactggttagcaagagtggggcgcgaaaggttatcaagaatggccatcagtggagatgattgtgttgtgaaacctttagatgacaggttcgcaagcgctttaacagctctaaatgacatgggaaagattaggaaagacatacaacaatgggaaccttcaagaggatggaatgattggacacaagtgcccttctgttcacaccatttccatgagttaatcatgaaagacggtcgcgtactcgttgttccatgtagaaaccaagatgaactgattggcagagcccgaatctcccaaggagcagggtggtctttgcgggagacggcctgtttggggaagtcttacgcccaaatgtggagcttgatgtacttccacagacgcgacctcaggctggcggcaaatgctatttgctcggcagtaccatcacattgggttccaacaagtcgaacaacctggtccatacatgctaaacatgaatggatgacaacggaagacatgctgacagtctggaacagggtgtggattcaagaaaacccatggatggaagacaaaactccagtggaatcatgggaggaaatcccatacttggggaaaagagaagaccaatggtgcggctcattgattgggttaacaagcagggccacctgggcaaagaacatccaagcagcaataaatcaagttagatcccttataggcaatgaagaatacacagattacatgccatccatgaaaagattcagaagagaagaggaagaagcaggagttctgtggtagaaagcaaaactaacatgaaacaaggctagaagtcaggtcggattaagccatagtacggaaaaaactatgctacctgtgagccccgtccaaggacgttaaaagaagtcaggccatcataaatgccatagcttgagtaaactatgcagcctgtagctccacctgagaaggtgtaaaaaatccgggaggccacaaaccatggaagctgtacgcatggcgtagtggactagcggttagaggagacccctcccttacaaatcgcagcaacaatgggggcccaaggcgagatgaagctgtagtctcgctggaaggactagaggttagaggagacccccccgaaacaaaaaacagcatattgacgctgggaaagaccagagatcctgctgtctcctcagcatcattccaggcacagaacgccagaaaatggaatggtgctgttgaatcaacaggttct

SEQ ID NO: 6 - protein, 3389 aa, synthetic construct
  source 1..3389: note = chimeric dengue serotype 2/3
MNNQRKKAKNTPFNMLKRERNRVSTVQQLTKRFSLGMLQGRGPLKLFMALVAFLRFLTIPPTAGILKRWGTIKKSKAINVLRGFRKEIGRMLNILNRRRRSAGMIIMLIPTVMAFHLTTRDGEPRMIVGKNERGKSLLFKTASGINMCTLIAMDLGEMCDDTVTYKCPHITEVEPEDIDCWCNLTSTWVTYGTCNQAGEHRRDKRSVALAPHVGMGLDTRTQTWMSAEGAWRQVEKVETWALRHPGFTILALFLAHYIGTSLTQKVVIFILLMLVTPSMTMRCVGVGNRDFVEGLSGATWVDVVLEHGGCVTTMAKNKPTLDIELQKTEATQLATLRKLCIEGKITNITTDSRCPTQGEAILPEEQDQNYVCKHTYVDRGWGNGCGLFGKGSLVTCAKFQCLESIEGKVVQHENLKYTVIITVHTGDQHQVGNETQGVTAEITPQASTAEAILPEYGTLGLECSPRTGLDFNEMISLTMKNKAWMVHRQWFFDLPLPWTSGASAETPTWNRKELLVTFKNAHAKKQEVVVLGSQEGAMHTALTGATEIQTSGGTSIFAGHLKCRLKMDKLELKGMSYAMCLSSFVLKKEVSETQHGTILIKVEYKGEDAPCKIPFSTEDGQGKALNGRLITANPVVTKKEEPVNIEAEPPFGESNIVIGIGDKALKINWYKKGSSIGKMFEATARGARRMAILGDTAWDFGSVGGVLNSLGKMVHQIFGSAYTALFGGVSWMMKIGIGVLLTWIGLNSKNTSMSFSCIAAGIVTLYLGVMVQADSGCVVSWKNKELKCGSGIFITDNVHTWTEQYKFQPESPSKLASAIQKAHEEDICGIRSVTRLENLMWKQITPELNHILSENEVKLTIMTGDIKGIMQAGKRSLRPQPTELKYSWKTWGKAKMLSTESHNQTFLIDGPETAECPNTNRAWNSLEVEDYGFGVFTTNIWLKLKEKQDVFCDSKLMSAAIKDNRAVHADMGYWIESALNDTWKIEKASFIEVKNCHWPKSHTLWSNGVLESEMIIPKNLAGPVSQHNYRPGYHTQITGPWHLGKLEMDFDFCDGTTVVVTEDCGNRGPSLRTTTASGKLITEWCCRSCTLPPLRYRGEDGCWYGMEIRPLKEKEENLVNSLVTAGHGQVDNFSLGVLGMALFLEEMLRTRVGTKHAILLVAVSFVTLITGNMSFRDLGRVMVMVGATMTDDIGMGVTYLALLAAFKVRPTFAAGLLLRKLTSKELMMTTIGIVLLSQSTIPETILELTDALALGMMVLKMVRNMEKYQLAVTIMAILCVPNAVILQNAWKVSCTILAVVSVSPLFLTSSQQKTDWIPLALTIKGLNPTAIFLTTLSRTSKKRSWPLNEAIMAVGMVSILASSLLKNDIPMTGPLVAGGLLTVCYVLTGRSADLELERAADVKWEDQAEISGSSPILSITISEDGSMSIKNEEEEQTLTILIRTGLLVISGLFPVSIPITAAAWYLWEVKKQRAGVLWDVPSPPPMGKAELEDGAYRIKQKGILGYSQIGAGVYKEGTFHTMWHVTRGAVLMHKGKRIEPSWADVKKDLISYGGGWKLEGEWKEGEEVQVLALEPGKNPRAVQTKPGLFKTNAGTIGAVSLDFSPGTSGSPIIDKKGKVVGLYGNGVVTRSGAYVSAIAQTEKSIEDNPEIEDDIFRKRRLTIMDLHPGAGKTKRYLPAIVREAIKRGLRTLILAPTRVVAAEMEEALRGLPIRYQTPAIRAVHTGREIVDLMCHATFTMRLLSPVRVPNYNLIIMDEAHFTDPASIAARGYISTRVEMGEAAGIFMTATPPGSRDPFPQSNAPIIDEEREIPERSWNSGHEWVTDFKGKTVWFVPSIKAGNDIAACLRKNGKKVIQLSRKTFDSEYVKTRTNDWDFVVTTDISEMGANFKAERVIDPRRCMKPVILTDGEERVILAGPMPVTHSSAAQRRGRIGRNPKNENDQYIYMGEPLENDEDCAHWKEAKMLLDNINTPEGIIPSMFEPEREKVDAIDGEYRLRGEARKTFVDLMRRGDLPVWLAYRVAAEGINYADRRWCFDGVKNNQILEENVEVEIWTKEGERKKLKPRWLDARIYSDPLALKEFKEFAAGRKSLTLNLITEMGRLPTFMTQKARDALDNLAVLHTAEAGGRAYNHALSELPETLETLLLLTLLATVTGGIFLFLMSARGIGKMTLGMCCIITASILLWYAQIQPHWIAASIILEFFLIVLLIPEPEKQRTPQDNQLTYVVIAILTVVAATMANEMGFLEKTKKDLGLGSIATQQPESNILDIDLRPASAWTLYAVATTFVTPMLRHSIENSSVNVSLTAIANQATVLMGLGKGWPLSKMDIGVPLLAIGCYSQVNPITLTAALFLLVAHYAIIGPGLQAKATREAQKRAAAGIMKNPTVDGITVIDLDPIPYDPKFEKQLGQVMLLVLCVTQVLMMRTTWALCEALTLATGPISTLWEGNPGRFWNTTIAVSMANIFRGSYLAGAGLLFSIMKNTTNTRRGTGNIGETLGEKWKSRLNALGKSEFQIYKKSGIQEVDRTLAKEGIKRGETDHHAVSRGSAKLRWFVERNMVTPEGKVVDLGCGRGGWSYYCGGLKNVREVKGLTKGGPGHEEPIPMSTYGWNLVRLQSGVDVFFIPPEKCDTLLCDIGESSPNPTVEAGRTLRVLNLVENWLNNNTQFCIKVLNPYMPSVIEKMEALQRKYGGALVRNPLSRNSTHEMYWVSNASGNIVSSVNMISRMLINRFTMRYKKATYEPDVDLGSGTRNIGIESEIPNLDIIGKRIEKIKQEHETSWHYDQDHPYKTWAYHGSYETKQTGSASSMVNGVVRLLTKPWDVVPMVTQMAMTDTTPFGQQRVFKEKVDTRTQEPKEGTKKLMKITAEWLWKELGKKKTPRMCTREEFTRKVRSNAALGAIFTDENKWKSAREAVEDSRFWELVDKERNLHLEGKCETCVYNMMGKREKKLGEFGKAKGSRAIWYMWLGARFLEFEALGFLNEDHWFSRENSLSGVEGEGLHKLGYILRDVSKKEGGAMYADDTAGWDTRITLEDLKNEEMVTNHMEGEHKKLAEAIFKLTYQNKVVRVQRPTPRGTVMDIISRRDQRGSGQVGTYGLNTFTNMEAQLIRQMEGEGVFKSIQHLTITEEIAVQNWLARVGRERLSRMAISGDDCVVKPLDDRFASALTALNDMGKIRKDIQQWEPSRGWNDWTQVPFCSHHFHELIMKDGRVLVVPCRNQDELIGRARISQGAGWSLRETACLGKSYAQMWSLMYFHRRDLRLAANAICSAVPSHWVPTSRTTWSIHAKHEWMTTEDMLTVWNRVWIQENPWMEDKTPVESWEEIPYLGKREDQWCGSLIGLTSRATWAKNIQAAINQVRSLIGNEEYTDYMPSMKRFRREEEEAGVLW

SEQ ID NO: 7 - DNA, 10723 bp, synthetic construct
  source 1..10723: mol_type = other DNA; note = chimeric dengue seroytpe 2/4
agttgttagtctacgtggaccgacaaagacagattctttgagggagctaagctcaatgtagttctaacagttttttaattagagagcagatctctgatgaataaccaacggaaaaaggcgaaaaacacgcctttcaatatgctgaaacgcgagagaaaccgcgtgtcgactgtgcaacagctgacaaagagattctcacttggaatgctgcagggacgaggacctttaaaactgttcatggccctggtggcgttccttcgtttcctaacaatcccaccaacagcagggatattgaagagatggggaacaattaaaaaatcaaaagctattaatgttttgagagggttcaggaaagagattggaaggatgctgaacatcttgaataggagacgcagctctgcaggcatgatcattatgctgattccaacagtgatggcgttccatttaaccacgcgtgatggcgaacccctcatgatagtggcaaaacatgaaagggggagacctctcttgtttaagacaacagaggggatcaacaaatgcactctcattgccatggacttgggtgaaatgtgtgaggacactgtcacgtataaatgccccttactggtcaataccgaacctgaagacattgattgctggtgcaatctcacgtctacctgggtcatgtatgggacatgcacccagagcggagaacggagacgagagaagcgctcagtagctttaacaccacattcaggaatgggattggaaacaagagctgagacatggatgtcatcggaaggggcttggaagcatgctcagagagtagagagctggatactcagaaacccaggattcgcgctcttggcaggatttatggcttatatgattgggcaaacaggaatccagcgaactgtcttctttgtcctaatgatgctggtcgccccatcctacggaatgcgatgcgtaggagtaggaaacagagactttgtggaaggagtctcaggtggagcatgggtcgatctggtgctagaacatggaggatgcgtcacaaccatggcccagggaaaaccaaccttggattttgaactgactaagacaacagccaaggaagtggctctgttaagaacctattgcattgaagcctcaatatcaaacataaccacggcaacaagatgtccaacgcaaggagagccttatctaaaagaggaacaagaccaacagtacatttgccggagagatgtggtagacagagggtggggcaatggctgtggcttgtttggaaaaggaggagttgtgacatgtgcgaagttttcatgttcggggaagataacaggcaatttggtccaaattgagaaccttgaatacacagtggttgtaacagtccacaatggagacacccatgcagtaggaaatgacacgtccaatcatggagttacagccacgataactcccaggtcaccatcggtggaagtcaaattgccggactatggagaactaacactcgattgtgaacccaggtctggaattgactttaatgagatgattctgatgaaaatgaaaaagaaaacatggcttgtgcataagcaatggtttttggatctacctctaccatggacagcaggagcagacacatcagaggttcactggaattacaaagagagaatggtgacatttaaggttcctcatgccaagagacaggatgtgacagtgctgggatctcaggaaggagccatgcattctgccctcgctggagccacagaagtggactccggtgatggaaatcacatgtttgcaggacatctcaagtgcaaagtccgtatggagaaattgagaatcaagggaatgtcatacacgatgtgttcaggaaagttctcaattgacaaagagatggcagaaacacagcatgggacaacagtggtgaaagtcaagtatgaaggtgctggagctccgtgtaaagtccccatagagataagagatgtgaacaaggaaaaagtggttgggcgtatcatctcatccacccctttggctgagaataccaacagtgtaaccaacatagagttagaacccccctttggggacagctacatagtgataggtgttggaaacagtgcattaacactccattggttcaggaaagggagttccattggcaagatgtttgagtccacatacagaggtgcaaaacgaatggccattctaggtgaaacagcttgggattttggttccgttggtggactgttcacatcattgggaaaggctgtgcaccaggtttttggaagtgtgtatacaaccctgtttggaggagtctcatggatgattagaatcctaattgggttcctagtgttgtggattggcacgaactcaaggaacacttcaatggctatgacgtgcatagctgccggcattgtgacactgtatttgggagtcatggtgcaggccgatagtggttgcgttgtgagctggaaaaacaaagaactgaaatgtggcagtgggattttcatcacagacaacgtgcacacatggacagaacaatacaagttccaaccagaatccccttcaaaactagcttcagctatccagaaagcccatgaagaggacatttgtggaatccgctcagtaacaagactggagaatctgatgtggaaacaaataacaccagaattgaatcacattctatcagaaaatgaggtgaagttaactattatgacaggagacatcaaaggaatcatgcaggcaggaaaacgatctctgcggcctcagcccactgagctgaagtattcatggaaaacatggggcaaagcaaaaatgctctctacagagtctcataaccagacctttctcattgatggccccgaaacagcagaatgccccaacacaaatagagcttggaattcgttggaagttgaagactatggctttggagtattcaccaccaatatatggctaaaattgaaagaaaaacaggatgtattctgcgactcaaaactcatgtcagcggccataaaagacaacagagccgtccatgccgatatgggttattggatagaaagtgcactcaatgacacatggaagatagagaaagcctctttcattgaagttaaaaactgccactggccaaaatcacacaccctctggagcaatggagtgctagaaagtgagatgataattccaaagaatctcgctggaccagtgtctcaacacaactatagaccaggctaccatacacaaataacaggaccatggcatctaggtaagcttgagatggactttgatttctgtgatggaacaacagtggtagtgactgaggactgcggaaatagaggaccctctttgagaacaaccactgcctctggaaaactcataacagaatggtgctgccgatcttgcacattaccaccgctaagatacagaggtgaggatgggtgctggtacgggatggaaatcagaccattgaaggagaaagaagagaatttggtcaactccttggtcacagctggacatgggcaggtcgacaacttttcactaggagtcttgggaatggcattgttcctggaggaaatgcttaggacccgagtaggaacgaaacatgcaatactactagttgcagtttcttttgtgacattgatcacagggaacatgtcctttagagacctgggaagagtgatggttatggtaggcgccactatgacgggtgacataggtatgggcgtgacttatcttgccctactagcagccttcaaagtcagaccaacttttgcagctggactactcttgagaaagctgacctccarggaattgatgatgactactataggaattgtactcctctcccagagcaccataccagagaccattcttgagttgactgatgcgttagccttaggcatgatggtcctcaaaatggtgagaaatatggaaaagtatcaattggcagtgactatcatggctatcttgtgcgtcccaaacgcagtgatattacaaaacgcatggaaagtgagttgcacaatattggcagtggtgtccgtttccccactgttcttaacatcctcacagcaaaaaacagattggataccattagcattgacgatcaaaggtctcaatccaacagctatttttctaacaaccctctcaagaaccagcaagaaaaggagctggccattaaatgaggctatcatggcagtcgggatggtgagcattttagccagttctctcctaaaaaatgatattcccatgacaggaccattagtggctggagggctcctcactgtgtgctacgtgctcactggacgatcggccgatttggaactggagagagcagccgatgtcaaatgggaagaccaggcagagatatcaggaagcagtccaatcctgtcaataacaatatcagaagatggtagcatgtcgataaaaaatgaagaggaagaacaaacactgaccatactcattagaacaggattgctggtgatctcaggactttttcctgtatcaataccaatcacggcagcagcatggtacctgtgggaagtgaagaaacaacgggccggagtattgtgggatgttccttcacccccacccatgggaaaggctgaactggaagatggagcctatagaattaagcaaaaagggattcttggatattcccagatcggagccggagtttacaaagaaggaacattccatacaatgtggcatgtcacacgtggcgctgttctaatgcataaaggaaagaggattgaaccatcatgggcggacgtcaagaaagacctaatatcatatggaggaggctggaagttagaaggagaatggaaggaaggagaagaagtccaggtattggcactggagcctggaaaaaatccaagagccgtccaaacgaaacctggtcttttcaaaaccaacgccggaacaataggtgctgtatctctggacttttctcctggaacgtcaggatctccaattatcgacaaaaaaggaaaagttgtgggtctttatggtaatggtgttgttacaaggagtggagcatatgtgagtgctatagcccagactgaaaaaagcattgaagacaacccagagatcgaagatgacattttccgaaagagaagactgaccatcatggacctccacccaggagcgggaaagacgaagagataccttccggccatagtcagagaagctataaaacggggtttgagaacattaatcttggcccccactagagttgtggcagctgaaatggaggaagcccttagaggacttccaataagataccagaccccagccatcagagctgtgcacaccgggcgggagattgtggacctaatgtgtcatgccacatttaccatgaggctgctatcaccagttagagtgccaaactacaacctgattatcatggacgaagcccatttcacagatccagcaagtatagcagctagaggatacatctcaactcgagtggagatgggtgaggcagctgggatttttatgacagccactcccccgggaagcagagacccatttcctcagagcaatgcaccaatcatagatgaagaaagagaaatccctgaacgctcgtggaattccggacatgaatgggtcacggattttaaagggaagactgtttggttcgttccaagtataaaagcaggaaatgatatagcagcttgcctgaggaaaaatggaaagaaagtgatacaactcagtaggaagacctttgattctgagtatgtcaagactagaaccaatgattgggacttcgtggttacaactgacatttcagaaatgggtgccaatttcaaggctgagagggttatagaccccagacgctgcatgaaaccagtcatactaacagatggtgaagagcgggtgattctggcaggacctatgccagtgacccactctagtgcagcacaaagaagagggagaataggaagaaatccaaaaaatgagaatgaccagtacatatacatgggggaacctctggaaaatgatgaagactgtgcacactggaaagaagctaaaatgctcctagataacatcaacacgccagaaggaatcattcctagcatgttcgaaccagagcgtgaaaaggtggatgccattgatggcgaataccgcttgagaggagaagcaaggaaaacctttgtagacttaatgagaagaggagacctaccagtctggttggcctacagagtggcagctgaaggcatcaactacgcagacagaaggtggtgttttgatggagtcaagaacaaccaaatcctagaagaaaacgtggaagttgaaatctggacaaaagaaggggaaaggaagaaattgaaacccagatggttggatgctaggatctattctgacccactggcgctaaaagaatttaaggaatttgcagccggaagaaagtctctgaccctgaacctaatcacagaaatgggtaggctcccaaccttcatgactcagaaggtaagagacgcactggacaacttagcagtgctgcacacggctgaggcaggtggaagggcgtacaaccatgctctcagtgaactgccggagaccctggagacattgcttttactgacacttctggctacagtcacgggagggatctttttattcttgatgagcgcaaggggcatagggaagatgaccctgggaatgtgctgcataatcacggctagcatcctcctatggtacgcacaaatacagccacactggatagcagcttcaataatactggagttttttctcatagttttgcttattccagaacctgaaaaacagagaacaccccaagacaaccaactgacctacgttgtcatagccatcctcacagtggtggccgcaaccatggcaaacgagatgggtttcctagaaaaaacgaagaaagatctcggattgggaagcattgcaacccagcaacccgagagcaacatcctggacatagatctacgtcctgcatcagcatggacgctgtatgccgtggccacaacatttgttacaccaatgttgagacatagcattgaaaattcctcagtgaatgtgtccctaacagccatagccaaccaagccacagtgttaatgggtctcgggaaaggatggccattgtcaaagatggacatcggagttccccttctcgccattggatgctactcacaagtcaaccccataactctcacagcagctcttttcttattggtagcacattatgccatcatagggccaggactccaagcaaaagcaaccagagaagctcagaaaagagcagcggcgggcatcatgaaaaacccaactgtcgatggaataacagtgattgacctagatccaataccttatgatccaaagtttgaaaagcagttgggacaagtaatgctcctagtcctctgcgtgactcaagtattgatgatgaggactacatgggctctgtgtgaggctttaaccttagctaccgggcccatctccacattgtgggaaggaaatccagggaggttttggaacactaccattgcggtgtcaatggctaacatttttagagggagttacttggccggagctggacttctcttttctattatgaagaacacaaccaacacaagaaggggaactggcaacataggagagacgcttggagagaaatggaaaagccgattgaacgcattgggaaaaagtgaattccagatctacaagaaaagtggaatccaggaagtggatagaaccttagcaaaagaaggcattaaaagaggagaaacggaccatcacgctgtgtcgcgaggctcagcaaaactgagatggttcgttgagagaaacatggtcacaccagaagggaaagtagtggacctcggttgtggcagaggaggctggtcatactattgtggaggactaaagaatgtaagagaagtcaaaggcctaacaaaaggaggaccaggacacgaagaacccatccccatgtcaacatatgggtggaatctagtgcgtcttcaaagtggagttgacgttttcttcatcccgccagaaaagtgtgacacattattgtgtgacataggggagtcatcaccaaatcccacagtggaagcaggacgaacactcagagtccttaacttagtagaaaattggttgaacaacaacactcaattttgcataaaggttctcaacccatatatgccctcagtcatagaaaaaatggaagcactacaaaggaaatatggaggagccttagtgaggaatccactctcacgaaactccacacatgagatgtactgggtatccaatgcttccgggaacatagtgtcatcagtgaacatgatttcaaggatgttgatcaacagatttacaatgagatacaagaaagccacttacgagccggatgttgacctcggaagcggaacccgtaacatcgggattgaaagtgagataccaaacctagatataattgggaaaagaatagaaaaaataaagcaagagcatgaaacatcatggcactatgaccaagaccacccatacaaaacgtgggcataccatggtagctatgaaacaaaacagactggatcagcatcatccatggtcaacggagtggtcaggctgctgacaaaaccttgggacgtcgtccccatggtgacacagatggcaatgacagacacgactccatttggacaacagcgcgtttttaaagagaaagtggacacgagaacccaagaaccgaaagaaggcacgaagaaactaatgaaaataacagcagagtggctttggaaagaattagggaagaaaaagacacccaggatgtgcaccagagaagaattcacaagaaaggtgagaagcaatgcagccttgggggccatattcactgatgagaacaagtggaagtcggcacgtgaggctgttgaagatagtaggttttgggagctggttgacaaggaaaggaatctccatcttgaaggaaagtgtgaaacatgtgtgtacaacatgatgggaaaaagagagaagaagctaggggaattcggcaaggcaaaaggcagcagagccatatggtacatgtggcttggagcacgcttcttagagtttgaagccctaggattcttaaatgaagatcactggttctccagagagaactccctgagtggagtggaaggagaagggctgcacaagctaggttacattctaagagacgtgagcaagaaagagggaggagcaatgtatgccgatgacaccgcaggatgggatacaagaatcacactagaagacctaaaaaatgaagaaatggtaacaaaccacatggaaggagaacacaagaaactagccgaggccattttcaaactaacgtaccaaaacaaggtggtgcgtgtgcaaagaccaacaccaagaggcacagtaatggacatcatatcgagaagagaccaaagaggtagtggacaagttggcacctatggactcaatactttcaccaatatggaagcccaactaatcagacagatggagggagaaggagtctttaaaagcattcagcacctaacaatcacagaagaaatcgctgtgcaaaactggttagcaagagtggggcgcgaaaggttatcaagaatggccatcagtggagatgattgtgttgtgaaacctttagatgacaggttcgcaagcgctttaacagctctaaatgacatgggaaagattaggaaagacatacaacaatgggaaccttcaagaggatggaatgattggacacaagtgcccttctgttcacaccatttccatgagttaatcatgaaagacggtcgcgtactcgttgttccctgtagaaaccaagatgaactgattggcagagcccgaatctcccaaggagcagggtggtctttgcgggagacggcctgtttggggaagtcttacgcccaaatgtggagcttgatgtacttccacagacgcgacctcaggctggcggcaaatgctatttgctcggcagtaccatcacattgggttccaacaagtcgaacaacctggtccatacatgctaaacatgaatggatgacaacggaagacatgctgacagtctggaacagggtgtggattcaagaaaacccatggatggaagacaaaactccagtggaatcatgggaggaaatcccatacttggggaaaagagaagaccaatggtgcggctcattgattgggttaacaagcagggccacctgggcaaagaacatccaagcagcaataaatcaagttagatcccttataggcaatgaagaatacacagattacatgccatccatgaaaagattcagaagagaagaggaagaagcaggagttctgtggtagaaagcaaaactaacatgaaacaaggctagaagtcaggtcggattaagccatagtacggaaaaaactatgctacctgtgagccccgtccaaggacgttaaaagaagtcaggccatcataaatgccatagcttgagtaaactatgcagcctgtagctccacctgagaaggtgtaaaaaatccgggaggccacaaaccatggaagctgtacgcatggcgtagtggactagcggttagaggagacccctcccttacaaatcgcagcaacaatgggggcccaaggcgagatgaagctgtagtctcgctggaaggactagaggttagaggagacccccccgaaacaaaaaacagcatattgacgctgggaaagaccagagatcctgctgtctcctcagcatcattccaggcacagaacgccagaaaatggaatggtgctgttgaatcaacaggttct

SEQ ID NO: 8 - protein, 3391 aa, synthetic construct
  source 1..3391: note = chimeric dengue serotype 2/4
  VARIANT 1226: note = X - R orK
MNNQRKKAKNTPFNMLKRERNRVSTVQQLTKRFSLGMLQGRGPLKLFMALVAFLRFLTIPPTAGILKRWGTIKKSKAINVLRGFRKEIGRMLNILNRRRSSAGMIIMLIPTVMAFHLTTRDGEPLMIVAKHERGRPLLFKTTEGINKCTLIAMDLGEMCEDTVTYKCPLLVNTEPEDIDCWCNLTSTWVMYGTCTQSGERRREKRSVALTPHSGMGLETRAETWMSSEGAWKHAQRVESWILRNPGFALLAGFMAYMIGQTGIQRTVFFVLMMLVAPSYGMRCVGVGNRDFVEGVSGGAWVDLVLEHGGCVTTMAQGKPTLDFELTKTTAKEVALLRTYCIEASISNITTATRCPTQGEPYLKEEQDQQYICRRDVVDRGWGNGCGLFGKGGVVTCAKFSCSGKITGNLVQIENLEYTVVVTVHNGDTHAVGNDTSNHGVTATITPRSPSVEVKLPDYGELTLDCEPRSGIDFNEMILMKMKKKTWLVHKQWFLDLPLPWTAGADTSEVHWNYKERMVTFKVPHAKRQDVTVLGSQEGAMHSALAGATEVDSGDGNHMFAGHLKCKVRMEKLRIKGMSYTMCSGKFSIDKEMAETQHGTTVVKVKYEGAGAPCKVPIEIRDVNKEKVVGRIISSTPLAENTNSVTNIELEPPFGDSYIVIGVGNSALTLHWFRKGSSIGKMFESTYRGAKRMAILGETAWDFGSVGGLFTSLGKAVHQVFGSVYTTLFGGVSWMIRILIGFLVLWIGTNSRNTSMAMTCIAAGIVTLYLGVMVQADSGCVVSWKNKELKCGSGIFITDNVHTWTEQYKFQPESPSKLASAIQKAHEEDICGIRSVTRLENLMWKQITPELNHILSENEVKLTIMTGDIKGIMQAGKRSLRPQPTELKYSWKTWGKAKMLSTESHNQTFLIDGPETAECPNTNRAWNSLEVEDYGFGVFTTNIWLKLKEKQDVFCDSKLMSAAIKDNRAVHADMGYWIESALNDTWKIEKASFIEVKNCHWPKSHTLWSNGVLESEMIIPKNLAGPVSQHNYRPGYHTQITGPWHLGKLEMDFDFCDGTTVVVTEDCGNRGPSLRTTTASGKLITEWCCRSCTLPPLRYRGEDGCWYGMEIRPLKEKEENLVNSLVTAGHGQVDNFSLGVLGMALFLEEMLRTRVGTKHAILLVAVSFVTLITGNMSFRDLGRVMVMVGATMTGDIGMGVTYLALLAAFKVRPTFAAGLLLRKLTSXELMMTTIGIVLLSQSTIPETILELTDALALGMMVLKMVRNMEKYQLAVTIMAILCVPNAVILQNAWKVSCTILAVVSVSPLFLTSSQQKTDWIPLALTIKGLNPTAIFLTTLSRTSKKRSWPLNEAIMAVGMVSILASSLLKNDIPMTGPLVAGGLLTVCYVLTGRSADLELERAADVKWEDQAEISGSSPILSITISEDGSMSIKNEEEEQTLTILIRTGLLVISGLFPVSIPITAAAWYLWEVKKQRAGVLWDVPSPPPMGKAELEDGAYRIKQKGILGYSQIGAGVYKEGTFHTMWHVTRGAVLMHKGKRIEPSWADVKKDLISYGGGWKLEGEWKEGEEVQVLALEPGKNPRAVQTKPGLFKTNAGTIGAVSLDFSPGTSGSPIIDKKGKVVGLYGNGVVTRSGAYVSAIAQTEKSIEDNPEIEDDIFRKRRLTIMDLHPGAGKTKRYLPAIVREAIKRGLRTLILAPTRVVAAEMEEALRGLPIRYQTPAIRAVHTGREIVDLMCHATFTMRLLSPVRVPNYNLIIMDEAHFTDPASIAARGYISTRVEMGEAAGIFMTATPPGSRDPFPQSNAPIIDEEREIPERSWNSGHEWVTDFKGKTVWFVPSIKAGNDIAACLRKNGKKVIQLSRKTFDSEYVKTRTNDWDFVVTTDISEMGANFKAERVIDPRRCMKPVILTDGEERVILAGPMPVTHSSAAQRRGRIGRNPKNENDQYIYMGEPLENDEDCAHWKEAKMLLDNINTPEGIIPSMFEPEREKVDAIDGEYRLRGEARKTFVDLMRRGDLPVWLAYRVAAEGINYADRRWCFDGVKNNQILEENVEVEIWTKEGERKKLKPRWLDARIYSDPLALKEFKEFAAGRKSLTLNLITEMGRLPTFMTQKVRDALDNLAVLHTAEAGGRAYNHALSELPETLETLLLLTLLATVTGGIFLFLMSARGIGKMTLGMCCIITASILLWYAQIQPHWIAASIILEFFLIVLLIPEPEKQRTPQDNQLTYVVIAILTVVAATMANEMGFLEKTKKDLGLGSIATQQPESNILDIDLRPASAWTLYAVATTFVTPMLRHSIENSSVNVSLTAIANQATVLMGLGKGWPLSKMDIGVPLLAIGCYSQVNPITLTAALFLLVAHYAIIGPGLQAKATREAQKRAAAGIMKNPTVDGITVIDLDPIPYDPKFEKQLGQVMLLVLCVTQVLMMRTTWALCEALTLATGPISTLWEGNPGRFWNTTIAVSMANIFRGSYLAGAGLLFSIMKNTTNTRRGTGNIGETLGEKWKSRLNALGKSEFQIYKKSGIQEVDRTLAKEGIKRGETDHHAVSRGSAKLRWFVERNMVTPEGKVVDLGCGRGGWSYYCGGLKNVREVKGLTKGGPGHEEPIPMSTYGWNLVRLQSGVDVFFIPPEKCDTLLCDIGESSPNPTVEAGRTLRVLNLVENWLNNNTQFCIKVLNPYMPSVIEKMEALQRKYGGALVRNPLSRNSTHEMYWVSNASGNIVSSVNMISRMLINRFTMRYKKATYEPDVDLGSGTRNIGIESEIPNLDIIGKRIEKIKQEHETSWHYDQDHPYKTWAYHGSYETKQTGSASSMVNGVVRLLTKPWDVVPMVTQMAMTDTTPFGQQRVFKEKVDTRTQEPKEGTKKLMKITAEWLWKELGKKKTPRMCTREEFTRKVRSNAALGAIFTDENKWKSAREAVEDSRFWELVDKERNLHLEGKCETCVYNMMGKREKKLGEFGKAKGSRAIWYMWLGARFLEFEALGFLNEDHWFSRENSLSGVEGEGLHKLGYILRDVSKKEGGAMYADDTAGWDTRITLEDLKNEEMVTNHMEGEHKKLAEAIFKLTYQNKVVRVQRPTPRGTVMDIISRRDQRGSGQVGTYGLNTFTNMEAQLIRQMEGEGVFKSIQHLTITEEIAVQNWLARVGRERLSRMAISGDDCVVKPLDDRFASALTALNDMGKIRKDIQQWEPSRGWNDWTQVPFCSHHFHELIMKDGRVLVVPCRNQDELIGRARISQGAGWSLRETACLGKSYAQMWSLMYFHRRDLRLAANAICSAVPSHWVPTSRTTWSIHAKHEWMTTEDMLTVWNRVWIQENPWMEDKTPVESWEEIPYLGKREDQWCGSLIGLTSRATWAKNIQAAINQVRSLIGNEEYTDYMPSMKRFRREEEEAGVLW

(19) United States
(12) Patent Application Publication
(10) Pub. No.: US 2026/0174812 A1
(43) Pub. Date: Jun. 25, 2026

(54) DENGUE VACCINE UNIT DOSE AND ADMINISTRATION THEREOF

(71) Applicant: Takeda Vaccines, Inc., Cambridge, MA (US)

(72) Inventor: Derek Wallace, Brookline, MA (US)

(73) Assignee: Takeda Vaccines, Inc., Cambridge, MA (US)

(21) Appl. No.: 19/088,420

(22) Filed: Mar. 24, 2025

Related U.S. Application Data
(63) Continuation of application No. 17/818,610, filed on Aug. 9, 2022, now abandoned.
Application No. 17/818,610 is a continuation of application No. 16/295,611, filed on Mar. 7, 2019, now Pat. No. 11,464,815.

(30) Foreign Application Priority Data
Sep. 5, 2018 (EP) 18192701.3
Jan. 29, 2019 (EP) 19154334.7

(51) Int. Cl. A61K 35/76

(52) U.S. Cl. CPC A61K 35/76

(57) ABSTRACT

The invention relates to a unit dose of a dengue vaccine composition and methods and uses for preventing dengue disease and methods for stimulating an immune response to all four dengue virus serotypes in a subject or subject population. The unit dose of a dengue vaccine composition includes constructs of each dengue serotype, such as TDV-1, TDV-2, TDV-3 and TDV-4, at various concentrations in order to improve protection from dengue infection.

### CROSS REFERENCE

This application is Continuation of U.S. application Ser. No. 16/295,611, filed Mar. 7, 2019, which claims the benefit of priority from European Application No. 19154334.7, filed on Jan. 29, 2019, and European Application No. 18192701.3, filed on Sep. 5, 2018, the entire contents of these applications are incorporated herein by reference in their entireties.

### SEQUENCE LISTING

This application incorporates by reference in its entirety, the Sequence Listing XML entitled “T08269USC1C1_Final_updated.xml” (66,283 bytes), which was created on Oct. 30, 2025.

### FIELD OF THE INVENTION

The present invention relates to unit doses of a dengue vaccine composition and methods for administering a unit dose of a dengue vaccine composition to a subject or a subject population. The unit dose according to this invention provides immune responses against all serotypes of dengue virus, i.e. DENV-1, DENV-2, DENV-3 and DENV-4.

### BACKGROUND OF THE INVENTION

Vaccines for protection against viral infections have been effectively used to reduce the incidence of human disease. One of the most successful technologies for viral vaccines is to immunize animals or humans with a weakened or attenuated virus strain (a “live attenuated virus”). Due to limited replication after immunization, the attenuated virus strain does not cause disease. However, the limited viral replication is sufficient to express the full repertoire of viral antigens and can generate potent and long-lasting immune responses to the virus. Thus, upon subsequent exposure to a pathogenic virus strain, the immunized individual is protected from the disease. These live attenuated viral vaccines are among the most successful vaccines used in public health.

Dengue disease is a mosquito-borne disease caused by infection with a dengue virus. Dengue virus infections can lead to debilitating and painful symptoms, including a sudden high fever, headaches, joint and muscle pain, nausea, vomiting and skin rashes. To date, four serotypes of dengue virus have been identified: dengue-I (DENV-I), dengue-2 (DENV-2), dengue-3 (DENV-3) and dengue-4 (DENV-4). Dengue virus serotypes 1-4 can also cause dengue hemorrhagic fever (DHF) and dengue shock syndrome (DSS). In the most severe cases, DHF and DSS can be life threatening. Dengue viruses cause 50-100 million cases of debilitating dengue fever, 500,000 cases of DHF/DSS, and more than 20,000 deaths each year, a large portion of which are children. All four dengue virus serotypes are endemic throughout the tropical regions of the world and constitute the most significant mosquito-borne viral threat to humans there. Dengue viruses are transmitted to humans primarily by Aedes aegypti mosquitoes, but also by Aedes albopictus mosquitoes. Infection with one dengue virus serotype results in life-long protection from re-infection by that serotype, but does not prevent secondary infection by one of the other three dengue virus serotypes. In fact, previous infection with one dengue virus serotype may lead to an increased risk of severe disease (DHF/DSS) upon secondary infection with a different serotype.

To date, only one vaccine, a tetravalent dengue vaccine based on a yellow fever backbone, CYD-TDV (Dengvaxia®, Sanofi Pasteur, Lyon, France), has been licensed in several countries based on the clinical demonstration of an overall vaccine efficacy (VE) against virologically-confirmed dengue (VCD) of 56-61% in children in Asia and Latin America (Capeding M R et al. Clinical efficacy and safety of a novel tetravalent dengue vaccine in healthy children in Asia: a phase 3, randomised, observer-masked, placebo-controlled trial. Lancet 2014, 384:1358-65; Villar L A et al. Safety and immunogenicity of a recombinant tetravalent dengue vaccine in 9-16 year olds: a randomized, controlled, phase II trial in Latin America. Pediatr Infect Dis J 2013, 32:1102-9). However, clinical trials have shown that Dengvaxia® can enhance, rather than reduce, the risk of severe disease due to dengue infection in individuals who had not been previously infected by a dengue virus (seronegative populations). Therefore, Dengvaxia® is only recommended for use in individuals who had been previously infected with at least one dengue virus serotype (seropositive populations). More specifically, according to the European Medicine Agencys European Public Assessment report (EPAR) for the product, Dengvaxia® is only for use in people from 9 to 45 years of age who have been infected with dengue virus before and who live in areas where this infection is endemic. Endemic areas are areas where the disease occurs regularly throughout the year. See also Sridhar S et al. Effect of Dengue Serostatus on Dengue Vaccine Safety and Efficacy. N Engl J Med 2018, 379:327-40; and World Health Organization. Dengue vaccine: WHO position paper—September 2018. Wkly Epidemiol Rec 2018, 93:457-76. S. R. Hadinegoro et al. report in the New England Journal of Medicine, Vol. 373, page 1195, in “Efficacy and Long-Term Safety of a Dengue Vaccine in Regions of Endemic Disease” a pooled risk of hospitalization for virologically-confirmed dengue disease among those under the age of 9 years of 1.58 indicating an increased risk for the vaccinated group with respect to severe dengue. This leaves a substantial unmet need for an effective vaccine with a good safety profile in both dengue-naïve and seropositive individuals, including those dengue-naïve populations living in endemic areas, younger individuals who may not have developed any seropositive response to dengue or been exposed to dengue, and travelers and individuals from non-endemic regions. There is also a need for outbreak control or travel vaccination, offering a reduction in the risk of dengue after only one dose.

One further disadvantage of the only currently approved dengue vaccine, Dengvaxia®, is that it must only be given to people who have had a positive test result showing a previous infection with dengue virus (EPAR), i.e. individuals with known serostatus for dengue. Thus, individuals with unknown serostatus for dengue cannot be vaccinated with Dengvaxia®. There is thus a medical need for a dengue vaccine which, as well as being safe and efficacious for seropositive individuals, can also be administered to individuals with unknown dengue serostatus, children under 9 years old and seronegative individuals.

There is also a need for a vaccine that is administered in fewer doses than the current Dengvaxia® dosing schedule of 3 doses, 6 months apart, such as a vaccine that can be administered in only two doses or one dose to be efficacious.

Also, there is a need for a dengue vaccine that stimulates an immune response to all dengue serotypes, preferably a balanced immune response to all serotypes, and protects against dengue disease of any severity (including DSS, DHF), preferably both in seronegative and seropositive populations, which is safe for a larger group of ages, in particular for subjects of 9 years and younger. The development of a safe and effective vaccine capable of protecting all populations, including both seronegative and seropositive populations, and in particular children and young adults in endemic settings, represents an important approach to the prevention and control of this global disease.

### OBJECTS AND SUMMARY

It is an object of the present invention to provide a safe and effective vaccine against all serotypes of dengue virus for dengue-endemic and dengue non-endemic populations and for a broad range of ages, in particular for subjects between 2 months and 60 years of age, and independent of previous exposure to dengue virus and corresponding seropositive or seronegative status before vaccination.

It is an object of the present invention to minimize the risk of DHF and DSS caused by infection with DENV-1, DENV-2, DENV-3 or DENV-4, in particular following vaccination in children of young age and individuals of any age who have never been previously exposed to dengue, or who are seronegative to dengue before vaccination.

It is an object of the present invention to provide a vaccine which stimulates a balanced immune response to all four dengue serotypes in a subject.

The present invention is therefore directed in part to a reconstituted dengue vaccine composition for use in a method of preventing virologically confirmable dengue disease in a subject comprising consecutively administering at least a first and a second unit dose of the dengue vaccine composition to the subject, wherein said first and second unit dose are administered subcutaneously within 3 months and at least 4 weeks apart, optionally at about day 1 and at about day 90, wherein the dengue vaccine composition is a tetravalent dengue virus composition including four dengue virus strains representing dengue serotype 1, dengue serotype 2, dengue serotype 3 and dengue serotype 4, optionally wherein the dengue virus strains are live, attenuated, and wherein upon reconstitution with 0.5 ml of a pharmaceutically acceptable diluent
(i) dengue serotype 1 has a concentration of at least 3.3 log 10 pfu/0.5 mL and optionally to 5.0 log 10 pfu/0.5 mL,
(ii) dengue serotype 2 has a concentration of at least 2.7 log 10 pfu/0.5 mL and optionally to 4.9 log 10 pfu/0.5 mL,
(iii) dengue serotype 3 has a concentration of at least 4.0 log 10 pfu/0.5 mL and optionally to 5.7 log 10 pfu/0.5 mL, and
(iv) dengue serotype 4 has a concentration of at least 4.5 log 10 pfu/0.5 mL and optionally to 6.2 log 10 pfu/0.5 mL.

The present invention is therefore directed in part to a dengue vaccine composition for use in a method of preventing virologically confirmable dengue disease (VCD) in a subject comprising consecutively administering at least a first and a second unit dose of the dengue vaccine composition to the subject, wherein said first and second unit dose are administered subcutaneously within 3 months and at least 4 weeks apart, optionally at about day 1 and at about day 90, and wherein the dengue vaccine composition is a tetravalent dengue virus composition including four live, attenuated dengue virus strains representing dengue serotype 1, dengue serotype 2, dengue serotype 3 and dengue serotype 4, wherein the attenuated dengue virus strains comprise chimeric dengue viruses and preferably at least one non-chimeric dengue virus, and wherein the dengue serotype 1 and the dengue serotype 2 are present each in a concentration based on the total concentration in pfu/0.5 mL which is within 5%-points of each other and/or are together less than about 10% of the total concentration in pfu/0.5 mL, in particular wherein the dengue serotype 3 is at least about 10% of the total concentration in pfu/0.5 mL and in particular wherein the dengue serotype 4 is at least about 70% of the total concentration in pfu/0.5 mL.

The present invention is therefore directed in part to a unit dose of a dengue vaccine composition and use thereof, the unit dose comprising:

a tetravalent dengue virus composition including four live attenuated dengue serotypes (e.g. virus strains):
(i) a dengue serotype 1 (e.g. chimeric dengue serotype 2/1 strain),
(ii) a dengue serotype 2 (e.g. dengue serotype 2 strain),
(iii) a dengue serotype 3 (e.g. chimeric dengue serotype 2/3 strain),
(iv) a dengue serotype 4 (e.g. chimeric dengue serotype 2/4 strain),
   and one or more pharmaceutically acceptable excipients thereof.

The present invention is further directed in part to a unit dose of a dengue vaccine composition and use thereof, the unit dose comprising:

a tetravalent virus composition including four live attenuated dengue virus strains:
(i) a dengue serotype 1 (e.g. chimeric dengue serotype 2/1 strain) in a concentration of at least 3.3 log 10 pfu/0.5 ml,
(ii) a dengue serotype 2 (e.g. dengue serotype 2 strain) in a concentration of at least 2.7 log 10 pfu/0.5 ml,
(iii) a dengue serotype 3 (e.g. chimeric dengue serotype 2/3 strain) in a concentration of at least 4.0 log 10 pfu/0.5 ml, and
(iv) a dengue serotype 4 (e.g. chimeric dengue serotype 2/4 strain) in a concentration of at least 4.5 log 10 pfu/0.5 ml,
   and one or more pharmaceutically acceptable excipients.

The present invention is further directed in part to a unit dose of a dengue vaccine composition and use thereof, the unit dose comprising:

a tetravalent virus composition including four live attenuated dengue virus strains, wherein the unit dose is lyophilized and upon reconstitution with 0.5 mL of a pharmaceutically acceptable diluent comprises:
(i) a dengue serotype 1 (e.g. chimeric dengue serotype 2/1 strain) in a concentration of at least 3.3 log 10 pfu/0.5 ml,
(ii) a dengue serotype 2 (e.g. dengue serotype 2 strain) in a concentration of at least 2.7 log 10 pfu/0.5 ml,
(iii) a dengue serotype 3 (e.g. chimeric dengue serotype 2/3 strain) in a concentration of at least 4.0 log 10 pfu/0.5 ml, and
(iv) a dengue serotype 4 (e.g. chimeric dengue serotype 2/4 strain) in a concentration of at least 4.5 log 10 pfu/0.5 ml.

The present invention is further directed in part to a unit dose of a dengue vaccine composition and use thereof, wherein said unit dose is lyophilized and obtained by lyophilizing 0.5 mL of a solution comprising:

a tetravalent virus composition including four live attenuated dengue virus strains:
(i) a dengue serotype 1 (e.g. chimeric dengue serotype 2/1 strain) in a concentration of at least 3.3 log 10 pfu/0.5 ml,
(ii) a dengue serotype 2 (e.g. dengue serotype 2 strain) in a concentration of at least 2.7 log 10 pfu/0.5 ml,
(iii) a dengue serotype 3 (e.g. chimeric dengue serotype 2/3 strain) in a concentration of at least 4.0 log 10 pfu/0.5 ml, and
(iv) a dengue serotype 4 (e.g. chimeric dengue serotype 2/4 strain) in a concentration of at least 4.5 log 10 pfu/0.5 ml,
   and one or more pharmaceutically acceptable excipients.

The present invention is further directed in part to a kit for preparing a reconstituted unit dose and use thereof, the kit comprising the following components:
a) a lyophilized unit dose of the present invention as described herein, and
b) a pharmaceutically acceptable diluent for reconstitution.

The present invention is further directed in part to a container, such as a vial, comprising one to ten unit doses of the present invention as described herein.

The present invention is further directed to a method of preventing dengue disease in a subject comprising administering to the subject a reconstituted unit dose of a dengue vaccine composition as described herein, for example by subcutaneous injection.

The present invention is further directed in part to a method of preventing virologically confirmable dengue disease in a subject comprising administering to the subject a tetravalent dengue virus composition including four live, attenuated dengue virus strains representing serotype 1, serotype 2, serotype 3 and serotype 4, in particular without determining the serostatus of the subject at baseline, said method being safe and effective.

The present invention is further directed in part directed to such a method having a combined vaccine efficacy against all four dengue serotypes with a 2-sided 95% confidence interval, wherein the lower bound is more than 25%, when measured against placebo in a subject population of at least 5,000 healthy subjects, or at least 10,000 healthy subjects, or at least 15,000 healthy subjects irrespective of serostatus at baseline, wherein preferably said unit dose or said placebo is administered at least twice within less than 6 months, such as within 3 months, and optionally at least 4 weeks apart, about 30 days after the second administration of the administration schedule until at least 12 months after the second administration of the administration schedule.

The present invention is further directed in part to such a method having a combined vaccine efficacy against all four dengue serotypes of more than 30%, when measured against placebo in a subject population of at least 5,000 healthy subjects, or at least 10,000 healthy subjects, or at least 15,000 healthy subjects irrespective of serostatus at baseline, wherein said unit dose or said placebo is administered at least twice within less than 6 months, such as within 3 months, and optionally at least 4 weeks apart, 30 days after the second administration until at least 12 months after the second administration.

The present invention is further directed to a method of preventing dengue disease in a subject population, comprising administering to the subject population a reconstituted unit dose of a vaccine composition as described herein, wherein the subject population is seronegative to all dengue serotypes. In said method the geometric mean neutralizing antibody titers (GMTs) when tested in at least 40, or at least 50, or at least 60 subjects at day 180 or day 365 after at least a first administration of said unit dose, and optionally a second administration of said unit dose 90 days after said first administration, may provide a ratio of not more than 50, or not more than 40, or nor more than 30, or not more than 20 for the GMT of dengue serotype 2 to the GMT of dengue serotype 4 (GMT DENV-2:GMT DENV-4), and optionally a ratio of not more than 20 for the GMT of dengue serotype 2 to the GMT of dengue serotype 1 (GMT DENV-2:GMT DENV-1), and optionally a ratio of not more than 20 for the GMT of dengue serotype 2 to the GMT of dengue serotype 3 (GMT DENV-2:GMT DENV-3).

The present invention is further directed to a method of preventing dengue disease in a subject, comprising administering to the subject a reconstituted unit dose of a vaccine composition as described herein, wherein the subject is seronegative to all dengue serotypes. In said method the neutralizing antibody titers when tested in the subject at day 180 or day 365 after at least a first administration of said unit dose, and optionally a second administration of said unit dose 90 days after said first administration, may provide a ratio of not more than 50, or not more than 40, or nor more than 30, or not more than 20 for the neutralizing antibody titer of dengue serotype 2 to the neutralizing antibody titer of dengue serotype 4, and optionally a ratio of not more than 20 for the neutralizing antibody titer of dengue serotype 2 to the neutralizing antibody titer of dengue serotype 1, and optionally a ratio of not more than 20 for the neutralizing antibody titer of dengue serotype 2 to the neutralizing antibody titer of dengue serotype 3.

In one preferred embodiment, the methods of preventing dengue disease of the present invention are not associated with an increased likelihood of solicited systemic adverse events, such as in children under 9 or seronegative individuals.

The present invention is further directed in part to the use of the reconstituted unit dose of a dengue vaccine composition as described herein for the manufacture of a medicament for preventing dengue disease in a subject.

The present invention is further directed in part to the reconstituted unit dose of a dengue vaccine composition as described herein for use in a method of preventing dengue disease in a subject as described herein.

### Definitions

In describing the present invention, the following terms are to be used as indicated below. As used herein, the singular forms “a,” “an,” and “the” include plural references unless the context clearly indicates otherwise.

As used herein, the terms “unit dose of a dengue vaccine composition”, “unit dose” and “unit dose of the invention as described herein” refer to the amount of a dengue vaccine which is administered to a subject in a single dose. In one embodiment, one unit dose is present in a vial and this unit dose is administered to a subject, optionally after reconstitution. In one embodiment, more than one unit dose of the dengue vaccine composition may be present in a vial so that with the content of one vial more than one subject can be vaccinated.

A “lyophilized unit dose” or “unit dose in lyophilized form” refers to the unit dose that is obtained by subjecting a given volume of the liquid dengue vaccine composition, such as 0.5 mL, to lyophilization. Thus, the aqueous formulations of the dengue vaccine composition being produced by combining the pharmaceutically acceptable excipients and the dengue virus composition comprising the four dengue virus strains, preferably TDV-1 to TDV-4, is subjected to lyophilization to obtain the lyophilized unit dose.

A “reconstituted unit dose” or “unit dose in reconstituted form” is obtained from the lyophilized dose by reconstitution with a pharmaceutically acceptable diluent. The diluent does not contain dengue virus. The reconstituted unit dose is a liquid which can be administered to a subject, for example by injection, such as subcutaneous injection.

As used herein, the term “upon reconstitution with 0.5 mL” is not limiting the reconstitution to be performed using 0.5 mL of the diluent, but refers to the concentration of the dengue viruses that will be present in the reconstituted unit dose when 0.5 mL diluent are used for reconstitution. While using a different volume for reconstitution (e.g. 0.8 mL) will result in a different concentration of dengue viruses in the reconstituted unit dose, the administration of the total volume of the unit dose (e.g. 0.8 mL) will result in the same total amount of dengue virus being administered.

As used herein, a “concentration of at least X log 10 pfu/0.5 mL” refers to the concentration of a dengue serotype in 0.5 mL, but is not limiting the unit dose to be 0.5 mL. If the unit dose has a volume different than 0.5 mL, or is lyophilized from a volume different than 0.5 mL, or is reconstituted with a volume different than 0.5 mL, said concentration will differ from the “concentration of at least X log 10 pfu/0.5 mL”. However, if the unit dose has a volume of 0.5 mL, or is lyophilized from a volume of 0.5 mL, or is reconstituted with a volume of 0.5 mL, said concentration will be the “concentration of at least X log 10 pfu/0.5 mL”. Thus, while the concentration may differ, the total amount of virus in the unit dose remains the same.

As used herein, the term “dengue serotype” refers to a species of dengue virus which is defined by its cell surface antigens and therefore can be distinguished by serological methods known in the art. At present, four serotypes of dengue virus are known, i.e. dengue serotype 1 (DENV-1), dengue serotype 2 (DENV-2), dengue serotype 3 (DENV-3) and dengue serotype 4 (DENV-4).

As used herein, the term “tetravalent dengue virus composition” refers to a dengue virus composition comprising four different immunogenic components from the four different dengue serotypes DENV-1, DENV-2, DENV-3 and DENV-4, preferably comprising four different live, attenuated dengue viruses, each representing one dengue serotype, and which aims to stimulate immune responses to all four dengue serotypes.

As used herein, the term “live attenuated dengue virus” refers to a viable dengue virus which is mutated to provide reduced virulence. The live attenuated dengue virus can be a dengue virus in which all components are derived from the same dengue serotype or it can be a chimeric dengue virus having parts from two or more dengue serotypes.

A “virus strain” and in particular a “dengue virus strain” is a genetic subtype of a virus, in particular of a dengue virus, which is characterized by a specific nucleic acid sequence. A dengue serotype may comprise different strains with different nucleic acid sequences which have the same cell surface antigens. A dengue virus strain can be a dengue virus in which all components are derived from the same dengue serotype or it can be a chimeric dengue virus having parts from two or more dengue serotypes.

As used herein, “TDV-2” refers to a molecularly characterized and cloned dengue serotype 2 strain derived from the live attenuated DEN-2 PDK-53 virus strain. The PDK-53 strain is described for example in Bhamarapravati et al. (1987) Bulletin of the World Health Organization 65 (2): 189-195. In one embodiment, the TDV-2 strain served as a backbone for the chimeric TDV-1, TDV-3 and TDV-4 strains into which parts from the TDV-1, TDV-3 and TDV-4 strains were introduced.

A “non-chimeric dengue virus” or “non-chimeric dengue serotype strain” or “non-chimeric dengue strain” comprises only parts from one dengue serotype. In particular, a non-chimeric dengue virus does not include parts from a different flavivirus such as yellow fever virus, Zika virus, West Nile virus, Japanese encephalitis virus, St. Louis encephalitis virus, tick-borne encephalitis virus. TDV-2 is an example of a non-chimeric dengue virus.

A “chimeric dengue virus” or “chimeric dengue serotype strain” or “chimeric dengue strain” comprises parts from at least two different dengue serotypes. As used herein, the chimeric dengue virus does not include parts from a different flavivirus such as yellow fever virus, Zika virus, West Nile virus, Japanese encephalitis virus, St. Louis encephalitis virus, tick-borne encephalitis virus. In particular, the chimeric dengue virus described herein does not include parts from the yellow fever virus. As used herein, a “chimeric dengue serotype 2/1 strain” or “DENV-2/1 chimera” or “TDV-1” refers to a dengue virus chimeric construct which comprises parts from both DENV-2 and DENV-1. In particular, in the chimeric dengue serotype 2/1 strain the prM and E proteins from DENV-1 replace the prM and E proteins from DENV-2 as detailed below. As used herein, a “chimeric dengue serotype 2/3 strain” or “DENV-2/3 chimera” or “TDV-3” refers to a dengue virus chimeric construct which comprises parts from both DENV-2 and DENV-3. In particular, in the chimeric dengue serotype 2/3 strain the prM and E proteins from DENV-3 replace the prM and E proteins from DENV-2 as detailed below. As used herein, a “chimeric dengue serotype 2/4 strain” or “DENV-2/4 chimera” or “TDV-4” refers to a dengue virus chimeric construct which comprises parts from both DENV-2 and DENV-4. In particular, in the chimeric dengue serotype 2/4 strain the prM and E proteins from DENV-4 replace the prM and E proteins from DENV-2 as detailed below.

As used herein, “TDV” refers to a tetravalent live attenuated dengue vaccine that comprises a mixture of the four live attenuated dengue virus strains TDV-1, TDV-2, TDV-3 and TDV-4 expressing surface antigens from the four dengue serotypes DENV-1, DENV-2, DENV-3 and DENV-4, respectively. In one embodiment, TDV-1 has the nucleotide sequence according to SEQ ID No. 1 and/or the amino acid sequence according to SEQ ID No. 2. In one embodiment, TDV-2 has the nucleotide sequence according to SEQ ID No. 3 and/or the amino acid sequence according to SEQ ID No. 4. In one embodiment, TDV-3 has the nucleotide sequence according to SEQ ID No. 5 and/or the amino acid sequence according to SEQ ID No. 6. In one embodiment, TDV-4 has the nucleotide sequence according to SEQ ID No. 7 and/or the amino acid sequence according to SEQ ID No. 8.

As used herein, the term “dengue disease” refers to the disease which is caused by infection with dengue virus. Symptoms of dengue disease include sudden high fever, headaches, joint and muscle pain, nausea, vomiting and skin rashes. The term dengue disease also includes the more severe forms of dengue hemorrhagic fever (DHF) and dengue shock syndrome (DSS). Symptoms of DHF include increased vascular permeability, hypovolemia and abnormal blood clotting mechanisms. Subjects with DHF may present with severe manifestations of plasma leakage and hemorrhage. When a subject with DHF experiences shock he or she will be categorized as having DSS. Symptoms of DSS include bleeding that may appear as tiny spots of blood on the skin and larger patches of blood under the skin. Prolonged shock is the main factor associated with complications including massive gastrointestinal hemorrhage that can lead to death. As used herein, DHF cases are defined as VCD cases meeting WHO 1997 DHF criteria.

As used herein, “preventing dengue disease” refers to preventing a subject from developing one or more symptoms of dengue disease because of an infection with a dengue virus. In particular, preventing dengue disease is achieved by vaccinating or inoculating a subject with a dengue vaccine composition, such as the reconstituted unit dose described herein. As used herein, the term “prophylactically treating dengue disease” is equivalent to “preventing dengue disease”. In a particular embodiment, preventing dengue disease includes preventing DHS and/or DSS.

As used herein, the terms “virologically-confirmed dengue disease”, “VCD case”, or “VCD fever” refer to febrile illness or illness clinically suspected to be dengue disease with a positive serotype-specific reverse transcriptase polymerase chain reaction (RT-PCR). The term “virologically confirmable dengue” disease refers to a subject having febrile illness or illness clinically suspected to be dengue disease, wherein testing the subject, e.g. using RT-PCR, would confirm the presence of at least one dengue serotype. Severe forms of VCD fever will be identified as follows: Dengue Hemorrhagic Fever (DHF) was defined according to the WHO 1997 criteria. Severe dengue was defined through an assessment of an independent Dengue Case Adjudication Committee which will assess all hospitalized VCD cases (severe/non-severe) based on criteria redefined in a charter. All non-hospitalized cases are considered non-severe.

As used herein, the term “febrile illness” is defined as temperature≥38° C. on any 2 of 3 consecutive days.

As used herein, the terms “virologically-confirmed dengue disease with hospitalization”, is considered to be a surrogate for severe dengue and the “incidence of virologically-confirmed dengue disease with hospitalization” is used as a safety parameter. As used herein, the “relative risk with respect to virologically-confirmed dengue disease with hospitalization” means the number of events of virologically confirmed dengue disease with hospitalization divided by the number of subjects treated with the unit dose as disclosed herein over the number of events of virologically confirmed dengue disease with hospitalization divided by the number of subjects treated with placebo. If the “relative risk with respect to virologically-confirmed dengue disease with hospitalization” is 1 or lower the vaccine provides for the same or less risk for virologically-confirmed dengue disease with hospitalization as placebo and is considered “safe”. In this context the risk of virologically-confirmed dengue disease with hospitalization may be also 0.9 or less, 0.8 or less, 0.7 or less, 0.6 or less, 0.5 or less, 0.4 or less, 0.3 or less, 0.2 or less, or 0.1 or less, in particular when determined from 30 days after a second administration until 12 months after a second administration, in particular when determined in age groups selected from the age group of 4 to 16 year old subjects, the age group of 4 to under 9 year old subjects, the age group of 2 to under 9 year old subjects, the age group of 4 to 5 year old subjects, the age group of 6 to 11 year old subjects, and the age group of 12 to 16 year old subjects.

As used herein, alternatively a vaccine is considered “safe” when the vaccine efficacy (VE) with respect to virologically-confirmed dengue disease with hospitalization is 0% or higher. This means that the vaccine provides for the same likelihood or less for virologically-confirmed dengue disease with hospitalization as placebo. In particular considered “safe” is the combined vaccine efficacy against virologically-confirmed dengue with hospitalization against all four serotypes with a 2-sided 95% confidence interval, wherein the lower bound is more than 25%, in particular when measured against placebo in a subject population of at least 2,000 healthy subjects (in particular when measured in age groups selected from the age group of 4 to 16 year old subjects, the age group of 4 to under 9 year old subjects, the age group of 2 to under 9 year old subjects, the age group of 4 to 5 year old subjects, the age group of 6 to 11 year old subjects, and the age group of 12 to 16 year old subjects) being seronegative against all serotypes at baseline or being seropositive against at least one serotype at baseline, in particular when said unit dose or said placebo is administered at least twice within less than 6 months, such as within 3 months, about 30 days after the second administration of the administration schedule until at least 12 months after the second administration of the administration schedule. In particular, the lower bound may be more than 30%, more than 40%, more than 50%, more than 60%, more than 70%, or more than 75%. In particular, the 2-sided 95% confidence interval of the combined vaccine efficacy against virologically-confirmed dengue with hospitalization against all four serotypes when comparing seropositive and seronegative subjects provides for lower bounds of the 2-sided confidence interval which are within 10% points.

If one of the criteria as defined above for the term “safe” is fulfilled, the vaccine is considered safe within the meaning of this invention. In this context, safe in particular refers to a vaccine that is safe for all subjects irrespective of their serostatus at baseline. This means that the vaccine can be administered without the need to determine the occurrence of a previous dengue infection in the subject before administration. Preferably, the vaccine is safe as defined above with respect to all age groups starting from 4 years of age and preferably irrespective of the serostatus, in particular from 4 years of age to 60 years of age, or 4 years of age to 16 years of age. Relevant subgroups in this context are under 9 years of age, from 2 years of age to under 9 years of age, from 4 years of age to under 9 years of age, 4 to 5 years of age, 6 to 11 years of age and 12 to 16 years of age or any age group within 4 to 16 years of age.

As used herein, “vaccine efficacy” or “VE” measure the proportionate reduction in cases among vaccinated persons. Vaccine efficacy (VE) is measured by calculating the risk of disease among vaccinated and unvaccinated persons and determining the percentage reduction in risk of disease among vaccinated persons relative to unvaccinated persons. The greater the percentage reduction of illness in the vaccinated group, the greater the vaccine efficacy. For example, a VE of 90% indicates a 90% reduction in disease occurrence among the vaccinated group, or a 90% reduction from the number of cases you would expect if they have not been vaccinated. The vaccine efficiency is calculated by the formula: 100*(1−HR), wherein HR is the Hazard Ratio which is defined as the Hazard rate of vaccine (λv) divided by the Hazard rate of placebo (λc), i.e. HR=λv/λc. λv denote the hazard rate for the subjects vaccinated with a tetravalent dengue vaccine composition as disclosed herein and λc denote the hazard rate for unvaccinated subjects, i.e. subjects receiving placebo. The hazard rate ratio HR is estimated from a Cox proportional hazard model with study vaccine as a factor, adjusted for age, and stratified by region. As used herein the term “combined vaccine efficacy against all four serotypes” is defined as the vaccine efficacy in relation to the risk of dengue disease irrespective of the serotype being responsible for the virologically-confirmed dengue disease and the subject baseline serostatus. A vaccine is considered “effective” in case the combined vaccine efficacy is above 30%. In this context the combined vaccine efficacy may be also 40% or more, 50% or more, 60% or more, 70% or more, or 80% or more, in particular when determined from 30 days after a second administration until 12 months after a second administration, in particular when determined in age groups selected from the age group of 4 to 16 year old subjects, the age group of 4 to under 9 year old subjects, the age group of 2 to under 9 year old subjects, the age group of 4 to 5 year old subjects, the age group of 6 to 11 year old subjects, and the age group of 12 to 16 year old subjects. In this context, effective in particular refers to a vaccine that is effective for all subjects irrespective of their serostatus at baseline. Preferably, the vaccine is effective with respect to all age groups starting from 4 years of age and preferably irrespective of the serostatus, in particular from 4 years of age to 60 years of age or from 4 years of age to 16 years of age and irrespective of the serostatus. Relevant subgroups in this context are under 9 years of age, from 2 years of age to under 9 years of age, from 4 years of age to under 9 years of age, 4 to 5 years of age, 6 to 11 years of age and 12 to 16 years of age or any age group within 4 to 16 years of age. Further specific efficacies can be defined. As used herein, “combined vaccine efficacy against all four serotypes in seronegative subjects” refers to the efficacy measured in subjects which are seronegative at baseline. As used herein, “vaccine efficacy against a specific serotype, e.g. serotype 1” refers to the efficacy in relation to a specific serotype being responsible for the virologically-confirmed dengue disease. As used herein, “combined vaccine efficacy against all four serotypes against virologically-confirmed dengue with hospitalization” refers to the efficacy wherein only virologically-confirmed dengue cases with hospitalization are considered. Such vaccine efficacies can be determined with respect to subjects being seronegative or seropositive at baseline and for different age groups.

As used herein, the “relative risk” means the number of events of virologically confirmed dengue disease divided by the number of subjects treated with the unit dose as disclosed herein over the number of events of virologically confirmed dengue disease divided by the number of subjects treated with placebo. As used herein the term “combined relative risk against all four serotypes” is defined as the relative risk in relation to the risk of dengue disease irrespective of the serotype being responsible for the virologically-confirmed dengue disease and the subject baseline serostatus.

As used herein, “vaccinating” or “inoculating” refers to the administration of a vaccine to a subject with the aim to prevent the subject from developing one or more symptoms of a disease. As used herein, “vaccinating against dengue disease” or “inoculating against dengue disease” refers to the administration of a dengue vaccine composition to a subject with the aim to prevent the subject from developing one or more symptoms of dengue disease.

As used herein, the terms “subject” or “subjects” are limited to human subjects (e.g. infants, children or adults).

As used herein, “subject population” refers to a group of subjects. The subject population may refer to least 40 subjects, at least 50 subjects, at least 60 subjects, at least 100 subjects or at least 1000 subjects and is defined by certain parameters. The parameters that may be used to define a subject population include, but are not limited to, the age of the subjects, whether the subjects are from a dengue endemic region or from a dengue non-endemic region and the serostatus of the subjects.

As used herein, “endemic region” refers to a region where a disease or infectious agent is constantly present and/or usually prevalent in a population within this region. As used herein, “non-endemic region” refers to a region from which the disease is absent or in which it is usually not prevalent. Accordingly, a “dengue endemic region” refers to geographic areas in which an infection with dengue virus is constantly maintained at a baseline level. A “dengue non-endemic region” is a geographic area in which an infection with dengue virus is not constantly maintained at a baseline level. Accordingly, subject populations or subjects “from a dengue endemic region” or “from a dengue non-endemic region” refer to subject populations or subjects living in geographic areas as defined above. Whether a geographic area or a subject population is dengue-endemic or not can be determined by different calculatory methods such as the ones described in Bhatt et al. (2013) Nature 496 (7446): 504-507 and supplementary material and in Stanaway et al. (2016) Lancet Infect Dis. 16 (6): 712-723 and supplementary material. Overviews of dengue endemic regions and dengue epidemiology are regularly published, for example, by the WHO or CDC. Typical dengue-endemic regions are in Latin America, Southeast Asia and the Pacific islands and dengue endemic countries include, but are not limited to, Australia, Brazil, Bangladesh, Colombia, China, Dominican Republic, Indonesia, India, Mexico, Malaysia, Nicaragua, Nigeria, Pakistan, Panama, Philippines, Puerto Rico, Singapore, Sri Lanka, Thailand and Vietnam.

As used herein, “serostatus” refers to the amount of antibodies a subject has with respect to a certain infectious agent, in particular dengue virus. As used herein, “seronegative” or “seronaïve” means that the subject does not have neutralizing antibodies against any one of dengue serotypes DENV-1, DENV-2, DENV-3 and DENV-4 in the serum. A seronegative or seronaïve subject or subject population is defined by a neutralizing antibody titer of less than 10 for each one of the four dengue serotypes. A subject or subject population having a neutralizing antibody titer of equal to or more than 10 for at least one dengue serotype is defined as being “seropositive” with respect to said dengue serotype. Serostatus at baseline refers to the serostatus before the administration of a dengue vaccine composition as described herein.

As used herein, a “neutralizing antibody titer” refers to the amount of antibodies in the serum of a subject that neutralize the respective dengue serotype. The neutralizing antibody titer against DENV-1, DENV-2, DENV-3 and DENV-4 is determined in a serum sample of the subject using known methods such as the plaque reduction neutralization test (PRNT) as described in the WHO Guidelines (World Health Organization Department of Immunization Vaccines Biologicals (2007) Guidelines for plaque reduction neutralization testing of human antibodies to dengue viruses, WHO/IVB/07.07) or a microneutralization (MNT50) assay as described herein. As used herein, the “ratio of not more than 20 for the neutralizing antibody titer of dengue serotype 2 to the neutralizing antibody titer of dengue serotype 4” means that the neutralizing antibody titer of dengue serotype 2 is divided by the neutralizing antibody titer of dengue serotype 4 and that the ratio obtained hereby is no more than 20. In other words, the neutralizing antibody titer of dengue serotype 2 is not more than 20-times higher than the neutralizing antibody titer of dengue serotype 4 in the subject.

As used herein, the terms “geometric mean neutralizing antibody titer” and “GMT” refer to the geometric mean value of the titer of neutralizing antibodies against the corresponding dengue serotype in the serum of subjects in a subject population. The geometric mean value is calculated by a well-known formula. As used herein, the “ratio of not more than 20 for the GMT of dengue serotype 2 to the GMT of dengue serotype 4” means that the geometric mean neutralizing antibody titer of dengue serotype 2 (GMT DENV-2) is divided by the geometric mean neutralizing antibody titer of dengue serotype 4 (GMT DENV-4) and that the ratio obtained hereby is no more than 20. In other words, the geometric mean neutralizing antibody titer of dengue serotype 2 is not more than 20-times higher than the geometric mean neutralizing antibody titer of dengue serotype 4 in the subject population.

As used herein, an “immune response” refers to a subject's response to the administration of the dengue vaccine. In particular, the immune response includes the formation of neutralizing antibodies to one or more dengue serotypes. It may also include the stimulation of a cell-mediated response or the formation of antibodies to non-structural proteins such as NS1. An immune response is stimulated by the administration of a unit dose of the invention as described herein, if the titer of neutralizing antibodies against at least one dengue virus serotype and preferably against all four dengue virus serotypes is increased after said administration of said unit dose. An immune response is stimulated by the administration of a unit dose of the invention as described herein, if the secretion of interferon gamma by peripheral blood mononuclear cells stimulated with peptides from dengue virus proteins is increased after said administration of said unit dose. An immune response is stimulated by the administration of a unit dose of the invention as described herein, if the titer of antibodies to non-structural proteins such as NS1 is increased after said administration of said unit dose. In a particular embodiment, the administration of a reconstituted unit dose of the present invention as described herein stimulates the formation of neutralizing antibodies to one or more dengue serotypes, a cell-mediated response and the formation of antibodies to non-structural proteins such as NS1.

As used herein, a “balanced immune response” means that the immune response to the four dengue serotypes is sufficient to provide protection against infection by all four dengue serotypes and preferably the immune response to the four dengue serotypes has a similar strength. In particular, the neutralizing antibody titer against the four dengue serotypes at day 180 or day 365 after administration of a first reconstituted unit dose of the invention as described herein is similar, i.e. it differs by less than factor 30, by less than factor 25 or by less than factor 20.

As used herein, “solicited systemic adverse events” in children under 6 years are defined as fever, irritability/fussiness, drowsiness and loss of appetite that occurred within 14 days after each vaccination, and in children of 6 years or more are defined as fever, headache, asthenia, malaise and myalgia that occurred within 14 days after each vaccination.

As used herein, “solicited local adverse events” are injection site pain, injection site erythema and injection site swelling that occurred within 7 days after each vaccination.

As used herein, “unsolicited adverse events” are any adverse events (AEs) that are not solicited local or systemic AEs, as defined above.

As used herein, a “serious adverse event” or “SAE” is any untoward medical occurrence or effect that at any dose results in death, is life-threatening, requires inpatient hospitalization or prolongation of existing hospitalization, results in persistent or significant disability/incapacity, is a congenital anomaly/birth defect or is medically important due to other reasons than the above mentioned criteria.

The relationship of each AE, including solicited systemic AEs (solicited local AEs are considered as related) to trial vaccine(s) will be assessed using the following categories: As used herein, “IP-Related AE” or “vaccine related AE” means that there is suspicion that there is a relationship between the vaccine and the AE (without determining the extent of probability); there is a reasonable possibility that the vaccine contributed to the AE. As used herein, “Non-IP Related” or “non-vaccine related” means that there is no suspicion that there is a relationship between the vaccine and the AE; there are other more likely causes and administration of the vaccine is not suspected to have contributed to the AE.

As used herein, a subject or subject population being “2 to 17 years of age” refers to a subject or subject population being 2 to 17 years of age on the first day of the administration of the dengue vaccine composition as described herein.

As used herein “%-points” refers to the difference of two %-values in a %-value. For example two values in % which are within 5%-points refers to e.g. one value at 1% and a second value at 6%.

As used herein, the term “determination of the previous dengue infection in the subject before administration” means that a previous dengue infection has to be assessed before vaccination in that there is a laboratory confirmed history of dengue or through an appropriately validated serological test e.g. by the method as disclosed herein such as the MNT50 test described in Example 2 or any serotesting with adequate performance in terms of specificity and cross reactivity based on the locale disease epidemiology.

### BRIEF DESCRIPTION OF THE DRAWINGS

FIG. 1: Genetic structure of the four dengue strains contained in TDV. The solid red triangles indicate the three attenuating mutations present in the 5′NCR, NS1 and NS3 proteins. The TDV-1, TDV-3 and TDV-4 strains are chimeric viruses where the prM and E genes from dengue serotype 1, 3 and 4, respectively, are inserted into the TDV-2 backbone.

FIG. 2: Schematic drawing illustrating the microneutralization test (MNT) used to determine the titer of neutralizing antibodies.

FIGS. 3A & 3B: Percentage of subjects (+95% confidence intervals) who were seropositive (reciprocal neutralizing titer 10) for each of the dengue serotypes at different time points of the trial in the HD-TDV group (dark colored, left bar) and TDV group (light colored, right bar) throughout the trial. Time points shown are baseline, day 15 (d15), day 30 (d30), day 90 (d90), day 180 (d180) and day 365 (d365). FIG. 3A shows the results for participants seropositive (set of graphs on the left) and seronegative (set of graphs on the right) at baseline, per-protocol set. FIG. 3B shows the results for the entire trial population (all) per-protocol set.

FIGS. 4A & 4B: Geometric mean titers (GMTs) (+95% confidence intervals) of neutralizing antibodies against each of the four dengue serotypes during the course of the trial for HD-TDV (dark line with triangles) and TDV (light line with circles) recipients, for the entire trial population (FIG. 4B) and for participants seropositive and seronegative at baseline (FIG. 4A), per-protocol set.

FIG. 5: IFNγ ELISpot analysis of peripheral blood mononuclear cells before vaccination (baseline) and at different time points after administration of TDV. Shown are the response frequencies to the entire DENV-2 proteome. A subject was considered responsive if response to more than one peptide pool from DENV-2 was positive (i.e. ≥4× mean of negative control and ≥50 SFC/106 PBMCs).

FIGS. 6A & 6B: IFNγ ELISpot analysis of peripheral blood mononuclear cells before vaccination (baseline) and at different time points after administration of TDV. Shown are the response frequencies to peptide pools matching selected DENV-derived proteins as indicated. A subject was considered responsive if response to more than one peptide pool from DENV-2 was positive (i.e. >4× mean of negative control and >50 SFC/106 PBMCs). A=DENV-2 C; B=DENV-1 prM+E; C=DENV-2 prM+E; D=DENV-3 prM+E; E=DENV-4 prM+E; F=DENV-2 NS1; G=DENV-2 NS2; H=DENV-2 NS3; I=DENV-2 NS4; J=DENV-2 NS5.

FIGS. 7A & 7B: Effect of sera from a seronegative subject (FIG. 7A) and a seropositive subject (FIG. 7B) to whom TDV was administered on DENV-2 NS1-induced hyperpermeability as determined by TEER. HPMEC were grown on Transwell semi-permeable membranes (0.4 μm pore size), and serum samples (30 μl) were added to the apical chamber in the presence or absence of DENV2 NS1 (5 μg/ml). DENV2 NS1 is depicted as squares; day 0 serum alone is depicted as diamonds; day 120 serum alone is depicted as triangles; day 0 serum+DENV2 NS1, is depicted as upside-down triangles; day 120 serum+DENV2 NS1 is depicted as X's. (\hat{ }) represents media change. Endothelial permeability was measured at indicated time-points over 48 hours. Relative TEER values from one independent experiment performed in duplicate are plotted. Error bars indicate standard error of the mean (SEM).

FIGS. 8A & 8B: Effect of sera from seronegative and seropositive subjects to which TDV was administered on NS1-induced sialic acid and heparan sulfate degradation. Shown is the quantification of mean fluorescence intensity (MFI) of (FIG. 8A) sialic acid and (FIG. 8B) heparan sulfate expression after staining with sialic acid- and heparan sulfate-specific fluorescent antibodies as visualized by confocal microscopy. Values are normalized to MFI from the NS1+positive control serum group (represented by dotted line at 100%) and expressed as percentage of control. Error bars indicate SEM. The left bar for each subject shows the results at day 0 (d0), the right car for each subject shows the results at day 120 (d120).

FIG. 9: Flow diagram of the clinical trial of Example 6.

FIGS. 10A & 10B: Cumulative incidence of FIG. 10A) virologically-confirmed dengue cases and FIG. 10B) hospitalized virologically-confirmed dengue cases over time during Part 1 study period by baseline serostatus (safety set data; data presented truncated at Month 18). Tables show numbers of participants under follow-up at various time points to end of Part 1 study period.

### DETAILED DESCRIPTION

### Dengue Virus Strains

The dengue virus is a single stranded, positive sense RNA virus of the family flaviviridae. The taxonomy is outlined in Table 1. The family flaviviridae includes three genera, flavivirus, hepacivirus and pestivirus. The genus flavivirus contains highly pathogenic and potentially hemorrhagic fever viruses, such as yellow fever virus and dengue virus, encephalitic viruses, such as Japanese encephalitis virus, Murray Valley encephalitis virus and West Nile virus, and a number of less pathogenic viruses.

TABLE 1
Dengue Virus Taxonomy of the GMO Parental Strain

| | |
|---|---|
| Family | Flaviviridae |
| Genus | Flavivirus |
| Species | Dengue virus |
| Strains | Dengue Serotype 2 (Strain 16681), Strain DEN-2 PDK-53 |
| GMO parent | TDV-2 |

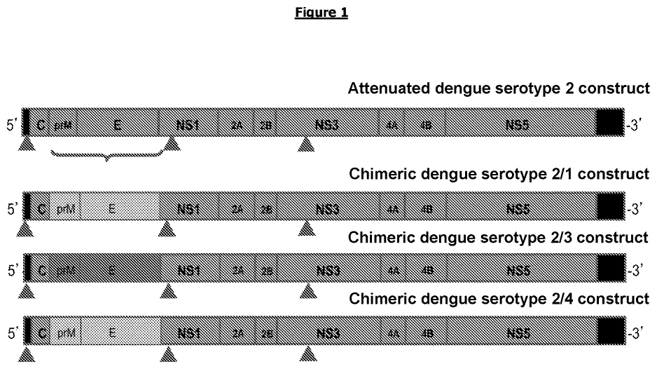

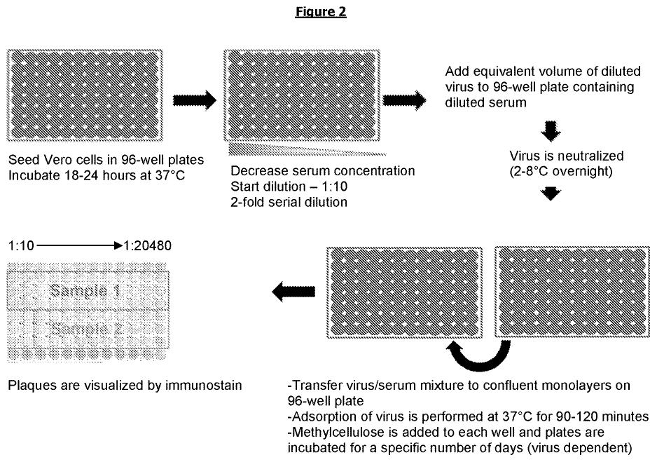

The flavivirus genome comprises in 5′ to 3′ direction (see FIG. 1):
a 5′-noncoding region (5′-NCR),
a capsid protein (C) encoding region,
a pre-membrane protein (prM) encoding region,
an envelope protein (E) encoding region,
a region encoding nonstructural proteins (NSI, NS2A, NS2B, NS3, NS4A, NS4B, NS5) and
a 3′ noncoding region (3′-NCR).

The viral structural proteins are C, prM and E, and the nonstructural proteins are NSI to NS5. The structural and nonstructural proteins are translated as a single polyprotein and processed by cellular and viral proteases.

The unit dose of the invention as described herein comprises a dengue virus composition that comprises four live attenuated dengue virus strains (tetravalent dengue virus composition) representing dengue serotype 1, dengue serotype 2, dengue serotype 3 and dengue serotype 4. Preferably the composition comprises chimeric dengue viruses and optionally at least one non-chimeric dengue virus, in particular a molecularly characterized and cloned dengue serotype 2 strain derived from the live attenuated DEN-2 PDK-53 virus strain (TDV-2), and three chimeric dengue strains derived from the TDV-2 strain by replacing the structural proteins prM and E from TDV-2 with the corresponding structural proteins from the other dengue serotypes, resulting in the following chimeric dengue strains:
a DENV-2/1 chimera (TDV-1),
a DENV-2/3 chimera (TDV-3) and
a DENV-2/4 chimera (TDV-4).

The genetically modified tetravalent dengue vaccine TDV is based on a molecularly characterized and cloned dengue-2 virus strain (TDV-2). This attenuated TDV-2 strain was generated by cDNA cloning of the attenuated laboratory-derived DEN-2 PDK-53 virus strain that was originally isolated at Mahidol University, Bangkok, Thailand (Kinney et al. (1997) Virology 230 (2): 300-308). DEN-2 PDK-53 was generated by 53 serial passages in primary dog kidney (PDK) cells at 32° C. (Bhamarapravati et al. (1987) Bull. World Health Organ. 65 (2): 189-195).

The attenuated DEN-2 PDK-53 strain (the precursor of TDV-2) was derived from the wild type virus strain DEN-2 16681 and differs in nine nucleotides from the wild type as follows (Kinney et al. (1997) Virology 230 (2): 300-308):
(i) 5′-noncoding region (NCR)-57 (nt-57 C-to-T): major attenuation locus
(ii) prM-29 Asp-to-Val (nt-524 A-to-T)
(iii) nt-2055 C-to-T (E gene) silent mutation
(iv) NS1-53 Gly-to-Asp (nt-2579 G-to-A): major attenuation locus
(v) NS2A-181 Leu-to-Phe (nt-4018 C-to-T)
(vi) NS3-250 Glu-to-Val (nt-5270 A-to-T): major attenuation locus
(vii) nt-5547 (NS3 gene) T-to-C silent mutation
(viii) NS4A-75 Gly-to-Ala (nt-6599 G-to-C)
* nt-8571 C-to-T (NS5 gene) silent mutation

The three nucleotide changes located in the 5′ noncoding region (NCR) (nucleotide 57) (mutation (i)), the NS-1 (amino acid 828 of SEQ ID NO. 4) (mutation (iv)) and NS-3 genes (amino acid 1725 of SEQ ID NO. 4) (mutation (vi)) form the basis for the attenuation phenotype of the DEN-2 PDK-53 strain (Butrapet et al. (2000) J. Virol. 74 (7): 3111-3119) (Table 2). These three mutations are referred to herein as the “attenuating mutations” and are comprised in TDV-1, TDV-2, TDV-3 and TDV-4.

TABLE 2
Attenuating mutations in the common
genetic backbone of all TDV strains

| | Nucleotide | Amino Acid |
|---|---|---|
| Location of Mutation | Change in TDV-2 | Change in TDV-2 |
| 5′ Noncoding Region (5′NCR) | 57 C to T | Not applicable (silent) |
| Nonstructural Protein 1 (NS1) | 2579 G to A | 828 Gly to Asp |
| Nonstructural Protein 3 (NS3) | 5270 A to T | 1725 Glu to Val |

In one embodiment, TDV-2 comprises in addition to the three attenuating mutations one or more mutations selected from:
a) a mutation in the prM gene at nucleotide 524 from adenine to thymine resulting in an amino acid change at position 143 from aspartic acid to valine, and/or
b) a silent mutation in the E gene at nucleotide 2055 from cytosine to thymine, and/or
c) a mutation in the NS2A gene at nucleotide 4018 from cytosine to thymine resulting in an amino acid change at position 1308 from leucine to phenylalanine, and/or
d) a silent mutation in the NS3 gene at nucleotide 5547 from thymine to cytosine, and/or
e) a mutation in the NS4A gene at nucleotide 6599 from guanine to cytosine resulting in an amino acid change at position 2168 from glycine to alanine, and/or
f) a silent mutation in the prM gene at nucleotide 900 from thymine to cytosine.

The silent mutation in the NS5 gene at nucleotide 8571 from cytosine to thymine of DEN-2 PDK-53 is not present in the TDV-2 strain.

In another embodiment, TDV-2 comprises in addition to the three attenuating mutations one or more mutations selected from:
g) a mutation in the prM gene at nucleotide 592 from adenine to guanine resulting in an amino acid change at position 166 from lysine to glutamic acid, and/or
h) a mutation in the NS5 gene at nucleotide 8803 from adenine to guanine resulting in an amino acid change at position 2903 from isoleucine to valine.

In another embodiment, TDV-2 comprises in addition to the three attenuating mutations the mutations a) and g), preferably the mutations a), g), c), e) and h), more preferably the mutations a), g), c), e), h) and b), even more preferably the mutations a), g), c), e), h), b) and d), and most preferably the mutations a) to h). The nucleotide positions and amino acids positions of TDV-2 refer to the nucleotide sequence as shown in SEQ ID NO. 3 and amino acid sequence as shown in SEQ ID NO. 4.

The dengue virus structural envelope (E) protein and pre-membrane (prM) protein have been identified as the primary antigens that elicit a neutralizing protective antibody response (Plotkin 2001). For creation of the tetravalent dengue vaccine (TDV), TDV-2 was modified by replacing the nucleic acid sequence encoding the DENV-2 prM and E glycoproteins with the nucleic acid sequence encoding the corresponding wild type prM and E glycoproteins from the DENV-1, DENV-3, and DENV-4 wild type strains DENV-1 16007, DENV-3 16562 or DENV-4 1036 virus, respectively, (see Table 3) using standard molecular genetic engineering methods (Huang et al. (2003) J. Virol. 77 (21): 11436-11447).

TABLE 3
Viral origin of prM/E gene regions of the TDV virus strains

| Virus | Strain | Origin | Source | Reference |
|---|---|---|---|---|
| DENV-1 | 16007 | Thailand, 1964 | DHF/DSS | Halstead and |
| | | | patient | Simasthien, 1970 |
| DENV-2 | 16681 | Thailand, 1964 | DHF/DSS | Halstead and |
| | | | patient | Simasthien, 1970 |
| DENV-3 | 16562 | Philippines, 1964 | DHF | Halstead and |
| | | | patient | Simasthien, 1970 |
| DENV-4 | 1036 | Indonesia, 1976 | DF patient | Gubler et al., 1979 |

A diagram of the four TDV strains comprised in the dengue vaccine composition is shown in FIG. 1.

The chimeric dengue strains TDV-1, TDV-3 and TDV-4 express the surface antigens prM and E of the DENV-1, DENV-3 or DENV-4 viruses, as depicted in Table 3 respectively, and retain the genetic alterations responsible for the attenuation of TDV-2. Thus, each of the TDV-1, TDV-3 and TDV-4 strains comprises the attenuating mutations described in Table 2.

In one embodiment, TDV-1 comprises in addition to the three attenuating mutations one or more mutations selected from:
c) a mutation in the NS2A gene at nucleotide 4018 from cytosine to thymine resulting in an amino acid change at position 1308 from leucine to phenylalanine, and/or
d) a silent mutation in the NS3 gene at nucleotide 5547 from thymine to cytosine, and/or
e) a mutation in the NS4A gene at nucleotide 6599 from guanine to cytosine resulting in an amino acid change at position 2168 from glycine to alanine, and/or
i) a silent mutation in the E gene at nucleotide 1575 from thymine to cytosine, and/or
j) a silent mutation in the junction site between the prM-E gene and the DEN-2 PDK-53 backbone at nucleotide 453 from adenine to guanine, and/or
k) a mutation in the junction site between the prM-E gene and the DEN-2 PDK-53 backbone at nucleotides 2381/2382 from thymine-guanine to cytosine-cytosine resulting in an amino acid change at position 762 from valine to alanine.

In another embodiment, TDV-1 comprises in addition to the three attenuating mutations one or more mutations selected from:
l) a mutation in the NS2A gene at nucleotide 3823 from adenine to cytosine resulting in an amino acid change at position 1243 from isoleucine to leucine, and/or
m) a mutation in the NS2B gene at nucleotide 4407 from adenine to thymine resulting in an amino acid change at position 1437 from glutamic acid to aspartic acid, and/or
n) a silent mutation in the NS4B gene at nucleotide 7311 from adenine to guanine.

In another embodiment, the TDV-1 strain comprises in addition to the three attenuating mutations the mutations l) and m), preferably the mutations l), m), c) and e), even more preferably the mutations l), m), c), e), d) and n), and most preferably the mutations l), m), c), e), d), n), i), j) and k). The nucleotide positions and amino acids positions of TDV-1 refer to the nucleotide sequence as shown in SEQ ID NO. 1 and amino acid sequence as shown in SEQ ID NO. 2.

In one embodiment, TDV-3 comprises in addition to the three attenuating mutations one or more mutations selected from:
c) a mutation in the NS2A gene at nucleotide 4012 from cytosine to thymine resulting in an amino acid change at position 1306 from leucine to phenylalanine, and/or
d) a silent mutation in the NS3 gene at nucleotide 5541 from thymine to cytosine, and/or
e) a mutation in the NS4A gene at nucleotide 6593 from guanine to cytosine resulting in an amino acid change at position 2166 from glycine to alanine, and/or
j) a silent mutation in the junction site between the prM-E gene and the DEN-2 PDK-53 backbone at nucleotide 453 from adenine to guanine, and/or
k) a mutation in the junction site between the prM-E gene and the DEN-2 PDK-53 backbone at nucleotides 2375/2376 from thymine-guanine to cytosine-cytosine resulting in an amino acid change at position 760 from valine to alanine, and/or
o) a silent mutation in the prM gene at nucleotide 552 from cytosine to thymine, and/or
p) a mutation in the E gene at nucleotide 1970 from adenine to thymine resulting in an amino acid change at position 625 from histidine to leucine.

In another embodiment, TDV-3 comprises in addition to the three attenuating mutations one or more mutations selected from:
q) a mutation in the E gene at nucleotide 1603 from adenine to thymine resulting in an amino acid change at position 503 from threonine to serine, and/or
r) a silent mutation in the NS5 gene at nucleotide 7620 from adenine to guanine.

In another embodiments, TDV-3 comprises in addition to the three attenuating mutations the mutations p) and q), preferably the mutations p), q), c) and e), even more preferably the mutations p), q), c), e), d) and r), and most preferably the mutations p), q), c), e), d), r), j), k) and o). The nucleotide positions and amino acids positions of TDV-3 refer to the nucleotide sequence as shown in SEQ ID NO. 5 and amino acid sequence as shown in SEQ ID NO. 6.

In one embodiment, TDV-4 comprises in addition to the three attenuating mutations one or more mutations selected from:
c) a mutation in the NS2A gene at nucleotide 4018 from cytosine to thymine resulting in an amino acid change at position 1308 from leucine to phenylalanine, and/or
d) a silent mutation in the NS3 gene at nucleotide 5547 from thymine to cytosine, and/or
e) a mutation in the NS4A gene at nucleotide 6599 from guanine to cytosine resulting in an amino acid change at position 2168 from glycine to alanine, and/or
j) a silent mutation in the junction site between the prM-E gene and the DEN-2 PDK-53 backbone at nucleotide 453 from adenine to guanine, and/or
k) a mutation in the junction site between the prM-E gene and the DEN-2 PDK-53 backbone at nucleotides 2381/2382 from thymine-guanine to cytosine-cytosine resulting in an amino acid change at position 762 from valine to alanine, and/or
s) a mutation in the C gene at nucleotide 396 from adenine to cytosine resulting in an amino acid change at position 100 from arginine to serine, and/or
t) a silent mutation in the E gene at nucleotide 1401 from adenine to guanine, and/or
u) a mutation in the E gene at nucleotide 2027 from cytosine to thymine resulting in an amino acid change at position 644 from alanine to valine, and/or
v) a mutation in the E gene at nucleotide 2275 from adenine to cytosine resulting in an amino acid change at position 727 from methionine to leucine.

In another embodiment, TDV-4 comprises in addition to the three attenuating mutations one or more mutations selected from:
w) a silent mutation in the C gene at nucleotide 225 from adenine to thymine, and/or
x) a mutation in the NS2A gene at nucleotide 3674 from adenine to guanine resulting in an amino acid change at position 1193 from aspartic acid to glycine, and/or
y) a mutation in the NS2A gene at nucleotide 3773 from adenine to an adenine/guanine mix resulting in an amino acid change at position 1226 from lysine to a lysine/arginine mix, and/or
z) a silent mutation in the NS3 gene at nucleotide 5391 from cytosine to thymine, and/or
aa) a mutation in the NS4A gene at nucleotide 6437 from cytosine to thymine resulting in an amino acid change at position 2114 from alanine to valine, and/or
bb) a silent mutation in the NS4B gene at nucleotide 7026 from thymine to a thymine/cytosine mix, and/or
cc) a silent mutation in the NS5 gene at nucleotide 9750 from adenine to cytosine.

In another embodiments, TDV-4 comprises in addition to the three attenuating mutations the mutation s), u) and v), preferably the mutations s), u), v), c), e), x), y) and aa), even more preferably the mutations s), u), v), c), e), x), y), aa) and w), even more preferably the mutations s), u), v), c), e), x), y), aa), w), d), z), bb) and cc), and most preferably the mutations s), u), v), c), e), x), y), aa), w), d), z), bb), cc), j), k) and t). The nucleotide positions and amino acids positions of TDV-4 refer to the nucleotide sequence as shown in SEQ ID NO. 7 and amino acid sequence as shown in SEQ ID NO. 8.

In a preferred embodiment, TDV-1 has the nucleotide sequence of SEQ ID NO. 1, TDV-2 has the nucleotide sequence of SEQ ID NO. 3, TDV-3 has the nucleotide sequence of SEQ ID NO. 5, and/or TDV-4 has the nucleotide sequence of SEQ ID NO. 7. In a further preferred embodiment, TDV-1 has the amino acid sequence of SEQ ID NO. 2, TDV-2 has the amino acid sequence of SEQ ID NO. 4, TDV-3 has the amino acid sequence of SEQ ID NO. 6, and TDV-4 has the amino acid sequence of SEQ ID NO. 8. In a further preferred embodiment, TDV-1 has a nucleotide sequence encoding the amino acid sequence of SEQ ID NO. 2, TDV-2 has a nucleotide sequence encoding the amino acid sequence of SEQ ID NO. 4, TDV-3 has a nucleotide sequence encoding the amino acid sequence of SEQ ID NO. 6, and TDV-4 has a nucleotide sequence encoding the amino acid sequence of SEQ ID NO. 8.

TABLE 4
Sequences of the TDV virus strains

| SEQ ID NO. | dengue virus strain | sequence type |
|---|---|---|
| SEQ ID NO. 1 | TDV-1 | nucleotide sequence |
| SEQ ID NO. 2 | TDV-1 | amino acid sequence |
| SEQ ID NO. 3 | TDV-2 | nucleotide sequence |
| SEQ ID NO. 4 | TDV-2 | amino acid sequence |
| SEQ ID NO. 5 | TDV-3 | nucleotide sequence |
| SEQ ID NO. 6 | TDV-3 | amino acid sequence |
| SEQ ID NO. 7 | TDV-4 | nucleotide sequence |
| SEQ ID NO. 8 | TDV-4 | amino acid sequence |

Thus, in a particularly preferred embodiment, the unit dose of the invention as described herein comprises the live attenuated dengue virus strains TDV-1, TDV-2, TDV-3 and TDV-4, wherein TDV-1, TDV-3 and TDV-4 are based on TDV-2 and comprise the prM and E regions of DENV-1, -3 and -4, respectively. In another particularly preferred embodiment, TDV-1 is characterized by the nucleotide sequence according to SEQ ID No. 1 and the amino acid sequence according to SEQ ID No. 2, TDV-2 is characterized by the nucleotide sequence according to SEQ ID No. 3 and the amino acid sequence according to SEQ ID No. 4, TDV-3 is characterized by the nucleotide sequence according to SEQ ID No. 5 and the amino acid sequence according to SEQ ID No. 6 and TDV-4 is characterized by the nucleotide sequence according to SEQ ID No. 7 and the amino acid sequence according to SEQ ID No. 8.

The E protein of DENV-3 has two fewer amino acids than the E protein of DENV-2. Therefore, the nucleotides and encoded amino acid backbone of TDV-2 starting after the E region of DENV-3 at nucleotide 2374 of SEQ ID NO. 5 and amino acid 760 of SEQ ID NO. 6 are 6 nucleotides less and 2 amino acids less than the original TDV-2 nucleotide and amino acid positions, respectively.

### Dengue Vaccine Composition

The present invention is in part directed to a unit dose of a dengue vaccine composition as described. The dengue vaccine composition comprises a tetravalent dengue virus composition, also referred to as dengue virus composition, and pharmaceutically acceptable excipients.

### Dengue Virus Composition, Virus Concentrations and %-Concentrations

The present invention is in part directed to a unit dose of a dengue vaccine composition, wherein the dengue vaccine composition comprises a tetravalent dengue virus composition including four live attenuated dengue virus strains:
(i) a dengue serotype 1 preferably in a concentration of at least 3.3 log 10 pfu/0.5 mL,
(ii) a dengue serotype 2 preferably in a concentration of at least 2.7 log 10 pfu/0.5 mL,
(iii) a dengue serotype3 preferably in a concentration of at least 4.0 log 10 pfu/0.5 mL, and
(iv) a dengue serotype 4 preferably strain in a concentration of at least 4.5 log 10 pfu/0.5 mL.

The present invention is further in part directed to a unit dose of a dengue vaccine composition, wherein the dengue vaccine composition comprises a tetravalent dengue virus composition including four live attenuated dengue virus strains:
(i) a chimeric dengue serotype 2/1 strain in a concentration of at least 3.3 log 10 pfu/0.5 mL,
(ii) a dengue serotype 2 strain in a concentration of at least 2.7 log 10 pfu/0.5 mL,
(iii) a chimeric dengue serotype 2/3 strain in a concentration of at least 4.0 log 10 pfu/0.5 mL, and
(iv) a chimeric dengue serotype 2/4 strain in a concentration of at least 4.5 log 10 pfu/0.5 mL.

Preferably, the chimeric dengue serotype 2/1 strain is TDV-1, the dengue serotype 2 strain is TDV-2, the chimeric dengue serotype 2/3 strain is TDV-3 and the chimeric dengue serotype 2/4 strain is TDV-4.

In one embodiment, the dengue vaccine composition comprises a tetravalent dengue virus composition including four live attenuated dengue virus strains wherein:
(i) the dengue serotype 1 (e.g. chimeric dengue serotype 2/1 strain) has a concentration of 3.3 log 10 pfu/0.5 mL to 5.3 log 10 pfu/0.5 mL,
(ii) the dengue serotype 2 (e.g. dengue serotype 2 strain) has a concentration of 2.7 log 10 pfu/0.5 mL to 5.0 log 10 pfu/0.5 mL,
(iii) the dengue serotype 3 (e.g. chimeric dengue serotype 2/3 strain) has a concentration of 4.0 log 10 pfu/0.5 mL to 6.0 log 10 pfu/0.5 mL, and
(iv) the dengue serotype 4 (e.g. chimeric dengue serotype 2/4 strain) has a concentration of 4.5 log 10 pfu/0.5 mL to 6.5 log 10 pfu/0.5 mL.

In one such embodiment, the dengue vaccine composition comprises a tetravalent dengue virus composition including four live attenuated dengue virus strains wherein:
(i) the dengue serotype 1 (e.g. chimeric dengue serotype 2/1 strain) has a concentration of 3.3 log 10 pfu/0.5 mL to 5.0 log 10 pfu/0.5 mL,
(ii) the dengue serotype 2 (e.g. dengue serotype 2 strain) has a concentration of 2.7 log 10 pfu/0.5 mL to 4.9 log 10 pfu/0.5 mL,
(iii) the dengue serotype 3 (e.g. chimeric dengue serotype 2/3 strain) has a concentration of 4.0 log 10 pfu/0.5 mL to 5.7 log 10 pfu/0.5 mL, and
(iv) the dengue serotype 4 (e.g. chimeric dengue serotype 2/4 strain) has a concentration of 4.5 log 10 pfu/0.5 mL to 6.2 log 10 pfu/0.5 mL.

In a further such embodiment, the dengue vaccine composition comprises a tetravalent dengue virus composition including four live attenuated dengue virus strains wherein:
(i) a dengue serotype 1 (e.g. chimeric dengue serotype 2/1 strain) has a concentration of 3.3 log 10 pfu/0.5 mL to 5.0 log 10 pfu/0.5 mL,
(ii) a dengue serotype 2 (e.g. dengue serotype 2 strain) has a concentration of 2.7 log 10 pfu/0.5 mL to 4.9 log 10 pfu/0.5 mL,
(iii) a dengue serotype 3 (e.g. chimeric dengue serotype 2/3 strain) has a concentration of 4.0 log 10 pfu/0.5 mL to 5.7 log 10 pfu/0.5 mL, and
(iv) a dengue serotype 4 (e.g. chimeric dengue serotype 2/4 strain) has a concentration of 4.5 log 10 pfu/0.5 mL to 5.5 log 10 pfu/0.5 mL.

In a further such embodiment, the dengue vaccine composition comprises a tetravalent dengue virus composition including four live attenuated dengue virus strains wherein:
(i) a dengue serotype 1 (e.g. chimeric dengue serotype 2/1 strain) has a concentration of 3.3 log 10 pfu/0.5 mL to 4.1 log 10 pfu/0.5 mL,
(ii) a dengue serotype 2 (e.g. dengue serotype 2 strain) has a concentration of 2.7 log 10 pfu/0.5 mL to 3.6 log 10 pfu/0.5 mL,
(iii) a dengue serotype 3 (e.g. chimeric dengue serotype 2/3 strain) has a concentration of 4.0 log 10 pfu/0.5 mL to 4.7 log 10 pfu/0.5 mL, and
(iv) a dengue serotype 4 (e.g. chimeric dengue serotype 2/4 strain) has a concentration of 4.5 log 10 pfu/0.5 mL to 5.3 log 10 pfu/0.5 mL.

In a further such embodiment, the dengue vaccine composition comprises a tetravalent dengue virus composition including four live attenuated dengue virus strains wherein:
(i) a dengue serotype 1 (e.g. chimeric dengue serotype 2/1 strain) has a concentration of 3.3 log 10 pfu/0.5 mL to 3.6 log 10 pfu/0.5 mL,
(ii) a dengue serotype 2 (e.g. dengue serotype 2 strain) has a concentration of 2.7 log 10 pfu/0.5 mL to 4.0 log 10 pfu/0.5 mL,
(iii) a dengue serotype 3 (e.g. chimeric dengue serotype 2/3 strain) has a concentration of 4.0 log 10 pfu/0.5 mL to 4.6 log 10 pfu/0.5 mL, and
(iv) a dengue serotype 4 (e.g. chimeric dengue serotype 2/4 strain) has a concentration of 4.5 log 10 pfu/0.5 mL to 5.1 log 10 pfu/0.5 mL.

In another embodiment, the dengue vaccine composition comprises a tetravalent dengue virus composition including four live attenuated dengue virus strains wherein:
(i) the dengue serotype 1 (e.g. chimeric dengue serotype 2/1 strain) has a concentration of 4.3 log 10 pfu/0.5 mL to 4.4 log 10 pfu/0.5 mL,
(ii) the dengue serotype 2 (e.g. dengue serotype 2 strain) has a concentration of 3.7 log 10 pfu/0.5 mL to 3.8 log 10 pfu/0.5 mL,
(iii) the dengue serotype 3 (e.g. chimeric dengue serotype 2/3 strain) has a concentration of 4.5 log 10 pfu/0.5 mL to 5.0 log 10 pfu/0.5 mL, and
(iv) the dengue serotype 4 (e.g. chimeric dengue serotype 2/4 strain) has a concentration of 5.5 log 10 pfu/0.5 mL to 5.6 log 10 pfu/0.5 mL.

In a particularly preferred embodiment, the dengue vaccine composition comprises a tetravalent dengue virus composition including four live attenuated dengue virus strains wherein:
(i) the dengue serotype 1 (e.g. chimeric dengue serotype 2/1 strain) has a concentration of 4.4 log 10 pfu/0.5 mL,
(ii) the dengue serotype 2 (e.g. dengue serotype 2 strain) has a concentration of 3.8 log 10 pfu/0.5 mL,
(iii) the dengue serotype 3 (e.g. chimeric dengue serotype 2/3 strain) has a concentration of 4.5 log 10 pfu/0.5 mL, and
(iv) the dengue serotype 4 (e.g. chimeric dengue serotype 2/4 strain) has a concentration of 5.6 log 10 pfu/0.5 mL.

In another particularly preferred embodiment, the dengue vaccine composition comprises a tetravalent dengue virus composition including four live attenuated dengue virus strains wherein:
(i) the dengue serotype 1 (e.g. chimeric dengue serotype 2/1 strain) has a concentration of 3.6 log 10 pfu/0.5 mL,
(ii) the dengue serotype 2 (e.g. dengue serotype 2 strain) has a concentration of 4.0 log 10 pfu/0.5 mL,
(iii) the dengue serotype 3 (e.g. chimeric dengue serotype 2/3 strain) has a concentration of 4.6 log 10 pfu/0.5 mL, and
(iv) the dengue serotype 4 (e.g. chimeric dengue serotype 2/4 strain) has a concentration of 5.1 log 10 pfu/0.5 mL.

In another preferred embodiment, the dengue vaccine composition comprises a tetravalent dengue virus composition including four live attenuated dengue virus strains wherein the arithmetic sum of all four serotypes is less than 6.7 log 10 pfu/0.5 mL, preferably less than 5.5 log 10 pfu/0.5 mL. In certain such embodiments, the arithmetic sum of all four serotypes is at least 4.6 log 10 pfu/0.5 mL. In a preferred embodiment, the dengue vaccine composition comprises a tetravalent dengue virus composition including four live attenuated dengue virus strains wherein the arithmetic sum of all four serotypes is in the range of 4.6 log 10 pfu/0.5 mL to 6.7 log 10 pfu/0.5 mL, preferably in the range of 4.6 log 10 pfu/0.5 ml to 5.5 log 10 pfu/0.5 mL.

Preferably, in said embodiments the chimeric dengue serotype 2/1 strain is TDV-1, the dengue serotype 2 strain is TDV-2, the chimeric dengue serotype 2/3 strain is TDV-3 and the chimeric dengue serotype 2/4 strain is TDV-4. More preferably, TDV-1 is characterized by the nucleotide sequence according to SEQ ID No. 1 and the amino acid sequence according to SEQ ID No. 2, TDV-2 is characterized by the nucleotide sequence according to SEQ ID No. 3 and the amino acid sequence according to SEQ ID No. 4, TDV-3 is characterized by the nucleotide sequence according to SEQ ID No. 5 and the amino acid sequence according to SEQ ID No. 6 and TDV-4 is characterized by the nucleotide sequence according to SEQ ID No. 7 and the amino acid sequence according to SEQ ID No. 8.

The present invention is in part directed to a unit dose of a dengue vaccine composition, wherein the dengue vaccine composition comprises a tetravalent dengue virus composition including four live attenuated dengue virus strains:
(i) a dengue serotype 1 (e.g. chimeric dengue serotype 2/1 strain) in a concentration of at least 3.3 log 10 pfu/dose,
(ii) a dengue serotype 2 (e.g. dengue serotype 2 strain) in a concentration of at least 2.7 log 10 pfu/dose,
(iii) a dengue serotype 3 (e.g. chimeric dengue serotype 2/3 strain) in a concentration of at least 4.0 log 10 pfu/dose, and
(iv) a dengue serotype 4 (e.g. chimeric dengue serotype 2/4 strain) in a concentration of at least 4.5 log 10 pfu/dose.

In one embodiment, the dengue vaccine composition comprises a tetravalent dengue virus composition including four live attenuated dengue virus strains wherein:
(i) the dengue serotype 1 (e.g. chimeric dengue serotype 2/1 strain) has a concentration of 3.3 log 10 pfu/dose to 5.3 log 10 pfu/dose,
(ii) the dengue serotype 2 (e.g. dengue serotype 2 strain) has a concentration of 2.7 log 10 pfu/dose to 5.0 log 10 pfu/dose,
(iii) the dengue serotype 3 (e.g. chimeric dengue serotype 2/3 strain) has a concentration of 4.0 log 10 pfu/dose to 6.0 log 10 pfu/dose, and
(iv) the dengue serotype 4 (e.g. chimeric dengue serotype 2/4 strain) has a concentration of 4.5 log 10 pfu/dose to 6.5 log 10 pfu/dose.

In one such embodiment, the dengue vaccine composition comprises a tetravalent dengue virus composition including four live attenuated dengue virus strains wherein:
(i) the dengue serotype 1 (e.g. chimeric dengue serotype 2/1 strain) has a concentration of 3.3 log 10 pfu/dose to 5.0 log 10 pfu/dose,
(ii) the dengue serotype 2 (e.g. dengue serotype 2 strain) has a concentration of 2.7 log 10 pfu/dose to 4.9 log 10 pfu/dose,
(iii) the dengue serotype 3 (e.g. chimeric dengue serotype 2/3 strain) has a concentration of 4.0 log 10 pfu/dose to 5.7 log 10 pfu/dose, and
(iv) the dengue serotype 4 (e.g. chimeric dengue serotype 2/4 strain) has a concentration of 4.5 log 10 pfu/dose to 6.2 log 10 pfu/dose.

In a further such embodiment, the dengue vaccine composition comprises a tetravalent dengue virus composition including four live attenuated dengue virus strains wherein:
(i) a dengue serotype 1 (e.g. chimeric dengue serotype 2/1 strain) has a concentration of 3.3 log 10 pfu/dose to 5.0 log 10 pfu/dose,
(ii) a dengue serotype 2 (e.g. dengue serotype 2 strain) has a concentration of 2.7 log 10 pfu/dose to 4.9 log 10 pfu/dose,
(iii) a dengue serotype 3 (e.g. chimeric dengue serotype 2/3 strain) has a concentration of 4.0 log 10 pfu/dose to 5.7 log 10 pfu/dose, and
(iv) a dengue serotype 4 (e.g. chimeric dengue serotype 2/4 strain) has a concentration of 4.5 log 10 pfu/dose to 5.5 log 10 pfu/dose.

In a further such embodiment, the dengue vaccine composition comprises a tetravalent dengue virus composition including four live attenuated dengue virus strains wherein:
(i) a dengue serotype 1 (e.g. chimeric dengue serotype 2/1 strain) has a concentration of 3.3 log 10 pfu/dose to 4.1 log 10 pfu/dose,
(ii) a dengue serotype 2 (e.g. dengue serotype 2 strain) has a concentration of 2.7 log 10 pfu/dose to 3.6 log 10 pfu/dose,
(iii) a dengue serotype 3 (e.g. chimeric dengue serotype 2/3 strain) has a concentration of 4.0 log 10 pfu/dose to 4.7 log 10 pfu/dose, and
(iv) a dengue serotype 4 (e.g. chimeric dengue serotype 2/4 strain) has a concentration of 4.5 log 10 pfu/dose to 5.3 log 10 pfu/dose.

In a further such embodiment, the dengue vaccine composition comprises a tetravalent dengue virus composition including four live attenuated dengue virus strains wherein:
(i) a dengue serotype 1 (e.g. chimeric dengue serotype 2/1 strain) has a concentration of 3.3 log 10 pfu/dose to 3.6 log 10 pfu/dose,
(ii) a dengue serotype 2 (e.g. dengue serotype 2 strain) has a concentration of 2.7 log 10 pfu/dose to 4.0 log 10 pfu/dose,
(iii) a dengue serotype 3 (e.g. chimeric dengue serotype 2/3 strain) has a concentration of 4.0 log 10 pfu/dose to 4.6 log 10 pfu/dose, and
(iv) a dengue serotype 4 (e.g. chimeric dengue serotype 2/4 strain) has a concentration of 4.5 log 10 pfu/dose to 5.1 log 10 pfu/dose.

In another embodiment, the dengue vaccine composition comprises a tetravalent dengue virus composition including four live attenuated dengue virus strains wherein:
(i) the dengue serotype 1 (e.g. chimeric dengue serotype 2/1 strain) has a concentration of 4.3 log 10 pfu/dose to 4.4 log 10 pfu/dose,
(ii) the dengue serotype 2 (e.g. dengue serotype 2 strain) has a concentration of 3.7 log 10 pfu/dose to 3.8 log 10 pfu/dose,
(iii) the dengue serotype 3 (e.g. chimeric dengue serotype 2/3 strain) has a concentration of 4.5 log 10 pfu/dose to 5.0 log 10 pfu/dose, and
(iv) the dengue serotype 4 (e.g. chimeric dengue serotype 2/4 strain) has a concentration of 5.5 log 10 pfu/dose to 5.6 log 10 pfu/dose.

In a particularly preferred embodiment, the dengue vaccine composition comprises a tetravalent dengue virus composition including four live attenuated dengue virus strains wherein:
(i) the dengue serotype 1 (e.g. chimeric dengue serotype 2/1 strain) has a concentration of 4.4 log 10 pfu/dose,
(ii) the dengue serotype 2 (e.g. dengue serotype 2 strain) has a concentration of 3.8 log 10 pfu/dose,
(iii) the dengue serotype 3 (e.g. chimeric dengue serotype 2/3 strain) has a concentration of 4.5 log 10 pfu/dose, and
(iv) the dengue serotype 4 (e.g. chimeric dengue serotype 2/4 strain) has a concentration of 5.6 log 10 pfu/dose.

In another particularly preferred embodiment, the dengue vaccine composition comprises a tetravalent dengue virus composition including four live attenuated dengue virus strains wherein:
(i) the dengue serotype 1 (e.g. chimeric dengue serotype 2/1 strain) has a concentration of 3.6 log 10 pfu/dose,
(ii) the dengue serotype 2 (e.g. dengue serotype 2 strain) has a concentration of 4.0 log 10 pfu/dose,
(iii) the dengue serotype 3 (e.g. chimeric dengue serotype 2/3 strain) has a concentration of 4.6 log 10 pfu/dose, and
(iv) the dengue serotype 4 (e.g. chimeric dengue serotype 2/4 strain) has a concentration of 5.1 log 10 pfu/dose.

In another preferred embodiment, the dengue vaccine composition comprises a tetravalent dengue virus composition including four live attenuated dengue virus strains wherein the arithmetic sum of all four serotypes is less than 6.7 log 10 pfu/dose, preferably less than 5.5 log 10 pfu/dose. In certain such embodiments, the arithmetic sum of all four serotypes is at least 4.6 log 10 pfu/dose. in a preferred embodiment, the dengue vaccine composition comprises a tetravalent dengue virus composition including four live attenuated dengue virus strains wherein the arithmetic sum of all four serotypes is in the range of 4.6 log 10 pfu/dose to 6.7 log 10 pfu/dose, preferably in the range of 4.6 log 10 pfu/dose to 5.5 log 10 pfu/dose.

Preferably, in said embodiments the chimeric dengue serotype 2/1 strain is TDV-1, the dengue serotype 2 strain is TDV-2, the chimeric dengue serotype 2/3 strain is TDV-3 and the chimeric dengue serotype 2/4 strain is TDV-4. More preferably, TDV-1 is characterized by the nucleotide sequence according to SEQ ID No. 1 and the amino acid sequence according to SEQ ID No. 2, TDV-2 is characterized by the nucleotide sequence according to SEQ ID No. 3 and the amino acid sequence according to SEQ ID No. 4, TDV-3 is characterized by the nucleotide sequence according to SEQ ID No. 5 and the amino acid sequence according to SEQ ID No. 6 and TDV-4 is characterized by the nucleotide sequence according to SEQ ID No. 7 and the amino acid sequence according to SEQ ID No. 8.

The concentration of the different dengue viruses is preferably determined by an immuno-focus assay known in the art. For example, the concentration may be determined by an immuno-focus assay wherein serial dilutions of dengue virus are applied to monolayers of adherent cells, such as Vero cells. After a period of time which allows infectious viruses to bind to the cells and to be taken up by the cells, an overlay containing thickening agents, such as agarose or carboxymethylcellulose, is added to prevent diffusion of viruses so that progeny viruses can only infect cells adjacent to the original infected cells. After a period of incubation to allow viral replication, cells are fixed and stained using serotype-specific anti-dengue monoclonal antibodies and a secondary antibody such as an antibody labeled with alkaline phosphatase. The foci are stained by adding a suitable substrate for the enzyme attached to the secondary antibody, such as 5-bromo-4-chloro-3-indolyl-phosphate/nitro blue tetrazolium phosphatase substrate. The number of plaques on the plate corresponds to the plaque forming units of the virus in the solutions applied to the cells. For example, a concentration of 1.000 pfu/μl indicates that 1 μl of the solution applied to the cells contains enough viruses to produce 1.000 plaques in a cell monolayer.

The dengue vaccine composition comprises a tetravalent dengue virus composition including four live attenuated dengue virus strains, wherein a chimeric dengue serotype 2/1 strain, a dengue serotype 2 strain, a chimeric dengue serotype 2/3 strain, and a chimeric dengue serotype 2/4 strain provide a total concentration in pfu/0.5 mL. The term “total concentration in pfu/0.5 mL” or “total concentration in pfu/dose” is the sum of the concentrations of the dengue serotype 1 (e.g. chimeric dengue serotype 2/1 strain), dengue serotype 2 (e.g. the dengue serotype 2 strain), the dengue serotype 3 (e.g. chimeric dengue serotype 2/3 strain) and the dengue serotype 4 (e.g. chimeric dengue serotype 2/4 strain), preferably the sum of the concentrations of TDV-1, TDV-2, TDV-3 and TDV-4, and is defined as 100% of the dengue virus concentration as determined by pfu (plaque forming units) in 0.5 mL or in a dose.

In one embodiment, the dengue vaccine composition comprises a tetravalent dengue virus composition including four live attenuated dengue virus strains, wherein a dengue serotype 1 (e.g. chimeric dengue serotype 2/1 strain), a dengue serotype 2 (e.g. dengue serotype 2 strain), a dengue serotype 3 (e.g. chimeric dengue serotype 2/3 strain), and a dengue serotype 4 (e.g. chimeric dengue serotype 2/4 strain) provide a total concentration in pfu/0.5 mL, wherein based on said total concentration the concentration of a dengue serotype 2 (e.g. dengue serotype 2 strain) measured in pfu/0.5 mL is less than 10% of the total concentration, or less than 8%, or less than 6% of the total concentration, and wherein the concentration of a dengue serotype 4 (e.g. chimeric dengue serotype 2/4 strain) measured in pfu/0.5 mL is at least 50% or at least 60% or at least 65% of the total concentration. In one embodiment, based on said total concentration the concentration of a dengue serotype 2 (e.g. dengue serotype 2 strain) measured in pfu/0.5 mL is 0.3 to 10% or 0.5 to 8% of the total concentration and the concentration of a dengue serotype 4 (e.g. chimeric dengue serotype 2/4 strain) measured in pfu/0.5 mL is 50% to 90% or 60% to 88% of the total concentration. This means that the concentration of the dengue serotype 2 (e.g. dengue serotype 2 strain) is lower than the concentration of the dengue serotype 4 (e.g. chimeric dengue serotype 2/4 strain).

In one such embodiment, the concentration of a dengue serotype 1 (e.g. chimeric dengue serotype 2/1 strain) measured in pfu/0.5 mL is at least 1% of the total concentration, and/or the concentration of a dengue serotype 3 (e.g. chimeric dengue serotype 2/3 strain) measured in pfu/0.5 mL is at least 6% of the total concentration, or at least 7% or 8%, 10%, 12%, 14%, 16% or 18% of the total concentration. In one such embodiment, the concentration of a dengue serotype 2 (e.g. chimeric dengue serotype 2/1 strain) measured in pfu/0.5 mL is 1% to 7% or 2% to 6% or 2.0% to 5.0% of the total concentration, and/or the concentration of a dengue serotype 3 (e.g. chimeric dengue serotype 2/3 strain) measured in pfu/0.5 mL is 6% to 25% or 7% to 25% or 10% to 25% or 18% to 25% of the total concentration. This means that the concentration of the dengue serotype 1 (e.g. chimeric dengue serotype 2/1 strain) is lower than the concentration of the dengue serotype 3 (e.g. chimeric dengue serotype 2/3 strain).

In a preferred embodiment, the concentration of a dengue serotype 2 strain, such as TDV-2, measured in pfu/0.5 mL is less than 10% of the total concentration, preferably less than 6% or less than 2%, the concentration of a dengue serotype 4 (e.g. chimeric dengue serotype 2/4 strain), such as TDV-4, measured in pfu/0.5 mL is at least 50% of the total concentration, preferably at least 65%, the concentration of a dengue serotype 1 (e.g. chimeric dengue serotype 2/1 strain), such as TDV-1, measured in pfu/0.5 mL is at least 1% of the total concentration, preferably between 1% and 7% or 2.0% to 5.0%, and the concentration of a dengue serotype 3 (e.g. chimeric dengue serotype 2/3 strain), such as TDV-3, measured in pfu/0.5 mL is at least 6% of the total concentration, preferably between 6% and 25% or 10% to 25% or 18% to 25%.

In a further preferred embodiment, a dengue virus composition comprising a dengue serotype 1 (e.g. chimeric dengue serotype 2/1 strain), a dengue serotype 2 (e.g. dengue serotype 2 strain), a dengue serotype 1 (e.g. chimeric dengue serotype 2/3 strain), and a dengue serotype 4 (e.g. chimeric dengue serotype 2/4 strain), such as TDV-1, TDV-2, TDV-3 and TDV-4, is provided, wherein the concentration of the dengue serotype 1 (e.g. chimeric dengue serotype 2/1 strain) measured in pfu/0.5 mL is at least 1% of the total concentration, preferably between 1% and 7% or 2.0% and 5.0%, the concentration of the dengue serotype 2 (e.g. dengue serotype 2 strain) measured in pfu/0.5 mL is less than 10% of the total concentration, preferably less than 6% or less than 2% and the concentration of the dengue serotype 3 (e.g. chimeric dengue serotype 2/3 strain) measured in pfu/0.5 mL is at least 6% of the total concentration, preferably between 6% and 25% or 10% to 25% or 18% to 25%. It is particularly preferred that the dengue serotype 4 (e.g. chimeric dengue serotype 2/4 strain) has the highest concentration of all four dengue serotypes.

In a further preferred embodiment, the dengue vaccine composition comprises a tetravalent dengue virus composition including four live attenuated dengue virus strains, wherein the concentration of the dengue serotype 1 (e.g. chimeric dengue serotype 2/1 strain) measured in pfu/0.5 mL is 1% to 7% of the total concentration, the concentration of the dengue serotype 2 (e.g. dengue serotype 2 strain) measured in pfu/0.5 mL is less than 8% of the total concentration, such as in the range of 1% to 8% of the total concentration, the concentration of the dengue serotype 3 (e.g. chimeric dengue serotype 2/3 strain) measured in pfu/0.5 mL is at least 10% of the total concentration, and the concentration of the dengue serotype 4 (e.g. chimeric dengue serotype 2/4 strain) measured in pfu/0.5 mL is at least 65% of the total concentration, such as in the range of 65% to 80%. In certain such embodiments, the arithmetic sum of all four serotypes is in the range of 4.6 log 10 pfu/0.5 mL to 6.7 log 10 pfu/0.5 mL, preferably in the range of 4.6 log 10 pfu/0.5 mL to 5.5 log 10 pfu/0.5 mL.

In a further preferred embodiment the dengue serotype 1 (e.g. chimeric dengue serotype 2/1 strain) such as TDV-1 and the dengue serotype 2 (e.g. dengue serotype 2 strain) such as TDV-2 are present each in a concentration based on the total concentration in pfu/0.5 mL which is within 5%-points of each other and/or are together less than about 10% of the total concentration in pfu/0.5 mL. In certain such embodiments the dengue serotype 3 (e.g. chimeric dengue serotype 2/3 strain) such as TDV-3 is preferably at least about 10% of the total concentration in pfu/0.5 mL and more preferably the dengue serotype 4 (e.g. chimeric dengue serotype 2/4 strain) such as TDV-4 is at least about 70% of the total concentration in pfu/0.5 mL. In certain such embodiments the dengue serotype 4 (e.g. chimeric dengue serotype 2/4 strain) such as TDV-4 represents the highest concentration in the composition of all four serotypes, preferably with at least about 70% of the total concentration in pfu/0.5 mL, dengue serotype 3 (e.g. chimeric dengue serotype 2/3 strain) such as TDV-3 represents the second highest concentration in the composition of all four serotypes, preferably with at least about 10% of the total concentration in pfu/0.5 mL, and dengue serotype 1 (e.g. chimeric dengue serotype 2/1 strain) such as TDV-1 and dengue serotype 2 (e.g. dengue serotype 2 strain) such as TDV-2 each represent lower concentrations than the concentration of serotype 3 (e.g. chimeric dengue serotype 2/3 strain) such as TDV-3, and optionally together represent less than about 10% of the total concentration in pfu/0.5 mL.

Preferably, in said embodiments the chimeric dengue serotype 2/1 strain is TDV-1, the dengue serotype 2 strain is TDV-2, the chimeric dengue serotype 2/3 strain is TDV-3 and the chimeric dengue serotype 2/4 strain is TDV-4. More preferably, TDV-1 is characterized by the nucleotide sequence according to SEQ ID No. 1 and the amino acid sequence according to SEQ ID No. 2, TDV-2 is characterized by the nucleotide sequence according to SEQ ID No. 3 and the amino acid sequence according to SEQ ID No. 4, TDV-3 is characterized by the nucleotide sequence according to SEQ ID No. 5 and the amino acid sequence according to SEQ ID No. 6 and TDV-4 is characterized by the nucleotide sequence according to SEQ ID No. 7 and the amino acid sequence according to SEQ ID No. 8.

According to a further embodiment, the chimeric dengue serotype 2/4 strain, preferably TDV-4, has the highest concentration in the dengue vaccine composition, followed by the chimeric dengue serotype 2/3 strain, preferably TDV-3, followed by the chimeric dengue serotype 2/1 strain, preferably TDV-1, followed by the dengue serotype 2 strain, preferably TDV-2. It is particularly preferred that the dengue serotype 2 strain has the lowest concentration of the four strains present in the dengue vaccine composition.

### Pharmaceutically Acceptable Excipients

The present invention is in part directed to a unit dose of a dengue vaccine composition, wherein the dengue vaccine composition comprises one or more pharmaceutically acceptable excipients. In one embodiment, the dengue vaccine composition comprises a non-reducing sugar, a surfactant, a protein and an inorganic salt. Preferably, the non-reducing sugar is trehalose, the surfactant is poloxamer 407, the protein is human serum albumin and the inorganic salt is sodium chloride.

In one embodiment, the unit dose of a dengue vaccine composition comprises the following pharmaceutically acceptable excipients:
from about 10% w/v to about 20% w/v α,α-trehalose dihydrate or an equimolar amount of other forms of α,α-trehalose,
from about 0.5% w/v to about 1.5% w/v poloxamer 407,
from about 0.05% w/v to about 2% w/v human serum albumin, and from about 70 mM to 140 mM sodium chloride.

In a preferred embodiment, the lyophilized unit dose of the invention as described herein comprises the following pharmaceutically acceptable excipients:
about 15% w/v α,α-trehalose dihydrate,
about 1% w/v poloxamer 407,
about 0.1% w/v human serum albumin, and
about 100 mM sodium chloride.

In a preferred embodiment, the reconstituted unit dose of the invention as described herein comprises the following pharmaceutically acceptable excipients:
about 15% w/v α,α-trehalose dihydrate,
about 1% w/v poloxamer 407,
about 0.1% w/v human serum albumin, and
about 137 mM sodium chloride.

The human serum albumin may be a native or recombinant human serum albumin (rHSA). The poloxamer 407 may be e.g. Pluronic F127.

In one embodiment, the unit dose further comprises a buffer. The buffer may be phosphate buffered saline (PBS). The buffer may include at least one of sodium chloride (NaCl), monosodium dihydrogen phosphate (NaH₂PO₄), disodium hydrogen phosphate (Na₂HPO₄), potassium chloride (KCl), and potassium dihydrogen phosphate (KH₂PO₄). In a preferred embodiment, the buffer may include disodium hydrogen phosphate (Na₂HPO₄), potassium chloride (KCl), and potassium dihydrogen phosphate (KH₂PO₄). The buffer may have a pH in the range of 7.0 to 8.5 at 25° C.

### Unit Dose

The present invention is directed in part to a unit dose of a dengue vaccine composition comprising a tetravalent dengue virus composition as described herein and pharmaceutically acceptable excipients as described herein.

The present invention is directed in part to a unit dose of a dengue vaccine composition as described above e.g. of
(i) a dengue serotype 1 (e.g. chimeric dengue serotype 2/1 strain) with a concentration of at least 3.3 log 10 pfu/0.5 mL,
(ii) a dengue serotype 2 (e.g. dengue serotype 2 strain) with a concentration of at least 2.7 log 10 pfu/0.5 mL,
(iii) a dengue serotype 3 (e.g. chimeric dengue serotype 2/3 strain) with a concentration of at least 4.0 log 10 pfu/0.5 mL, and
(iv) a dengue serotype 4 (e.g. chimeric dengue serotype 2/4 strain) with a concentration of at least 4.5 log 10 pfu/0.5 mL.

Preferably, the chimeric dengue serotype 2/1 strain is TDV-1, the dengue serotype 2 strain is TDV-2, the chimeric dengue serotype 2/3 strain is TDV-3, and the chimeric dengue serotype 2/4 strain is TDV-4. More preferably, TDV-1 is characterized by the nucleotide sequence according to SEQ ID No. 1 and the amino acid sequence according to SEQ ID No. 2, TDV-2 is characterized by the nucleotide sequence according to SEQ ID No. 3 and the amino acid sequence according to SEQ ID No. 4, TDV-3 is characterized by the nucleotide sequence according to SEQ ID No. 5 and the amino acid sequence according to SEQ ID No. 6 and TDV-4 is characterized by the nucleotide sequence according to SEQ ID No. 7 and the amino acid sequence according to SEQ ID No. 8.

In one embodiment, the unit dose is lyophilized. In one such embodiment, the lyophilized unit dose is obtained by subjecting a volume of 0.5 mL of the aqueous dengue vaccine composition produced by combining pharmaceutically acceptable excipients as described herein and the dengue vaccine composition as described herein comprising the four dengue virus strains, in particular TDV-1 to TDV-4, to lyophilization. In a preferred embodiment the residual moisture content as determined by Karl Fischer Determination is equal to or less than 5.0%, preferably equal to or less than 3%.

In another embodiment, the unit dose is reconstituted. The reconstituted unit dose is obtained by subjecting the lyophilized unit dose to reconstitution with a pharmaceutically acceptable diluent, preferably before administration of the dengue vaccine. In one such embodiment, reconstitution will be accomplished by adding a pharmaceutically acceptable diluent, such as water for injection, phosphate buffered saline or an aqueous sodium chloride solution, to the lyophilized unit dose. In one embodiment, an aqueous sodium chloride solution, such as a 37 mM aqueous sodium chloride solution, is added to the lyophilized unit dose for reconstitution. In one such embodiment, the lyophilized unit dose will be reconstituted with 0.3 to 0.8 mL, or 0.4 to 0.7 mL, or 0.5 mL of diluent. In a preferred embodiment, the lyophilized unit dose is reconstituted with 0.3 to 0.8 mL, 0.4 to 0.7 mL or 0.5 mL of 37 mM aqueous sodium chloride solution. In a more preferred embodiment, the lyophilized unit dose is reconstituted with 0.5 ml of 37 mM aqueous sodium chloride solution. The reconstituted unit dose can subsequently be administered subcutaneously.

It is preferred that the unit dose in lyophilized form is the final product after manufacture of the unit dose and the storage form of the unit dose, wherein the unit dose in reconstituted form is prepared before administration of the unit dose to a subject.

In one embodiment, the present invention is directed to a lyophilized unit dose of a dengue vaccine composition comprising upon reconstitution with 0.5 ml of a pharmaceutically acceptable diluent a dengue serotype 1 (e.g. chimeric dengue serotype 2/1 strain) with a concentration of at least 3.3 log 10 pfu/0.5 mL, a dengue serotype 2 (e.g. dengue serotype 2 strain) with a concentration of at least 2.7 log 10 pfu/0.5 mL, a dengue serotype 3 (e.g. chimeric dengue serotype 2/3 strain) with a concentration of at least 4.0 log 10 pfu/0.5 mL, and a dengue serotype 4 (e.g. chimeric dengue serotype 2/4 strain) with a concentration of at least 4.5 log 10 pfu/0.5 mL and pharmaceutically acceptable excipients as described herein, wherein the unit dose is preferably formulated in 0.5 mL before lyophilization. Preferably, the chimeric dengue serotype 2/1 strain is TDV-1, the dengue serotype 2 strain is TDV-2, the chimeric dengue serotype 2/3 strain is TDV-3 and the chimeric dengue serotype 2/4 strain is TDV-4. More preferably, TDV-1 is characterized by the nucleotide sequence according to SEQ ID No. 1 and the amino acid sequence according to SEQ ID No. 2, TDV-2 is characterized by the nucleotide sequence according to SEQ ID No. 3 and the amino acid sequence according to SEQ ID No. 4, TDV-3 is characterized by the nucleotide sequence according to SEQ ID No. 5 and the amino acid sequence according to SEQ ID No. 6 and TDV-4 is characterized by the nucleotide sequence according to SEQ ID No. 7 and the amino acid sequence according to SEQ ID No. 8.

In one such embodiment, the lyophilized unit dose is obtained by lyophilizing 0.5 ml of a dengue vaccine composition comprising a dengue serotype 1 (e.g. chimeric dengue serotype 2/1 strain) in a concentration of 3.3 log 10 pfu/dose to 5.0 log 10 pfu/0.5 mL, a dengue serotype 2 (e.g. dengue serotype 2 strain) in a concentration of 2.7 log 10 pfu/dose to 4.9 log 10 pfu/0.5 mL, a dengue serotype 3 (e.g. chimeric dengue serotype 2/3 strain) in a concentration of 4.0 log 10 pfu/dose to 5.7 log 10 pfu/0.5 mL, and a dengue serotype 4 (e.g. chimeric dengue serotype 2/4 strain) in a concentration of 4.5 log 10 pfu/dose to 5.5 log 10 pfu/0.5 mL and pharmaceutically acceptable excipients as described herein. Preferably, the chimeric dengue serotype 2/1 strain is TDV-1, the dengue serotype 2 strain is TDV-2, the chimeric dengue serotype 2/3 strain is TDV-3 and the chimeric dengue serotype 2/4 strain is TDV-4.

In one such embodiment, the lyophilized unit dose is obtained by lyophilizing 0.5 mL of a dengue vaccine composition comprising a dengue serotype 1 (e.g. chimeric dengue serotype 2/1 strain) in a concentration of 3.3 log 10 pfu/0.5 mL to 3.6 log 10 pfu/0.5 mL, a dengue serotype 2 (e.g. dengue serotype 2 strain) in a concentration of 2.7 log 10 pfu/0.5 mL to 4.0 log 10 pfu/0.5 mL, a dengue serotype 3 (e.g. chimeric dengue serotype 2/3 strain) in a concentration of 4.0 log 10 pfu/0.5 mL to 4.6 log 10 pfu/0.5 mL, and a dengue serotype 4 (e.g. chimeric dengue serotype 2/4 strain) in a concentration of 4.5 log 10 pfu/0.5 mL to 5.1 log 10 pfu/0.5 mL and pharmaceutically acceptable excipients as described herein. Preferably, the chimeric dengue serotype 2/1 strain is TDV-1, the dengue serotype 2 strain is TDV-2, the chimeric dengue serotype 2/3 strain is TDV-3 and the chimeric dengue serotype 2/4 strain is TDV-4.

In certain embodiments, the lyophilized unit dose refers to 0.5 mL before lyophilization, wherein TDV-2 and TDV-4 are present in certain relative amounts, based on the total concentration of TDV-1, TDV-2, TDV-3 and TDV-4 in pfu/0.5 mL, and the concentration of TDV-2 measured in pfu/0.5 mL is less than 10% or less than 8% or less than 6%, and the concentration of TDV-4 measured in pfu/0.5 mL is at least 50% or at least 65%. In some of these embodiments, the concentration of TDV-1 measured in pfu/0.5 mL is at least 1% and/or the concentration of TDV-3 measured in pfu/0.5 mL is at least 6%, 7%, 8%, 10%, 12%, 14%, 16% or at least 18%.

In certain embodiments, the reconstituted unit dose has a volume of 0.5 mL and TDV-2 and TDV-4 are present in certain relative amounts, based on the total concentration of TDV-1, TDV-2, TDV-3 and TDV-4 in pfu/0.5 mL, and the concentration of TDV-2 measured in pfu/0.5 mL is less than 10% or less than 8% or less than 6%, and the concentration of TDV-4 measured in pfu/0.5 mL is at least 50% or at least 65%. In some of these embodiments, the concentration of TDV-1 measured in pfu/0.5 mL is at least 1% and/or the concentration of TDV-3 measured in pfu/0.5 mL is at least 6%, 7%, 8%, 10%, 12%, 14%, 16% or at least 18%.

In a further preferred embodiment, the reconstituted unit dose has a volume of 0.5 mL and comprises a tetravalent dengue virus composition including four live attenuated dengue virus strains, wherein the concentration of the dengue serotype 1 (e.g. dengue serotype 2/1 strain) measured in pfu/0.5 mL is 1% to 7% of the total concentration, the concentration of the dengue serotype 2 (e.g. dengue serotype 2 strain) measured in pfu/0.5 mL is less than 8% of the total concentration, such as in the range of 1% to 8% of the total concentration, the concentration of the dengue serotype 3 (e.g. dengue serotype 2/3 strain) measured in pfu/0.5 mL is at least 10% of the total concentration, and the concentration of the dengue serotype 4 (e.g. dengue serotype 2/4 strain) measured in pfu/0.5 mL is at least 65% of the total concentration, such as in the range of 65% to 80%. In certain such embodiments, the arithmetic sum of all four serotypes is in the range of 4.6 log 10 pfu/0.5 mL to 6.7 log 10 pfu/0.5 mL, preferably in the range of 4.6 log 10 pfu/0.5 mL to 5.5 log 10 pfu/0.5 mL.

In a further preferred embodiment, the reconstituted unit dose has a volume of 0.5 ml and comprises a tetravalent dengue virus composition including four live attenuated dengue virus strains, wherein the dengue serotype 1 (e.g. chimeric dengue serotype 2/1 strain) such as TDV-1 and the dengue serotype 2 (e.g. dengue serotype 2 strain) such as TDV-2 are present each in a concentration based on the total concentration in pfu/0.5 mL which is within 5%-points of each other and/or are together less than about 10% of the total concentration in pfu/0.5 mL. In certain such embodiments the dengue serotype 3 (e.g. chimeric dengue serotype 2/3 strain) such as TDV-3 is preferably at least about 10% of the total concentration in pfu/0.5 mL and more preferably the dengue serotype 4 (e.g. chimeric dengue serotype 2/4 strain) such as TDV-4 is at least about 70% of the total concentration in pfu/0.5 mL. In certain such embodiments the dengue serotype 4 (e.g. chimeric dengue serotype 2/4 strain) such as TDV-4 represents the highest concentration in the composition of all four serotypes, preferably with at least about 70% of the total concentration in pfu/0.5 mL, dengue serotype 3 (e.g. chimeric dengue serotype 2/3 strain) such as TDV-3 represents the second highest concentration in the composition of all four serotypes, preferably with at least about 10% of the total concentration in pfu/0.5 mL, and dengue serotype 1 (e.g. chimeric dengue serotype 2/1 strain) such as TDV-1 and dengue serotype 2 (e.g. dengue serotype 2 strain) such as TDV-2 each represent lower concentrations than the concentration of serotype 3 (e.g. chimeric dengue serotype 2/3 strain) such as TDV-3, and optionally together represent less than about 10% of the total concentration in pfu/0.5 mL.

The lyophilized unit dose reconstituted in 0.5 mL will provide the above concentrations for the four dengue serotypes. While the unit dose of a dengue vaccine composition as described herein refers to the concentrations of the dengue serotypes in 0.5 mL, the lyophilized unit dose can be reconstituted with other volumes of a pharmaceutically acceptable diluent, such as an aqueous sodium chloride solution, without changing the absolute virus amount administered or the ratios of the viruses to one another.

In certain embodiments, the lyophilized unit dose of the invention is prepared from a solution comprising a non-reducing sugar, a surfactant, a protein and an inorganic salt.

In certain embodiments, the lyophilized unit dose of the invention is prepared from a solution comprising trehalose, poloxamer 407, human serum albumin and sodium chloride.

In certain embodiments, the lyophilized unit dose of the invention is prepared from a solution comprising about 10% w/v to about 20% w/v α,α-trehalose dihydrate or an equimolar amount of other forms of α,α-trehalose, from about 0.5% w/v to about 1.5% w/v poloxamer 407, from about 0.05% w/v to about 2% w/v human serum albumin, and about 70 mM to about 120 mM sodium chloride.

In preferred embodiments, the lyophilized unit dose of the invention as described herein is prepared from a solution comprising about 15% w/v α,α-trehalose dihydrate, about 1% w/v poloxamer 407, about 0.1% w/v human serum albumin and about 100 mM sodium chloride.

In one embodiment, the solution from which the lyophilized unit dose is prepared further comprises a buffer. The buffer may be phosphate buffered saline (PBS). The buffer may include at least one of sodium chloride (NaCl), monosodium dihydrogen phosphate (NaH₂PO₄), disodium hydrogen phosphate (Na₂HPO₄), potassium chloride (KCl), and potassium dihydrogen phosphate (KH₂PO₄). In a preferred embodiment, the buffer may include disodium hydrogen phosphate (Na₂HPO₄), potassium chloride (KCl), and potassium dihydrogen phosphate (KH₂PO₄). The buffer may have a pH in the range of about 7.0 to about 8.5 at 25° C. or a pH of about 6.8 to about 7.6 at 25° C., preferably a pH of about 7.2 at 25° C.

In preferred embodiments, the reconstituted unit dose of the invention as described herein comprising about 15% w/v α,α-trehalose dihydrate, about 1% w/v poloxamer 407, about 0.1% w/v human serum albumin and about 137 mM sodium chloride. The reconstituted unit dose may have a pH of about 7.0 to about 8.5 at 25° C., preferably a pH of about 7.2 at 25° C.

The unit dose of the invention as described herein activates multiple arms of the immune system—neutralizing antibodies, cellular immunity and anti-NS1 antibodies—in both seronegative and seropositive subject populations or in both seronegative and seropositive subjects. Thus, the unit dose of the invention as described herein protects both dengue seronegative and dengue seropositive subject populations or subjects against dengue disease.

In one embodiment, one unit dose is present in a container, preferably a vial, and said unit dose is administered to a subject after reconstitution. In one embodiment, more than one unit dose of the dengue vaccine composition may be present in a container, preferably a vial, so that with the content of one container, preferably a vial, more than one subject can be vaccinated. In one embodiment, the container comprising more than one unit doses of the invention as described herein is used for providing the reconstituted unit dose to be used in the methods of the invention as described herein.

The certain embodiments, the container comprising the unit dose of the invention is part of a kit. Thus, the invention is directed in part to a kit for preparing a reconstituted unit dose comprising a lyophilized unit dose of the present invention as described herein, and a pharmaceutically acceptable diluent for reconstitution.

In certain embodiments, the diluent for reconstitution provided in a container, preferably a vial, or a pre-filled syringe. In some embodiments, the diluent for reconstitution is selected from water for injection, phosphate buffered saline or an aqueous sodium chloride solution. In a preferred embodiment, the diluent for reconstitution is 30 to 40 mM sodium chloride, such as 37 mM sodium chloride.

### Method of Preventing and Uses

### Method of Preventing

The present invention is directed in part to a method of preventing dengue disease (in particular virologically confirmable dengue, VCD) in a subject. Thus, in certain embodiments the invention is directed to a method of preventing dengue disease in a subject, comprising administering to the subject a unit dose, in particular a reconstituted unit dose of the invention as described herein.

The present invention is directed in part to a method of preventing dengue disease (in particular virologically confirmable dengue, VCD) in a subject population. Thus, in certain embodiments the invention is directed to a method of preventing dengue disease in a subject population, comprising administering to the subject population a unit dose, in particular a reconstituted unit dose of the invention as described herein.

The present invention is in part directed to said method for preventing dengue disease (in particular virologically confirmable dengue, VCD) in a subject population comprising administering to the subject population at least a first reconstituted unit dose of the invention as described herein, wherein certain ratios of geometric mean neutralizing antibody titers (GMTs) at day 180 or 365 after administration of said first unit dose to the subject population are achieved. According to some embodiments, the geometric mean neutralizing antibody titer for dengue serotype 2 (GMT DENV-2) and the geometric mean neutralizing antibody titer for dengue serotype 4 (GMT DENV-4) when tested in at least 40, or at least 50, or at least 60 subjects at day 180 or day 365 after at least a first administration of said reconstituted unit dose of the invention as described herein, and optionally a second administration of a reconstituted unit dose of the invention as described herein 90 days after said first administration, provide a ratio of GMT DENV-2:GMT DENV-4 of not more than 50, or not more than 40, or not more than 30, or not more than 20. In some of these embodiments, the ratio of GMT DENV-2:GMT DENV-1 is not more than 20, or not more than 18, or not more than 15 at day 180 or 365 after administration of said first reconstituted unit dose, and/or the ratio of GMT DENV-2:GMT DENV-3 is not more than 20, or not more than 18, or not more than 15 at day 180 or 365 after administration of said first reconstituted unit dose.

The present invention is in part directed to said method for preventing dengue disease (in particular virologically confirmable dengue, VCD) in a subject comprising administering to the subject at least a first reconstituted unit dose of the invention as described herein, wherein certain ratios of neutralizing antibody titers at day 180 or 365 after administration of said first unit dose to the subject are achieved. According to some embodiments, the neutralizing antibody titer for dengue serotype 2 and the neutralizing antibody titer for dengue serotype 4 at day 180 or day 365 after at least a first administration of the reconstituted unit dose of the invention as described herein, and optionally a second administration of a reconstituted unit dose of the invention as described herein 90 days after said first administration, provide a ratio of neutralizing antibody titer for DENV-2:neutralizing antibody titer for GMT DENV-4 of not more than 50, or not more than 40, or not more than 30, or not more than 20. In some of these embodiments, the ratio of the neutralizing antibody titers of DENV-2: DENV-1 is not more than 20, or not more than 18, or not more than 15 at day 180 or 365 after administration of said first reconstituted unit dose, and/or the ratio of the neutralizing antibody titers of DENV-2: DENV-3 is not more than 20, or not more than 18, or not more than 15 at day 180 or 365 after administration of said first reconstituted unit dose.

The geometric mean neutralizing antibody titers (GMTs) of a subject population or the neutralizing antibody titers of a subject are determined in accordance with the microneutralization test disclosed herein, for example according to the method described in Example 2. Without wishing to be bound to any theory, it is presently understood that a method inducing a more balanced immune response due to the administration of the reconstituted unit dose of the invention as described herein, in terms of less differences between the geometric mean neutralizing antibody titers (GMTs) against the four dengue serotypes or the neutralizing antibody titers against the four dengue serotypes, is beneficial to the subject or subject population to be vaccinated. In particular, it is understood that a much greater response to any one of the four serotypes, such as to DENV-2 in comparison to the other serotypes, is less beneficial.

The present invention is in part directed to said method for preventing dengue disease (in particular virologically confirmable dengue, VCD) in a subject or subject population wherein the method provides a seropositivity rate in a subject population of at least 50 subjects including the administration of two unit doses subcutaneously at day 1 and at day 90, wherein the subjects of the subject population are seronegative to all dengue serotypes at baseline. In certain such embodiments, at least 80% of the subject population are seropositive for all four dengue serotypes at least one month after administration of the first unit dose, such as at day 30, and/or at least 80% of the subject population are seropositive for all four dengue serotypes before or at the time of the administration of the second unit dose, such as at day 90, and/or at least 80%, or at least 85%, or at least 90%, or at least 95% of the subject population are seropositive for all four dengue serotypes after the administration of the second unit dose, such as at day 120, and/or at least 80%, or at least 85%, or at least 90% of the subject population are seropositive for all four dengue serotypes after the administration of the second unit dose, such as at day 270.

The present invention is in part directed to said method for preventing dengue disease (in particular virologically confirmable dengue, VCD) in a subject or subject population wherein the method provides a seropositivity rate in a subject population of at least 100 subjects including administration of two unit doses subcutaneously at day 1 and at day 90, wherein the subjects of the subject population comprises from 20% to 40% subjects who are seronegative to all dengue serotypes and from 60% to 80% subjects who are seropositive to at least one dengue serotype at base line, wherein at day 120 and/or day 270 the seropositivity rate for all four dengue serotypes in the seronegative part of the subject population and the seropositivity rate for all four dengue serotypes in the seropositive part of the subject population do not deviate more than 10%-points and/or wherein at day 120 the seropositivity rate for all four dengue serotypes in the seronegative part of the subject population and the seropositivity rate for all four dengue serotypes in the seropositive part of the subject population do not deviate more than 5%-points.

The present invention is in part directed to a method of preventing virologically confirmable dengue disease in a subject or subject population comprising administering to the subject or subject population a reconstituted unit dose of a tetravalent dengue virus composition including four live, attenuated dengue serotypes, in particular the virus strains as described herein.

The present invention is in part directed to a method of preventing virologically confirmable dengue disease with hospitalization in a subject or subject population comprising administering to the subject or subject population a reconstituted unit dose of a tetravalent dengue virus composition including four live, attenuated dengue serotypes, in particular the virus strains as described herein.

In certain embodiments, the invention is directed to said methods, wherein said dose unit comprises a tetravalent dengue virus composition including four live attenuated dengue serotypes, in particular the virus strains described herein wherein the serotypes have certain concentrations as described herein with respect to the virus composition and unit dose such as:
(i) a dengue serotype 1 (e.g. chimeric dengue serotype 2/1 strain) has a concentration of 3.3 log 10 pfu/0.5 mL to 5.0 log 10 pfu/0.5 mL,
(ii) a dengue serotype 2 (e.g. dengue serotype 2 strain) has a concentration of 2.7 log 10 pfu/0.5 mL to 4.9 log 10 pfu/0.5 mL,
(iii) a dengue serotype 3 (e.g. chimeric dengue serotype 2/3 strain) has a concentration of 4.0 log 10 pfu/0.5 mL to 5.7 log 10 pfu/0.5 mL, and
(iv) a dengue serotype 4 (e.g. chimeric dengue serotype 2/4 strain) has a concentration of 4.5 log 10 pfu/0.5 mL to 5.5 log 10 pfu/0.5 mL.

In preferred such embodiments, the subject or subject population is of 2 to 17 years of age, such as 4 to 16 years of age, and preferably less than 9 years of age. In other preferred embodiments, the subject or subject population is 4-5 years of age, 6-11 years of age or 12-16 years of age.

In certain embodiments, the invention is directed to said methods, wherein said unit dose upon reconstitution with 0.5 mL of a pharmaceutically acceptable diluent has a concentration of 3.3 log 10 pfu/0.5 mL to 3.6 log 10 pfu/0.5 mL for dengue serotype 1 (e.g. chimeric dengue serotype 2/1 strain), has a concentration of 2.7 log 10 pfu/0.5 mL to 4.0 log 10 pfu/0.5 mL for dengue serotype 2 (e.g. dengue serotype 2 strain), has a concentration of 4.0 log 10 pfu/0.5 mL to 4.6 log 10 pfu/0.5 mL for dengue serotype 3 (e.g. chimeric dengue serotype 2/3 strain) and has a concentration of 4.5 log 10 pfu/0.5 mL to 5.1 log 10 pfu/0.5 mL for dengue serotype 4 (e.g. chimeric dengue serotype 2/4 strain). In preferred such embodiments, the subject or subject population is of 2 to 17 years of age, such as 4 to 16 years of age, and preferably less than 9 years of age. In other preferred embodiments, the subject or subject population is 4-5 years of age, 6-11 years of age or 12-16 years of age.

In certain embodiments, the invention is directed to said methods, wherein the concentration of the dengue serotype 1 (e.g. chimeric dengue serotype 2/1 strain) measured in pfu/0.5 mL is 1% to 7% of the total concentration, the concentration of the dengue serotype 2 (e.g. dengue serotype 2 strain) measured in pfu/0.5 mL is less than 8% of the total concentration, such as in the range of 1% to 8% of the total concentration, the concentration of the dengue serotype 3 (e.g. chimeric dengue serotype 2/3 strain) measured in pfu/0.5 mL is at least 10% of the total concentration, and the concentration of the dengue serotype 4 (e.g. chimeric dengue serotype 2/4 strain) measured in pfu/0.5 mL is at least 65% of the total concentration, such as in the range of 65% to 80%. In certain such embodiments, the arithmetic sum of all four serotypes is in the range of 4.6 log 10 pfu/0.5 mL to 6.7 log 10 pfu/0.5 mL, preferably in the range of 4.6 log 10 pfu/0.5 mL to 5.5 log 10 pfu/0.5 mL Preferably, in said embodiments the subject or subject population is of 2 to 17 years of age, such as 4 to 16 years of age, and even more preferably less than 9 years of age. In other preferred embodiments, the subject or subject population is 4-5 years of age, 6-11 years of age or 12-16 years of age.

In a further preferred embodiment, the invention is directed to said methods, wherein the dengue serotype 1 (e.g. chimeric dengue serotype 2/1 strain) such as TDV-1 and the dengue serotype 2 (e.g. dengue serotype 2 strain) such as TDV-2 are present each in a concentration based on the total concentration in pfu/0.5 mL which is within 5%-points of each other and/or are together less than about 10% of the total concentration in pfu/0.5 mL. In certain such embodiments the dengue serotype 3 (e.g. chimeric dengue serotype 2/3 strain) such as TDV-3 is preferably at least about 10% of the total concentration in pfu/0.5 mL and more preferably the dengue serotype 4 (e.g. chimeric dengue serotype 2/4 strain) such as TDV-4 is at least about 70% of the total concentration in pfu/0.5 mL. In certain such embodiments the dengue serotype 4 (e.g. chimeric dengue serotype 2/4 strain) such as TDV-4 represents the highest concentration in the composition of all four serotypes, preferably with at least about 70% of the total concentration in pfu/0.5 mL, dengue serotype 3 (e.g. chimeric dengue serotype 2/3 strain) such as TDV-3 represents the second highest concentration in the composition of all four serotypes, preferably with at least about 10% of the total concentration in pfu/0.5 mL, and dengue serotype 1 (e.g. chimeric dengue serotype 2/1 strain) such as TDV-1 and dengue serotype 2 (e.g. dengue serotype 2 strain) such as TDV-2 each represent lower concentrations than the concentration of serotype 3 (e.g. chimeric dengue serotype 2/3 strain) such as TDV-3, and optionally together represent less than about 10% of the total concentration in pfu/0.5 mL.

Preferably, the chimeric dengue serotype 2/1 strain is TDV-1, the dengue serotype 2 strain is TDV-2, the chimeric dengue serotype 2/3 strain is TDV-3 and the chimeric dengue serotype 2/4 strain is TDV-4. More preferably, TDV-1 is characterized by the nucleotide sequence according to SEQ ID No. 1 and the amino acid sequence according to SEQ ID No. 2, TDV-2 is characterized by the nucleotide sequence according to SEQ ID No. 3 and the amino acid sequence according to SEQ ID No. 4, TDV-3 is characterized by the nucleotide sequence according to SEQ ID No. 5 and the amino acid sequence according to SEQ ID No. 6 and TDV-4 is characterized by the nucleotide sequence according to SEQ ID No. 7 and the amino acid sequence according to SEQ ID No. 8.

In certain embodiments, the invention is directed to said methods, wherein the reconstituted unit dose of the invention as described herein is administered by subcutaneous injection. According to some of these embodiments, the subcutaneous injection is administered to the arm, preferably to the deltoid region of the arm.

In certain embodiments, the invention is directed to said methods, wherein the reconstituted unit dose is administered to a subject of unknown serostatus and/or wherein no test has been carried out to determine whether the subject is seropositive or seronegative before the unit dose as described herein is administered.

In certain embodiments, the invention is directed to said methods, wherein the subject or subject population is seronegative to all dengue serotypes.

In certain embodiments, the invention is directed to said methods, wherein two unit doses of the invention as described herein are administered. In some embodiments the two unit doses are administered within 12 months or more, or within six months, or within three months, and optionally at least 4 weeks apart such as at day 0 and day 90 or at day 1 and day 90. According to some of these embodiments, a further third unit dose of the invention as described herein is administered after the second administration. Such a third administration may act as a booster and may be administered between 6 to 12 months after the first administration, such as 12 months after the first administration, or later than 12 month after the first administration, such as 12 months after the second administration.

In certain embodiments, the method of the invention comprises or consists of a single unit dose of the invention being administered.

In certain embodiments, the invention is directed to said methods, wherein the reconstituted unit dose of the invention as described herein is administered subcutaneously to a subject or subject population that is seronegative with respect to all dengue serotypes. In other embodiments, the subject or subject population is seropositive with respect to at least one dengue serotype.

In certain embodiments, the invention is directed to said methods, wherein the unit dose of the invention as described herein is administered to a subject or subject population from a dengue endemic region. In some of these embodiments, the subject or subject population is from Singapore, Dominican Republic, Panama, Philippines, Colombia, Puerto Rico or Thailand, in particular from Singapore, Dominican Republic, Panama, or Philippines. In a preferred embodiment, the subject or subject population is from Asia Pacific or from Latin America. In some other of these embodiments, the subject or subject population is from Thailand, Sri Lanka, Philippines, Panama, Nicaragua, Dominican Republic, Colombia or Brazil. In other embodiments, the subject or subject population is from a dengue non-endemic region. Such a subject population or such a subject may be vaccinated according to the present invention in the context of traveling to a dengue endemic region. In certain embodiments, the reconstituted unit dose of the invention as described herein is administered subcutaneously to a subject or subject population that is from a dengue endemic region or a dengue non-endemic region.

In certain embodiments, the invention is directed to said methods, wherein the reconstituted unit dose of the invention as described herein is administered subcutaneously to a subject or subject population of 2 to 60 years of age. In some embodiments, the subjects or subject population are adults of 18 to 60 years.

In certain embodiments, the invention is directed to said methods, wherein the reconstituted unit dose of the invention as described herein is administered subcutaneously to children and adolescents of 2 to 17 years of age. In some embodiments, the subjects or subject population are less than 9 years of age, or less than 4 years of age. In some embodiments, the subjects or subject population are from 2 to 9 years of age, or from 2 to 5 years of age, or from 4 to 9 years of age or from 6 to 9 years of age. In other embodiment, the subject or subject population is 4 to 16 years of age. In some such embodiments, the subject or subject population is 4-5 years of age, 6-11 years of age or 12-16 years of age. Optionally, the subject or subject population is seronegative with respect to all dengue serotypes.

In certain embodiments, the invention is directed to said methods, wherein the unit dose of the invention as described herein is administered to a pediatric subject or pediatric subject population of less than 2 years of age, preferably of 2 months to 2 years or 2 months to 1.5 years or 2 months to 1 year. According to some of these embodiments, the pediatric subject or pediatric subject population is seronegative and from a dengue endemic region.

In certain embodiments, the invention is directed to said methods, wherein the reconstituted unit dose of the invention as described herein is administered to a pediatric subject or pediatric subject population of less than 2 years of age, preferably of 2 months to 2 years or 2 months to 1.5 years or 2 months to 1 year, preferably by subcutaneous injection. According to some of these embodiments, the pediatric subject or pediatric subject population is seronegative and from a dengue endemic region.

In a certain embodiments, the invention is directed to said methods, wherein the subject or subject population is 4-5 years of age and from Asia Pacific, 6-11 years of age and from Asia Pacific, or 12-16 years of age and from Asia Pacific. In other embodiments, the subject or subject population is 4-5 years of age and from Latin America, 6-11 years of age and from Latin America, or 12-16 years of age and from Latin America.

In a certain embodiments, the invention is directed to said methods, wherein the subject or subject population is 4-5 years of age and seropositive for at least 1 dengue serotype, 6-11 years of age and seropositive for at least 1 dengue serotype, or 12-16 years of age and seropositive for at least 1 dengue serotype. In other embodiments, the subject or subject population is 4-5 years of age and seronegative for all dengue serotypes, 6-11 years of age and seronegative for all dengue serotypes, or 12-16 years of age and seronegative for all dengue serotypes.

In a certain embodiments, the invention is directed to said methods, wherein the subject or subject population is from Asia Pacific or Latin America and seropositive for at least one dengue serotype at baseline. In other embodiments, the subject or subject population is from Asia Pacific or Latin America and seronegative for at all dengue serotype at baseline.

In certain embodiments, the invention is directed to said methods, wherein the subject or subject population is from Asia Pacific, seropositive for at least one dengue serotype at baseline and 4-5 years of age, 6-11 years of age, or 12-16 years of age. In other embodiments, the subject or subject population is from Asia Pacific, seronegative for all dengue serotypes at baseline and 4-5 years of age, 6-11 years of age, or 12-16 years of age. In yet other embodiments, the subject or subject population is from Latin America, seropositive for at least one dengue serotype at baseline and 4-5 years of age, 6-11 years of age, or 12-16 years of age. In other embodiments, the subject or subject population is from America, seronegative for all dengue serotypes at baseline and 4-5 years of age, 6-11 years of age, or 12-16 years of age.

In certain embodiments, the invention is directed to said methods, wherein the subject or subject population had prior vaccination against Yellow Fever. In other embodiments, the subject or subject population had prior vaccination against Japanese Encephalitis. In yet other embodiments, the subject or subject population had no prior vaccination against Yellow Fever. In other embodiments, the subject or subject population had no prior vaccination against Japanese Encephalitis. Prior vaccination indicates a vaccination prior to 30 days after a second administration, such as within 4 months after the first administration, with the reconstituted unit dose as described herein. For example for vaccine efficacy (VE) as determined in Example 6 from 30 days post-second vaccination, a prior vaccination of Yellow Fever is defined as a Yellow Fever vaccination occurring before 30 days post-second vaccination. In certain embodiments, the subject or subject population received Denvaxia within the administration regimen as described herein or within 4.5 years after administration of the first dose.

Particularly unbalanced titers of neutralizing antibodies against the four dengue serotypes are observed in seronegative populations or subjects after administration of the commercially available dengue vaccine. The present invention shows that in particular seronegative subjects show a more balanced immune response to the four dengue serotypes after administration of the reconstituted unit dose of the invention as described herein. It is therefore contemplated that the unit dose of the invention as described herein and methods of the present invention as described herein may provide a more robust immune response in a subject population including both seropositive and seronegative subjects.

The present invention is directed in part to a method of preventing virologically confirmable dengue disease in a subject comprising administering to the subject a tetravalent dengue virus composition including four dengue virus strains representing serotype 1, serotype 2, serotype 3 and serotype 4, wherein the virus strains are optionally live, attenuated dengue virus strains.

The present invention is directed in part to a method of preventing virologically confirmable dengue disease in a subject consisting of administering to the subject a tetravalent dengue virus composition including four dengue virus strains representing serotype 1, serotype 2, serotype 3 and serotype 4, wherein the virus strains are optionally live, attenuated dengue virus strains.

In certain embodiments, the invention is directed to said methods, wherein there is no step of determining the serostatus of the subject at baseline, in other words, said methods do not comprise a determination of a previous dengue infection of the subject at baseline before the administration of the tetravalent dengue virus composition. In particular, such methods are safe and effective. Thus, in certain such embodiments, the subject has not been tested for the presence a previous dengue infection.

In certain embodiments, the invention is directed to said methods, wherein the vaccine administration is safe irrespective of whether there is a determination that the subject had a previous dengue infection before the administration of the tetravalent dengue virus composition. In particular, such methods are also effective.

In certain embodiments, the invention is directed to said methods, wherein the method is safe and/or effective.

In certain embodiments, the invention is directed to said methods, wherein the composition includes at least one chimeric dengue virus. In certain such embodiments, the invention is directed to said methods, wherein the composition includes at least one non-chimeric dengue virus and at least one chimeric dengue virus, in particular a chimeric dengue serotype 2/1 strain and a dengue serotype 2 strain and a chimeric dengue serotype 2/3 strain and a chimeric dengue serotype 2/4 strain. The details of the composition are described above.

Therefore, in certain embodiments, the invention is directed to said methods having a vaccine efficacy, preferably a combined vaccine efficacy against all four serotypes, in preventing virologically confirmable dengue disease with a 2-sided 95% confidence interval, wherein the lower bound is more than 25%, when measured against placebo in a subject population of at least 5,000 healthy subjects (or at least 10,000, or at least 15,000 healthy subjects) irrespective of serostatus at baseline, wherein a reconstituted unit dose as described herein or placebo is administered at least twice within less than 6 months, such as within 3 months, 30 days after the second administration until at least 12 months after the second administration. In embodiments, the invention is directed to said methods having a vaccine efficacy, preferably a combined vaccine efficacy against all four serotypes, in preventing virologically confirmable dengue disease with a 2-sided 95% confidence interval, wherein the lower bound is more than 25%, when measured against placebo in a subject population of at least 5,000 healthy subjects (or at least 10,000, or at least 15,000 healthy subjects) irrespective of serostatus at baseline, wherein a reconstituted unit dose as described herein or placebo is administered at least once, until 15 months after the first administration of the administration schedule. In certain such embodiments, the lower bound is more than 30%, more than 40%, more than 50%, more than 55%, more than 60%, more than 65%, more than 70% or more than 72%. Preferably said reconstituted unit dose or placebo is administered subcutaneously within about 3 month, such as on days 0 and 90.

In certain embodiments, the invention is directed to said methods having a vaccine efficacy, preferably a combined vaccine efficacy against all four serotypes, in preventing virologically confirmable dengue disease of more than 30%, when measured against placebo in a subject population of at least 5,000 healthy subjects (or at least 10,000, or at least 15,000 healthy subjects) irrespective of serostatus at baseline, wherein a reconstituted unit dose as described herein or placebo is administered at least twice within less than 6 months, such as within 3 months, 30 days after the second administration until at least 12 months after the second administration. In certain embodiments, the invention is directed to said methods having a vaccine efficacy, preferably a combined vaccine efficacy against all four serotypes, in preventing virologically confirmable dengue disease of more than 30%, when measured against placebo in a subject population of at least 5,000 healthy subjects (or at least 10,000, or at least 15,000 healthy subjects) irrespective of serostatus at baseline, wherein a reconstituted unit dose as described herein or placebo is administered at least once, until 15 months after the first administration of the administration schedule. In certain such embodiments, the vaccine efficacy is more than 40%, more than 50%, more than 55%, more than 60%, more than 65%, more than 70%, more than 75%, more than 78%, more than 79% or about 80%. Preferably said reconstituted unit dose or placebo is administered subcutaneously within about 3 month, such as on days 0 and 90.

In certain embodiments, the invention is directed to said methods having a vaccine efficacy, preferably a combined vaccine efficacy against all four serotypes, in preventing virologically confirmable dengue disease with hospitalization with a 2-sided 95% confidence interval, wherein the lower bound is more than 0%, when measured against placebo in a subject population of at least 5,000 healthy subjects (or at least 10,000, or at least 15,000 healthy subjects) irrespective of serostatus at baseline, wherein a reconstituted unit dose as described herein or placebo is administered at least twice within less than 6 months, such as within 3 months, 30 days after the second administration until at least 18 months after the second administration. In certain such embodiments, the lower bound is more than 10%, is more than 20%, is more than 30%, is more than 40%, is more than 50%, is more than 55%, is more than 60%, is more than 65%, is more than 70% or is more than 80%, or more than 90%.

In certain embodiments, the invention is directed to said methods having a combined vaccine efficacy against all four dengue serotypes in seronegative subjects with a 2-sided 95% confidence interval, wherein the lower bound is more than 25%, when measured against placebo in a subject population of at least 2,000 healthy subjects being seronegative against all serotypes at baseline, wherein said unit dose or said placebo is administered at least twice within less than 6 months, such as within 3 months, about 30 days after the second administration of the administration schedule until at least 12 months after the second administration of the administration schedule. In certain such embodiments, the lower bound is more than 30%, is more than 40%, is more than 50%, or is more than 55%.

In certain embodiments, the invention is directed to said methods having a combined vaccine efficacy against all four dengue serotypes in seronegative subjects of more than 30%, when measured against placebo in a subject population of at least 2,000 healthy subjects being seronegative against all serotypes at baseline, wherein said unit dose or said placebo is administered at least twice within less than 6 months, such as within 3 months, 30 days after the second administration until at least 12 months after the second administration. In certain such embodiments, the combined vaccine efficacy against all four dengue serotypes in seronegative subjects is more than 40%, is more than 50%, is more than 60%, is more than 65%, or is more than 70%.

In certain embodiments, the invention is directed to said methods having a combined vaccine efficacy against all four dengue serotypes with a 2-sided 95% confidence interval, wherein the lower bound is more than 25%, when measured against placebo in a subject population of at least 1,000 healthy subjects 4 to 5 years of age at the time of randomization and irrespective of serostatus at baseline, wherein said unit dose or said placebo is administered at least twice within less than 6 months, such as within 3 months, about 30 days after the second administration of the administration schedule until at least 12 months after the second administration of the administration schedule. In certain such embodiments, the lower bound is more than 30%, is more than 40%, is more than 45%.

In certain embodiments, the invention is directed to said methods having a combined vaccine efficacy against all four dengue serotypes of more than 30%, when measured against placebo in a subject population of at least 1,000 healthy subjects 4 to 5 years of age at the time of randomization and irrespective of serostatus at baseline, wherein said unit dose or said placebo is administered at least twice within less than 6 months, such as within 3 months, 30 days after the second administration until at least 12 months after the second administration. In certain such embodiments, the combined vaccine efficacy against all four dengue serotypes is more than 40%, is more than 50%, is more than 60%, is more than 65%, or is more than 70%.

In certain embodiments, the invention is directed to said methods having a combined vaccine efficacy against all four dengue serotypes with a 2-sided 95% confidence interval, wherein the lower bound is more than 25%, when measured against placebo in a subject population of at least 1,000 healthy subjects 6 to 11 years of age at the time of randomization and irrespective of serostatus at baseline, wherein said unit dose or said placebo is administered at least twice within less than 6 months, such as within 3 months, about 30 days after the second administration of the administration schedule until at least 12 months after the second administration of the administration schedule. In certain such embodiments, the lower bound is more than 30%, is more than 40%, is more than 50%, is more than 60%, or is more than 70%.

In certain embodiments, the invention is directed to said methods having a combined vaccine efficacy against all four dengue serotypes of more than 30%, when measured against placebo in a subject population of at least 1,000 healthy subjects 6 to 11 years of age at the time of randomization and irrespective of serostatus at baseline, wherein said unit dose or said placebo is administered at least twice within less than 6 months, such as within 3 months, 30 days after the second administration until at least 12 months after the second administration. In certain such embodiments, the combined vaccine efficacy against all four dengue serotypes is more than 40%, is more than 50%, is more than 60%, is more than 70%, is more than 75%, or is more than 80%.

In certain embodiments, the invention is directed to said methods having a combined vaccine efficacy against all four dengue serotypes with a 2-sided 95% confidence interval, wherein the lower bound is more than 25%, when measured against placebo in a subject population of at least 1,000 healthy subjects 12 to 16 years of age at the time of randomization and irrespective of serostatus at baseline, wherein said unit dose or said placebo is administered at least twice within less than 6 months, such as within 3 months, about 30 days after the second administration of the administration schedule until at least 12 months after the second administration of the administration schedule. In certain such embodiments, the lower bound is more than 30%, is more than 40%, is more than 50%, is more than 60%, is more than 65%, or is more than 68%.

In certain embodiments, the invention is directed to said methods having a combined vaccine efficacy against all four dengue serotypes of more than 30%, when measured against placebo in a subject population of at least 1,000 healthy subjects 12 to 16 years of age at the time of randomization and irrespective of serostatus at baseline, wherein said unit dose or said placebo is administered at least twice within less than 6 months, such as within 3 months, 30 days after the second administration until at least 12 months after the second administration. In certain such embodiments, the combined vaccine efficacy against all four dengue serotypes is more than 40%, is more than 50%, is more than 60%, is more than 70%, is more than 75%, or is more than 80%.

In certain embodiments, the invention is directed to said methods having a vaccine efficacy against dengue serotype 1 with a 2-sided 95% confidence interval, wherein the lower bound is more than 25%, when measured against placebo in a subject population of at least 5,000 healthy subjects, or at least 10,000 healthy subjects, or at least 15,000 healthy subjects irrespective of serostatus at baseline, wherein said unit dose or said placebo is administered at least twice within less than 6 months, such as within 3 months, about 30 days after the second administration of the administration schedule until at least 12 months after the second administration of the administration schedule. In certain such embodiments, the lower bound is more than 30%, is more than 40%, or is more than 50%.

In certain embodiments, the invention is directed to said methods having a vaccine efficacy against dengue serotype 1 of more than 30%, when measured against placebo in a subject population of at least 5,000 healthy subjects, or at least 10,000 healthy subjects, or at least 15,000 healthy subjects irrespective of serostatus at baseline, wherein said unit dose or said placebo is administered at least twice within less than 6 months, such as within 3 months, 30 days after the second administration until at least 12 months after the second administration. In certain such embodiments, the vaccine efficacy against dengue serotype 1 is more than 40%, is more than 50%, is more than 60%, is more than 65%, or is more than 70%.

In certain embodiments, the invention is directed to said methods having a vaccine efficacy against dengue serotype 2 with a 2-sided 95% confidence interval, wherein the lower bound is more than 25%, when measured against placebo in a subject population of at least 5,000 healthy subjects, or at least 10,000 healthy subjects, or at least 15,000 healthy subjects irrespective of serostatus at baseline, wherein said unit dose or said placebo is administered at least twice within less than 6 months, such as within 3 months, about 30 days after the second administration of the administration schedule until at least 12 months after the second administration of the administration schedule. In certain such embodiments, the lower bound is more than 30%, is more than 40%, is more than 50, is more than 60, is more than 70, is more than 80, or is more than 90%.

In certain embodiments, the invention is directed to said methods having a vaccine efficacy against dengue serotype 2 of more than 30%, when measured against placebo in a subject population of at least 5,000 healthy subjects, or at least 10,000 healthy subjects, or at least 15,000 healthy subjects irrespective of serostatus at baseline, wherein said unit dose or said placebo is administered at least twice within less than 6 months, such as within 3 months, 30 days after the second administration until at least 12 months after the second administration. In certain such embodiments, the vaccine efficacy against dengue serotype 2 is more than 40%, is more than 50%, is more than 60%, is more than 70%, is more than 80, or is more than 90%.

In certain embodiments, the invention is directed to said methods having a vaccine efficacy against dengue serotype 3 with a 2-sided 95% confidence interval, wherein the lower bound is more than 25%, when measured against placebo in a subject population of at least 5,000 healthy subjects, or at least 10,000 healthy subjects, or at least 15,000 healthy subjects irrespective of serostatus at baseline, wherein said unit dose or said placebo is administered at least twice within less than 6 months, such as within 3 months, about 30 days after the second administration of the administration schedule until at least 12 months after the second administration of the administration schedule. In certain such embodiments, the lower bound is more than 30%, is more than 40%.

In certain embodiments, the invention is directed to said methods having a vaccine efficacy against dengue serotype 3 of more than 30%, when measured against placebo in a subject population of at least 5,000 healthy subjects, or at least 10,000 healthy subjects, or at least 15,000 healthy subjects irrespective of serostatus at baseline, wherein said unit dose or said placebo is administered at least twice within less than 6 months, such as within 3 months, 30 days after the second administration until at least 12 months after the second administration. In certain such embodiments, the vaccine efficacy against dengue serotype 3 is more than 40%, is more than 50%, is more than 55%, or is more than 60%.

In certain embodiments, the invention is directed to said methods having a combined vaccine efficacy against virologically-confirmed dengue with hospitalization against all four serotypes with a 2-sided 95% confidence interval, wherein the lower bound is more than 25%, when measured against placebo in a subject population of at least 2,000 healthy subjects being seronegative against all serotypes at baseline, wherein said unit dose or said placebo is administered at least twice within less than 6 months, such as within 3 months, about 30 days after the second administration of the administration schedule until at least 12 months after the second administration of the administration schedule. In certain such embodiments, the lower bound is more than 30%, is more than 40%, is more than 50%, is more than 60%, is more than 70%, or is more than 75%.

In certain embodiments, the invention is directed to said methods having a combined vaccine efficacy against virologically-confirmed dengue with hospitalization against all four serotypes of more than 30%, when measured against placebo in a subject population of at least 2,000 healthy subjects, healthy subjects being seronegative against all serotypes at baseline, wherein said unit dose or said placebo is administered at least twice within less than 6 months, such as within 3 months, 30 days after the second administration until at least 12 months after the second administration. In certain such embodiments, the combined vaccine efficacy against virologically-confirmed dengue with hospitalization against all four serotypes is more than 40%, is more than 50%, is more than 60%, is more than 70%, is more than 80%, or is more than 90%.

In certain embodiments, the invention is directed to said methods having a combined vaccine efficacy against virologically-confirmed dengue with hospitalization against all four serotypes with a 2-sided 95% confidence interval, wherein the lower bound is more than 25%, when measured against placebo in a subject population of at least 2,000 healthy subjects being seropositive at baseline, wherein said unit dose or said placebo is administered at least twice within less than 6 months, such as within 3 months, about 30 days after the second administration of the administration schedule until at least 12 months after the second administration of the administration schedule. In certain such embodiments, the lower bound is more than 30%, is more than 40%, is more than 50%, is more than 60%, is more than 70%, or is more than 80%.

In certain embodiments, the invention is directed to said methods having a combined vaccine efficacy against virologically-confirmed dengue with hospitalization against all four serotypes of more than 30%, when measured against placebo in a subject population of at least 2,000 healthy subjects, healthy subjects being seropositive at baseline, wherein said unit dose or said placebo is administered at least twice within less than 6 months, such as within 3 months, 30 days after the second administration until at least 12 months after the second administration. In certain such embodiments, the combined vaccine efficacy against virologically-confirmed dengue with hospitalization against all four serotypes is more than 40%, is more than 50%, is more than 60%, is more than 70%, is more than 80%, or is more than 90%.

In certain embodiments, the invention is directed to said methods having a relative risk, preferably a combined relative risk against all four serotypes, with a 2-sided 95% confidence interval, wherein the upper bound is less than 0.75, when measured against placebo in a subject population of at least 5,000 healthy subjects (or at least 10,000, or at least 15,000 healthy subjects) irrespective of serostatus at baseline, wherein a reconstituted unit dose as described herein or placebo is administered at least twice within less than 6 months, such as within 3 months, 30 days after the second administration until at least 12 months after the second administration. In certain such embodiments, the upper bound is less than 0.70, less than 0.65, less than 0.60, less than 0.55, less than 0.50, less than 0.45, less than 0.40, less than 0.35, less than 0.30 or less than 0.28. Preferably said reconstituted unit dose or placebo is administered subcutaneously within about 3 month, such as on days 0 and 90.

In certain embodiments, the invention is directed to said methods having a relative risk, preferably a combined relative risk against all four serotypes, of less than 0.70, when measured against placebo in a subject population of at least 5,000 healthy subjects (or at least 10,000, or at least 15,000 healthy subjects) irrespective of serostatus at baseline, wherein a reconstituted unit dose as described herein or placebo is administered at least twice within less than 6 months, such as within 3 months, 30 days after the second administration until at least 12 months after the second administration. In certain such embodiments, the relative risk is less than 0.65, less than 0.60, less than 0.55, less than 0.50, less than 0.45, less than 0.40, less than 0.35, less than 0.30, less than 0.25 or less than 0.23. Preferably said reconstituted unit dose or placebo is administered subcutaneously within about 3 month, such as on days 0 and 90.

In certain embodiments, the invention is directed to said methods, wherein virologically confirmable dengue disease occurs in less than 2.5% of the subjects, when measured against placebo in a subject population of at least 5,000 healthy subjects (or at least 10,000, or at least 15,000 healthy subjects) irrespective of serostatus at baseline, wherein a reconstituted unit dose as described herein or placebo is administered at least twice within less than 6 months, such as within 3 months, 30 days after the second administration until at least 12 months or at least 18 months after the second administration. In certain such embodiments, virologically confirmable dengue disease occurs in less than 2.0% of the subjects, less than 1.5% of the subjects, less than 1.0% of the subjects, less than 0.8% of the subjects, or less than 0.6% of the subjects. Preferably said reconstituted unit dose or placebo is administered subcutaneously within about 3 month, such as on days 0 and 90.

In certain embodiments, the invention is directed to said methods having a combined vaccine efficacy against all four serotypes with a 2-sided 95% confidence interval, wherein the lower bound is more than 61.0%, or more than 65.0 or more than 70.0% or more than 72.0% when measured against placebo in a subject population of at least 5,000 healthy subjects (or at least 10,000, or at least 15,000 healthy subjects) from endemic irrespective of serostatus at baseline and being selected from the group consisting of 4 to 16 year old subjects at the time of randomization, wherein said unit dose or said placebo is administered at least twice within 6 months or less, about 30 days after the last administration of the administration schedule until at least 12 or 13 months after the last administration of the administration schedule.

In certain embodiments, the invention is directed to said methods having a combined vaccine efficacy against all four serotypes of more than 66%, or of more than 70%, or of more than 75%, or of more than 77%, or of more than 80.0%, when measured against placebo in a subject population of at least 5,000 healthy subjects (or at least 10,000, or at least 15,000 healthy subjects) from endemic areas irrespective of serostatus at baseline and being selected from the group consisting of 4 to 16 year old subjects at the time of randomization, wherein said unit dose or said placebo is administered at least twice within 6 months or less, about 30 days after the last administration of the administration schedule until at least 12 months or 13 month after the last administration of the administration schedule.

In certain embodiments, the invention is directed to said methods, wherein the combined vaccine efficacy against all four serotypes is measured about 30 days after the last administration of the administration schedule until 12 or 13 months after the last administration of the administration schedule.

In certain embodiments, the invention is directed to said methods, wherein said unit dose or said placebo is administered at twice within three months, in particular at about day 1 and about day 90, and wherein the combined vaccine efficacy against all four serotypes is measured 30 days after the second administration until 12 or 13 months after the second administration of the administration schedule.

In certain embodiments, the invention is directed to said methods, wherein said methods are effective and safe. In some of these embodiments, the subject or subject population is under 9 years of age, under 4 years of age, or under 2 years of age or from 2 to 9 years of age, or from 2 to 5 years of age, or from 4 to 9 years of age or from 6 to 9 years of age. Optionally the subject is seronegative with respect to all dengue serotypes.

In certain embodiments, the invention is directed to said methods, wherein said methods having a relative risk for virologically confirmed dengue with hospitalization of 1 or less, or 0.8 or less, or 0.6 or less, when measured against placebo in a subject population of at least 5,000 healthy subjects (or at least 10,000, or at least 15,000 healthy subjects). In some of these embodiments, the subject or subject population is under 9 years of age, under 4 years of age, or under 2 years of age or from 2 to 9 years of age, or from 2 to 5 years of age, or from 4 to 9 years of age or from 6 to 9 years of age. Optionally the subject is seronegative with respect to all dengue serotypes.

In certain embodiments, the invention is directed to said methods, wherein the healthy subjects of the subject population are 4 to 16 years of age. In some of such embodiments, the healthy subjects of the subject population are 4 to 5 years of age, 6 to 11 years of age, or 12 to 16 years of age.

In certain embodiments, the invention is directed to said methods, wherein the healthy subjects of the subject population are defined as being healthy in view of the exclusion criteria specified in Example 6.

In certain embodiments, the invention is directed to said methods, wherein the healthy subjects of the subject population are from Asia Pacific or Latin America.

In certain embodiments, the invention is directed to said methods, wherein the healthy subjects of the subject population are seropositive with respect to at least one serotype. In other embodiments, the healthy subjects of the subject population are seronegative with respect to all serotypes.

In certain embodiments, the invention is directed to said methods, wherein the healthy subjects of the subject population are 4-5 years of age and from Asia Pacific, 6-11 years of age and from Asia Pacific, or 12-16 years of age and from Asia Pacific. In other embodiments, the healthy subjects of the subject population are 4-5 years of age and from Latin America, 6-11 years of age and from Latin America, or 12-16 years of age and from Latin America.

In certain embodiments, the invention is directed to said methods, wherein the healthy subjects of the subject population are 4-5 years of age and seropositive for at least 1 dengue serotype, 6-11 years of age and seropositive for at least 1 dengue serotype, or 12-16 years of age and seropositive for at least 1 dengue serotype. In other embodiments, the healthy subjects of the subject population are 4-5 years of age and seronegative for all dengue serotypes, 6-11 years of age and seronegative for all dengue serotypes, or 12-16 years of age and seronegative for all dengue serotypes.

In certain embodiments, the invention is directed to said methods, wherein the healthy subjects of the subject population are from Asia Pacific or Latin America and seropositive for at least one dengue serotype at baseline. In other embodiments, the healthy subjects of the subject population are from Asia Pacific or Latin America and seronegative for at all dengue serotype at baseline.

In certain embodiments, the invention is directed to said methods, wherein the healthy subjects of the subject population are from Asia Pacific, seropositive for at least one dengue serotype at baseline and 4-5 years of age, 6-11 years of age, or 12-16 years of age. In other embodiments, the healthy subjects of the subject population are from Asia Pacific, seronegative for all dengue serotypes at baseline and 4-5 years of age, 6-11 years of age, or 12-16 years of age. In yet other embodiments, the healthy subjects of the subject population are from Latin America, seropositive for at least one dengue serotype at baseline and 4-5 years of age, 6-11 years of age, or 12-16 years of age. In other embodiments, the healthy subjects of the subject population are from America, seronegative for all dengue serotypes at baseline and 4-5 years of age, 6-11 years of age, or 12-16 years of age.

In certain embodiments, the invention is directed to said methods, wherein the healthy subjects of the subject population had prior vaccination against Yellow Fever. In other embodiments, the healthy subjects of the subject population had no prior vaccination against Yellow Fever. Prior vaccination indicates a vaccination prior to the first vaccination with the reconstituted unit dose as described herein. For example for vaccine efficacy (VE) as determined in Example 6 from 30 days post-second vaccination, a prior vaccination of Yellow Fever is defined as a Yellow Fever vaccination occurring before 30 days post-second vaccination.

In certain embodiments, the invention is directed to said methods, wherein the healthy subjects of the subject population had prior vaccination against Japanese Encephalitis. In other embodiments, the healthy subjects of the subject population had no prior vaccination against Japanese Encephalitis.

In certain embodiments, the invention is directed to said methods, wherein the healthy subjects of the subject population received Denvaxia within the administration regimen as described herein or within 4.5 years after administration of the first dose. In certain embodiments, the invention is directed to said methods, wherein the occurrence of vaccine related serious adverse events is less than 0.1%.

In certain embodiments, the invention is directed to said methods, wherein the occurrence of vaccine related unsolicited adverse events occurring within 4 weeks of administration is less than 2%.

In certain embodiments, the invention is directed to said methods, wherein the occurrence of vaccine related solicited adverse events occurring within 2 weeks of administration is less than 35%.

In certain embodiments, the invention is directed to said methods, wherein the occurrence of vaccine related solicited local reactions occurring within 1 weeks of administration is less than 40%.

In certain embodiments, the invention is directed to said methods, wherein the method does not increase the risk of virologically-confirmed dengue with hospitalization in the individual, such as in a seronegative individual.

### Unit Dose for Use in a Method of Preventing Dengue Disease

The present invention is directed in part to the composition or unit dose of the invention as described herein for use in a method of preventing dengue disease (in particular virologically confirmable dengue, VCD) in a subject.

The present invention is directed in part to the composition or unit dose of the invention as described herein for use in a method of preventing dengue disease (in particular virologically confirmable dengue, VCD) in a subject population.

Any method described herein above under the heading “Method of preventing” is to be understood to be also disclosed as unit dose for use in such a method of preventing dengue disease in a subject or subject population irrespective of being expressly stated below.

The present invention is directed in part to a reconstituted unit dose of a dengue vaccine composition as described herein for use in a method of preventing virologically confirmable dengue disease in a subject comprising administering at least a first unit dose of the dengue vaccine composition to the subject, wherein the dengue vaccine composition is a tetravalent dengue virus composition including four dengue virus strains representing dengue serotype 1, dengue serotype 2, dengue serotype 3 and dengue serotype 4, optionally wherein the dengue virus strains are live, attenuated dengue virus strains and/or comprise chimeric dengue viruses and/or at least one non-chimeric dengue virus, and wherein upon reconstitution with 0.5 mL of a pharmaceutically acceptable diluent
(i) dengue serotype 1 has a concentration of at least 3.3 log 10 pfu/0.5 mL and optionally to 5.0 log 10 pfu/0.5 mL,
(ii) dengue serotype 2 has a concentration of at least 2.7 log 10 pfu/0.5 mL and optionally to 4.9 log 10 pfu/0.5 mL,
(iii) dengue serotype 3 has a concentration of at least 4.0 log 10 pfu/0.5 mL and optionally to 5.7 log 10 pfu/0.5 mL, and
(iv) dengue serotype 4 has a concentration of at least 4.5 log 10 pfu/0.5 mL and optionally to 6.2 log 10 pfu/0.5 mL.

The present invention is directed in part to a reconstituted unit dose of a dengue vaccine composition as described herein for use in a method of preventing virologically confirmable dengue disease in a subject comprising consecutively administering at least a first and a second unit dose of the dengue vaccine composition to the subject, wherein said first and second unit dose are administered subcutaneously within 3 months and at least 4 weeks apart, optionally at about day 1 and at about day 90, wherein the dengue vaccine composition is a tetravalent dengue virus composition including four dengue virus strains representing dengue serotype 1, dengue serotype 2, dengue serotype 3 and dengue serotype 4, optionally wherein the dengue virus strains are live, attenuated, and wherein upon reconstitution with 0.5 mL of a pharmaceutically acceptable diluent
(v) dengue serotype 1 has a concentration of at least 3.3 log 10 pfu/0.5 mL and optionally to 5.0 log 10 pfu/0.5 mL,
(vi) dengue serotype 2 has a concentration of at least 2.7 log 10 pfu/0.5 mL and optionally to 4.9 log 10 pfu/0.5 mL,
(vii) dengue serotype 3 has a concentration of at least 4.0 log 10 pfu/0.5 mL and optionally to 5.7 log 10 pfu/0.5 mL, and
(viii) dengue serotype 4 has a concentration of at least 4.5 log 10 pfu/0.5 mL and optionally to 6.2 log 10 pfu/0.5 mL.

In certain embodiments, the invention is directed to a reconstituted unit dose of a dengue vaccine composition for use in a method of preventing virologically confirmable dengue disease in a subject comprising consecutively administering at least a first and a second unit dose of the dengue vaccine composition to the subject, wherein said first and second unit dose are administered subcutaneously within 3 months and at least 4 weeks apart, optionally at about day 1 and at about day 90, wherein the dengue vaccine composition is a tetravalent dengue virus composition including four dengue virus strains representing dengue serotype 1, dengue serotype 2, dengue serotype 3 and dengue serotype 4, optionally wherein the dengue virus strains are live, attenuated, wherein the subject is under 9 years of age and/or when the serostatus of the subject is unknown or seronegative and wherein upon reconstitution with 0.5 mL of a pharmaceutically acceptable diluent
(i) dengue serotype 1 has a concentration of at least 3.3 log 10 pfu/0.5 mL and optionally to 5.0 log 10 pfu/0.5 mL,
(ii) dengue serotype 2 has a concentration of at least 2.7 log 10 pfu/0.5 mL and optionally to 4.9 log 10 pfu/0.5 mL,
(iii) dengue serotype 3 has a concentration of at least 4.0 log 10 pfu/0.5 mL and optionally to 5.7 log 10 pfu/0.5 mL, and
(iv) dengue serotype 4 has a concentration of at least 4.5 log 10 pfu/0.5 mL and optionally to 6.2 log 10 pfu/0.5 mL.

In certain embodiments, the reconstituted unit dose is administered to a subject of unknown serostatus and/or wherein no test has been carried out to determine whether the subject is seropositive or seronegative before the unit dose as described herein is administered.

In certain embodiments, the subject is under 9 years of age and/or the serostatus of the subject is unknown or seronegative. In certain such embodiments, the subject is under 9 years of age and the serostatus of the subject is unknown or seronegative, preferably seronegative.

In certain embodiments, the method is safe. In certain such embodiments, the subject is under 9 years of age or from 4 years of age and/or the serostatus of the subject is unknown or seronegative. In certain such embodiments, the subject is from 4 years of age and the serostatus of the subject is unknown or seronegative, preferably seronegative.

In certain embodiments, the method is effective. In certain such embodiments, the subject is under 9 years of age and/or the serostatus of the subject is unknown or seronegative. In certain such embodiments, the subject is under 9 years of age and the serostatus of the subject is unknown or seronegative, preferably seronegative.

In certain embodiments, the dengue serotype 1 and the dengue serotype 2 are present each in a concentration based on the total concentration in pfu/0.5 mL which is within 5%-points of each other and/or are together less than about 10% of the total concentration in pfu/0.5 mL. In certain such embodiments, the dengue serotype 3 is at least about 10% of the total concentration in pfu/0.5 mL and/or the dengue serotype 4 is at least about 70% of the total concentration in pfu/0.5 mL.

In certain embodiments, the dengue serotype 4 represents the highest concentration in the composition of all four serotypes, preferably with at least about 70% of the total concentration in pfu/0.5 mL, dengue serotype 3 represents the second highest concentration in the composition of all four serotypes, preferably with at least about 10% of the total concentration in pfu/0.5 mL, and dengue serotype 1 and dengue serotype 2 each represent lower concentrations than the concentration of serotype 3, and optionally together represent less than about 10% of the total concentration in pfu/0.5 mL.

In certain embodiments, the composition includes at least one chimeric dengue virus. In certain such embodiments, the composition includes at least one non-chimeric dengue virus and at least one chimeric dengue virus.

In certain embodiments, the subject is seronegative to all dengue serotypes at baseline and/or is from 4 years of age, optionally to 60 years of age. In certain such embodiments, the subject is 4 to 16 years of age, under 9 years of age, from 2 years of age to under 9 years of age, from 4 years of age to under 9 years of age, 4 to 5 years of age, 6 to 11 years of age, or 12 to 16 years of age. In other embodiments, the subject is seropositive to at least one dengue serotypes at baseline and/or is from 4 years of age, optionally to 60 years of age. In certain such embodiments, the subject is 4 to 16 years of age, under 9 years of age, from 2 years of age to under 9 years of age, from 4 years of age to under 9 years of age, 4 to 5 years of age, 6 to 11 years of age, or 12 to 16 years of age.

In certain embodiments, the method does not comprise a determination of a previous dengue infection in the subject before the administration of the first unit dose of the tetravalent dengue virus composition. Thus, in certain embodiments, the subject has not been tested for the presence a previous dengue infection.

In certain embodiments, the dengue serotype 1 is a chimeric dengue serotype 2/1 strain, the dengue serotype 2 is a non-chimeric dengue serotype 2 strain, the dengue serotype 3 is a chimeric dengue serotype 2/3 strain and the dengue serotype 4 is a chimeric dengue serotype 2/4 strain and the dengue serotype 1 has the amino acid sequence of SEQ ID NO. 2, the dengue serotype 2 has the amino acid sequence of SEQ ID NO. 4, the dengue serotype 3 has the amino acid sequence of SEQ ID NO. 6, and the dengue serotype 4 has the amino acid sequence of SEQ ID NO. 8.

In certain embodiments, the unit dose further comprises from about 10% w/v to about 20% w/v α,α-trehalose dihydrate or an equimolar amount of other forms of α,α-trehalose, from about 0.5% w/v to about 1.5% w/v poloxamer 407, from about 0.05% w/v to about 2% w/v human serum albumin, and from about 70 mM to 140 mM sodium chloride when measured in 0.5 mL. In certain such embodiments, the unit dose comprises about 15% (w/v) α,α-trehalose dihydrate, about 1% (w/v) poloxamer 407, about 0.1% (w/v) human serum albumin, and about 100 mM sodium chloride when measured in 0.5 mL.

In certain embodiments, the method is for preventing dengue hemorrhagic fever (DHF) or dengue shock syndrome (DSS).

In certain embodiments, the subject is from a dengue endemic region. In other embodiments, the subject is from a dengue non-endemic region.

In certain embodiments, the subject is from Asia Pacific or Latin America.

In certain embodiments, the reconstituted unit dose provides a seropositivity rate when it is administered to a subject population of at least 50 subjects in two unit doses subcutaneously at day 1 and at day 90, wherein the subjects of the subject population are seronegative to all dengue serotypes at baseline. In certain such embodiments, at least 80% of the subject population are seropositive for all four dengue serotypes at least one month after administration of the first unit dose, such as at day 30, and/or at least 80% of the subject population are seropositive for all four dengue serotypes before or at the time of the administration of the second unit dose, such as at day 90, and/or at least 80%, or at least 85% or at least 90%, or at least 95% of the subject population are seropositive for all four dengue serotypes after the administration of the second unit dose, such as at day 120, and/or at least 80%, or at least 85%, or at least 90% of the subject population are seropositive for all four dengue serotypes after the administration of the second unit dose, such as at day 270. In certain such embodiments, at least 80% of the subject population are seropositive for all four dengue serotypes at least one month after administration of the first unit dose, such as at day 30, and before or at the time of the administration of the second unit dose, such as at day 90, and after the administration of the second unit dose, such as at day 120 and at day 270.

In certain embodiments, the reconstituted unit dose provides a seropositivity rate, when it is administered to a subject population of at least 100 subjects in two unit doses subcutaneously at day 1 and at day 90, wherein the subjects of the subject population comprises from 20% to 40% subjects who are seronegative to all dengue serotypes and from 60% to 80% subjects who are seropositive to at least one dengue serotype at base line, wherein at day 120 and/or day 270 the seropositivity rate for all four dengue serotypes in the seronegative part of the subject population and the seropositivity rate for all four dengue serotypes in the seropositive part of the subject population do not deviate more than 10%-points and/or wherein at day 120 the seropositivity rate for all four dengue serotypes in the seronegative part of the subject population and the seropositivity rate for all four dengue serotypes in the seropositive part of the subject population do not deviate more than 5%-points.

In certain particular embodiments, the invention is directed to a dengue vaccine composition as described herein for use in a method of preventing virologically confirmable dengue disease in a subject comprising consecutively administering at least a first and a second unit dose of the dengue vaccine composition to the subject, wherein said first and second unit dose are administered subcutaneously within 3 months and at least 4 weeks apart, optionally at about day 1 and at about day 90, and wherein the dengue vaccine composition is a tetravalent dengue virus composition including four live, attenuated dengue virus strains representing dengue serotype 1, dengue serotype 2, dengue serotype 3 and dengue serotype 4, wherein the attenuated dengue virus strains comprise chimeric dengue viruses and at least one non-chimeric dengue virus, and wherein the dengue serotype 1 and the dengue serotype 2 are present each in a concentration based on the total concentration in pfu/0.5 mL which is within 5%-points of each other and/or are together less than about 10% of the total concentration in pfu/0.5 mL.

In certain embodiments, the method does not comprise a determination of a previous dengue infection of the subject before the administration of the first unit dose of the tetravalent dengue virus composition and wherein the method is safe and effective. Thus, in certain embodiments, the subject has not been tested for the presence a previous dengue infection.

In certain embodiments, the dengue serotype 3 is at least about 10% of the total concentration in pfu/0.5 mL and/or the dengue serotype 4 is at least about 70% of the total concentration in pfu/0.5 mL.

In certain embodiments, the dengue serotype 4 represents the highest concentration in the composition of all four serotypes, preferably with at least about 70% of the total concentration in pfu/0.5 mL, dengue serotype 3 represents the second highest concentration in the composition of all four serotypes, preferably with at least about 10% of the total concentration in pfu/0.5 mL, and dengue serotype 1 and dengue serotype 2 each represent lower concentrations than the concentration of serotype 3, and optionally together represent less than about 10% of the total concentration in pfu/0.5 mL.

In certain embodiments, the dengue serotype 1 is a chimeric dengue serotype 2/1 strain, the dengue serotype 2 is a non-chimeric dengue serotype 2 strain, the dengue serotype 3 is a chimeric dengue serotype 2/3 strain and the dengue serotype 4 is a chimeric dengue serotype 2/4 strain and the dengue serotype 1 has the amino acid sequence of SEQ ID NO. 2, the dengue serotype 2 has the amino acid sequence of SEQ ID NO. 4, the dengue serotype 3 has the amino acid sequence of SEQ ID NO. 6, and the dengue serotype 4 has the amino acid sequence of SEQ ID NO. 8.

In certain embodiments, in the unit dose upon reconstitution with 0.5 ml of a pharmaceutically acceptable diluent
(i) dengue serotype 1 has a concentration of 3.3 log 10 pfu/0.5 mL to 5.0 log 10 pfu/0.5 mL,
(ii) dengue serotype 2 has a concentration of 2.7 log 10 pfu/0.5 mL to 4.9 log 10 pfu/0.5 mL,
(iii) dengue serotype 3 has a concentration of 4.0 log 10 pfu/0.5 mL to 5.7 log 10 pfu/0.5 mL, and
(iv) dengue serotype 4 has a concentration of 4.5 log 10 pfu/0.5 mL to 6.2 log 10 pfu/0.5 mL, and optionally the composition further comprises about 15% (w/v) α,α-trehalose dihydrate, about 1% (w/v) poloxamer 407, about 0.1% (w/v) human serum albumin, and about 100 mM sodium chloride when measured in 0.5 mL.

In certain embodiments, the unit doses are administered to the deltoid region of the arm.

In certain embodiments, the composition is administered without determining the serostatus of the subject at baseline and wherein the administration is safe and effective regardless of the serostatus at base line.

In certain embodiments, the subject is seronegative to all dengue serotypes at baseline and/or is from 4 years of age, optionally to 60 years of age. In certain such embodiments, the subject is 4 to 16 years of age, under 9 years of age, from 2 years of age to under 9 years of age, from 4 years of age to under 9 years of age, 4 to 5 years of age, 6 to 11 years of age, or 12 to 16 years of age. In particular the subject may be under 9 years of age and seronegative to all four dengue serotypes at baseline. In other embodiments, the subject is seropositive to at least one dengue serotypes at baseline and/or is from 4 years of age, optionally to 60 years of age. In certain such embodiments, the subject is 4 to 16 years of age, under 9 years of age, from 2 years of age to under 9 years of age, from 4 years of age to under 9 years of age, 4 to 5 years of age, 6 to 11 years of age, or 12 to 16 years of age. In particular the subject may be under 9 years of age and seropositive to at least one dengue serotypes at baseline. In certain preferred embodiments, the subject is 4 to 5 years of age, 6 to 11 years of age or 12 to 16 years of age.

In certain embodiments, the method is for preventing dengue hemorrhagic fever (DHF) or dengue shock syndrome (DSS).

In certain embodiments, the subject is from a dengue endemic region or from a dengue non-endemic region.

In certain embodiments, the subject is from Asia Pacific or Latin America.

In certain embodiments, the composition provides a seropositivity rate when it is administered to a subject population of at least 50 subjects in two unit doses subcutaneously at day 1 and at day 90, wherein the subjects of the subject population are seronegative to all dengue serotypes at baseline, in particular wherein at least one month after administration of the first unit dose, such as at day 30, at least 80% of the subject population are seropositive for all four dengue serotypes, and/or at least 80% of the subject population are seropositive for all four dengue serotypes before or at the time of the administration of the second unit dose, such as at day 90, and/or at least 80%, or at least 85% or at least 90%, or at least 95% of the subject population are seropositive for all four dengue serotypes after the administration of the second unit dose, such as at day 120, and/or at least 80%, or at least 85%, or at least 90% of the subject population are seropositive for all four dengue serotypes after the administration of the second unit dose, such as at day 270.

In certain embodiments, the composition provides a seropositivity rate, when it is administered to a subject population of at least 100 subjects in two unit doses subcutaneously at day 1 and at day 90, wherein the subjects of the subject population comprises from 20% to 40% subjects who are seronegative to all dengue serotypes and from 60% to 80% subjects who are seropositive to at least one dengue serotype at base line, wherein at day 120 and/or day 270 the seropositivity rate for all four dengue serotypes in the seronegative part of the subject population and the seropositivity rate for all four dengue serotypes in the seropositive part of the subject population do not deviate more than 10%-points and/or wherein at day 120 the seropositivity rate for all four dengue serotypes in the seronegative part of the subject population and the seropositivity rate for all four dengue serotypes in the seropositive part of the subject population do not deviate more than 5%-points.

The present invention is in part directed to the unit dose of the invention as described herein for use in a method of preventing dengue disease (in particular virologically confirmable dengue, VCD) in a subject population comprising administering to the subject population at least a first reconstituted unit dose of the invention as described herein, wherein certain ratios of geometric mean neutralizing antibody titers (GMTs) at day 180 or 365 after administration of said first unit dose to the subject population are achieved. According to some embodiments, the geometric mean neutralizing antibody titer for dengue serotype 2 (GMT DENV-2) and the geometric mean neutralizing antibody titer for dengue serotype 4 (GMT DENV-4) when tested in at least 40, or at least 50, or at least 60 subjects at day 180 or day 365 after at least a first administration of said reconstituted unit dose of the invention as described herein, and optionally a second administration of a reconstituted unit dose of the invention as described herein 90 days after said first administration, provide a ratio of GMT DENV-2:GMT DENV-4 of not more than 50, or not more than 40, or not more than 30, or not more than 20. In some of these embodiments, the ratio of GMT DENV-2:GMT DENV-1 is not more than 20, or not more than 18, or not more than 15 at day 180 or 365 after administration of said first reconstituted unit dose, and/or the ratio of GMT DENV-2:GMT DENV-3 is not more than 20, or not more than 18, or not more than 15 at day 180 or 365 after administration of said first reconstituted unit dose.

The present invention is in part directed to the unit dose of the invention as described herein for use in a method of preventing dengue disease (in particular virologically confirmable dengue, VCD) in a subject comprising administering to the subject at least a first reconstituted unit dose of the invention as described herein, wherein certain ratios of neutralizing antibody titers at day 180 or 365 after administration of said first unit dose to the subject are achieved. According to some embodiments, the neutralizing antibody titer for dengue serotype 2 and the neutralizing antibody titer for dengue serotype 4 at day 180 or day 365 after at least a first administration of the reconstituted unit dose of the invention as described herein, and optionally a second administration of a reconstituted unit dose of the invention as described herein 90 days after said first administration, provide a ratio of neutralizing antibody titer for DENV-2:neutralizing antibody titer for GMT DENV-4 of not more than 50, or not more than 40, or not more than 30, or not more than 20. In some of these embodiments, the ratio of the neutralizing antibody titers of DENV-2: DENV-1 is not more than 20, or not more than 18, or not more than 15 at day 180 or 365 after administration of said first reconstituted unit dose, and/or the ratio of the neutralizing antibody titers of DENV-2: DENV-3 is not more than 20, or not more than 18, or not more than 15 at day 180 or 365 after administration of said first reconstituted unit dose.

The geometric mean neutralizing antibody titers (GMTs) of a subject population or the neutralizing antibody titers of a subject are determined in accordance with the microneutralization test disclosed herein, for example according to the method described in Example 2.

In certain embodiments the invention is directed to the reconstituted unit dose of the invention as described herein for said uses, wherein said unit dose is administered by subcutaneous injection. According to some of these embodiments the subcutaneous injection is administered to the arm, preferably to the deltoid region of the arm.

In certain embodiments the invention is directed to a reconstituted unit dose of the invention as described herein for said uses, wherein the subject or subject population is seronegative to all dengue serotypes.

In certain embodiments the invention is directed to a reconstituted unit dose of the invention as described herein for said uses, wherein a single unit dose of the invention as described herein is administered.

In certain embodiments the invention is directed to a reconstituted unit dose of the invention as described herein for said uses, wherein two reconstituted unit doses of the invention as described herein are administered. In some embodiments, the two reconstituted unit doses are administered within 12 months or more, or within six months, or within three months, such as at day 0 and day 90 or at day 1 and day 90. According to some of these embodiments, a third reconstituted unit dose of the invention as described herein may be administered after the second administration. Such a third administration may act as a booster and may be administered between 6 to 12 months after the first administration, such as 12 months after the first administration, or later than 12 month after the first administration, such as 12 months after the second administration.

In certain embodiments the invention is directed to a reconstituted unit dose of the invention as described herein for said uses, wherein the reconstituted unit dose of the invention as described herein is administered at most in two doses or in one dose.

In certain embodiments of the invention the subject is seronegative with respect to all dengue serotypes. In certain embodiments of the invention the subject is seronegative with respect to all dengue serotypes and the reconstituted unit dose is administered to the seronegative subject by subcutaneous injection.

In certain other embodiments of the invention the subject is seropositive with respect to at least one dengue serotype.

In certain embodiments the invention is directed to the reconstituted unit dose of the invention as described herein for said uses, wherein the reconstituted unit dose of the invention as described herein is administered to a subject or subject population from a dengue endemic region. In some of these embodiments, the subject or subject population is from Singapore, Dominican Republic, Panama, Philippines, Colombia, Puerto Rico or Thailand, in particular from Singapore, Dominican Republic, Panama, or Philippines. In other embodiments, the subject or subject population is from a dengue non-endemic region. Such a subject population or such a subject may be vaccinated according to the invention in the context of traveling to a dengue-endemic region. In certain embodiments, the reconstituted unit dose of the invention as described herein is administered subcutaneously to a subject or subject population from a dengue endemic region or from a dengue non-endemic region.

In some embodiments the invention is directed to the unit dose of the invention as described herein for said uses, wherein the subject or subject population is of 2 to 60 years of age or 18 to 60 years of age. In certain embodiments, the subject or subject population is of 1 to 17 years of age, or less than 9 years of age, or less than 4 years of age or less than 2 years of age. According to some of these embodiments the subject or subject population is seronegative and from a dengue-endemic region.

In certain embodiments, the invention is directed to the reconstituted unit dose of the invention as described herein for said uses, wherein the unit dose of the invention as described herein is administered to a pediatric subject or pediatric subject population of less than 2 years of age, preferably of 2 months to 2 years of age or 2 months to 1.5 years of age or 2 months to 1 year of age. According to some of these embodiments, the pediatric subject or pediatric subject population is seronegative and from a dengue endemic region.

In certain embodiments, the invention is directed to the reconstituted unit dose of the invention as described herein for said uses, wherein the reconstituted unit dose is administered subcutaneously to a pediatric subject or pediatric subject population of less than 2 years of age, preferably of 2 months to 2 years of age or 2 months to 1.5 years of age or 2 months to 1 year of age. According to some of these embodiments, the pediatric subject or pediatric subject population is seronegative and from a dengue endemic region.

The unit dose for use in the methods described above may be any unit dose of a dengue vaccine composition as described above under the headings “Unit dose” or “Dengue vaccine composition” and comprise any dengue virus strain as described above under the heading “Dengue virus strain”.

### Use for the Manufacture of a Medicament for Preventing Dengue Disease

The present invention is directed in part to the use of a unit dose of the invention as described herein for the manufacture of a medicament for preventing dengue disease (in particular virologically confirmable dengue, VCD) in a subject.

The present invention is directed in part to the use of a unit dose of the invention as described herein for the manufacture of a medicament for preventing dengue disease (in particular virologically confirmable dengue, VCD) in a subject population.

Any method described herein above under the heading “Method of preventing” is to be understood to be also disclosed as the use of a unit dose for the manufacture of a medicament for preventing dengue disease in a subject or subject population with such a method irrespective of being expressly stated below.

The present invention is in part directed to the use of a unit dose of the invention as described herein for the manufacture of a medicament for preventing dengue disease in a subject population, comprising administering to the subject population at least a first reconstituted unit dose of the invention as described herein, wherein certain ratios of geometric mean neutralizing antibody titers (GMTs) at day 180 or 365 after administration of said first unit dose to the subject population are achieved. According to some embodiments, the geometric mean neutralizing antibody titer for dengue serotype 2 (GMT DENV-2) and the geometric mean neutralizing antibody titer for dengue serotype 4 (GMT DENV-4) when tested in at least 40, or at least 50, or at least 60 subjects at day 180 or day 365 after at least a first administration of said reconstituted unit dose of the invention as described herein, and optionally a second administration of a reconstituted unit dose of the invention as described herein 90 days after said first administration, provide a ratio of GMT DENV-2:GMT DENV-4 of not more than 50, or not more than 40, or not more than 30, or not more than 20. In some of these embodiments, the ratio of GMT DENV-2:GMT DENV-1 is not more than 20, or not more than 18, or not more than 15 at day 180 or 365 after administration of said first reconstituted unit dose, and/or the ratio of GMT DENV-2:GMT DENV-3 is not more than 20, or not more than 18, or not more than 15 at day 180 or 365 after administration of said first reconstituted unit dose.

The present invention is in part directed to the use of a unit dose of the invention as described herein for the manufacture of a medicament for preventing dengue disease in a subject, comprising administering to the subject at least a first reconstituted unit dose of the invention as described herein, wherein certain ratios of neutralizing antibody titers at day 180 or 365 after administration of said first unit dose to the subject are achieved. According to some embodiments, the neutralizing antibody titer for dengue serotype 2 and the neutralizing antibody titer for dengue serotype 4 at day 180 or day 365 after at least a first administration of the reconstituted unit dose of the invention as described herein, and optionally a second administration of a reconstituted unit dose of the invention as described herein 90 days after said first administration, provide a ratio of neutralizing antibody titer for DENV-2:neutralizing antibody titer for GMT DENV-4 of not more than 50, or not more than 40, or not more than 30, or not more than 20. In some of these embodiments, the ratio of the neutralizing antibody titers of DENV-2: DENV-1 is not more than 20, or not more than 18, or not more than 15 at day 180 or 365 after administration of said first reconstituted unit dose, and/or the ratio of the neutralizing antibody titers of DENV-2: DENV-3 is not more than 20, or not more than 18, or not more than 15 at day 180 or 365 after administration of said first reconstituted unit dose.

In some embodiments, the geometric mean neutralizing antibody titers (GMTs) of a subject population or the neutralizing antibody titers of a subject are determined in accordance with the microneutralization test disclosed herein, for example according to the method described in Example 2.

In certain embodiments the invention is directed to said uses, wherein the reconstituted unit dose of the invention as described herein is administered by subcutaneous injection. According to some of these embodiments the subcutaneous injection is administered to the arm, preferably to the deltoid region of the arm.

In certain embodiments the invention is directed to said uses, wherein one reconstituted unit dose of the invention as described is administered,

In certain embodiments the invention is directed to said uses, wherein two reconstituted unit doses of the invention as described herein are administered. In one embodiment, the two unit doses are administered within 12 months or more, or within six months, or within three months, such as at day 0 and day 90 or at day 1 and day 90. According to some of these embodiments a third unit dose of the invention as described herein may be administered after the second administration. Such a third administration may act as a booster and may be administered between 6 to 12 months after the first administration, such as 12 months after the first administration, or later than 12 month after the first administration, such as 12 months after the second administration.

In certain embodiments of the invention the subject is seronegative with respect to all dengue serotypes.

In certain other embodiments of the invention the subject is seropositive with respect to at least one dengue serotype.

In certain embodiments the invention is directed to said uses, wherein the reconstituted unit dose is administered to the seronegative subject by subcutaneous injection.

In certain embodiments the invention is directed to said uses, wherein the reconstituted unit dose is administered to a subject of unknown serostatus and/or wherein no test has been carried out to determine whether the subject is seropositive or seronegative before the unit dose is administered.

In certain embodiments the invention is directed to said uses, wherein the reconstituted unit dose of the invention as described herein is administered to a subject or subject population from a dengue endemic region. In some of these embodiments, the subject or subject population is from Singapore, Dominican Republic, Panama, Philippines, Colombia, Puerto Rico or Thailand, in particular from Singapore, Dominican Republic, Panama, or Philippines. In other embodiments, the subject or subject population is from a dengue non-endemic region. Such a subject population or subject may be vaccinated according to the invention in the context of traveling to a dengue endemic region. In certain embodiments, the reconstituted unit dose of the invention as described herein is administered subcutaneously to a subject or subject population from a dengue endemic region or from a dengue non-endemic region.

In certain embodiments the invention is directed to said uses, wherein the subject is of 2 to 60 years of age or 18 to 60 years of age. In certain embodiments the subject is 1 to 17 years of age, or less than 9 years of age, or less than 4 years of age or less than 2 years of age. According to some of these embodiments the subject is seronegative and from a dengue-endemic region.

In certain embodiments, the invention is directed to said uses, wherein the unit dose of the invention as described herein is administered to a pediatric subject or pediatric subject population of less than 2 years of age, preferably of 2 months to 2 years of age or 2 months to 1.5 years of age or 2 months to 1 year of age. According to some of these embodiments, the pediatric subject or pediatric subject population is seronegative and from a dengue endemic region.

In certain embodiments, the invention is directed to said uses, wherein the reconstituted unit dose of the invention as described herein is administered subcutaneously to a pediatric subject or pediatric subject population of less than 2 years of age, preferably of 2 months to 2 years of age or 2 months to 1.5 years of age or 2 months to 1 year of age. According to some of these embodiments, the pediatric subject or pediatric subject population is seronegative and from a dengue endemic region.

### Method of Stimulating an Immune Response and Uses

### Method of Stimulating an Immune Response

In certain embodiments the invention is directed to a method for stimulating an immune response, preferably a balanced immune response, to all four dengue serotypes in a subject, comprising administering to the subject a reconstituted unit dose of the invention as described herein.

The present invention is in part directed to a method for stimulating an immune response to all four serotypes of dengue virus in a subject, comprising administering to the subject a reconstituted unit dose of the invention as described herein by subcutaneous injection.

In certain embodiments, the invention is directed to said method, wherein the immune response to all four serotypes of dengue virus is balanced.

In certain embodiments, the invention is directed to said method, wherein the reconstituted unit dose is administered by subcutaneous injection to the arm, preferably to the deltoid region of the arm.

In certain embodiments, the invention is directed to said method, wherein the subject is seronegative to all dengue serotypes.

In certain embodiments, the invention is directed to said method, wherein two reconstituted unit doses of the invention as described herein are administered. In some embodiments, the two reconstituted doses are administered within 12 months or more, or within six months, or within three months, such as at day 0 and day 90 or at day 1 and day 90. According to some of these embodiments, a third unit dose of the invention as described herein is administered between 6 and 12 months after the administration of said first unit dose, such as 12 months after the first administration, or later than 12 month after the first administration, such as 12 months after the second administration.

In certain embodiments, the invention is directed to said method, wherein the unit dose of the invention as described herein is administered to a subject from a dengue endemic region. In some of these embodiments, the subject is from Singapore, Dominican Republic, Panama, Philippines, Colombia, Puerto Rico or Thailand, in particular from Singapore, Dominican Republic, Panama, or Philippines. In other embodiments, the subject is from a dengue non-endemic region. Such a subject may be subject to a vaccination according to the invention in the context of traveling to a dengue endemic region. In certain embodiments, the reconstituted unit dose of the invention as described herein is administered subcutaneously to a subject that is from a dengue endemic region or a dengue non-endemic region.

In certain embodiments, the invention is directed to said method, wherein the reconstituted unit dose of the invention as described herein is administered subcutaneously to a subject that is seronegative with respect to all dengue serotypes. In other embodiments, the subject is seropositive with respect to at least one dengue serotype.

In certain embodiments, the invention is directed to said method, wherein the neutralizing antibody titers of the subject when tested at day 180 or day 365 after at least a first administration of said unit dose, and optionally a second administration of said unit dose 90 days after said first administration, provide a ratio of not more than 50, or not more than 40, or nor more than 30, or not more than 20 for the neutralizing antibody titer of dengue serotype 2 to the neutralizing antibody titer of dengue serotype 4. In certain embodiments, said neutralizing antibody titers of the subject further provide a ratio of not more than 20 for the neutralizing antibody titer of dengue serotype 2 to the neutralizing antibody titer of dengue serotype 1, and/or a ratio of not more than 20 for the neutralizing antibody titer of dengue serotype 2 to the neutralizing antibody titer of dengue serotype 3.

In certain embodiments, the invention is directed to said method, wherein the unit dose of the invention as described herein is administered to a subject of 2 to 60 years of age or 18 to 60 years of age. In certain embodiments the subject is 1 to 17 years of age, or less than 9 years of age, or less than 4 years of age or less than 2 years of age. According to some of these embodiments the subject is seronegative and from a dengue-endemic region.

In certain embodiments, the invention is directed to said method, wherein the unit dose of the invention as described herein is administered to a pediatric subject of less than 2 years of age, preferably of 2 months to 2 years of age or 2 months to 1.5 years of age or 2 months to 1 year of age. According to some of these embodiments, the pediatric subject is seronegative and from a dengue endemic region.

In certain embodiments, the invention is directed to said method, wherein the reconstituted unit dose of the invention as described herein is administered subcutaneously to a pediatric subject of less than 2 years of age, preferably of 2 months to 2 years of age or 2 months to 1.5 years of age or 2 months to 1 year of age. According to some of these embodiments, the pediatric subject is seronegative and from a dengue endemic region.

### Unit Dose for Use in a Method of Stimulating an Immune Response

The present invention is in part directed to the reconstituted unit dose of the invention as described herein for use in a method for stimulating an immune response to all four serotypes of dengue virus in a subject.

The present invention is in part directed to the reconstituted unit dose of the invention as described herein for use in a method for stimulating an immune response to all four serotypes of dengue virus in a subject, wherein a reconstituted unit dose of the invention as described herein is administered to the subject, preferably by subcutaneous injection.

In certain embodiments, the invention is directed to the reconstituted unit dose of the invention as described herein for said use, wherein the immune response to all four serotypes of dengue virus is balanced.

In certain embodiments, the invention is directed to the reconstituted unit dose of the invention as described herein for said use, wherein the reconstituted unit dose is administered by subcutaneous injection to the arm, preferably to the deltoid region of the arm.

In certain embodiments, the invention is directed the reconstituted unit dose of the invention as described herein for said use, wherein the subject is seronegative to all dengue serotypes.

In certain embodiments, the invention is directed to the unit dose of the invention as described herein for said use, wherein two reconstituted unit doses of the invention as described herein are administered. In some embodiments, the two reconstituted unit doses are administered within 12 months or more, or within six months, or within three months, such as at day 0 and day 90 or at day 1 and day 90. According to some of these embodiments, a third reconstituted unit dose is administered 6 to 12 months after the administration of the first reconstituted unit dose, such as 12 months after the first administration, or later than 12 month after the first administration, such as 12 months after the second administration.

In certain embodiments, the invention is directed to the unit dose of the invention as described herein for said use, wherein the subject is from a dengue endemic region. In other embodiments, the subject is from a dengue non-endemic region. Such a subject may be subject to a vaccination according to the invention in the context of traveling to a dengue endemic region. In certain embodiments, the reconstituted unit dose of the invention as described herein is administered subcutaneously to a subject that is from a dengue endemic region or a dengue non-endemic region.

In certain embodiments, the invention is directed to the unit dose of the invention as described herein for said use, wherein the reconstituted unit dose of the invention as described herein is administered subcutaneously to a subject that is seronegative with respect to all dengue serotypes. In other embodiments, the subject is seropositive with respect to at least one dengue serotype.

In certain embodiments, the invention is directed to the unit dose of the invention as described herein for said use, wherein the neutralizing antibody titers of the subject when tested at day 180 or day 365 after at least a first administration of said unit dose, and optionally a second administration of said unit dose 90 days after said first administration, provide a ratio of not more than 50, or not more than 40, or nor more than 30, or not more than 20 for the neutralizing antibody titer of dengue serotype 2 to the neutralizing antibody titer of dengue serotype 4. In certain embodiments, said neutralizing antibody titers of the subject further provide a ratio of not more than 20 for the neutralizing antibody titer of dengue serotype 2 to the neutralizing antibody titer of dengue serotype 1, and/or a ratio of not more than 20 for the neutralizing antibody titer of dengue serotype 2 to the neutralizing antibody titer of dengue serotype 3.

In certain embodiments, the invention is directed to the reconstituted unit dose of the invention as described herein for said use, wherein the unit dose of the invention as described herein is administered to a subject of 2 to 60 years of age or 18 to 60 years of age. In certain embodiments the subject is 1 to 17 years of age, or less than 9 years of age, or less than 4 years of age or less than 2 years of age.

According to some of these embodiments the subject is seronegative and from a dengue-endemic region.

In certain embodiments, the invention is directed to the reconstituted unit dose of the invention as described herein for said use, wherein the unit dose of the invention as described herein is administered to a pediatric subject of less than 2 years of age, preferably of 2 months to 2 years of age or 2 months to 1.5 years of age or 2 months to 1 year of age. According to some of these embodiments, the pediatric subject is seronegative and from a dengue endemic region.

In certain embodiments, the invention is directed to the reconstituted unit dose of the invention as described herein for said use, wherein the reconstituted unit dose of the invention as described herein is administered subcutaneously to a pediatric subject of less than 2 years of age, preferably of 2 months to 2 years of age or 2 months to 1.5 years of age or 2 months to 1 year of age. According to some of these embodiments, the pediatric subject is seronegative and from a dengue endemic region.

### Use for the Manufacture of a Medicament for Stimulating an Immune Response

The present invention is in part directed to the use of the reconstituted unit dose of the invention as described herein for the manufacture of a medicament for stimulating an immune response to all four serotypes of dengue virus in a subject. In one embodiment a reconstituted unit dose of the invention as described herein is administered by subcutaneous injection.

In certain embodiments, the invention is directed to said use, wherein the immune response to all four serotypes of dengue virus is balanced.

In certain embodiments, the invention is directed to said use, wherein the reconstituted unit dose is administered by subcutaneous injection to the arm, preferably to the deltoid region of the arm.

In certain embodiments, the invention is directed the reconstituted unit dose of the invention as described herein for said use, wherein the subject is seronegative to all dengue serotypes.

In certain embodiments, the invention is directed to said use, wherein two reconstituted unit doses of the invention as described herein are administered. In some embodiments, the two reconstituted unit doses are administered within 12 months or more, or within six months, or within three months, such as at day 0 and day 90 or at day 1 and day 90. According to some of these embodiments, a third reconstituted unit dose is administered 6 to 12 months after the administration of the first reconstituted unit dose, such as 12 months after the first administration, or later than 12 month after the first administration, such as 12 months after the second administration.

In certain embodiments, the invention is directed to said use, wherein the subject is from a dengue endemic region. In other embodiments, the subject is from a dengue non-endemic region. Such a subject may be subject to a vaccination according to the invention in the context of traveling to a dengue endemic region. In certain embodiments, the reconstituted unit dose of the invention as described herein is administered subcutaneously to a subject that is from a dengue endemic region or a dengue non-endemic region.

In certain embodiments, the invention is directed to said use, wherein the reconstituted unit dose of the invention as described herein is administered subcutaneously to a subject that is seronegative with respect to all dengue serotypes. In other embodiments, the subject is seropositive with respect to at least one dengue serotype.

In certain embodiments, the invention is directed to said use, wherein the neutralizing antibody titers of the subject when tested at day 180 or day 365 after at least a first administration of said unit dose, and optionally a second administration of said unit dose 90 days after said first administration, provide a ratio of not more than 50, or not more than 40, or nor more than 30, or not more than 20 for the neutralizing antibody titer of dengue serotype 2 to the neutralizing antibody titer of dengue serotype 4. In certain embodiments, said neutralizing antibody titers of the subject further provide a ratio of not more than 20 for the neutralizing antibody titer of dengue serotype 2 to the neutralizing antibody titer of dengue serotype 1, and/or a ratio of not more than 20 for the neutralizing antibody titer of dengue serotype 2 to the neutralizing antibody titer of dengue serotype 3.

In certain embodiments, the invention is directed to said use, wherein the reconstituted unit dose of the invention as described herein is administered to a subject of 2 to 60 years of age or 18 to 60 years of age. In certain embodiments the subject is 1 to 17 years of age, or less than 9 years of age, or less than 4 years of age or less than 2 years of age. According to some of these embodiments the subject is seronegative and from a dengue-endemic region.

In certain embodiments, the invention is directed to said use, wherein the unit dose of the invention as described herein is administered to a pediatric subject of less than 2 years of age, preferably of 2 months to 2 years of age or 2 months to 1.5 years of age or 2 months to 1 year of age. According to some of these embodiments, the pediatric subject is seronegative and from a dengue endemic region.

In certain embodiments, the invention is directed to said use, wherein the reconstituted unit dose of the invention as described herein is administered subcutaneously to a pediatric subject of less than 2 years of age, preferably of 2 months to 2 years of age or 2 months to 1.5 years of age or 2 months to 1 year of age. According to some of these embodiments, the pediatric subject is seronegative and from a dengue endemic region.

### Method for Determining the Titer of Neutralizing Antibodies

The present invention is directed in part to a method for determining the titer of neutralizing antibodies against each of dengue serotypes 1, 2, 3 and 4 in a blood serum sample, the method comprising the steps of:
(a) seeding cells from a dengue-susceptible cell line on 96-well assay plates and culturing the cells for a culture period;
(b) preparing serial dilutions of the blood serum sample;
(c) separately mixing the serially diluted blood serum samples prepared in step (b) with dengue serotype 1, dengue serotype 2, dengue serotype 3 and dengue serotype 4 to obtain separate mixtures for each dengue serotype and incubating the separate mixtures;
(d) adding the separate mixtures prepared in (c) to the cells seeded and cultured in step (a) and incubating the cells with the separate mixtures;
(e) providing an overlay for the inoculated cells and incubating the cells for an incubation period of 40 to 75 hours;
(f) determining the number of plaques in each well and comparing the number of plaques in each well to a control to determine the level of neutralizing antibodies against each of dengue serotypes 1, 2, 3 and 4.

In one embodiment, different incubation periods are used in step (e) for the mixtures of different dengue serotypes. In some embodiments, the incubation period for mixtures of dengue serotype 4 is shorter than the incubation period for mixtures of dengue serotypes 1, 2 and 3, for example the incubation period for mixtures of dengue serotype 4 is less than 50 hours, preferably 46±2 hours. In some embodiments, the incubation period for mixtures of dengue serotype 2 is longer than the incubation period for mixtures of dengue serotypes 1, 3 and 4, for example the incubation period for mixtures of dengue serotype 2 is between 60 and 70 hours, preferably 70±2 hours.

In one embodiment, the dengue-susceptible cell line used in step (a) is selected from Vero cells, LLC-MK2 cells and BHK-21 cells. In some embodiments, the culture period of the cells is 12 to 36 hours.

In one embodiment, in step (c) the dengue serotype 1 is DENV-1 strain 16007, dengue serotype 2 is DENV-2 strain 16681, dengue serotype 3 is DENV-3 strain 16562 and dengue serotype 4 is DENV-4 strain 1036.

In one embodiment, the separate mixtures in step (c) are incubated overnight at a temperature of 2 to 8° C.

In one embodiment, the overlay in step (e) is selected from the group consisting of methylcellulose, carboxymethylcellulose and agarose. In some embodiments, the cells with the overlay are incubated at a temperature of 33° C. to 35° C.

In one embodiment, the number of plaques in each well is determined using serotype-specific anti-dengue monoclonal antibodies.

In one embodiment, the invention is directed to a method for determining the titer of neutralizing antibodies against each of dengue serotypes 1, 2, 3 and 4 in a blood serum sample, the method comprising the steps of:
(a) seeding Vero cells on 96-well assay plates and culturing the Vero cells for a period of 20 to 30 hours;
(b) preparing serial dilutions of the serum sample;
(c) separately mixing the serially diluted serum samples with dengue serotype 1, dengue serotype 2, dengue serotype 3 and dengue serotype 4 to prepare separate mixtures and incubating the separate mixtures overnight at a temperature of 2 to 8° C.;
(d) incubating the cells seeded and cultured in step (a) with the separate mixtures prepared in step (c) in separate wells for 90 to 120 minutes;
(e) providing a methylcellulose overlay for the inoculated cells and incubating the cells for an incubation period of 40 to 75 hours at 34° C.;
(f) determining the number of plaques in each well using serotype-specific anti-dengue monoclonal antibodies and comparing the number of plaques in each well to a control to determine the level of neutralizing antibodies against each of dengue serotypes 1, 2, 3 and 4.

In one embodiment, the invention is directed to the use of said method for determining the dengue serostatus of a subject before vaccination with a dengue virus vaccine or for analyzing a subject's immune response after vaccination with a dengue virus vaccine.

### EXAMPLES

The following Examples are included to demonstrate certain aspects and embodiments of the invention as described in the claims. It should be appreciated by those of skill in the art, however, that the following description is illustrative only and should not be taken in any way as a restriction of the invention.

### Example 1: Preparation of the Dengue Virus Strains

The methods used to generate the chimeric dengue strains TDV-1, -3 and -4 were standard molecular cloning and DNA engineering methods and are described in Huang et al. (2003) J. Virology 77 (21): 11436-11447. The following well-known methods were used to construct and introduce the prM-E genes of dengue serotypes 1, 3 and 4 into the TDV-2 backbone: Reverse-transcriptase PCR (RT-PCR), PCR, restriction enzyme digestion, DNA fragment ligation, bacterial transformations by electroporation, plasmid DNA preparations, in vitro transcription by T7 RNA polymerase, and transfection of Vero cells by electroporation.

After growing and purifying the different dengue serotypes separately as described in Huang et al. (2013) PLOS Neglected Dis, 7 (5): e2243, they are mixed in the concentrations provided in Table 5. The mixture of dengue serotypes is present in a dengue vaccine composition and combined with a composition of pharmaceutically acceptable excipients resulting in a dengue vaccine composition comprising 15% w/v α,α trehalose dihydrate, 1% w/v poloxamer 407, 0.1% w/v human serum albumin and 100 mM sodium chloride. The dengue vaccine composition is lyophilized and represents a lyophilized unit dose of TDV. The lyophilized unit dose is reconstituted with 37 mM aqueous sodium chloride solution and the reconstituted unit dose comprises 15% w/v α,α trehalose dihydrate, 1% w/v poloxamer 407, 0.1% w/v human serum albumin and 137 mM sodium chloride.

### Example 2: Microneutralization Test

Immunogenicity was measured by a microneutralization assay to each one of the four dengue serotypes with titers defined as the dilution resulting in a 50% reduction in plaque values (MNT50). Briefly, on day 1 Vero cells were seeded on 96-well assay plates in DMEM and 10% FBS at a density of 2.5×10⁵ cells/ml and incubated at 37° C. for 24 hours. On day 2 serial dilutions of the heat-inactivated antibody-containing test and control sera samples (dilutions range 1:10 to 1:20480) were prepared and mixed with a constant concentration of dengue viruses, in particular DENV-1 strain 16007, DENV-2 strain 16681, DENV-3 strain 16562 and DENV-4 strain 1036, (target 60-80 pfu/well) in a 96 well microtiter plate and incubated overnight at 2-8° C. to enable the neutralization of the virus by the antibodies present in the sera. After the incubation the mixture of virus and antibodies was transferred onto the 96 well plates with Vero cells and the plates were incubated at 37° C. for 90-120 minutes to infect the Vero cells. A 1% methylcellulose overlay in DMEM was applied to the plate to restrict spread of progeny virus and the plate was incubated for 46-70 hours at 34° C. depending on the Dengue serotype:
DENV1—66±2 hours
DENV2—70±2 hours
DENV3—66±2 hours
DENV4—46±2 hours

After the incubation the cells were washed twice with PBS and fixed by adding cold methanol and incubating for 60 minutes at a temperature of ≤−20° C. After fixing the plates were dried and washed three times with washing buffer (1×PBS, pH 7.4 with 0.5% Tween), before 50 μl of serotype-specific anti-dengue monoclonal antibodies in blocking solution (2.5% nonfat dry milk in PBST) per well were added and incubated with the cells for 18±4 hours at 2-8° C.

The monoclonal antibodies were made as described in Gentry et al. (1982) Am. J. Trop. Med. Hyg. 31, 548-555; Henchal et al. (1985) Am. J. Trop. Med. Hyg. 34, 162-169; and Henchal et al. (1982) Am. J. Trop. Med. Hyg. 31 (4): 830-6). Briefly, the anti-DENV-1 HBD was made against dengue 1 strain Hawaii, Envelope, the anti-DENV-2 was made against dengue 2 strain New Guinea C, Envelope, isotype 1, the anti-DENV-3 HBD was made against dengue 3 strain H87, Envelope, isotype 2A, and the anti-DENV-4 HBD was made against dengue 4 strain H241, Envelope, isotype 2A.

After incubation, the plates were washed three times with washing buffer and 50 μl of a secondary peroxidase labelled goat anti-mouse IgG (H+L) (KPL Cat #074-1806) in blocking solution was added and incubated for 90 to 120 minutes at 37° C. Then the plates were washed three times with washing buffer and 50 μl of precipitant substrate (2-amino-9-ethyl carbazole (AEC) tablet in 2.5 ml DMSO, 47.5 ml 50 mM acetate buffer and 250 μl hydrogen peroxide) were added and the mixture was incubated for 20 minutes at room temperature. Finally, the substrate was removed, the plates were rinsed with dH₂O and dried.

Sample titers are calculated using the linear regression method and reported as MNT50 titers for each sample. Clinical data are reported as a geometric mean titer for all the individual MNT50 titers in each treatment group. Briefly, the number of infectious foci in each well was counted and the titer of neutralizing antibodies was determined by comparing the percent reduction of infectious foci centers in wells containing antibody (test samples) in comparison to wells containing virus alone. The MNT50 was calculated using the following linear regression equation:

MNT50=10\hat{ }[(50−c)/m]) where c=y intercept of regression line and m=slope of regression line

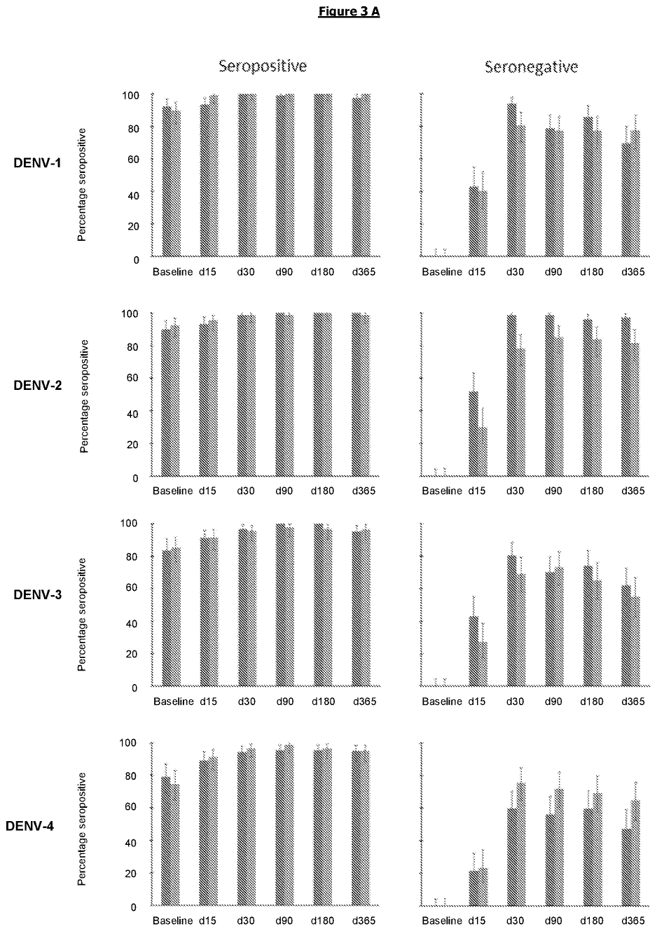

Each test sample was tested in triplicates and the titer was calculated from the average of the triplicates. A schematic drawing of the steps performed in this test is provided in FIG. 2.

### Example 3: Clinical Trial Comparing Two Different Unit Doses

A descriptive Phase II, double-blind, randomized, and controlled trial in 351 subjects aged 21 to 45 years living in Singapore was performed comparing two different formulations of a tetravalent dengue vaccine. Subjects were randomized (1:1), stratified by baseline dengue serostatus, into two treatment groups: groups 1 received one subcutaneous (SC) dose of HD-TDV and group 2 received one subcutaneous dose of TDV. The dengue vaccine formulations were prepared as described in Example 1. The concentration of the four dengue serotypes in the HD-TDV vaccine (high dose tetravalent dengue vaccine) and in the TDV vaccine (corresponding to the unit dose of the invention as described herein) is outlined in Table 5.

TABLE 5
Unit doses used in the trial.

| | |
|---|---|
| Comparative | 0.5 mL comprising TDV-1, TDV-2, TDV-3 and TDV-4 |
| unit dose | containing 3.2 × 10⁴ PFU, 1.3 × 10⁵ PFU, 2.5 × 10⁵ PFU, |
| (HD-TDV) | and 4 × 10⁵ PFU, respectively. Administered by |
| | subcutaneous injection on day 1. |
| Example 1 | 0.5 mL comprising TDV-1, TDV-2, TDV-3 and TDV-4 |
| unit dose | containing 2.5 × 10⁴ PFU, 6.3 × 10³ PFU, 3.2 × 10⁴ PFU, |
| (TDV) | and 4 × 10⁵ PFU, respectively. Administered by |
| | subcutaneous injection on day 1. |

Immunogenicity was evaluated at Days 15, 30, 90, 180, and 365 post-vaccination as geometric mean titers (GMTs) and seropositivity rates. Immunogenicity of the vaccines against each of the four dengue serotypes was assessed using a microneutralization assay, with titers corresponding to the dilution resulting in a 50% reduction in plaque reduction (MNT50) as described in Example 2. Primary immunogenicity endpoints were reported in terms of geometric mean titers (GMTs) of neutralizing antibodies, and seropositivity rates (which were defined as percentages of subjects with reciprocal neutralizing titers≥10 for each of the DENV serotypes) in the overall trial population. As a secondary endpoint, GMTs and seropositivity rates were analyzed by dengue baseline seropositivity status. Seropositive at baseline was defined as being seropositive for at least one DENV serotype, whereas seronegative at baseline was defined as being seronegative for all four DENV serotypes.

Solicited and unsolicited adverse events (AEs) were assessed by severity and causality.

### a) Seropositivity

Dengue seropositivity is based on the result of the microneutralization test (MNT) described in Example 2 and is defined as a reciprocal neutralizing antibody titer≥10 for one or more dengue serotype at baseline. The baseline seropositivity rate for each dengue serotype is defined as the percentage of seropositive subjects for the given dengue serotype and was derived from the neutralizing antibodies titers of the dengue serotypes as measured in the subjects before administration of the first unit dose. The seropositivity rate at day 180 or day 365 is defined as the percentage of seropositive subjects and was derived from the neutralizing antibodies titers of the dengue serotypes as measured in the subjects 180 and 365 days after administration of the first unit dose, respectively.

In total, 187 subjects (53.6%) were seropositive, based on MNT50, for at least one dengue serotype at baseline: 48.7% were seropositive for DENV-1, 49.0% for DENV-2, 45.2% for DENV-3, and 41.2% for DENV-4. The seropositive status and rate at baseline of the two different vaccination groups is shown in Table 6.

TABLE 6
Serostatus and seropositivity rate at baseline

| | Comparative | Example |
|---|---|---|
| | unit dose | 1 unit dose |
| Baseline seropositivity status | | |
| (count of participants) | | |
| Seropositive for at least one | 92 | 95 |
| dengue serotype | | |
| Seronegative for all dengue | 83 | 80 |
| serotypes | | |
| Baseline seropositivity rate | | |
| for each serotype | | |
| (percentage of participants) | | |
| TDV-1 | 48.6 | 48.6 |
| TDV-2 | 47.4 | 50.3 |
| TDV-3 | 44.0 | 46.3 |
| TDV-4 | 41.7 | 40.6 |

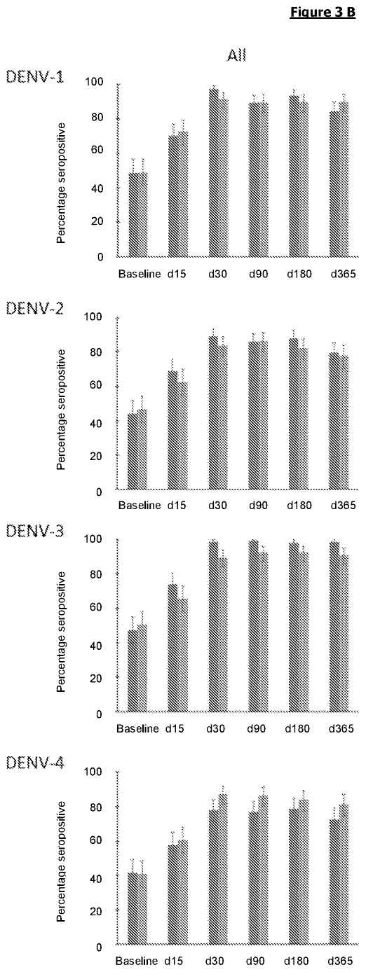

Seropositivity rates increased to Day 30 after administration of the unit doses, and remained high through to Day 365 for each of the four serotypes (FIG. 3). For the overall trial population, the percentages of subjects who were seropositive for DENV-1 and DENV-3 were similar in the HD-TDV and TDV groups, whereas higher post-baseline seropositivity rates were seen for the HD-TDV group against DENV-2, and for the TDV group against DENV-4 (FIG. 3B). In general, higher seropositivity rates were seen in subjects already seropositive at baseline than in seronegative subjects. Seropositivity rates rose to nearly 100% against all four dengue serotypes in the seropositive vaccine groups by Day 30, and remained at this level through to Day 365; no difference was seen between HD-TDV and TDV (FIG. 3A). In the seronegative group, the seropositivity rates increased more gradually to a peak at Day 30, with limited decline until Day 365. The rates were consistently higher for HD-TDV than TDV against DENV-2, but were higher for TDV than HD-TDV against DENV-4, through to Day 365 (FIG. 3A).

TABLE 7
Seropositivity rate at day 180

| | Comparative | Example |
|---|---|---|
| | unit dose | 1 unit dose |
| Overall number of | 166 | 163 |
| participants analyzed | | |
| Seropositivity rate | | |
| at day 180 (95% Confidence | | |
| Interval) | | |
| Day 180, TDV-1 | 93.4 (88.5 to 96.6) | 89.6 (83.8 to 93.8) |
| Day 180, TDV-2 | 98.2 (94.8 to 99.6) | 92.6 (87.5 to 96.1) |
| Day 180, TDV-3 | 88.0 (82.0 to 92.5) | 82.2 (75.5 to 87.7) |
| Day 180, TDV-4 | 78.9 (71.9 to 84.9) | 84.0 (77.5 to 89.3) |

TABLE 8
Seropositivity rate at day 365

| | Comparative | Example 1 |
|---|---|---|
| | unit dose | unit dose |
| Overall number of | 160 | 156 |
| participants analyzed | | |
| Seropositivity rate | | |
| at day 365 (95% Confidence | | |
| Interval) | | |
| Day 365, TDV-1 | 84.4 (77.8 to 89.6) | 89.7 (83.9 to 94.0) |
| Day 365, TDV-2 | 98.8 (95.6 to 99.8) | 91.0 (85.4 to 95.0) |
| Day 365, TDV-3 | 79.4 (72.3 to 85.4) | 77.6 (70.2 to 83.8) |
| Day 365, TDV-4 | 72.5 (64.9 to 79.3) | 81.4 (74.4 to 87.2) |

### b) Geometric Mean Neutralizing Antibody Titers (GMTs)

Neutralizing antibody titers (GMTs) for each dengue serotype were determined in a serum sample of a subject taken before administration of the first unit dose of the dengue vaccine composition and 180 or 365 days after administration of the first unit dose of the dengue vaccine composition using the microneutralization (MNT) assay as described in Example 2.

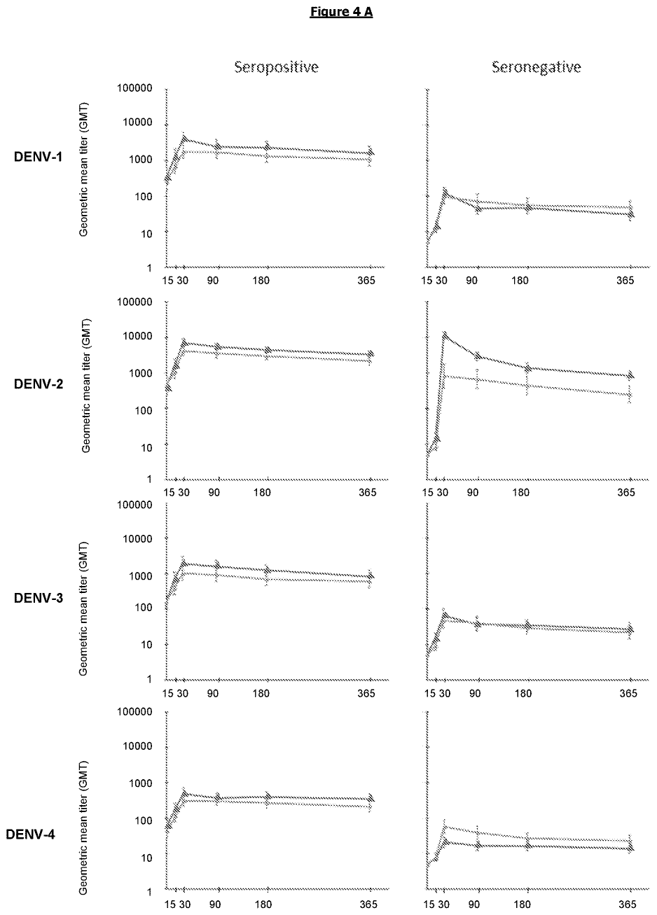

For both HD-TDV and TDV, an increase in GMTs was observed by Day 15, reaching a maximum by Day 30 (FIG. 4). Antibody titers remained above baseline levels throughout the trial for both unit doses. In the overall trial population, no substantial differences were seen in GMT titers between the two unit dose groups, except against DENV-2, where the response was higher for the HD-TDV group compared with the TDV group (8640.3 versus 1992.7 at Day 30). When assessed by baseline seropositivity status, the GMT profiles were similar as for the entire population, with a rise by Day 15, peak at Day 30, and gradual decline thereafter (FIG. 4B). In the group who were seropositive at baseline, the difference between the unit dose groups in response to DENV-2 was reduced, with GMTs of 6970.3 and 4193.3 at Day 30 for the HD-TDV and TDV groups, respectively. Responses were higher against DENV-1, DENV-3, and DENV-4 in the baseline seropositive group, compared with the baseline seronegative group, across both unit doses. Against DENV-2, a lower response was seen in baseline seronegative subjects receiving TDV, compared with HD-TDV; Day 30 GMTs were 812.9 in the TDV group, compared with 10965.9 in the HD-TDV group. The response in these subjects also differed against DENV-4, with a higher response being observed in the TDV group (Day 30 GMTs of 57.7, compared with 20.9 in the HD-TDV group); this difference persisted to Day 365 (FIG. 4A).

TABLE 9
Geometric mean neutralizing antibody titers (GMTs) at day 180

| | Comparative unit dose | | Example 1 unit dose | |
|---|---|---|---|---|
| Overall number of | 166 | | 163 | |
| participants analyzed | | | | |
| GMTs (95% Confidence | | | | |
| Interval) [units: Titer] | | | | |
| Day 180, TDV-1 | 379.4 | (252.3 to 570.3) | 312.2 | (212.2 to 459.2) |
| Day 180, TDV-2 | 2585.5 | (2088.8 to 3200.3) | 1235.0 | (890.7 to 1712.5) |
| Day 180, TDV-3 | 236.2 | (162.2 to 344.0) | 161.0 | (110.5 to 234.6) |
| Day 180, TDV-4 | 91.0 | (65.7 to 125.9) | 92.9 | (68.9 to 125.4) |

TABLE 10
Ratio of geometric mean neutralizing
antibody titers (GMTs) at day 180

| | Comparative | Example 1 |
|---|---|---|
| | unit dose | unit dose |
| TDV-2:TDV-1 | 7 | 4 |
| TDV-2:TDV-3 | 11 | 8 |
| TDV-2:TDV-4 | 28 | 13 |

TABLE 11
Geometric mean neutralizing antibody titers (GMTs) at day 365

| | Comparative unit dose | | Example 1 unit dose | |
|---|---|---|---|---|
| Overall number of | 160 | | 156 | |
| participants analyzed | | | | |
| GMTs (95% Confidence | | | | |
| Interval) [units: Titer] | | | | |
| Day 365, TDV-1 | 247.3 | (160.9 to 380.2) | 264.1 | (181.1 to 385.1) |
| Day 365, TDV-2 | 1726.0 | (1392.6 to 2139.3) | 809.5 | (576.6 to 1136.4) |
| Day 365, TDV-3 | 163.2 | (110.0 to 242.3) | 132.6 | (89.9 to 195.5) |
| Day 365, TDV-4 | 75.3 | (53.8 to 105.4) | 77.0 | (56.9 to 104.2) |

TABLE 12
Ratio of geometric mean neutralizing
antibody titers (GMTs) at day 365

| | Comparative | Example 1 |
|---|---|---|
| | unit dose | unit dose |
| TDV-2:TDV-1 | 7 | 3 |
| TDV-2:TDV-3 | 11 | 6 |
| TDV-2:TDV-4 | 23 | 11 |

TABLE 13
Geometric mean neutralizing antibody titers (GMTs) of all four dengue
serotypes assessed by dengue baseline seropositivity status at day 180

| | Comparative unit dose | | Example 1 unit dose | |
|---|---|---|---|---|
| Seropositive | 89 Participants | | 88 Participants | |
| Day 180, TDV-1 (Seropositive) | 2327.2 | (1550.4 to 3493.3) | 1356.2 | (905.5 to 2031.2) |
| Day 180, TDV-2 (Seropositive) | 4412.0 | (3586.6 to 5427.4) | 2952.0 | (2358.2 to 3695.4) |
| Day 180, TDV-3 (Seropositive) | 1248.3 | (879.7 to 1771.3) | 693.6 | (459.6 to 1046.6) |
| Day 180, TDV-4 (Seropositive) | 399.5 | (291.3 to 547.9) | 268.3 | (190.2 to 378.6) |
| Seronegative | 77 Participants | | 75 Participants | |
| Day 180, TDV-1 (Seronegative) | 46.6 | (32.0 to 67.9) | 55.7 | (35.6 to 87.1) |
| Day 180, TDV-2 (Seronegative) | 1394.1 | (983.2 to 1976.6) | 444.3 | (247.2 to 798.5) |
| Day 180, TDV-3 (Seronegative) | 34.5 | (23.4 to 50.7) | 29.0 | (19.4 to 43.3) |
| Day 180, TDV-4 (Seronegative) | 16.4 | (12.3 to 22.0) | 26.8 | (19.0 to 37.7) |

TABLE 14
Ratio of geometric mean neutralizing antibody titers (GMTs) assessed
by dengue baseline seropositivity status at day 180 and 365

| | |
|---|---|
| Comparative | Example 1 |
| unit dose | unit dose |

Seropositive 180 Days

| | | |
|---|---|---|
| TDV-2:TDV-1 | 1.9 | 2.2 |
| TDV-2:TDV-3 | 3.5 | 4.3 |
| TDV-2:TDV-4 | 11.0 | 11.0 |

Seronegative 180 Days

| | | |
|---|---|---|
| TDV-2:TDV-1 | 29.9 | 8.0 |
| TDV-2:TDV-3 | 40.4 | 15.3 |
| TDV-2:TDV-4 | 85.0 | 16.6 |

TABLE 15
Geometric mean neutralizing antibody titers (GMTs) of all four dengue
serotypes assessed by dengue baseline seropositivity status at day 365

| | Comparative unit dose | | Example 1 unit dose | |
|---|---|---|---|---|
| Seropositive | 84 Participants | | 85 Participants | |
| Day 365, TDV-1 (Seropositive) | 1633.3 | (1055.8 to 2526.7) | 1081.5 | (724.0 to 1615.6) |
| Day 365, TDV-2 (Seropositive) | 3316.0 | (2623.8 to 4190.9) | 2177.3 | (1613.5 to 2938.1) |
| Day 365, TDV-3 (Seropositive) | 830.6 | (551.2 to 1251.5) | 600.2 | (402.3 to 895.3) |
| Day 365, TDV-4 (Seropositive) | 346.3 | (249.2 to 481.1) | 212.6 | (152.2 to 296.9) |
| Seronegative | 76 Participants | | 71 Participants | |
| Day 365, TDV-1 (Seronegative) | 30.7 | (20.4 to 46.2) | 48.8 | (32.1 to 74.2) |
| Day 365, TDV-2 (Seronegative) | 838.7 | (621.9 to 1131.1) | 247.6 | (143.9 to 426.1) |
| Day 365, TDV-3 (Seronegative) | 27.0 | (17.8 to 41.1) | 21.7 | (14.3 to 33.1) |
| Day 365, TDV-4 (Seronegative) | 13.9 | (10.3 to 19.0) | 22.9 | (15.8 to 33.1) |

TABLE 16
Ratio of geometric mean neutralizing antibody titers (GMTs)
assessed by dengue baseline seropositivity status at day 365

| | |
|---|---|
| Comparative | Example 1 |
| unit dose | unit dose |

Seropositive 365 Days

| | | |
|---|---|---|
| TDV-2:TDV-1 | 2.0 | 2.0 |
| TDV-2:TDV-3 | 4.0 | 3.6 |
| TDV-2:TDV-4 | 9.6 | 10.2 |

Seronegative 365 Days

| | | |
|---|---|---|
| TDV-2:TDV-1 | 27.3 | 5.1 |
| TDV-2:TDV-3 | 31.1 | 11.4 |
| TDV-2:TDV-4 | 60.3 | 10.8 |

### c) Safety

Overall, rates of solicited local and systemic adverse events (AEs), unsolicited AEs, and serious 5 adverse events (SAEs) were similar across the two unit dose groups. No deaths or AEs leading to discontinuation were recorded in the trial. Three subjects in each unit dose group experienced SAEs, one of these events in the HD-TDV group was considered by the sponsor to be vaccine-related based on temporal association. The SAE was polyarthritis which began six days following receipt of the vaccine. Rates of solicited reactions were similar across unit dose groups, and seropositivity status at baseline. Overall, 47.4% and 53.7% of subjects reported local reactions, and 52.0% and 50.9% reported solicited systemic AEs, in the HD-TDV and TDV groups, respectively. Most reactions were mild or moderate. The most commonly reported local adverse reaction was injection site pain (46.3% in the HD-TDV group, 52.0% in the TDV group) and the most common systemic AE was headache (28.6% in the HD-TDV group, 34.9% in the TDV group).

### d) Conclusion

Both unit doses showed an acceptable safety profile. The results show a more balanced immune response with the new TDV unit dose compared to the early HD-TDV unit dose, particularly in the subjects who were seronegative prior to vaccination: (i) in baseline seronegative subjects, response to DENV-2 was less dominant with TDV and (ii) DENV-4 seropositivity rates and GMTs were also higher with TDV in these subjects.

### Example 4: Cell-Mediated Immunity Stimulated by the Dengue Vaccine

A gamma interferon (IFNγ) enzyme-linked immunosorbent spot (ELISPOT) assay was performed using peripheral blood mononuclear cells (PBMCs) from the subjects taking part in the clinical trial of Example 3 and the commercial ELISpot assay kit available from Mabtech according to the manufacturer's instructions. Briefly, cryopreserved PBMCs were thawed and left to rest overnight, then incubated with various peptide pools for 18-22 hours in plates coated with anti-IFNγ antibodies. PBMCs were then removed and spots were developed and then counted. Results were reported as mean spot forming cells (SFC) per 106 PBMCs. Peptide pools matched selected DENV-derived proteins, covering the entire DENV-2 proteome with NS1, NS3, and NS5 proteins from New Guinea C (NGC) and Thailand/16681/84 strains; and C, prM+E, NS2 and NS4 proteins from Thailand/16681/84 only plus TDV-1, TDV-3 and TDV-4 prM+E inserts from DENV-1, -3 and -4 strains Thailand/16007/1964, Philippines/16562/1964 and Indonesia/1036/1976, respectively.

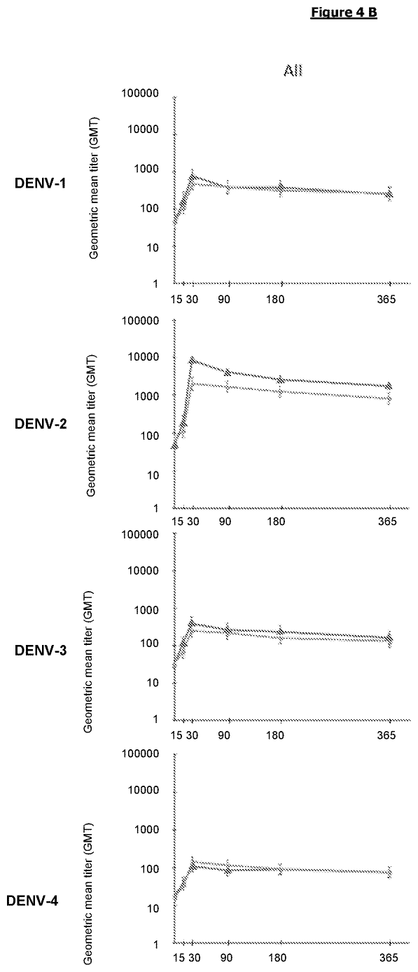

Response rates to DENV-2 proteome at 6 and 12 months post-single dose of TDV were >90%, and were comparable between subjects seronegative and seropositive at baseline (FIG. 5).

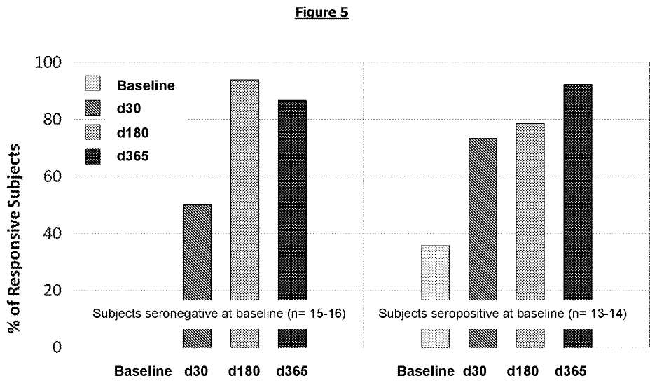

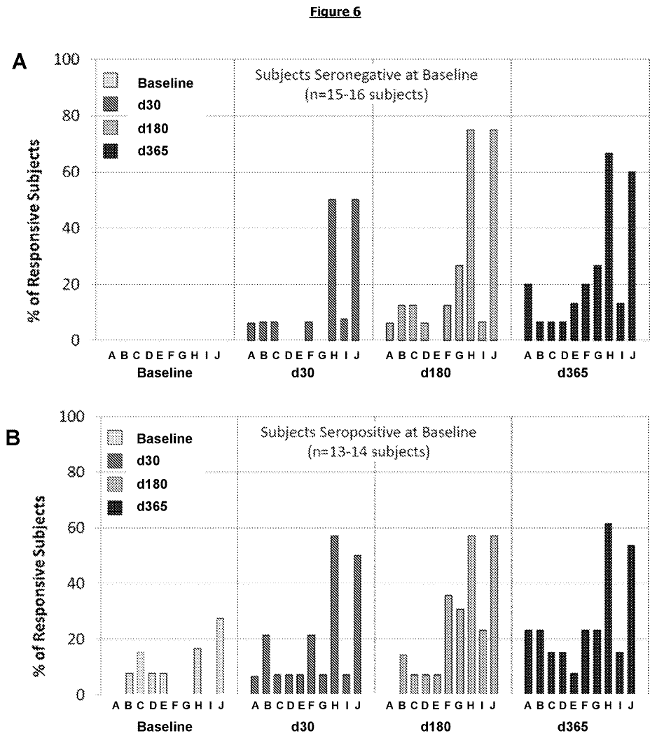

The response was primarily directed to the NS proteins, particularly in subjects seronegative at baseline (FIG. 6).

The NS3 and NS5 proteins were the most recognized antigens (by 50-75% of subjects). Immunodominance of NS3 and NS5 was independent of baseline serostatus. Durability of the response was maintained equally between NS3 and NS5 throughout the 12-month post-single vaccination follow-up.

### Example 5: Antibody Responses to Non-Structural Proteins

The non-structural protein NS-1 from all four dengue serotypes can induce endothelial hyperpermeability of human pulmonary microvascular endothelial cells (HPMEC) as measured by transendothelial electrical resistance (TEER) (Puerta-Guardo et al. (2016) PloS Pathog. 12 (7): e1005738). It also interacts with endothelial cells to induce degradation of the glycocalyx via activation of sialidases and the cathepsin L/heparanase pathway (Glasner et al. (2017) PloS Pathog. 13 (11): e1006673). In view of these effects, it was investigated whether the comparative unit dose induces antibodies against NS1 and inhibits NS1-mediated physiological effects.

### a) Anti-NS1 Antibodies

Serum samples were collected at day 0 before vaccination and day 120 after administration of the first unit dose. Serum was collected from 6 dengue seronegative and 6 dengue seropositive subjects at both day 0 and day 120, and Ab concentrations were determined by ELISA.

The anti-NS1 antibody concentration in seronegative and seropositive subjects at day 0 and day 120 is shown in Tables 17 and 18:

TABLE 17
Anti-NS1 antibody concentration in seronegative subjects at day 0 and day 120
Dengue seronegative subjects Anti-NS1 antibody concentration (RU/ml)

| | DENV1 | | DENV2 | | DENV3 | | DENV4 | |
|---|---|---|---|---|---|---|---|---|
| Subj. | d0 | d120 | d0 | d120 | d0 | d120 | d0 | d120 |
| 1023014 | 13.49 | 602.56 | 16.22 | 2570.40 | 10.00 | 489.78 | 28.18 | 302.00 |
| 1025011 | 66.07 | 173.78 | 35.48 | 794.33 | 67.61 | 117.49 | 42.66 | 85.11 |
| 1025013 | 5.62 | 380.19 | 24.55 | 2454.71 | 16.98 | 316.23 | 10.00 | 186.21 |
| 1035002 | 34.67 | 177.83 | 31.62 | 977.24 | 17.78 | 114.82 | 19.05 | 44.67 |
| 1035005 | 50.12 | 467.74 | 20.42 | 1659.59 | 104.71 | 309.03 | 66.07 | 288.40 |
| 1035001 | 40.74 | 186.21 | 52.48 | 489.78 | 44.67 | 169.82 | 51.29 | 177.83 |

TABLE 18
Anti-NS1 antibody concentration in seronegative subjects at day 0 and day 120
Dengue seropositive subjects Anti-NS1 antibody concentration (RU/ml)

| | DENV1 | | DENV2 | | DENV3 | | DENV4 | |
|---|---|---|---|---|---|---|---|---|
| Subj. | d0 | d120 | d0 | d120 | d0 | d120 | d0 | d120 |
| 1052010 | 691.83 | 11481.54 | 309.03 | 12022.64 | 436.52 | 7585.78 | 245.47 | 4677.35 |
| 1052014 | 758.58 | 1445.44 | 407.38 | 891.25 | 758.58 | 1122.02 | 724.44 | 707.95 |
| 1052015 | 3890.45 | 3467.37 | 2570.40 | 2344.23 | 3235.94 | 2818.38 | 660.69 | 707.95 |
| 1071007 | 478.63 | 851.14 | 239.88 | 478.63 | 660.69 | 1202.26 | 870.96 | 1258.93 |
| 1071012 | 691.83 | 776.25 | 724.44 | 676.08 | 776.25 | 812.83 | 346.74 | 446.68 |
| 1082009 | 5888.44 | 5370.32 | 7413.10 | 6309.57 | 5248.07 | 4897.79 | 891.25 | 794.33 |

These data show that the vaccine induces antibodies against NS1 from all dengue serotypes in both seropositive and seronegative subjects.

### b) Transendothelial Electrical Resistance (TEER)

The effect of recombinant NS1 proteins from dengue serotypes 1, 2, 3 and 4 and sera from vaccinated seronegative and seropositive subjects on endothelial permeability was evaluated by measuring TEER of HPMEC grown on a 24-well Transwell polycarbonate membrane system (Transwell® permeable support, 0.4 μM, 6.5 mm insert; Corning Inc.) as previously described (Beatty et al. (2015) Sci. Transl. Med. 7 (304): 304ra141; Puerta-Guardo et al. (2016) PloS Pathog. 12 (7): e1005738). Briefly, TEER was measured in Ohms (Ω) at sequential 2-hour time-points following the addition of test proteins using an Epithelial Volt Ohm Meter (EVOM) with “chopstick” electrodes (World Precision Instruments). Untreated endothelial cells grown on Transwell inserts were used as negative untreated controls, and inserts with medium alone were used for blank resistance measurements. Relative TEER represents a ratio of resistance values (Ω) as follows: (Ω experimental condition−Ω medium alone)/(Ω non-treated endothelial cells−Ω medium alone). After 24 hours of treatment, 50% of upper and lower chamber media was replaced by fresh endothelial cell medium. For experiments using sera, 30 μl of culture supernatant was removed from the apical chamber and replaced with 30 μl of serum samples immediately before the addition of 5 μg/ml DENV-2 NS1.

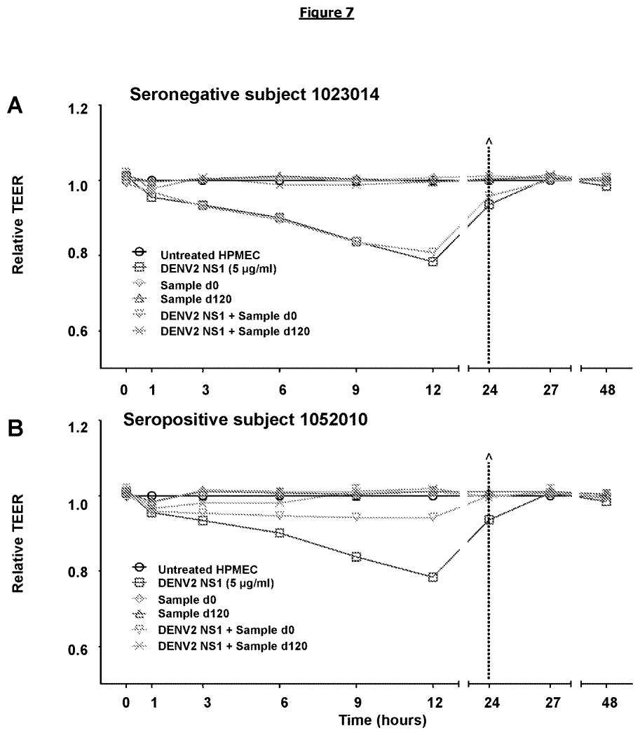

Day 0 serum samples from seronegative subjects did not protect against NS1-mediated barrier dysfunction, but day 120 samples from all seronegative subjects blocked decreases in TEER values induced by NS1 (see FIG. 7A). Day 0 samples from seropositive subjects produced varying levels of protection, and all day 120 samples from seropositive subjects completely abrogated NS1-induced hyperpermeability (see FIG. 7B).

### c) Degradation of Glycocalyx-Like Layer (EGL)

Microscopy was performed as previously described (Puerta-Guardo et al. (2016) PloS Pathog. 12 (7): e1005738). For imaging experiments, HPMEC were grown on coverslips coated with 0.2% gelatin (Sigma) and imaged on a Zeiss LSM 710 Axio Observer inverted fluorescence microscope equipped with a 34-channel spectral detector. Images acquired using the Zen 2010 software (Zeiss) were processed and analyzed with ImageJ software. All RGB images were converted to grayscale, then mean grayscale values and integrated density from selected areas were taken, along with adjacent background readings, and plotted as mean fluorescence intensity (MFI). To assess the effect of sera from vaccinated subjects on DENV2 NS1-induced EGL disruption, the distribution of sialic acid and heparan sulfate was examined on confluent HPMEC monolayers treated with DENV2 NS1 (5 μg/ml)+negative control serum (30 μl), NS1+positive control serum (30 μl), or NS1+serum from vaccinated subjects (30 μl) and fixed with 4% paraformaldehyde (PFA) at 6 hours post-treatment. Primary antibodies (Wheat germ agglutinin (WGA) lectin conjugated to Alexa Fluor 647 (WGA-A647, Molecular Probes) to stain N-acetyl neuraminic acid (sialic acid); Ab Heparan Sulfate, purified (clone F58-10E4, Amsbio) were incubated overnight at 4° C., and detection was performed using secondary species-specific anti-IgG or anti-IgM antibodies conjugated to Alexa fluorophores (488 and 647).

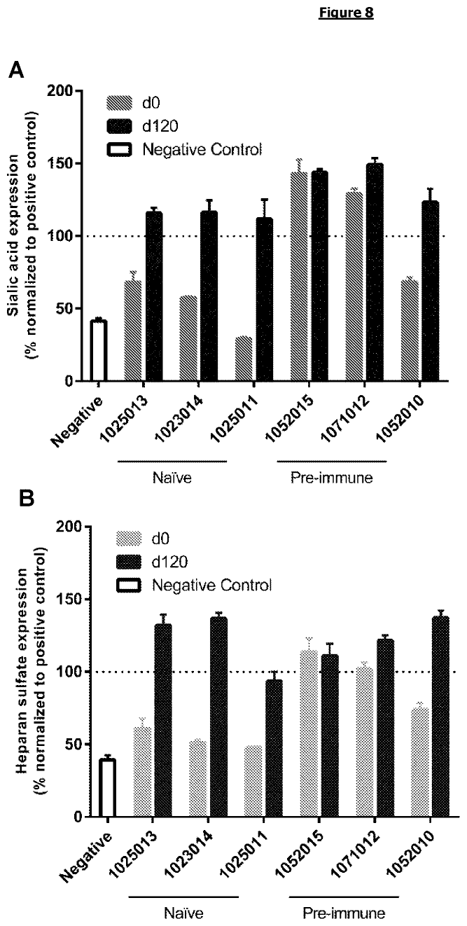

Day 0 sera from seronegative subjects had no substantial protective effect, while day 120 sera from seronegative subjects completely blocked degradation of both sialic acid and heparan sulfate. Similarly, day 0 samples from seropositive subjects exhibited varying levels of protection, and sera from seropositive subjects at day 120 were completely protective (see FIG. 8). Positive control serum was used as a baseline for protection, and negative control serum represented maximum NS1-mediated disruption. These results show that the anti-NS1 antibody response stimulated by the dengue vaccine can protect against NS1-induced hyperpermeability by preventing the degradation of key EGL components.

Taken together, these results suggest that the dengue vaccine stimulates robust and protective anti-DENV2 NS1 Ab responses following vaccination.

### Example 6: Phase III Clinical Trial in Children

A Phase III, double-blind, randomized, and placebo-controlled trial in 20100 subjects aged 4 to 16 years living in Thailand, Sri Lanka, Philippines, Panama, Nicaragua, Dominican Republic, Colombia or Brazil was performed evaluating the efficacy, safety and immunogenicity of a tetravalent dengue vaccine referred to hereinafter as TDV. The trial includes 3 parts. Part 1 evaluates vaccine efficacy (VE) and lasts until both of the following 2 criteria are fulfilled: (i) 120 cases of dengue fever are confirmed and (ii) minimum duration of subject follow-up of 12 months post-second vaccination. Part 2 is for an additional 6 months to evaluate VE and for secondary efficacy analyses. Part 3 will evaluate long-term safety by following participants for side effects and will last an additional 3 years.
Part 1: Active surveillance for the primary assessment of efficacy in all subjects. During this time subjects were contacted at least weekly to ensure identification of febrile illness that could potentially be due to dengue. This part commenced on the day of vaccination and finished once both of the following 2 criteria were fulfilled: (i) 120 cases of dengue fever are confirmed and (ii) minimum duration of subject follow-up of 12 months post-second vaccination. The end of Part 1 was defined for each subject so that the duration of follow up after the second vaccination was approximately the same for all subjects. Virologically-confirmed cases in Part 1 count towards the primary efficacy objective if occurring at least 30 days post-second vaccination.
Part 2: Active surveillance for an additional 6 months for each subject following the completion of Part 1. During this time subjects were contacted at least weekly to ensure identification of febrile illness that could potentially be due to dengue. Virologically-confirmed cases in Parts 1 and 2 contribute towards the secondary efficacy objectives.
Part 3: Modified active surveillance for the assessment of safety in all subjects following the completion of Part 2 and lasting 3 years for each subject. The modified surveillance during Part 3 will maintain at least weekly contacts through Part 3 of the trial, but the intensity of investigation will be modified based on the need for hospitalization. Surveillance will identify febrile illness of any severity that could potentially be due to dengue.

Criteria for Inclusion include:
The subject was aged 4 to 16 years inclusive, at the time of randomization.
Individuals who were in good health at the time of entry into the trial as determined by medical history, physical examination (including vital signs) and clinical judgment of the Investigator.
The subject and/or the subject's parent/guardian signed and dated an assent/written informed consent form where applicable, and any required privacy authorization prior to the initiation of any trial procedures, after the nature of the trial has been explained according to local regulatory requirements.
Individuals who can comply with trial procedures and are available for the duration of follow-up.

Exclusion criteria include:
1. Febrile illness (temperature≥38° C.) or moderate or severe acute illness or infection at the time of randomization.
2. History or any illness that, in the opinion of the Investigator, might interfere with the results of the trial or pose an additional risk to the subject due to participation in the trial, including but not limited to:
   a. Known hypersensitivity or allergy to any of the vaccine components.
   b. Female subjects (post-menarche) who are pregnant or breastfeeding.
   c. Individuals with any serious chronic or progressive disease according to judgment of the Investigator (eg, neoplasm, insulin-dependent diabetes, cardiac, renal or hepatic disease, neurologic or seizure disorder or Guillain-Barré syndrome).
   d. Known or suspected impairment/alteration of immune function, including:
      i. Chronic use of oral steroids (equivalent to 20 mg/day prednisone≥12 weeks/≥2 mg/kg body weight/day prednisone≥2 weeks) within 60 days prior to Day 1 (Month 0) (use of inhaled, intranasal, or topical corticosteroids is allowed).
      ii. Receipt of parenteral steroids (equivalent to 20 mg/day prednisone≥12 weeks/≥2 mg/kg body weight/day prednisone≥2 weeks) within 60 days prior to Day 1 (Month 0).
      iii. Administration of immunoglobulins and/or any blood products within the 3 months prior to Day 1 (Month 0) or planned administration during the trial.
      iv. Receipt of immunostimulants within 60 days prior to Day 1 (Month 0).
      v. Immunosuppressive therapy such as anti-cancer chemotherapy or radiation therapy within 6 months prior to Day 1 (Month 0).
      vi. Human Immunodeficiency Virus (HIV) infection or HIV-related disease.
      vii. Genetic immunodeficiency.
3. Receipt of any other vaccine within 14 days (for inactivated vaccines) or 28 days (for live vaccines) prior to Day 1 (Month 0) or planning to receive any vaccine within 28 days after Day 1 (Month 0).
4. Participation in any clinical trial with another investigational product 30 days prior to Day 1 (Month 0) or intent to participate in another clinical trial at any time during the conduct of this trial.
5. Previous participation in any clinical trial of a dengue candidate vaccine, or previous receipt of a dengue vaccine.
6. First degree relatives of individuals involved in trial conduct.
7. Females of childbearing potential who are sexually active, and who have not used any of the acceptable contraceptive method for at least 2 months prior to Day 1 (Month 0).
8. Females of childbearing potential who are sexually active, and who refuse to use an acceptable contraceptive method up to 6 weeks post-second vaccination.
9. Deprived of freedom by administrative or court order, or in an emergency setting, or hospitalized involuntarily.
10. Current alcohol abuse or drug addiction that may interfere with the subject's ability to comply with trial procedures.
11. Identified as an employee of the Investigator or trial center, with direct involvement in the proposed trial or other trials under the direction of that Investigator or trial center.

Eligible subjects were randomized (2:1) into two treatment groups: groups 1 received one subcutaneous (SC) dose of TDV in the upper arm on Day 1 and on Day 90, and group 2 received one subcutaneous dose of placebo in the upper arm on Day 1 and on Day 90. Randomization was stratified by region (Asia Pacific and Latin America) and age range (children aged 4-5 years, 6-11 years, and 12-16 years) to ensure each age range has the appropriate ratio of TDV to placebo in each region. After randomization dropouts were not replaced. Study Day 1 is defined to be the date of the first dose administration of TDV or placebo. The TDV was prepared as described in Example 1. Each subcutaneous dose of TDV was 0.5 mL and the concentration of the four dengue serotypes in the TDV vaccine in each dose was 3.6 log 10 PFU/dose, 4.0 log 10 PFU/dose, 4.6 log 10 PFU/dose and 5.1 log 10 PFU/dose of TDV-1, TDV-2, TDV-3 and TDV-4, respectively.

Primary Outcome Measures included the vaccine efficacy (VE) of two doses of TDV in preventing virologically-confirmed dengue (VCD) fever induced by any dengue serotype [time frame: 30 days post-second vaccination (Day 120) until the end of Part 1]. VE is defined as 1−(Av/Ac), wherein λv and λc denote the hazard rates for the TDV and placebo groups, respectively. A virologically-confirmed dengue case is defined as febrile illness (defined as temperature≥38° C. on any 2 of 3 consecutive days) or illness clinically suspected to be dengue by the Investigator with a positive serotype-specific reverse transcriptase polymerase chain reaction (RT-PCR). A febrile illness will require an interval of at least 14 days from a previous febrile illness to avoid overlap of acute and convalescent visits from one episode with those from a second episode.

### Secondary Outcome Measures Include:

1) VE of two doses of TDV in preventing virologically-confirmed dengue fever induced by each dengue serotype [time frame: from 30 days post-second vaccination (Day 120) until the end of Part 2].
2) VE of two doses of TDV in preventing virologically-confirmed dengue fever induced by any dengue serotype in participants dengue seronegative at baseline [time frame: from 30 days post-second vaccination (Day 120) until the end of Part 2 (up to 21 months)].
3) VE of two doses of TDV in preventing virologically-confirmed dengue fever induced by any dengue serotype in participants dengue seropositive at baseline [time frame: from 30 days post-second vaccination (Day 120) until the end of Part 2].
4) VE of two doses of TDV in preventing hospitalization due to virologically-confirmed dengue fever induced by any dengue serotype [time frame: from 30 days post-second vaccination (Day 120) until the end of Part 2].
5) VE of two doses of TDV in preventing virologically-confirmed severe dengue fever induced by any dengue serotype [time frame: from 30 days post-second vaccination (Day 120) until the end of Part 2].
6) Percentage of participants with solicited local injection site adverse events (AEs) in the safety subset [time frame: Days 1 through 7 after each vaccination] and severity of solicited local injection AEs. Solicited local AEs at injection site are defined as pain, erythema and swelling that occurred within 7 days after each vaccination.
7) Percentage of participants with solicited systemic adverse events (AEs) in the safety subset [time frame: Days 1 through 14 after each vaccination] and severity of solicited systemic AEs. Solicited systemic AEs in children (<6 years) are defined as fever, irritability/fussiness, drowsiness and loss of appetite that occurred within 14 days after each vaccination. Solicited systemic AEs in children (≥6 years) are defined as fever, headache, asthenia, malaise and myalgia that occurred within 14 days after each vaccination.
8) Percentage of participants with any unsolicited adverse events (AEs) in the safety subset [time frame: Days 1 through 28 after each vaccination]. Unsolicited AEs are any AEs that are not solicited local or systemic AEs, as defined above.
9) Percentage of participants with serious adverse events (SAEs) during Parts 1 and 2 [time frame: from Day 1 until the end of Parts 1 and 2]. A serious adverse event (SAE) is any untoward medical occurrence or effect that at any dose results in death, is life-threatening, requires inpatient hospitalization or prolongation of existing hospitalization, results in persistent or significant disability/incapacity, is a congenital anomaly/birth defect or is medically important due to other reasons than the above mentioned criteria.
10) Percentage of participants with fatal SAEs and SAEs related to study drug during the first and second half of Part 3 [time frame: for 3 years (18 month halves) beginning at the end of Part 2 (approximately 21 months after the first vaccination)].
11) Percentage of participants with a seropositive response for each of the four dengue serotypes in the immunogenicity subset [time frame: Day 1 and months 1, 3, 4, 9, 15 and then annually (up to 3 years)]. Seropositive response is defined as a reciprocal neutralizing titer≥10. The four DENV serotypes are DEN-1, DEN-2, DEN-3 and DEN-4.
12) Percentage of participants with a seropositive response for multiple dengue serotypes in the immunogenicity subset [time frame: Day 1 and months 1, 3, 4, 9, 15 and then annually (up to 3 years)].
13) Geometric Mean Titers (GMTs) of neutralizing antibodies for each of the four dengue serotypes in the immunogenicity subset [time frame: Day 1 and months 1, 3, 4, 9, 15 and then annually (up to 3 years)]. GMTs of neutralizing antibodies will be measured via microneutralization test (MNT) as described in Example 2.

### a) Study Population

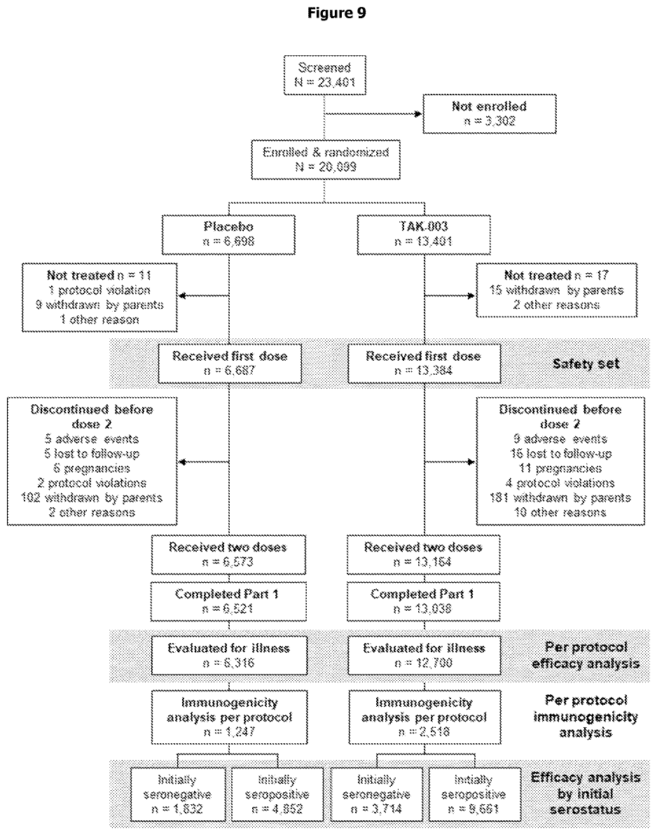

After screening, 20,099 participants were randomized, and 20,071 received at least one injection. In total, 97.4% of placebo participants (n/N: 6,521/6,698) and 97.3% of vaccinees (n/N: 13,038/13,401) completed Part 1 of the study (FIG. 9). Reasons for study withdrawals included AEs, participants lost to follow-up, pregnancy, protocol violations, and withdrawal by participants (or parents/guardians). Baseline characteristics were similar across both treatment groups (Table 19). Mean age of study participants was 9.6 years, with baseline seronegativity of 27.7%, and enrollment was broadly balanced across regions (46.5% in Asia, 53.5% in Latin America). The highest seronegative rate was in Panama (62.2%), followed by Sri Lanka (38.5%), Thailand (34.4%), Brazil (28.8%), Nicaragua (22.3%), Colombia (15.4%), the Philippines (12.4%), and the Dominican Republic (2.8%).

TABLE 19
Baseline characteristics of study population (number, %)

| | | |
|---|---|---|
| TDV | Placebo | Total |

Per Protocol Set

| Number of | 12,704 | | 6,317 | | 19,021 | |
|---|---|---|---|---|---|---|
| Participants | | | | | | |
| Mean Age | 9.6 | (3.35) | 9.6 | (3.34) | 9.6 | (3.35) |
| (Years, SD) | | | | | | |
| Baseline | 3,533 | (27.8) | 1,726 | (27.3) | 5,259 | (27.7) |
| Seronegative^{a} | | | | | | |
| Female | 6,314 | (49.7) | 3,098 | (49.0) | 9,412 | (49.5) |
| Male | 6,390 | (50.3) | 3,219 | (51.0) | 9,609 | (50.5) |
| Asia Pacific | 5,896 | (46.4) | 2,942 | (46.6) | 8,838 | (46.5) |
| Baseline | 1,503 | (25.5) | 773 | (26.3) | 2,276 | (25.8) |
| Seronegative^{a} | | | | | | |
| Latin America | 6,808 | (53.6) | 3,375 | (53.4) | 10,183 | (53.5) |
| Baseline | 2,030 | (29.8) | 953 | (28.2) | 2,983 | (29.3) |
| Seronegative^{a} | | | | | | |

Safety Set^{b}

| Number of | 13,380 | | 6,687 | | 20,071 | |
|---|---|---|---|---|---|---|
| Participants | | | | | | |
| Mean Age | 9.6 | (3.36) | 9.6 | (3.34) | 9.6 | (3.35) |
| (Years, SD) | | | | | | |
| Baseline | 3,714 | (27.8) | 1,832 | (27.4) | 5,547 | (27.6) |
| Seronegative^{a} | | | | | | |
| Female | 6,651 | (49.7) | 3,276 | (49.0) | 9,929 | (49.5) |
| Male | 6,729 | (50.3) | 3,411 | (51.0) | 10,142 | (50.5) |

Safety Set of Subset^{b}

| Number of | 2,663 | | 1,329 | | 3,993 | |
|---|---|---|---|---|---|---|
| Participants | | | | | | |
| Baseline | 740 | (27.8) | 369 | (27.8) | 1,109 | (27.8) |
| Seronegative^{a} | | | | | | |

^{a}Seronegative for all serotypes; seropositive defined as reciprocal neutralizing antibody titer ≥10; SD, standard deviation.
^{b}numbers of participants in TVD plus placebo groups are not equal to total numbers shown because misallocated participants (i.e. those who received both TVD and placebo due to an administrative error) are not included in the TDV and placebo group data.

### b) Febrile Illnesses and VCD

During Part 1, 5,754 and 4,663 episodes of febrile illness were reported in Asian and Latin American sites, respectively. Acute samples were obtained in 99.5% and 96.6% of these cases, with 98.3% and 85.1% of samples taken within five days, in Asia and Latin America, respectively. There were 278 VCD cases (76 hospitalized) in the safety set during the entire Part 1 period, of which 210 (58 hospitalized) were 30 days post-second vaccination in the PPS (Table 20; Table 22) and were included in primary endpoint analysis.

### c) Distribution of VCD Included in Primary Endpoint Analysis

DENV-1 was reported in all countries with VCD and included all the 21 cases in Panama. In Sri Lanka, 54 of 60 VCD were DENV-2, and 87 of 109 VCD in the Philippines were DENV-3. All seven DENV-4 VCD were reported in the Philippines. No VCD were reported in Nicaragua or the Dominican Republic. Of the associated 58 hospitalized VCD, 43 were reported in Sri Lanka. A total of two severe dengue (both DENV-3) and five dengue hemorrhagic fever (DHF; three DENV-2; two DENV-3) cases were reported (Table 21). These seven were also the only such cases in the entire part 1 safety set.

### d) Vaccine Efficacy

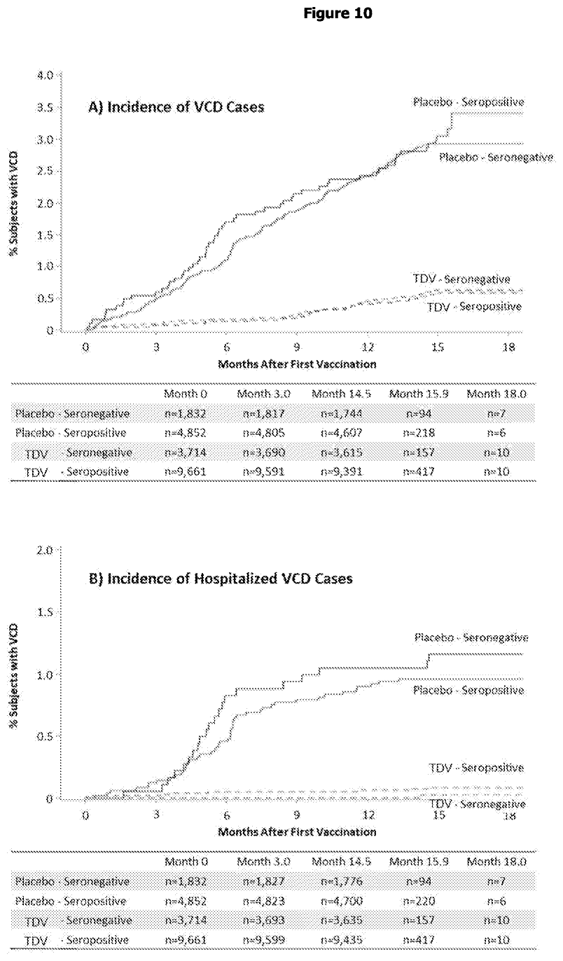

VE against VCD of any serotype was 80.2% (95% CI: 73.3-85.3; P<0.001). A similar efficacy of 81% (95% CI: 64.1-90.0) between the doses and from first dose onwards in the safety set (Table 20) suggests that the vaccine was efficacious after the first dose. Exploratory analysis of the secondary efficacy endpoints showed a trend of differential efficacy by serotype, with the highest efficacy against DENV-2 (97.7%), followed by DENV-1 (73.7%), DENV-4 (63.2% with CI containing zero), and DENV-3 (62.6%; Table 3). Overall, efficacy was similar in baseline seronegatives (74.9%) and seropositives (82.2%; FIG. 10A); however, this varied by serotype. Efficacy against DENV-2 was not impacted by serostatus; efficacy against DENV-1 was slightly higher in baseline seropositives (79.8%; 95% CI: 51.3-91.6) than baseline seronegatives (67.2%; 95% CI: 23.2-86.0). No efficacy was observed against DENV-3 in baseline seronegatives (−38.7%; 95% CI: −335.7-55.8) compared to baseline seropositives (71.3%; 95% CI: 54.2-82.0). Efficacy by serostatus could not be calculated for DENV-4 because no cases were observed in baseline seronegatives. In the primary endpoint timeframe of the PPS, only five VCD requiring hospitalization were reported in the vaccine group compared with 53 cases in the placebo group, with a VE of 95.4% (95% CI: 88.4-98.2; 97.2% for baseline seronegatives and 94.4% for baseline seropositives; Table 21; FIG. 10B), consistent with a VE of 93.3% (95% CI: 86.7-96.7) in the safety set from first dose onwards.

The primary vaccine efficacy (VE) of two doses of TDV in preventing virologically-confirmed dengue (VCD) fever induced by any dengue serotype is shown in Table 20.

TABLE 20
Vaccine efficacy of TDV in preventing virologically confirmed
dengue (VCD) fever against any serotype from 30 days post-second
vaccination until end of part 1 Per Protocol Set (PPS). Safety
set analysis from first dose to end of Part 1 study period

| | Placebo | TDV (PPS) |
|---|---|---|
| | n = 6317 | n = 12,704 |
| number of subject evaluated | 6,316 | 12,700 |
| number of subjects with | 1,712 | 3,195 |
| febrile illness | | |
| number of febrile illness | 2,591 | 4,692 |
| cases | | |
| virologically confirmed | 149 [2.4] | 61 [0.5] |
| dengue fever (n [%]) | | |
| Person-years at risk | 5,670.1 | 11,578.7 |
| incident density | 2.6 | 0.5 |

| | |
|---|---|
| relative risk | 0.20 |
| 95% CI of relative risk | (0.15, 0.27) |
| vaccine efficacy (%) | 80.2 |
| 95% CI of vaccine efficacy | (73.3, 85.3) |
| p-value for vaccine efficacy | <0.001 |

| | Placebo | TDV (Safety Set)* |
|---|---|---|
| number of subject evaluated | 6,687 | 13,380 |
| virologically confirmed | 199 [3.0] | 78 [0.6] |
| dengue fever (n [%]) | | |
| Person-years at risk | 8,072.0 | 16,351.5 |
| incident density | 2.5 | 0.5 |

| | |
|---|---|
| vaccine efficacy (%) | 80.9 |
| 95% CI of vaccine efficacy | (75.2, 85.3) |

Note 1:
Percentage of virologically confirmed dengue (VCD) fever are based on number of subjects evaluated.
Note 2:
Person-years at risks is defined as cumulative time in years until start of VCD fever or until end of Part 1 study period or discontinuation date, whichever comes first. Incident density is defined as the number of cases per 100 person-years at risk. Percentages are based on total number (denominator) of analysis set participants evaluated and may not be equal to the total number of participants in the per protocol analysis set.
*One participant had two instances of VCD during Part 1, only the first VCD was included in efficacy calculation
Note 3:
Vaccine efficacy (VE) and 2-sided 95% CIs are estimated from a Cox proportional hazard model with TDV as a factor, adjusted for age and stratified by region.
Note 4:
Statistical significance will be concluded if the lower bound of the 95% CI for VE is above 25%. Since the hypotheses will be tested in a confirmatory manner at a 2-sided significance level of 5%, the calculated p-value should be compared with 0.025.
Note 5:
Relative risk is calculated as the number of events divided by the number of subjects evaluated in the TDV group, over the number of events divided by the number of subjects evaluated in the placebo group.

For the efficacy evaluation shown in Table 20, a case of VCD was defined as febrile illness (defined as fever≥38° C. on any 2 of 3 consecutive days) with a positive serotype-specific RT-PCR (i.e., positive dengue detection RT-PCR) and occurring at any time starting from 30 days post-second vaccination (Day 120 [Month 4]) through the end of Part 1. The analysis was performed on the Per-Protocol Set (PPS) and Safety Set.

As used herein, the “Per-Protocol Set (PPS)” consist of all subjects in the Full Analysis Set (FAS) consisting of all randomized subjects who received at least one dose of TDV or placebo who had no major protocol violations. Major protocol violations are not receiving both doses of TDV or placebo administration, not receiving both doses in the correct interval, not having the correct administration of TDV or placebo, use of prohibited medications/vaccines by the subject, the subject meets any of the exclusion criteria of 2d, 3, 4 or 5 defined above or product preparation error.

The p-value is obtained by solving the critical value Z in the following equation:

$$Upper bound of 1 - sided ( 1 - p \% ) CI of HR = 0.75 ,$$

$$wherein HR is the hazard ratio and defined as HR = λ V / λ C .$$

$$e \hat{} [ {β}^{\hat{}} + Z * {S}^{\hat{}} E ] = 0.75 ,$$

$$wherein {β}^{\hat{}} defines the treatment and {S}^{\hat{}} E the related standard error .$$

The 1-sided p-value is 1−(area to the left of the critical value Z from a standard normal distribution). Since the hypotheses will be tested in a confirmatory manner 2-sided at a significance level of 5%, the calculated 1-sided p-value should be compared with 0.025.

In summary in Part 1 of this study, a high vaccine efficacy of 80.2% against virologically-confirmed dengue of any serotype in children 4-16 years of age was found. It included an efficacy of 74.9% in baseline seronegatives and a robust 95.4% reduction in hospitalizations. Onset of protection could be seen after the first dose with 81% efficacy between doses. Overall, these results suggest a potential benefit for each vaccine recipient regardless of prior dengue exposure or age. This finding is significant because vaccine development against dengue has been challenging, especially for dengue naïve individuals, and dengue remains one of the WHO's top ten threats to global health in 2019.19 Furthermore, the onset of protection after the first dose has potential utility in the context of outbreak control or travel vaccination, offering a reduction in the risk of dengue after only one dose.

Severe forms of dengue were assessed as follows: Dengue Hemorrhagic Fever (DHF) as defined by the 1997 WHO definition. Severe Dengue through the Dengue Case Adjudication Committee. The Dengue Case Adjudication Committee (DCAC) consisted of four members: a voting chairperson, two voting members, and an independent non-voting statistician. The three DCAC voting members are all physicians and clinical dengue experts. DCAC members are not study investigators and do not have any conflict of interest that would bias their review of the trial data. All non-hospitalized cases were considered non-severe. The DCAC severe dengue case criteria applied in a blinded manner to virologically-confirmed hospitalized dengue cases are as follows: 1) bleeding abnormality, for a case to be considered severe there needs to be a significant intervention required in response to the bleeding episode such as blood transfusion, nasal packing, hormonal therapy, or, bleeding occurred into critical organs such as the brain; 2) plasma leakage, for a case to be considered severe there needs to be evidence of both plasma leakage and functional impairment (plasma leakage includes clinical evidence, radiological evidence, or hematocrit elevated >20% above normal levels or baseline; functional impairment defined as shock or respiratory distress); 3) liver, for a case to be considered severe there needs to be evidence of both hepatitis and functional impairment (hepatitis defined as an aspartate aminotransferase [AST] or alanine aminotransferase [ALT]>10 upper limit of normal range [ULN]; functional impairment defined as prothrombin [PT]>1.5 ULN or hypoalbuminemia); 4) renal, serum creatinine>2.5 times ULN or requiring dialysis; 5) cardiac, abnormalities intrinsic to the heart (i.e. not resulting from intravascular volume depletion) and with evidence of functional impairment (examples of intrinsic abnormality: myocarditis, pericarditis, and myopericarditis; example of functional impairment: new conduction abnormality resulting in irregular heart rhythm [i.e. not transient first-degree heart block]); 6) central nervous system, any abnormality with the exception of a simple febrile convulsion or a brief delirium; 7) shock, all shock cases considered severe. At least 1 functional impairment (of criterion 3, 4, 5, 6), needs to be present but the totality of data were considered by the members in their assessment.

TABLE 21
Distribution of cases contributing to primary endpoint by per protocol set subgroup

| | | | | |
|---|---|---|---|---|
| | TDV | | Placebo | |
| TDV | Incidence | Placebo | Incidence | Vaccine Efficacy |
| Dengue Cases | Density | Dengue Cases | Density | (95% CI) |

VCD cases

| | | | | | | | |
|---|---|---|---|---|---|---|---|
| Baseline | 41/9,165 | (0.4%) | 0.5 | 110/4,587 | (2.4%) | 2.7 | 82.2% (74.5%-87.6%) |
| Seropositive^{a} | | | | | | | |
| Baseline | 20/3,531 | (0.6%) | 0.6 | 39/1,726 | (2.3%) | 2.5 | 74.9% (57.0%-85.4%) |
| Seronegative^{a} | | | | | | | |
| DENV-1 | 16/12,700 | (0.1%) | 0.1 | 30/6,316 | (0.5%) | 0.5 | 73.7% (51.7%-85.7%) |
| DENV-2 | 3/12,700 | (<0.1%) | <0.1 | 64/6,316 | (1.0%) | 1.1 | 97.7% (92.7%-99.3%) |
| DENV-3 | 39/12,700 | (0.3%) | 0.3 | 51/6,316 | (0.8%) | 0.9 | 62.6% (43.3%-75.4%) |
| DENV-4^{d} | 3/12,700 | (<0.1%) | <0.1 | 4/6,316 | (<0.1%) | <0.1 | 63.2% (−64.6%-91.8%) |
| 4-5 Years Old | 13/1,619 | (0.8%) | 0.9 | 23/801 | (2.9%) | 3.2 | 72.8% (46.2%-86.2%) |
| 6-11 Years Old | 34/7,009 | (0.5%) | 0.5 | 85/3,491 | (2.4%) | 2.7 | 80.7% (71.3%-87.0%) |
| 12-16 Years Old | 14/4,072 | (0.3%) | 0.4 | 41/2,024 | (2.0%) | 2.2 | 83.3% (69.3%-90.9%) |
| Asia | 54/5,894 | (0.9%) | 1.0 | 127/2,942 | (4.3%) | 4.9 | 79.5% (71.8%-85.1%) |
| Latin America | 7/6,806 | (0.1%) | 0.1 | 22/3,374 | (0.7%) | 0.7 | 84.3% (63.1%-93.3%) |

Hospitalized VCD cases

| | | | | | | | |
|---|---|---|---|---|---|---|---|
| Baseline | 4/9,165 | (<0.1%) | <0.1 | 35/4,587 | (0.8%) | 0.8 | 94.4% (84.3%-98.0%) |
| Seropositive^{a} | | | | | | | |
| Baseline | 1/3,531 | (<0.1%) | <0.1 | 18/1,726 | (1.0%) | 1.2 | 97.2% (79.1%-99.6%) |
| Seronegative^{a} | | | | | | | |

Cases of DHF^{b}

| | | | | | | | |
|---|---|---|---|---|---|---|---|
| All participants | 1/12,700 | (<0.1%) | <0.1 | 4/6,316 | (<0.1%) | <0.1 | 87.3% (−13.5%-98.6%) |

Severe VCD Cases^{c}

| | | | | | | | |
|---|---|---|---|---|---|---|---|
| All participants | 1/12,700 | (<0.1%) | <0.1 | 1/6,316 | (<0.1%) | <0.1 | 50.8% (−686.9%-96.9%) |

VCD, virologically-confirmed dengue;
DHF, dengue hemorrhagic fever
^{a}Seronegative for all serotypes; baseline seropositive defined as reciprocal neutralizing antibody titer ≥10 to one or more serotypes.
^{b}VCD cases meeting WHO 1997 DHF criteria; incidence density defined as the number of cases per 100 person-years at risk; percentages are based on total number (denominator) of per protocol set participants evaluated.
^{c}two severe VCD were not classified as DHF.
^{d}The number of cases identified was sufficient to provide reasonably precise estimates of vaccine efficacy against all individual serotypes, except DENV-4.

Clinical signs and symptoms of virologically-confirmed dengue cases during Part 1 study period in safety set data are shown in Table 22.

TABLE 22
Clinical signs and symptoms of virologically-confirmed dengue
cases during Part 1 study period (safety set data)

| | TDV | | Placebo | | Relative |
|---|---|---|---|---|---|
| | (N = 13,380) | | (N = 6,687) | | Risk |
| Number of VCD Cases | 78 | | 200 | | — |
| Median Duration of Febrile Illness (days; 95% CI)^{a} | 6.0 | (5.7-7.4) | 6.0 | (5.9-6.8) | — |
| Median Duration of Fever (days; 95% CI) | 4.0 | (3.9-4.6) | 5.0 | (4.5-5.0) | — |
| Number of Hospitalized VCD Cases | 9 | | 67 | | — |
| Median Duration of Hospitalization (days; 95% CI) | 5.0 | (2.8-5.4) | 5.0 | (4.6-5.4) | — |
| Evidence of Bleeding (%, n/N) | 3.8% | (3/78) | 3.5% | (7/200) | 1.10 |
| Plasma Leakage (%, n/N) | 2.6% | (2/78) | 6.5% | (13/200) | 0.39 |
| Plasma Leakage - Pleural Effusion (%, n/N) | 1.3% | (1/78) | 1.5% | (3/200) | — |
| Plasma Leakage - Ascites (%, n/N) | 1.3% | (1/78) | 3.0% | (6/200) | — |
| Plasma Leakage - Radiological Signs (%, n/N) | 40.0% | (2/5) | 19.6% | (10/51) | — |
| Plasma Leakage - Hematocrit Increase ≥20% (%, n/N)^{b} | 3.8% | (2/53) | 9.5% | (13/137) | — |
| Platelet Count ≤100 × 10⁹ (%, n/N)^{c} | 6.4% | (5/78) | 22.0% | (44/200) | 0.29 |
| Platelet Count ≤50 × 10⁹ (%, n/N)^{c} | 3.8% | (3/78) | 11.0% | (22/200) | 0.35 |
| ALT or AST ≥1000 U/L (%, n/N)^{c} | 0% | (0/78) | 0% | (0/200) | — |

VCD, virologically-confirmed dengue;
ALT, alanine aminotransferase;
AST, aspartate aminotransferase
^{a}Duration of febrile illness defined as start date of earliest symptom to end date of latest symptom plus one day (symptoms considered include fever and any general symptoms).
^{b}Hematocrit increase defined as maximum hematocrit between Day 3 and Day 7 inclusive, from onset of fever ≥20% increase over minimum hematocrit before Day 3 or after Day 7 from onset of fever.
^{c}For platelet, ALT, and AST data, assessments within 14 days of onset of febrile illness have been considered.
N refers to number of VCD cases with available data for the specific parameter

### e) Immunogenicity

The highest geometric mean titers (GMTs) were observed against DENV-2 regardless of baseline serostatus (Table 24). A very high tetravalent seropositivity rate (99.5%) in baseline seronegatives one month after the second dose (Tables 23 and 24) was observed.

Seropositivity rate (% of seropositive subjects) for each of the four dengue serotypes is determined at prevaccination on Day 1 (Month 0), post-first vaccination on Day 30 (Month 1), prevaccination on Day 90 (Month 3), post-second vaccination on Day 120 (Month 4), Day 270 (Month 9), Day 450 (Month 15), and then annually. Seropositivity rates (% participants, 95% CI) by dengue serotype per protocol set for immunogenicity data for Day 0, Day 30, Day 90, Day 120, and Day 270 are shown in Table 23.

Seropositivity rates (% participants, 95% CI) by dengue serotype against three or more serotypes (trivalent) and against all four serotypes (tetravalent) per protocol set for immunogenicity data for Day 0, Day 30, Day 90, Day 120, and Day 270 are shown in Table 23. The tetravalent seropositivity rates were high (>91%) in baseline seronegatives six months after second dose.

TABLE 23
Seropositivity rates (% participants, 95% CI) by dengue
serotype (per protocol set for immunogenicity data)

| BASELINE SEROPOSITIVE | | BASELINE SERONEGATIVE | |
|---|---|---|---|
| TDV | Placebo | TDV | Placebo |
| N = 1,816 | N = 902 | N = 702 | N = 345 |

DENV-1

| | | | | | | | |
|---|---|---|---|---|---|---|---|
| 89.1 | (87.6-90.5) | 90.6 | (88.5-92.4) | 0 | (0-0.5) | 0 | (0-1.1) |
| 99.5 | (99.1-99.8) | 88.6 | (86.3-90.7) | 94.1 | (92.0-95.8) | 4.9 | (2.8-7.8) |
| 99.3 | (98.8-99.6) | 90.2 | (88.1-92.1) | 91.6 | (89.3-93.5) | 6.1 | (3.8-9.2) |
| >99.9 | (99.7-100) | 90.3 | (88.1-92.3) | 99.5 | (98.6-99.9) | 8.3 | (5.5-11.9) |
| 99.6 | (99.1-99.8) | 89.8 | (87.5-91.8) | 95.1 | (93.0-96.6) | 9.0 | (6.0-12.8) |

DENV-2

| | | | | | | | |
|---|---|---|---|---|---|---|---|
| 96.5 | (95.6-97.3) | 97.2 | (95.9-98.2) | 0 | (0-0.5) | 0 | (0-1.1) |
| 99.9 | (99.6-100) | 93.3 | (91.4-94.9) | 98.6 | (97.4-99.4) | 10.7 | (7.5-14.5) |
| >99.9 | (99.7-100) | 94.0 | (92.2-95.5) | 99.0 | (98.0-99.6) | 12.2 | (8.9-16.1) |
| 99.9 | (99.6-100) | 93.6 | (91.7-95.2) | 100 | (99.4-100) | 14.7 | (11.0-19.1) |
| 100 | (99.8-100) | 94.6 | (92.8-96.1) | 100 | (99.4-100) | 18.3 | (14.1-23.2) |

DENV-3

| | | | | | | | |
|---|---|---|---|---|---|---|---|
| 88.1 | (86.5-89.6) | 88.0 | (85.7-90.1) | 0 | (0-0.5) | 0 | (0-1.1) |
| 99.8 | (99.4-99.9) | 87.6 | (85.1-89.7) | 96.1 | (94.3-97.4) | 4.0 | (2.1-6.7) |
| 99.5 | (99.1-99.8) | 87.3 | (84.9-89.4) | 94.4 | (92.5-96.0) | 2.0 | (0.8-4.1) |
| 99.8 | (99.5-100) | 87.9 | (85.5-90.1) | 100 | (99.4-100) | 5.1 | (2.9-8.2) |
| 99.7 | (99.4-99.9) | 87.1 | (84.6-89.4) | 96.4 | (94.6-97.7) | 7.7 | (4.9-11.3) |

DENV-4

| | | | | | | | |
|---|---|---|---|---|---|---|---|
| 88.1 | (86.5-89.6) | 87.4 | (85.0-89.5) | 0 | (0-0.5) | 0 | (0-1.1) |
| 99.6 | (99.2-99.9) | 86.6 | (84.1-88.8) | 90.5 | (88.0-92.6) | 1.8 | (0.7-3.9) |
| 99.3 | (98.8-99.7) | 86.9 | (84.5-89.0) | 92.0 | (89.8-93.9) | 2.9 | (1.4-5.3) |
| >99.9 | (99.7-100) | 88.3 | (85.9-90.4) | 99.8 | (99.1-100) | 4.8 | (2.7-7.8) |
| 99.7 | (99.3-99.9) | 87.6 | (85.1-89.9) | 97.0 | (95.4-98.2) | 6.3 | (3.9-9.7) |

Three or More Serotypes

| | | | | | | | |
|---|---|---|---|---|---|---|---|
| 87.5 | (85.9-89.0) | 87.3 | (84.9-89.4) | 0 | (0-0.5) | 0 | (0-1.1) |
| 99.8 | (99.5-100) | 87.2 | (84.7-89.4) | 96.5 | (94.8-97.8) | 1.2 | (0.3-3.1) |
| 99.7 | (99.3-99.9) | 87.7 | (85.3-89.7) | 94.9 | (93.0-96.4) | 1.7 | (0.6-3.7) |
| 99.9 | (99.6-100) | 88.4 | (86.0-90.5) | 99.8 | (99.1-100) | 4.2 | (2.2-7.0) |
| 99.7 | (99.4-99.9) | 87.3 | (84.7-89.5) | 97.5 | (96.0-98.6) | 5.7 | (3.3-8.9) |

All Four Serotypes

| | | | | | | | |
|---|---|---|---|---|---|---|---|
| 83.5 | (81.7-85.2) | 83.5 | (80.9-85.8) | 0 | (0-0.5) | 0 | (0-1.1) |
| 99.1 | (98.5-99.5) | 82.9 | (80.2-85.4) | 85.3 | (82.4-87.9) | 0.9 | (0.2-2.6) |
| 98.6 | (97.9-99.1) | 83.6 | (81.0-86.0) | 84.3 | (81.4-86.9) | 1.4 | (0.5-3.3) |
| 99.8 | (99.5-100) | 85.2 | (82.6-87.6) | 99.5 | (98.6-99.9) | 3.5 | (1.8-6.2) |
| 99.2 | (98.7-99.6) | 84.6 | (81.9-87.0) | 91.3 | (88.7-93.4) | 5.3 | (3.1-8.5) |

Seropositivity rates (% participants, 95% CI) by dengue serotype (per protocol set for immunogenicity data; seropositive defined as a reciprocal neutralizing antibody titer ≥10; baseline seronegative defined as seronegative to all serotype; baseline seropositive defined as seropositive to one or more serotypes; N refers to number of participants in the analysis set; number of participants evaluated at each timepoint may vary)

Seropositivity rates (% participants, 95% CI) by dengue serotype (per protocol set for immunogenicity data; seropositive defined as a reciprocal neutralizing antibody titer≥10; baseline seronegative defined as seronegative to all serotype; baseline seropositive defined as seropositive to one or more serotypes; N refers to number of participants in the analysis set; number of participants evaluated at each timepoint may vary)

Geometric mean titers (GMTs) of neutralizing antibodies (microneutralization test [MNT]) for each dengue serotype are determined at pre-vaccination on Day 1 (Month 0), post-first vaccination on Day 30 (Month 1), pre-vaccination on Day 90 (Month 3), post-second vaccination on Day 120 (Month 4), Day 270 (Month 9), Day 450 (Month 15), and then annually. Geometric mean titers (95% CI) by dengue serotype per protocol set for immunogenicity data for Day 0, Day 30, Day 90, Day 120, and Day 270 are shown in Table 24.

TABLE 24
Geometric mean titers (95% CI) by dengue serotype
(per protocol set for immunogenicity data)

| BASELINE SEROPOSITIVE | | BASELINE SERONEGATIVE | |
|---|---|---|---|
| TDV | Placebo | TDV | Placebo |
| N = 1,816 | N-902 | N = 702 | N = 345 |

DENV-1

| | | | | | | |
|---|---|---|---|---|---|---|
| Day 1 | 410 | (365-461) | 445 (377-524) | 5.0 | (5.0-5.0) | 5.0 (5.0-5.0) |
| Day 30 | 2,404 | (2,204-2,622) | 430 (361-512) | 118 | (106-131) | 5.8 (5.3-6.3) |
| Day 90 | 1,945 | (1,791-2,112) | 410 (349-481) | 91 | (82-102) | 5.9 (5.4-6.3) |
| Day 120 | 2,115 | (1,957-2,286) | 451 (381-534) | 184 | (169-201) | 6.3 (5.7-7.0) |
| Day 270 | 1,447 | (1,329-1,574) | 415 (350-492) | 87 | (79-97) | 6.3 (5.7-6.9) |

DENV-2

| | | | | | | |
|---|---|---|---|---|---|---|
| Day 1 | 745 | (674-825) | 802 (697-924) | 5.0 | (5.0-5.0) | 5.0 (5.0-5.0) |
| Day 30 | 6,697 | (6,301-7,117) | 744 (635-870) | 6,277 | (5,648-6,977) | 6.6 (6.0-7.3) |
| Day 90 | 4,826 | (4,571-5,096) | 729 (629-845) | 1,682 | (1,544-1,834) | 7.0 (6.3-7.9) |
| Day 120 | 4,897 | (4,646-5,163) | 766 (655-896) | 1,730 | (1,614-1,855) | 7.7 (6.7-8.8) |
| Day 270 | 3,692 | (3,496-3,898) | 776 (665-906) | 929 | (856-1,010) | 8.7 (7.4-10.2) |

DENV-3

| | | | | | | |
|---|---|---|---|---|---|---|
| Day 1 | 357 | (321-398) | 356 (305-415) | 5.0 | (5.0-5.0) | 5.0 (5.0-5.0) |
| Day 30 | 2,255 | (2,094-2,428) | 349 (298-409) | 194 | (173-218) | 5.5 (5.2-5.9) |
| Day 90 | 1,563 | (1,453-1,682) | 321 (277-374) | 94 | (85-104) | 5.5 (5.1-5.9) |
| Day 120 | 1,761 | (1,646-1,885) | 353 (301-414) | 228 | (212-246) | 6.0 (5.4-6.6) |
| Day 270 | 1,089 | (1,009-1,175) | 307 (261-360) | 72 | (66-78) | 6.3 (5.7-7.0) |

DENV-4

| | | | | | | |
|---|---|---|---|---|---|---|
| Day 1 | 218 | (198-241) | 234 (203-270) | 5.0 | (5.0-5.0) | 5.0 (5.0-5.0) |
| Day 30 | 1,303 | (1,221-1,391) | 222 (191-258) | 111 | (98-125) | 5.4 (5.0-5.7) |
| Day 90 | 1,002 | (940-1,069) | 215 (187-248) | 63 | (57-70) | 5.5 (5.1-5.9) |
| Day 120 | 1,129 | (1,066-1,196) | 241 (208-280) | 144 | (134-155) | 5.8 (5.3-6.4) |
| Day 270 | 778 | (730-830) | 229 (197-266) | 64 | (59-70) | 6.2 (5.6-6.9) |

Vaccine viremia is assessed by three PCRs: dengue detection RT-PCR, vaccine screening PCR and TDV sequencing in subjects with febrile illness within 30 days after each vaccination.

### f) Safety

Rates of serious adverse events (SAEs) were similar in the vaccine and placebo groups (3.1% and 3.8% of participants, respectively; Table 25). One vaccinee and four placebo recipients experienced SAEs considered to be related to receiving blinded investigational product by the investigator (two experienced hypersensitivity, two were diagnosed with dengue, and one with DHF). There were five deaths during Part 1, and all were considered unrelated to the investigational product or study procedures. Total rates of unsolicited AEs were similar between the vaccine and placebo groups. The most commonly (≥1% of vaccine-recipients) reported unsolicited AEs within four weeks of any dose by preferred term were pyrexia (vaccine group 1.5%; placebo 1.4%), nasopharyngitis (vaccine 2.7%; placebo 3.0%), upper respiratory tract infection (vaccine 2.6%; placebo 2.9%), and viral infection (vaccine 1.1%; placebo 0.9%). Solicited local reactions were reported more frequently in the vaccine group.

TABLE 25
Overview of safety data. Subjects with at least one adverse event after any vaccine
dose. Data presented as number of events (percentage of subjects; number [n]
of subjects/total [N] subjects) unless otherwise stated (safety set data)

| | TDV | | Placebo | |
|---|---|---|---|---|
| Safety Set | N = 13,380 | | N = 6,687 | |
| SAEs | 3.1% | (409/13,380) | 3.8% | (255/6,687) |
| Non-IP-Related^{a} SAEs | 3.0% | (408/13,380) | 3.8% | (251/6,687) |
| IP-Related^{a} SAEs | <0.1% | (1/13,380) | <0.1% | (4/6,687) |
| SAEs Leading to IP Withdrawal and/or | 0.1% | (18/13,380) | 0.1% | (8/6,687) |
| Trial Discontinuation | | | | |
| Deaths | <0.1% | (4/13,380) | <0.1% | (1/6,687) |
| IP-Related Deaths | 0% | (0/13,380) | 0% | (0/6,687) |
| Safety Subset | N = 2,663 | | N = 1,329 | |
| Unsolicited AEs Occurring Within 4 | 18.4% | (490/2,663) | 18.8% | (250/1,329) |
| Weeks of Any Dose | | | | |
| IP-Related^{a} Unsolicited AEs Occurring | 1.0% | (27/2,663) | 1.6% | (21/1,329) |
| Within 4 Weeks of Any Dose | | | | |
| Solicited Systemic AEs Occurring | 42.0% | (1,107/2,635) | 38.0% | (501/1,317) |
| Within 2 Weeks of Any Dose^{b} | | | | |
| IP-Related^{a} Solicited Systemic AEs | 31.2% | (821/2,635) | 28.2% | (371/1,317) |
| Occurring Within 2 Weeks of Any Dose | | | | |
| Solicited Local Reactions Occurring | 36.7% | (967/2,633) | 25.7% | (338/1,317) |
| Within 1 Week of Any Dose^{c} | | | | |

AE, adverse event;
SAE, serious adverse event;
IP, investigational product/TDV
^{a}IP-related, defined as related to the investigational product as assessed by investigator
bonly participants with diary data available were evaluated
^{c}all injection site (solicited local) reactions considered to be IP-related

### LIST OF ITEMS OF THE INVENTION

1. A unit dose of a dengue vaccine composition comprising:
a tetravalent dengue virus composition including four live, attenuated dengue virus strains wherein the unit dose is lyophilized and upon reconstitution with 0.5 mL of a pharmaceutically acceptable diluent comprises:
   (i) a chimeric dengue serotype 2/1 strain in a concentration of at least 3.3 log 10 pfu/0.5 mL,
   (ii) a dengue serotype 2 strain in a concentration of at least 2.7 log 10 pfu/0.5 mL,
   (iii) a chimeric dengue serotype 2/3 strain in a concentration of at least 4.0 log 10 pfu/0.5 mL, and
   (iv) a chimeric dengue serotype 2/4 strain in a concentration of at least 4.5 log 10 pfu/0.5 mL.
2. The unit dose of item 1, wherein upon reconstitution with 0.5 ml of a pharmaceutically acceptable diluent
   (i) has a concentration of 3.3 log 10 pfu/0.5 mL to 5.0 log 10 pfu/0.5 mL,
   (ii) has a concentration of 2.7 log 10 pfu/0.5 mL to 4.9 log 10 pfu/0.5 mL,
   (iii) has a concentration of 4.0 log 10 pfu/0.5 mL to 5.7 log 10 pfu/0.5 mL, and
   (iv) has a concentration of 4.5 log 10 pfu/0.5 mL to 6.2 log 10 pfu/0.5 mL.
3. The unit dose of item 1, wherein upon reconstitution with 0.5 mL of a pharmaceutically acceptable diluent:
   (i) has a concentration of 3.3 log 10 pfu/0.5 mL to 5.0 log 10 pfu/0.5 mL,
   (ii) has a concentration of 2.7 log 10 pfu/0.5 mL to 4.9 log 10 pfu/0.5 mL,
   (iii) has a concentration of 4.0 log 10 pfu/0.5 mL to 5.7 log 10 pfu/0.5 mL, and
   (iv) has a concentration of 4.5 log 10 pfu/0.5 mL to 5.5 log 10 pfu/0.5 mL.
4. The unit dose of any one of items 1 to 3, wherein upon reconstitution with 0.5 ml of a pharmaceutically acceptable diluent
   (i) has a concentration of 3.3 log 10 pfu/0.5 mL to 3.6 log 10 pfu/0.5 mL,
   (ii) has a concentration of 2.7 log 10 pfu/0.5 mL to 4.0 log 10 pfu/0.5 mL,
   (iii) has a concentration of 4.0 log 10 pfu/0.5 mL to 4.6 log 10 pfu/0.5 mL, and
   (iv) has a concentration of 4.5 log 10 pfu/0.5 mL to 5.1 log 10 pfu/0.5 mL.
5. The unit dose of any one of items 1 to 4, wherein upon reconstitution with a pharmaceutically acceptable diluent (i), (ii), (iii), and (iv) provide a total concentration of pfu/0.5 mL and based on said total concentration the concentration of (ii) in pfu/0.5 ml is less than 10% or less than 8%, and the concentration of (iv) in pfu/0.5 mL is at least 50%.
6. The unit dose of item 5, wherein upon reconstitution with a pharmaceutically acceptable diluent (i), (ii), (iii), and (iv) provide a total concentration of pfu/0.5 mL and based on said total concentration the concentration of (i) in pfu/0.5 mL is at least 1%, and the concentration of (iii) in pfu/0.5 mL is at least 7% or at least 8%.
7. The unit dose of any one of items 1 to 6, wherein upon reconstitution with a pharmaceutically acceptable diluent (i), (ii), (iii), and (iv) provide a total concentration of pfu/0.5 mL and based on said total concentration the concentration of (i) in pfu/0.5 mL is 1% to 7% of the total concentration, (ii) in pfu/0.5 mL is less than 8% of the total concentration, such as in the range of 1% to 8% of the total concentration, (iiii) in pfu/0.5 mL is at least 10% of the total concentration, and (iv) in pfu/0.5 mL is at least 65% of the total concentration, such as in the range of 65% to 80%.
8. The unit dose of any one of items 1 to 7, wherein the arithmetic sum of all four serotypes is in the range of 4.6 log 10 pfu/0.5 mL to 6.7 log 10 pfu/0.5 mL, preferably in the range of 4.6 log 10 pfu/0.5 mL to 5.5 log 10 pfu/0.5 mL.
9. The unit dose of any one of items 1 to 8, wherein reconstitution with a pharmaceutically acceptable diluent is made with 0.5 ml of the pharmaceutically acceptable diluent.
10. The unit dose of any one of items 1 to 9, wherein the lyophilized unit dose is prepared from a solution further comprising a non-reducing sugar, a surfactant, a protein and an inorganic salt.
11. The unit dose of item 10, wherein the non-reducing sugar is trehalose, the surfactant is poloxamer 407, the protein is human serum albumin and the inorganic salt is sodium chloride.
12. The unit dose of item 10 or 11, wherein the solution comprises:
   from about 10% (w/v) to about 20% (w/v) α,α-trehalose dihydrate or an equimolar amount of other forms of α,α-trehalose,
   from about 0.5% (w/v) to about 1.5% (w/v) poloxamer 407,
   from about 0.05% (w/v) to about 2% (w/v) human serum albumin, and
   from about 70 mM to about 140 mM sodium chloride.
13. The unit dose of any one of items 1 to 12, wherein the lyophilized unit dose is prepared from a solution comprising:
   about 15% (w/v) α,α-trehalose dihydrate,
   about 1% (w/v) poloxamer 407,
   about 0.1% (w/v) human serum albumin, and
   and about 100 mM sodium chloride.
14. The unit dose of any one of items 1 to 13, wherein each one of the four live attenuated dengue virus strains has attenuating mutations in the 5′-noncoding region (NCR) at nucleotide 57 from cytosine to thymine, in the NS1 gene at nucleotide 2579 from guanine to adenine resulting in an amino acid change at position 828 from glycine to asparagine, and in the NS3 gene at nucleotide 5270 from adenine to thymine resulting in an amino acid change at position 1725 from glutamine to valine, preferably further comprising one or more of the mutations selected from the list comprising:
   a) a mutation in the NS2A gene at nucleotide 4018 from cytosine to thymine resulting in an amino acid at position 1308 from leucine to phenylalanine,
   b) a silent mutation in the NS3 gene at nucleotide 5547 from thymine to cytosine, and
   c) a mutation in the NS4A gene at nucleotide 6599 from guanine to cytosine resulting in an amino acid change at position 2168 from glycine to alanine.
15. The unit dose of item 14, wherein (i) further comprises one or more of the mutations selected from the list comprising:
   a mutation in the NS2A gene at nucleotide 3823 from adenine to cytosine resulting in an amino acid change at position 1243 from isoleucine to leucine,
   a mutation in the NS2B gene at nucleotide 4407 from adenine to thymine resulting in an amino acid change at position 1437 from glutamic acid to aspartic acid, and
   a silent mutation in the NS4B gene at nucleotide 7311 from adenine to guanine.
16. The unit dose of item 14 or 15, wherein (ii) further comprises one or more of the mutations selected from the list comprising:
   a mutation in the prM gene at nucleotide 592 from adenine to guanine resulting in an amino acid change at position 166 from lysine to glutamic acid, and
   a mutation in the NS5 gene at nucleotide 8803 from adenine to guanine resulting in an amino acid change at position 2903 from isoleucine to valine.
17. The unit dose of any one of items 14 to 16, wherein (iii) further comprises one or more of the mutations selected from the list comprising:
   a mutation in the E gene at nucleotide 1603 from adenine to thymine resulting in an amino acid change at position 503 from threonine to serine, and
   a silent mutation in the NS5 gene at nucleotide 7620 from adenine to guanine.
18. The unit dose of any one of items 14 to 17, wherein (iv) further comprises one or more of the mutations selected from the list comprising
   a silent mutation in the C gene at nucleotide 225 from adenine to thymine,
   a mutation in the NS2A gene at nucleotide 3674 from adenine to guanine resulting in an amino acid change at position 1193 from aspartic acid to glycine,
   a mutation in the NS2A gene at nucleotide 3773 from adenine to an adenine/guanine mix resulting in an amino acid change at position 1226 from lysine to a lysine/arginine mix,
   a silent mutation in the NS3 gene at nucleotide 5391 from cytosine to thymine,
   a mutation in the NS4A gene at nucleotide 6437 from cytosine to thymine resulting in an amino acid change at position 2114 from alanine to valine,
   a silent mutation in the NS4B gene at nucleotide 7026 from thymine to a thymine/cytosine mix, and
   a silent mutation in the NS5 gene at nucleotide 9750 from adenine to cytosine.
19. The unit dose of any one of items 1 to 18, wherein
   (i) has the amino acid sequence of SEQ ID NO. 2,
   (ii) has the amino acid sequence of SEQ ID NO. 4,
   (iii) has the amino acid sequence of SEQ ID NO. 6, and
   (iv) has the amino acid sequence of SEQ ID NO. 8.
20. The unit dose of any one of items 1 to 19 reconstituted with 0.3 to 0.8 mL of liquid for reconstitution.
21. The unit dose of item 20 reconstituted with 0.5 ml of liquid for reconstitution.
22. The unit dose of item 20 or 21, wherein the liquid for reconstitution is 37 mM aqueous sodium chloride solution.
23. A kit for preparing a reconstituted unit dose comprising the following components:
a) a unit dose of any one of items 1 to 19, and
b) a pharmaceutically acceptable diluent for reconstitution.
24. The kit of item 23, wherein the pharmaceutically acceptable diluent for reconstitution is 37 mM sodium chloride.
25. Container, such as a vial, comprising one to ten unit doses of any one of items 1 to 22.
26. A method of preventing dengue disease in a subject population comprising administering to the subject population a reconstituted unit dose of any one of items 20 to 22.
27. A method of preventing virologically confirmable dengue disease in a subject population comprising administering to the subject population a reconstituted unit dose of a tetravalent dengue virus composition including four live, attenuated dengue virus strains.
28. A method of preventing virologically confirmable dengue disease with hospitalization in a subject population comprising administering to the subject population a reconstituted unit dose of a tetravalent dengue virus composition including four live, attenuated dengue virus strains.
29. The method of items 26 to 28, wherein the geometric mean neutralizing antibody titers (GMTs) of the subject population when tested in at least 40, or at least 50, or at least 60 subjects at day 180 or day 365 after at least a first administration of said unit dose, and optionally a second administration of said unit dose 90 days after said first administration, provide a ratio of not more than 50, or not more than 40, or nor more than 30, or not more than 20 for the GMT of dengue serotype 2 to the GMT of dengue serotype 4.
30. The method of item 29, wherein said GMTs of the subject population further provide a ratio of not more than 20 for the GMT of dengue serotype 2 to the GMT of dengue serotype 1, and/or a ratio of not more than 20 for the GMT of dengue serotype 2 to the GMT of dengue serotype 3.
31. A method of preventing dengue disease in a subject comprising administering to the subject a reconstituted unit dose of any one of items 20 to 22.
32. A method of preventing virologically confirmable dengue disease in a subject comprising administering to the subject a reconstituted unit dose of a tetravalent dengue virus composition including four live, attenuated dengue virus strains.
33. A method of preventing virologically confirmable dengue disease with hospitalization in a subject comprising administering to the subject a reconstituted unit dose of a tetravalent dengue virus composition including four live, attenuated dengue virus strains.
34. The method of items 31 to 33, wherein the neutralizing antibody titers of the subject when tested at day 180 or day 365 after at least a first administration of said unit dose, and optionally a second administration of said unit dose 90 days after said first administration, provide a ratio of not more than 50, or not more than 40, or nor more than 30, or not more than 20 for the neutralizing antibody titer of dengue serotype 2 to the neutralizing antibody titer of dengue serotype 4.
35. The method of item 34, wherein said neutralizing antibody titers of the subject further provide a ratio of not more than 20 for the neutralizing antibody titer of dengue serotype 2 to the neutralizing antibody titer of dengue serotype 1, and/or a ratio of not more than 20 for the neutralizing antibody titer of dengue serotype 2 to the neutralizing antibody titer of dengue serotype 3.
36. The method of any one of items 26 to 35, wherein the method is for preventing dengue hemorrhagic fever (DHF) or dengue shock syndrome (DSS).
37. The method of any one of items 26 to 36, wherein the reconstituted unit dose is administered by subcutaneous injection, preferably to the deltoid region of the arm.
38. The method of any one of items 26 to 37, wherein two reconstituted unit doses of any one of items 20 to 22 are administered within 12 months or more.
39. The method of any one of items 26 to 37, wherein two reconstituted unit doses of any one of items 20 to 22 are administered within six months, preferably within three months.
40. The method of item 39, wherein the two reconstituted unit doses are administered at day 0 and day 90 or at day 1 and day 90.
41. The method of item 38 to 40, wherein a third unit dose is administered after administration of the second unit dose, preferably within 12 months after administration of the first unit dose.
42. The method of item 38 to 40, wherein a third unit dose is administered after administration of the second unit dose, preferably within 12 months after administration of the second unit dose.
43. The method of any one of items 26 to 42, wherein the subject or subject population is seronegative with respect to all dengue serotypes.
44. The method of any one of items 26 to 42, wherein the subject population or subject is seropositive with respect to at least one dengue serotype.
45. The method of any one of items 26 to 44, wherein the subject or subject population is of 2 to 60 years of age.
46. The method of any one of items 26 to 44, wherein the subject or subject population is of 2 to 17 years of age.
47. The method of any one of items 26 to 44, wherein the subject or subject population is under 9 years of age, under 4 years of age, or under 2 years of age or from 2 to 9 years of age, or from 2 to 5 years of age, or from 4 to 9 years of age or from 6 to 9 years of age, and optionally wherein the subject is seronegative with respect to all dengue serotypes.
48. The method of any one of items 26 to 44, wherein the subject or subject population is of 4 to 16 years of age.
49. The method of item 48, wherein the subject or subject population is of 4 to 5 years of age
50. The method of item 48, wherein the subject or subject population is of 6 to 11 years of age.
51. The method of item 48, wherein the subject or subject population is of 12 to 16 years of age.
52. The method of any one of items 26 to 51, wherein the subject or subject population is from a dengue endemic region.
53. The method of any one of items 26 to 51, wherein the subject or subject population is from a dengue non-endemic region.
54. The method of any one of items 52 or 53, wherein the subject or subject population is from Asia Pacific or Latin America.
55. The method of any one of items 26 to 54, wherein the subject or subject population has been subject to prior vaccination against Yellow Fever, wherein prior vaccination against Yellow Fever refers to a vaccination prior to the second administration or prior to the first administration.
56. The method of any one of items 26 to 54, wherein the subject or subject population is has been subject to prior vaccination against Japanese Encephalitis, wherein prior vaccination against Japanese Encephalitis refers to a vaccination prior to the second administration or prior to the first administration.
57. The method of any one of items 26 to 54, wherein the subject or subject population is has not been subject to prior vaccination against Yellow Fever.
58. The method of any one of items 26 to 54, wherein the subject or subject population is has not been subject to prior vaccination against Japanese Encephalitis.
59. The method of any one of items 26 to 58 having a combined vaccine efficacy against all four serotypes with a 2-sided 95% confidence interval, wherein the lower bound is more than 25%, when measured against placebo in a subject population of at least 5,000 healthy subjects, or at least 10,000 healthy subjects, or at least 15,000 healthy subjects irrespective of serostatus at baseline, wherein said unit dose or said placebo is administered at least twice within less than 6 months, such as within 3 months, about 30 days after the second administration of the administration schedule until at least 12 months after the second administration of the administration schedule.
60. The method of item 59, wherein the lower bound is more than 30%, is more than 40%, is more than 50%, is more than 55%, is more than 60%, is more than 65%, is more than 70% or is more than 72%.
61. The method of any one of items 26 to 60 having a combined vaccine efficacy against all four serotypes of more than 30%, when measured against placebo in a subject population of at least 5,000 healthy subjects, or at least 10,000 healthy subjects, or at least 15,000 healthy subjects irrespective of serostatus at baseline, wherein said unit dose or said placebo is administered at least twice within less than 6 months, such as within 3 months, 30 days after the second administration until at least 12 months after the second administration.
62. The method of item 61, wherein the combined vaccine efficacy against all four serotypes is more than 40%, is more than 50%, is more than 55%, is more than 60%, is more than 65%, is more than 70%, is more than 75% is more than 78%, is more than 79% or is about 80%.
63. The method of any one of items 26 to 62 having a combined relative risk against all four serotypes with a 2-sided 95% confidence interval, wherein the upper bound is less than 0.75, when measured against placebo in a subject population of at least 5,000 healthy subjects, or at least 10,000 healthy subjects, or at least 15,000 healthy subjects irrespective of serostatus at baseline, wherein said unit dose or said placebo is administered at least twice within less than 6 months, such as within 3 months, 30 days after the second administration until at least 12 months after the second administration.
64. The method of item 63, wherein the upper bound is less than 0.70, is less than 0.65, is less than 0.60, is less than 0.55, is less than 0.50, is less than 0.45, is less than 0.40, is less than 0.35, is less than 0.30 or is less than 0.28.
65. The method of any one of items 26 to 64, wherein the combined relative risk against all four serotypes is less than 0.70, when measured against placebo in a subject population of at least 5,000 healthy subjects, or at least 10,000 healthy subjects, or at least 15,000 healthy subjects irrespective of serostatus at baseline, wherein said unit dose or said placebo is administered at least twice within less than 6 months, such as within 3 months, 30 days after the second administration until at least 12 months after the second administration.
66. The method of item 65, wherein the combined relative risk against all four serotypes is less than 0.65, is less than 0.60, is less than 0.55, is less than 0.50, is less than 0.45, is less than 0.40, is less than 0.35, is less than 0.30, is less than 0.25 or is less than 0.23.
67. The method of any one of items 26 to 58 having a combined vaccine efficacy against all four serotypes with a 2-sided 95% confidence interval, wherein the lower bound is more than 61.0%, or more than 65.0% or more than 70.0% or more than 72.0% when measured against placebo in a subject population of at least 5,000 healthy subjects, or at least 10,000 healthy subjects, or at least 15,000 healthy subjects from endemic irrespective of serostatus at baseline and being selected from the group consisting of 4 to 16 year old subjects at the time of randomization, wherein said unit dose or said placebo is administered at least twice within 6 months or less, about 30 days after the last administration of the administration schedule until at least 12 or 13 months after the last administration of the administration schedule.
68. The method of any one of items 26 to 58 having a combined vaccine efficacy against all four serotypes of more than 66%, or of more than 70%, or of more than 75%, or of more than 77%, or of more than 80%, when measured against placebo in a subject population of at least 5,000 healthy subjects, or at least 10,000 healthy subjects, or at least 15,000 healthy subjects from endemic areas irrespective of serostatus at baseline and being selected from the group consisting of 4 to 16 year old subjects at the time of randomization, wherein said unit dose or said placebo is administered at least twice within 6 months or less, about 30 days after the last administration of the administration schedule until at least 12 months or 13 month after the last administration of the administration schedule.
69. The method of item 67 or 68, wherein the combined vaccine efficacy against all four serotypes is measured about 30 days after the last administration of the administration schedule until 12 or 13 months after the last administration of the administration schedule.
70. The method of item 67 or 68, wherein said unit dose or said placebo is administered twice within three months, in particular at about day 1 and about day 90, and wherein the combined vaccine efficacy against all four serotypes is measured 30 days after the second administration until 12 or 13 months after the second administration of the administration schedule.
71. The method of any one of items 26 to 70 being effective and safe.
72. The method of any one of items 26 to 71 having a relative risk for virologically confirmed dengue with hospitalization which is 1 or less, or 0.8 or less, or 0.6 or less, when measured against placebo in a subject population of at least 5,000 healthy subjects, or at least 10,000 healthy subjects, or at least 15,000 healthy subjects.
73. The method of any one of items 59 to 72, wherein the healthy subjects of the subject population are of 4 to 16 years of age at the time of randomization.
74. The method of any one of items 59 to 72, wherein the healthy subjects of the subject population are of 4 to 5 years of age at the time of randomization
75. The method of any one of items 59 to 72, wherein the healthy subjects of the subject population are of 6 to 11 years of age at the time of randomization.
76. The method of any one of items 59 to 72, wherein the healthy subjects of the subject population are of 12 to 16 years of age at the time of randomization.
77. The method of any one of items 59 to 72, wherein the healthy subjects of the subject population are from Asia Pacific or Latin America.
78. The method of any one of items 59 to 77, wherein the healthy subjects of the subject population are seropositive with respect to at least one serotype at baseline.
79. The method of any one of items 59 to 77, wherein the healthy subjects of the subject population are seronegative with respect to all serotypes at baseline.
80. The method of any one of items 59 to 79, wherein the healthy subjects of the subject population have been subject to prior vaccination against Yellow Fever.
81. The method of any one of items 59 to 79, wherein the healthy subjects of the subject population have been subject to prior vaccination against Japanese Encephalitis.
82. The method of any one of items 59 to 79, wherein the healthy subjects of the subject population have not been subject to prior vaccination against Yellow Fever.
83. The method of any one of items 59 to 79, wherein the healthy subjects of the subject population have not been subject to prior vaccination against Japanese Encephalitis.
84. A method for stimulating an immune response to all four serotypes of dengue virus in a subject, comprising administering to the subject a reconstituted unit dose of items 20 to 22.
85. The method of item 84, wherein the immune response to all four serotypes of dengue virus is balanced.
86. The method of item 84 or 85, wherein the reconstituted unit dose is administered by subcutaneous injection, preferably to the deltoid region of the arm.
87. The method of any one of items 84 to 86, wherein two unit doses of any one of items 20 to 22 are administered within 12 months or more.
88. The method of any one of items 84 to 87, wherein two reconstituted unit doses of any one of items 20 to 22 are administered within six months, preferably within three months.
89. The method of item 88, wherein the two reconstituted unit doses are administered at day 0 and day 90 or at day 1 and day 90.
90. The method of item 87 to 89, wherein a third unit dose is administered after the administration of the second unit dose, preferably within 12 month of administration of the first unit dose.
91. The method of item 87 to 89, wherein a third unit dose is administered after the administration of the second unit dose, preferably within 12 month of administration of the second unit dose.
92. The method of any one of items 84 to 91, wherein the subject is from a dengue endemic region.
93. The method of any one of items 84 to 91, wherein the subject is from a dengue non-endemic region.
94. The method of any one of items 84 to 93, wherein the subject is seronegative with respect to all dengue serotypes.
95. The method of any one of items 84 to 93, wherein the subject is seropositive with respect to at least one dengue serotype.
96. The method of any one of items 84 to 95, wherein the neutralizing antibody titers of the subject when tested at day 180 or day 365 after at least a first administration of said reconstituted unit dose, and optionally a second administration of said reconstituted unit dose 90 days after said first administration, provide a ratio of not more than 50, or not more than 40, or nor more than 30, or not more than 20 for the neutralizing antibody titer of dengue serotype 2 to the neutralizing antibody titer of dengue serotype 4.
97. The method of item 96, wherein said neutralizing antibody titers of the subject further provide a ratio of not more than 20 for the neutralizing antibody titer of dengue serotype 2 to the neutralizing antibody titer of dengue serotype 1, and/or a ratio of not more than 20 for the neutralizing antibody titer of dengue serotype 2 to the neutralizing antibody titer of dengue serotype 3.
98. The method of any one of items 84 to 97, wherein the subject is of 2 and 60 years of age.
99. The method of any one of items 84 to 97, wherein the subject is under 9 years of age, under 4 years of age, or under 2 years of age.
100. The method of any one of items 26 to 99, wherein the reconstituted unit dose is obtained from the kit according to item 23 or 24.
101. The reconstituted unit dose of any one of item 20 to 22 for use in a method of items 26 to 100.
102. Use of a reconstituted unit dose of any one of items 20 to 22 for the manufacture of a medicament for a method according to items 26 to 100.
103. A method for determining the titer of neutralizing antibodies against each of dengue serotypes 1, 2, 3 and 4 in a blood serum sample, the method comprising the steps of:
   (a) seeding cells from a dengue-susceptible cell line on 96-well assay plates and culturing the cells for a culture period;
   (b) preparing serial dilutions of the blood serum sample;
   (c) separately mixing the serially diluted blood serum samples prepared in step (b) with dengue serotype 1, dengue serotype 2, dengue serotype 3 and dengue serotype 4 to obtain separate mixtures for each dengue serotype and incubating the separate mixtures;
   (d) adding the separate mixtures prepared in (c) to the cells seeded and cultured in step (a) and incubating the cells with the separate mixtures;
   (e) providing an overlay for the inoculated cells and incubating the cells for an incubation period of 40 to 75 hours;
   (f) determining the number of plaques in each well and comparing the number of plaques in each well to a control to determine the level of neutralizing antibodies against each of dengue serotypes 1, 2, 3 and 4.
104. Method according to item 103, wherein in step (e) different incubation periods are used for the mixtures of different dengue serotypes.
105. Method according to item 103 or 104 wherein in step (e) the incubation period for mixtures of dengue serotype 4 is shorter than the incubation period for mixtures of dengue serotypes 1, 2 and 3.
106. Method according to item 105, wherein the incubation period for mixtures of dengue serotype 4 is 46±2 hours.
107. Method according to any one of the items 103 to 106, wherein in step (e) the incubation period for mixtures of dengue serotype 2 is longer than the incubation period for mixtures of dengue serotypes 1, 3 and 4.
108. Method according to item 107, wherein the incubation period for mixtures of dengue serotype 2 is 70±2 hours.
109. Method according to any one of items 103 to 108, wherein the dengue-susceptible cell line is selected from Vero cells, LLC-MK2 cells and BHK-21 cells.
110. Method according to any one of items 103 to 109, wherein the culture period in step (a) is 12 to 36 hours.
111. Method according to any one of items 103 to 110 wherein in step (c) the dengue serotype 1 is DENV-1 strain 16007, dengue serotype 2 is DENV-2 strain 16681, dengue serotype 3 is DENV-3 strain 16562 and dengue serotype 4 is DENV-4 strain 1036.
112. Method according to any one of items 103 to 111, wherein the separate mixtures in step (c) are incubated overnight at a temperature of 2° C. to 8° C.
113. Method according to any one of items 103 to 112, wherein the overlay in step (e) is selected from the group consisting of methylcellulose, carboxymethylcellulose and agarose.
114. Method according to any one of items 103 to 113, wherein in step (e) the cells are incubated at a temperature of 33° C. to 35° C.
115. Method according to any one of items 103 to 114, wherein the number of plaques in each well is determined using serotype-specific anti-dengue monoclonal antibodies.
116. A method for determining the titer of neutralizing antibodies against each of dengue serotypes 1, 2, 3 and 4 in a blood serum sample, the method comprising the steps of:
   (a) seeding Vero cells on 96-well assay plates and culturing the Vero cells for a period of 20 to 30 hours;
   (b) preparing serial dilutions of the serum sample;
   (c) separately mixing the serially diluted serum samples with dengue serotype 1, dengue serotype 2, dengue serotype 3 and dengue serotype 4 to prepare separate mixtures and incubating the separate mixtures overnight at a temperature of 2° C. to 8° C.;
   (d) incubating the cells seeded and cultured in step (a) with the separate mixtures prepared in step (c) in separate wells for 90 to 120 minutes;
   (e) providing a methylcellulose overlay for the inoculated cells and incubating the cells for an incubation period of 40 to 75 hours at 34° C.;
   (f) determining the number of plaques in each well using serotype-specific anti-dengue monoclonal antibodies and comparing the number of plaques in each well to a control to determine the level of neutralizing antibodies against each of dengue serotypes 1, 2, 3 and 4.
117. Use of the method according to any one of items 103 to 116 for determining the dengue serostatus of a subject before vaccination with a dengue virus vaccine or for analyzing a subject's immune response after vaccination with a dengue virus vaccine.

### LIST OF FURTHER ITEMS OF THE INVENTION

1. A dengue vaccine composition for use in a method of preventing virologically confirmable dengue disease in a subject comprising consecutively administering at least a first and a second unit dose of the dengue vaccine composition to the subject, wherein said first and second unit dose are administered subcutaneously within 3 months and at least 4 weeks apart, optionally at about day 1 and at about day 90, and wherein the dengue vaccine composition is a tetravalent dengue virus composition including four live, attenuated dengue virus strains representing dengue serotype 1, dengue serotype 2, dengue serotype 3 and dengue serotype 4, wherein the attenuated dengue virus strains comprise chimeric dengue viruses and at least one non-chimeric dengue virus, and wherein the dengue serotype 1 and the dengue serotype 2 are present each in a concentration based on the total concentration in pfu/0.5 mL which is within 5%-points of each other and/or are together less than about 10% of the total concentration in pfu/0.5 mL.
2. The composition for use of item 1, wherein the method does not comprise a determination of a previous dengue infection in the subject before the administration of the first unit dose of the tetravalent dengue virus composition and wherein the method is safe and effective.
3. The composition for use of item 1 or 2, wherein the dengue serotype 3 is at least about 10% of the total concentration in pfu/0.5 mL and/or wherein the dengue serotype 4 is at least about 70% of the total concentration in pfu/0.5 mL.
4. The composition for use of any one of items 1 to 3, wherein the dengue serotype 4 represents the highest concentration in the composition of all four serotypes, preferably with at least about 70% of the total concentration in pfu/0.5 mL, dengue serotype 3 represents the second highest concentration in the composition of all four serotypes, preferably with at least about 10% of the total concentration in pfu/0.5 mL, and dengue serotype 1 and dengue serotype 2 each represent lower concentrations than the concentration of serotype 3, and optionally together represent less than about 10% of the total concentration in pfu/0.5 mL.
5. The composition for use of any one of items 1 to 4, wherein the dengue serotype 1 is a chimeric dengue serotype 2/1 strain, the dengue serotype 2 is a non-chimeric dengue serotype 2 strain, the dengue serotype 3 is a chimeric dengue serotype 2/3 strain and the dengue serotype 4 is a chimeric dengue serotype 2/4 strain.
6. The composition for use of any one of items 1 to 5, wherein the dengue serotype 1 has the amino acid sequence of SEQ ID NO. 2, the dengue serotype 2 has the amino acid sequence of SEQ ID NO. 4, the dengue serotype 3 has the amino acid sequence of SEQ ID NO. 6, and the dengue serotype 4 has the amino acid sequence of SEQ ID NO. 8.
7. The composition for use of any one of items 1 to 6, wherein the unit dose upon reconstitution with 0.5 mL of a pharmaceutically acceptable diluent
   (i) dengue serotype 1 has a concentration of 3.3 log 10 pfu/0.5 mL to 5.0 log 10 pfu/0.5 mL,
   (ii) dengue serotype 2 has a concentration of 2.7 log 10 pfu/0.5 mL to 4.9 log 10 pfu/0.5 mL,
   (iii) dengue serotype 3 has a concentration of 4.0 log 10 pfu/0.5 mL to 5.7 log 10 pfu/0.5 mL, and
   (iv) dengue serotype 4 has a concentration of 4.5 log 10 pfu/0.5 mL to 6.2 log 10 pfu/0.5 mL.
8. The composition for use of any one of items 1 to 7, wherein the composition further comprises about 15% (w/v) α,α-trehalose dihydrate, about 1% (w/v) poloxamer 407, about 0.1% (w/v) human serum albumin, and about 100 mM sodium chloride when measured in 0.5 ml.
9. The composition for use of any one of items 1 to 8, wherein the unit doses are administered to the deltoid region of the arm.
10. The composition for use of any one of items 1 to 9, wherein the subject is seronegative to all dengue serotypes at baseline and/or is under 9 years of age.
11. The composition for use of any one of items 1 to 10, wherein the subject is 4 to 5 years of age or 6 to 11 years of age or 12 to 16 years of age.
12. The composition for use of any one of items 1 to 11, wherein the method is for preventing dengue hemorrhagic fever (DHF) or dengue shock syndrome (DSS).
13. The composition for use of any one of items 1 to 12, wherein the subject is from a dengue endemic region.
14. The composition for use of any one of items 1 to 12, wherein the subject is from a dengue non-endemic region.
15. The composition for use of any one of items 1 or 14, wherein the subject is from Asia Pacific or Latin America.
16. The composition for use of any one items of 1 to 15, wherein the composition provides a seropositivity rate when it is administered to a subject population of at least 50 subjects in two unit doses subcutaneously at day 1 and at day 90, wherein the subjects of the subject population are seronegative to all dengue serotypes at baseline.
17. The composition for use of item 16, wherein at least one month after administration of the first unit dose, such as at day 30, at least 80% of the subject population are seropositive for all four dengue serotypes.
18. The composition for use of item 16 or 17, wherein before or at the time of the administration of the second unit dose, such as at day 90, at least 80% of the subject population are seropositive for all four dengue serotypes.
19. The composition for use of any one of items 16 to 18, wherein after the administration of the second unit dose, such as at day 120, at least 80%, or at least 85%, or at least 90% or at least 95% of the subject population are seropositive for all four dengue serotypes.
20. The composition for use of any one of items 11 to 14, wherein after the administration of the second unit dose, such as at day 270, at least 80%, or at least 85%, or at least 90% of the subject population are seropositive for all four dengue serotypes.
21. The composition for use of any one of items 16 to 20, wherein the composition provides a seropositivity rate, when it is administered to a subject population of at least 100 subjects in two unit doses subcutaneously at day 1 and at day 90, wherein the subjects of the subject population comprises from 20% to 40% subjects who are seronegative to all dengue serotypes and from 60% to 80% subjects who are seropositive to at least one dengue serotype at base line, wherein at day 120 and/or day 270 the seropositivity rate for all four dengue serotypes in the seronegative part of the subject population and the seropositivity rate for all four dengue serotypes in the seropositive part of the subject population do not deviate more than 10%-points and/or wherein at day 120 the seropositivity rate for all four dengue serotypes in the seronegative part of the subject population and the seropositivity rate for all four dengue serotypes in the seropositive part of the subject population do not deviate more than 5%-points.
22. A method of inoculating a subject against virologically confirmable dengue disease in a subject comprising administering to the subject a tetravalent dengue virus composition including four dengue virus strains representing serotype 1, serotype 2, serotype 3 and serotype 4.
23. A method of inoculating a subject against virologically confirmable dengue disease consisting of administering to the subject a tetravalent dengue virus composition including four dengue virus strains representing serotype 1, serotype 2, serotype 3 and serotype 4.
24. The method of item 22 or 23, wherein the method does not comprise a determination of a previous dengue infection in the subject before the administration of the tetravalent dengue virus composition.
25. The method of any one of items 22 to 24, wherein the inoculation is safe irrespective of whether there is a determination that the subject had a previous dengue infection before the administration of the tetravalent dengue virus composition.
26. The method of any one of items 22 to 25 which is safe.
27. The method of any one of items 22 to 26 which is effective.
28. The method of any one of items 22 to 27, wherein the virus strains are live, attenuated dengue virus strains.
29. The method of any one of items 22 to 28, wherein the composition includes at least one chimeric dengue virus and optionally at least one non-chimeric dengue virus.
30. The method of any one of items 22 to 29, wherein the composition includes a chimeric dengue serotype 2/1 strain and a dengue serotype 2 strain and a chimeric dengue serotype 2/3 strain and a chimeric dengue serotype 2/4 strain.
31. The method of any one of items 22 to 30, wherein the subject is seronegative to all dengue serotypes at base line and/or under 9 years of age, 4 to 5 years of age, 6 to 11 years of age or 12 to 16 years of age.
32. The method of any one of items 22 to 31, wherein the composition is administered by subcutaneous injection.
33. The method of any one of items 22 to 32 including consecutively administering at least a first and a second unit dose of the dengue vaccine composition to the subject, wherein said first and second unit dose are administered subcutaneously within 3 months and at least four 4 apart, optionally at about day 1 and at about day 90.
34. The method of any one of items 22 to 33, wherein the dengue serotypes 1 and 2 are present in similar amounts and/or make up less than about 10% of the total viral concentration.
35. The method of any one of items 22 to 34, wherein the dengue serotype 3 makes up at least about 10% of the total viral concentration.
36. The method of any one of items 22 to 35, wherein the dengue serotype 4 makes up at least about 70% of the total viral concentration.
37. The method of any one of items 22 to 36 wherein the dengue serotype 4 represents the highest concentration in the composition of all four serotypes, preferably with at least about 70% of the total concentration in pfu/0.5 mL, dengue serotype 3 represents the second highest concentration in the composition of all four serotypes, preferably with at least about 10% of the total concentration in pfu/0.5 mL, and dengue serotype 1 and dengue serotype 2 each represent lower concentrations than the concentration of serotype 3, and optionally together represent less than about 10% of the total concentration in pfu/0.5 mL.
38. The method of any one of items 22 to 37, wherein the method is for preventing dengue hemorrhagic fever (DHF) or dengue shock syndrome (DSS).
39. The method of any one of items 22 to 38 wherein the subject or subject population is from a dengue endemic region.
40. The method of any one of items 22 to 38, wherein the subject or subject population is from a dengue non-endemic region.
41. The method of any one of items 22 to 40, wherein the subject or subject population is from Asia Pacific or Latin America.
42. The method of any one of items 22 to 41 having a combined vaccine efficacy against all four dengue serotypes with a 2-sided 95% confidence interval, wherein the lower bound is more than 25%, when measured against placebo in a subject population of at least 5,000 healthy subjects, or at least 10,000 healthy subjects, or at least 15,000 healthy subjects irrespective of serostatus at baseline, wherein said unit dose or said placebo is administered at least twice within less than 6 months, such as within 3 months, and optionally at least 4 weeks apart, about 30 days after the second administration of the administration schedule until at least 12 months after the second administration of the administration schedule.
43. The method of item 42, wherein the lower bound is more than 30%, is more than 40%, is more than 50%, is more than 55%, is more than 60%, is more than 65%, is more than 70% or is more than 72%.
44. The method of any one of items 22 to 43 having a combined vaccine efficacy against all four dengue serotypes of more than 30%, when measured against placebo in a subject population of at least 5,000 healthy subjects, or at least 10,000 healthy subjects, or at least 15,000 healthy subjects irrespective of serostatus at baseline, wherein said unit dose or said placebo is administered at least twice within less than 6 months, such as within 3 months, and optionally at least 4 weeks apart, 30 days after the second administration until at least 12 months after the second administration.
45. The method of item 44, wherein the combined vaccine efficacy against all four dengue serotypes is more than 40%, is more than 50%, is more than 55%, is more than 60%, is more than 65%, is more than 70%, is more than 75% is more than 78%, is more than 79% or is about 80%.
46. The method of any one of items 22 to 45 having a combined vaccine efficacy against all four dengue serotypes in seronegative subjects with a 2-sided 95% confidence interval, wherein the lower bound is more than 25%, when measured against placebo in a subject population of at least 2,000 healthy subjects being seronegative against all serotypes at baseline, wherein said unit dose or said placebo is administered at least twice within less than 6 months, such as within 3 months, about 30 days after the second administration of the administration schedule until at least 12 months after the second administration of the administration schedule.
47. The method of item 46, wherein the lower bound is more than 30%, is more than 40%, is more than 50%, or is more than 55%.
48. The method of any one of items 22 to 47 having a combined vaccine efficacy against all four dengue serotypes in seronegative subjects of more than 30%, when measured against placebo in a subject population of at least 2,000 healthy subjects being seronegative against all serotypes at baseline, wherein said unit dose or said placebo is administered at least twice within less than 6 months, such as within 3 months, 30 days after the second administration until at least 12 months after the second administration.
49. The method of item 48, wherein the combined vaccine efficacy against all four dengue serotypes in seronegative subjects is more than 40%, is more than 50%, is more than 60%, is more than 65%, or is more than 70%.
50. The method of any one of items 22 to 49 having a combined vaccine efficacy against all four dengue serotypes with a 2-sided 95% confidence interval, wherein the lower bound is more than 25%, when measured against placebo in a subject population of at least 1,000 healthy subjects 4 to 5 years of age at the time of randomization and irrespective of serostatus at baseline, wherein said unit dose or said placebo is administered at least twice within less than 6 months, such as within 3 months, about 30 days after the second administration of the administration schedule until at least 12 months after the second administration of the administration schedule.
51. The method of item 50, wherein the lower bound is more than 30%, is more than 40%, is more than 45%.
52. The method of any one of items 22 to 51 having a combined vaccine efficacy against all four dengue serotypes of more than 30%, when measured against placebo in a subject population of at least 1,000 healthy subjects 4 to 5 years of age at the time of randomization and irrespective of serostatus at baseline, wherein said unit dose or said placebo is administered at least twice within less than 6 months, such as within 3 months, 30 days after the second administration until at least 12 months after the second administration.
53. The method of item 52, wherein the combined vaccine efficacy against all four dengue serotypes is more than 40%, is more than 50%, is more than 60%, is more than 65%, or is more than 70%.
54. The method of any one of items 22 to 53 having a combined vaccine efficacy against all four dengue serotypes with a 2-sided 95% confidence interval, wherein the lower bound is more than 25%, when measured against placebo in a subject population of at least 1,000 healthy subjects 6 to 11 years of age at the time of randomization and irrespective of serostatus at baseline, wherein said unit dose or said placebo is administered at least twice within less than 6 months, such as within 3 months, about 30 days after the second administration of the administration schedule until at least 12 months after the second administration of the administration schedule.
55. The method of item 54, wherein the lower bound is more than 30%, is more than 40%, is more than 50%, is more than 60%, or is more than 70%.
56. The method of any one of items 22 to 55 having a combined vaccine efficacy against all four dengue serotypes of more than 30%, when measured against placebo in a subject population of at least 1,000 healthy subjects 6 to 11 years of age at the time of randomization and irrespective of serostatus at baseline, wherein said unit dose or said placebo is administered at least twice within less than 6 months, such as within 3 months, 30 days after the second administration until at least 12 months after the second administration.
57. The method of item 56, wherein the combined vaccine efficacy against all four dengue serotypes is more than 40%, is more than 50%, is more than 60%, is more than 70%, is more than 75%, or is more than 80%.
58. The method of any one of items 26 to 57 having a combined vaccine efficacy against all four dengue serotypes with a 2-sided 95% confidence interval, wherein the lower bound is more than 25%, when measured against placebo in a subject population of at least 1,000 healthy subjects 12 to 16 years of age at the time of randomization and irrespective of serostatus at baseline, wherein said unit dose or said placebo is administered at least twice within less than 6 months, such as within 3 months, about 30 days after the second administration of the administration schedule until at least 12 months after the second administration of the administration schedule.
59. The method of item 58, wherein the lower bound is more than 30%, is more than 40%, is more than 50%, is more than 60%, is more than 65%, or is more than 68%.
60. The method of any one of items 26 to 59 having a combined vaccine efficacy against all four dengue serotypes of more than 30%, when measured against placebo in a subject population of at least 1,000 healthy subjects 12 to 16 years of age at the time of randomization and irrespective of serostatus at baseline, wherein said unit dose or said placebo is administered at least twice within less than 6 months, such as within 3 months, 30 days after the second administration until at least 12 months after the second administration.
61. The method of item 60, wherein the combined vaccine efficacy against all four dengue serotypes is more than 40%, is more than 50%, is more than 60%, is more than 70%, is more than 75%, or is more than 80%.
62. The method of any one of items 22 to 61 having a vaccine efficacy against dengue serotype 1 with a 2-sided 95% confidence interval, wherein the lower bound is more than 25%, when measured against placebo in a subject population of at least 5,000 healthy subjects, or at least 10,000 healthy subjects, or at least 15,000 healthy subjects irrespective of serostatus at baseline, wherein said unit dose or said placebo is administered at least twice within less than 6 months, such as within 3 months, about 30 days after the second administration of the administration schedule until at least 12 months after the second administration of the administration schedule.
63. The method of item 62, wherein the lower bound is more than 30%, is more than 40%, or is more than 50%.
64. The method of any one of items 22 to 63 having a vaccine efficacy against dengue serotype 1 of more than 30%, when measured against placebo in a subject population of at least 5,000 healthy subjects, or at least 10,000 healthy subjects, or at least 15,000 healthy subjects irrespective of serostatus at baseline, wherein said unit dose or said placebo is administered at least twice within less than 6 months, such as within 3 months, 30 days after the second administration until at least 12 months after the second administration.
65. The method of item 64, wherein the vaccine efficacy against dengue serotype 1 is more than 40%, is more than 50%, is more than 60%, is more than 65%, or is more than 70%.
66. The method of any one of items 22 to 65 having a vaccine efficacy against dengue serotype 2 with a 2-sided 95% confidence interval, wherein the lower bound is more than 25%, when measured against placebo in a subject population of at least 5,000 healthy subjects, or at least 10,000 healthy subjects, or at least 15,000 healthy subjects irrespective of serostatus at baseline, wherein said unit dose or said placebo is administered at least twice within less than 6 months, such as within 3 months, about 30 days after the second administration of the administration schedule until at least 12 months after the second administration of the administration schedule.
67. The method of item 66, wherein the lower bound is more than 30%, is more than 40%, is more than 50, is more than 60, is more than 70, is more than 80, or is more than 90%.
68. The method of any one of items 22 to 67 having a vaccine efficacy against dengue serotype 2 of more than 30%, when measured against placebo in a subject population of at least 5,000 healthy subjects, or at least 10,000 healthy subjects, or at least 15,000 healthy subjects irrespective of serostatus at baseline, wherein said unit dose or said placebo is administered at least twice within less than 6 months, such as within 3 months, 30 days after the second administration until at least 12 months after the second administration.
69. The method of item 68, wherein the vaccine efficacy against dengue serotype 2 is more than 40%, is more than 50%, is more than 60%, is more than 70%, is more than 80, or is more than 90%.
70. The method of any one of items 22 to 69 having a vaccine efficacy against dengue serotype 3 with a 2-sided 95% confidence interval, wherein the lower bound is more than 25%, when measured against placebo in a subject population of at least 5,000 healthy subjects, or at least 10,000 healthy subjects, or at least 15,000 healthy subjects irrespective of serostatus at baseline, wherein said unit dose or said placebo is administered at least twice within less than 6 months, such as within 3 months, about 30 days after the second administration of the administration schedule until at least 12 months after the second administration of the administration schedule.
71. The method of item 70, wherein the lower bound is more than 30%, is more than 40%.
72. The method of any one of items 22 to 71 having a vaccine efficacy against dengue serotype 3 of more than 30%, when measured against placebo in a subject population of at least 5,000 healthy subjects, or at least 10,000 healthy subjects, or at least 15,000 healthy subjects irrespective of serostatus at baseline, wherein said unit dose or said placebo is administered at least twice within less than 6 months, such as within 3 months, 30 days after the second administration until at least 12 months after the second administration.
73. The method of item 72, wherein the vaccine efficacy against dengue serotype 3 is more than 40%, is more than 50%, is more than 55%, or is more than 60%.
74. The method of any one of items 22 to 73 having a combined vaccine efficacy against virologically-confirmed dengue with hospitalization against all four serotypes with a 2-sided 95% confidence interval, wherein the lower bound is more than 25%, when measured against placebo in a subject population of at least 2,000 healthy subjects being seronegative against all serotypes at baseline, wherein said unit dose or said placebo is administered at least twice within less than 6 months, such as within 3 months, about 30 days after the second administration of the administration schedule until at least 12 months after the second administration of the administration schedule.
75. The method of item 74, wherein the lower bound is more than 30%, is more than 40%, is more than 50%, is more than 60%, is more than 70%, or is more than 75%.
76. The method of any one of items 22 to 75 having a combined vaccine efficacy against virologically-confirmed dengue with hospitalization against all four serotypes of more than 30%, when measured against placebo in a subject population of at least 2,000 healthy subjects, healthy subjects being seronegative against all serotypes at baseline, wherein said unit dose or said placebo is administered at least twice within less than 6 months, such as within 3 months, 30 days after the second administration until at least 12 months after the second administration.
77. The method of item 76, wherein the combined vaccine efficacy against virologically-confirmed dengue with hospitalization against all four serotypes is more than 40%, is more than 50%, is more than 60%, is more than 70%, is more than 80%, or is more than 90%.
78. The method of any one of items 21 to 77 having a combined vaccine efficacy against virologically-confirmed dengue with hospitalization against all four serotypes with a 2-sided 95% confidence interval, wherein the lower bound is more than 25%, when measured against placebo in a subject population of at least 2,000 healthy subjects being seropositive at baseline, wherein said unit dose or said placebo is administered at least twice within less than 6 months, such as within 3 months, about 30 days after the second administration of the administration schedule until at least 12 months after the second administration of the administration schedule.
79. The method of item 78, wherein the lower bound is more than 30%, is more than 40%, is more than 50%, is more than 60%, is more than 70%, or is more than 80%.
80. The method of any one of items 22 to 79 having a combined vaccine efficacy against virologically-confirmed dengue with hospitalization against all four serotypes of more than 30%, when measured against placebo in a subject population of at least 2,000 healthy subjects, healthy subjects being seropositive at baseline, wherein said unit dose or said placebo is administered at least twice within less than 6 months, such as within 3 months, 30 days after the second administration until at least 12 months after the second administration.
81. The method of item 80, combined vaccine efficacy against virologically-confirmed dengue with hospitalization against all four serotypes is more than 40%, is more than 50%, is more than 60%, is more than 70%, is more than 80%, or is more than 90%.
82. The method of any one of items 22 to 81 having a combined relative risk against all four dengue serotypes with a 2-sided 95% confidence interval, wherein the upper bound is less than 0.75, when measured against placebo in a subject population of at least 5,000 healthy subjects, or at least 10,000 healthy subjects, or at least 15,000 healthy subjects irrespective of serostatus at baseline, wherein said unit dose or said placebo is administered at least twice within less than 6 months, such as within 3 months, 30 days after the second administration until at least 12 months after the second administration.
83. The method of item 82, wherein the upper bound is less than 0.70, is less than 0.65, is less than 0.60, is less than 0.55, is less than 0.50, is less than 0.45, is less than 0.40, is less than 0.35, is less than 0.30 or is less than 0.28.

84. The method of any one of items 22 to 83, wherein the combined relative risk against all four dengue serotypes is less than 0.70, when measured against placebo in a subject population of at least 5,000 healthy subjects, or at least 10,000 healthy subjects, or at least 15,000 healthy subjects irrespective of serostatus at baseline, wherein said unit dose or said placebo is administered at least twice within less than 6 months, such as within 3 months, 30 days after the second administration until at least 12 months after the second administration.
85. The method of item 84, wherein the combined relative risk against all four serotypes is less than 0.65, is less than 0.60, is less than 0.55, is less than 0.50, is less than 0.45, is less than 0.40, is less than 0.35, is less than 0.30, is less than 0.25 or is less than 0.23.
86. The method of any one of items 22 to 85 having a combined vaccine efficacy against all four serotypes with a 2-sided 95% confidence interval, wherein the lower bound is more than 61.0%, or more than 65.0 or more than 70.0% or more than 72.0% when measured against placebo in a subject population of at least 5,000 healthy subjects, or at least 10,000 healthy subjects, or at least 15,000 healthy subjects from endemic regions irrespective of serostatus at baseline and being selected from the group consisting of 4 to 16 year old subjects at the time of randomization, wherein said unit dose or said placebo is administered at least twice within 6 months or less, about 30 days after the last administration of the administration schedule until at least 12 or 13 months after the last administration of the administration schedule.
87. The method of any one of items 22 to 86 having a combined vaccine efficacy against all four serotypes of more than 66%, or of more than 70%, or of more than 75%, or of more than 77%, or of more than 80.0%, when measured against placebo in a subject population of at least 5,000 healthy subjects, or at least 10,000 healthy subjects, or at least 15,000 healthy subjects from endemic regions irrespective of serostatus at baseline and being selected from the group consisting of 4 to 16 year old subjects at the time of randomization, wherein said unit dose or said placebo is administered at least twice within 6 months or less, about 30 days after the last administration of the administration schedule until at least 12 months or 13 month after the last administration of the administration schedule.
88. The method of any one of items 22 to 87, wherein said unit dose or said placebo is administered at day 1 and day 90.
89. The method of any one of items 22 to 88 having a relative risk for virologically confirmed dengue with hospitalization which is 1 or less, or 0.8 or less, or 0.6 or less, when measured against placebo in a subject population of at least 1,000 healthy subjects, or at least 5,000 healthy subjects, or at least 10,000 healthy subjects irrespective of serostatus at baseline and in age groups from 4 to 16 years, in particular in subjects 4 to 5 years of age at the time of randomization.
90. The method of any one of items 22 to 89, wherein the occurrence of vaccine related serious adverse events is less than 0.1%.
91. The method of any one of items 22 to 90, wherein the occurrence of vaccine related unsolicited adverse events occurring within 4 weeks of administration is less than 2%.
92. The method of any one of items 22 to 91, wherein the occurrence of vaccine related solicited adverse events occurring within 2 weeks of administration is less than 35%.
93. The method of any one of items 22 to 92, wherein the occurrence of solicited local reactions occurring within 1 weeks of administration is less than 40%.
94. The method of any one of items 22 to 93, wherein the unit dose upon reconstitution with 0.5 mL of a pharmaceutically acceptable diluent
   (i) dengue serotype 1 has a concentration of 3.3 log 10 pfu/0.5 mL to 5.0 log 10 pfu/0.5 mL,
   (ii) dengue serotype 2 has a concentration of 2.7 log 10 pfu/0.5 mL to 4.9 log 10 pfu/0.5 mL,
   (iii) dengue serotype 3 has a concentration of 4.0 log 10 pfu/0.5 mL to 5.7 log 10 pfu/0.5 mL, and
   (iv) dengue serotype 4 has a concentration of 4.5 log 10 pfu/0.5 mL to 6.2 log 10 pfu/0.5 mL,
   and optionally comprises about 15% (w/v) α,α-trehalose dihydrate, about 1% (w/v) poloxamer 407, about 0.1% (w/v) human serum albumin, and about 100 mM sodium chloride when measured in 0.5 mL.
95. A reconstituted unit dose of a dengue vaccine composition for use in a method of preventing virologically confirmable dengue disease in a subject comprising consecutively administering at least a first and a second unit dose of the dengue vaccine composition to the subject, wherein said first and second unit dose are administered subcutaneously within 3 months and at least 4 weeks apart, optionally at about day 1 and at about day 90, wherein the dengue vaccine composition is a tetravalent dengue virus composition including four dengue virus strains representing dengue serotype 1, dengue serotype 2, dengue serotype 3 and dengue serotype 4, optionally wherein the dengue virus strains are live, attenuated, and wherein upon reconstitution with 0.5 ml of a pharmaceutically acceptable diluent
   (i) dengue serotype 1 has a concentration of at least 3.3 log 10 pfu/0.5 mL,
   (ii) dengue serotype 2 has a concentration of at least 2.7 log 10 pfu/0.5 mL,
   (iii) dengue serotype 3 has a concentration of at least 4.0 log 10 pfu/0.5 mL, and
   (iv) dengue serotype 4 has a concentration of at least 4.5 log 10 pfu/0.5 mL.
96. The unit dose for use of item 95, wherein the subject is under 9 years of age and/or when the serostatus of the subject is unknown or seronegative.
97. The unit dose for use of item 95 or 96, which is effective.
98. The unit dose for use of any one of items 95 to 97, which is effective against all four dengue serotypes.
99. The unit dose for use of any one of items 95 to 98, which is safe.
100. The unit dose for use of any one of items 95 to 99, wherein the unit dose includes at least one chimeric dengue virus.
101. The unit dose for use of any one of items 95 to 100, wherein the unit dose includes at least one non-chimeric dengue virus and at least one chimeric dengue virus.
102. The unit dose for use of any one of items 95 to 101, wherein the subject is seronegative to all dengue serotypes at baseline and/or is under 9 years of age.
103. The unit dose for use of any one of items 95 to 102, wherein the subject is 4 to 5 years of age or 6 to 11 years of age or 12 to 16 years of age.
104. The unit dose for use of any one of items 95 to 103, wherein the method does not comprise a determination of a previous dengue infection in the subject before the administration of the first unit dose of the tetravalent dengue virus composition.
105. The unit dose for use of any one of items 95 to 104, wherein the dengue serotype 4 represents the highest concentration in the composition of all four serotypes, optionally with at least about 70% of the total concentration in pfu/0.5 mL, dengue serotype 3 represents the second highest concentration in the composition of all four serotypes with at least about 10% of the total concentration in pfu/0.5 mL, and dengue serotype 1 and dengue serotype 2 each represent lower concentrations than the concentration of serotype 3 and together represent less than about 10% of the total concentration in pfu/0.5 mL and/or which are within 5%-points of each other.
106. The unit dose for use of any one of items 95 to 105, wherein the dengue serotype 1 is a chimeric dengue serotype 2/1 strain, the dengue serotype 2 is a non-chimeric dengue serotype 2 strain, the dengue serotype 3 is a chimeric dengue serotype 2/3 strain and the dengue serotype 4 is a chimeric dengue serotype 2/4 strain.
107. The unit dose for use of any one of items 95 to 106, wherein the dengue serotype 1 has the amino acid sequence of SEQ ID NO. 2, the dengue serotype 2 has the amino acid sequence of SEQ ID NO. 4, the dengue serotype 3 has the amino acid sequence of SEQ ID NO. 6, and the dengue serotype 4 has the amino acid sequence of SEQ ID NO. 8.
108. The unit dose for use of any one of items 95 to 107, wherein the unit dose further comprises from about 10% w/v to about 20% w/v α,α-trehalose dihydrate or an equimolar amount of other forms of α,α-trehalose, from about 0.5% w/v to about 1.5% w/v poloxamer 407, from about 0.05% w/v to about 2% w/v human serum albumin, and from about 70 mM to 140 mM sodium chloride when measured in 0.5 mL.
109. The unit dose for use of any one of items 95 to 108, wherein the unit dose further comprises about 15% (w/v) α,α-trehalose dihydrate, about 1% (w/v) poloxamer 407, about 0.1% (w/v) human serum albumin, and about 100 mM sodium chloride when measured in 0.5 mL.
110. The unit dose for use of any one of items 95 to 109, wherein the method is for preventing dengue hemorrhagic fever (DHF) or dengue shock syndrome (DSS).
111. The unit dose for use of any one of items 95 to 110, wherein the subject is from a dengue endemic region.
112. The unit dose for use of any one of items 95 to 111, wherein the subject is from a dengue non-endemic region.
113. The unit dose for use of any one of items 95 to 112, wherein the subject is from Asia Pacific or Latin America.
114. The unit dose for use of any one items of 95 to 113, wherein the unit dose provides a seropositivity rate when it is administered to a subject population of at least 50 subjects in two unit doses subcutaneously at day 1 and at day 90, wherein the subjects of the subject population are seronegative to all dengue serotypes at baseline.
115. The unit dose for use of item 114, wherein at least one month after administration of the first unit dose, such as at day 30, at least 80% of the subject population are seropositive for all four dengue serotypes.
116. The unit dose for use of item 114 or 115, wherein before or at the time of the administration of the second unit dose, such as at day 90, at least 80% of the subject population are seropositive for all four dengue serotypes.
117. The unit dose for use of any one of items 114 to 116, wherein after the administration of the second unit dose, such as at day 120, at least 80%, or at least 85%, or at least 90% or at least 95% of the subject population are seropositive for all four dengue serotypes.
118. The unit dose for use of any one of items 114 to 117, wherein after the administration of the second unit dose, such as at day 270, at least 80%, or at least 85%, or at least 90% of the subject population are seropositive for all four dengue serotypes.
119. The unit dose for use of any one of items 114 to 118, wherein the unit dose provides a seropositivity rate, when it is administered to a subject population of at least 100 subjects in two unit doses subcutaneously at day 1 and at day 90, wherein the subjects of the subject population comprises from 20% to 40% subjects who are seronegative to all dengue serotypes and from 60% to 80% subjects who are seropositive to at least one dengue serotype at base line, wherein at day 120 and/or day 270 the seropositivity rate for all four dengue serotypes in the seronegative part of the subject population and the seropositivity rate for all four dengue serotypes in the seropositive part of the subject population do not deviate more than 10%-points and/or wherein at day 120 the seropositivity rate for all four dengue serotypes in the seronegative part of the subject population and the seropositivity rate for all four dengue serotypes in the seropositive part of the subject population do not deviate more than 5%-points.
120. The unit dose for use of any one of items 95 to 119, wherein upon reconstitution with 0.5 ml of a pharmaceutically acceptable diluent
   (i) dengue serotype 1 has a concentration of 3.3 log 10 pfu/0.5 mL to 5.0 log 10 pfu/0.5 mL,
   (ii) dengue serotype 2 has a concentration of 2.7 log 10 pfu/0.5 mL to 4.9 log 10 pfu/0.5 mL,
   (iii) dengue serotype 3 has a concentration of 4.0 log 10 pfu/0.5 mL to 5.7 log 10 pfu/0.5 mL, and
   (iv) dengue serotype 4 has a concentration of 4.5 log 10 pfu/0.5 mL to 6.2 log 10 pfu/0.5 mL.
121. The unit dose for use of any one of items 95 to 120, wherein upon reconstitution with 0.5 ml of a pharmaceutically acceptable diluent:
   (i) dengue serotype 1 has a concentration of 3.3 log 10 pfu/0.5 mL to 5.0 log 10 pfu/0.5 mL,
   (ii) dengue serotype 2 has a concentration of 2.7 log 10 pfu/0.5 mL to 4.9 log 10 pfu/0.5 mL,
   (iii) dengue serotype 3 has a concentration of 4.0 log 10 pfu/0.5 mL to 5.7 log 10 pfu/0.5 mL, and
   (iv) dengue serotype 4 has a concentration of 4.5 log 10 pfu/0.5 mL to 5.5 log 10 pfu/0.5 mL.

1. (canceled)

2. A method to reduce the risk of dengue disease in a subject or subject population traveling to a dengue endemic region comprising selecting the subject or subject population based upon a plan of the subject or subject population to travel to a dengue endemic region and administering one dose of a dengue vaccine composition to the subject or subject population based upon the plan,
wherein the administration of the one dose results in a reduction of risk of dengue disease after said one dose,
wherein the dengue vaccine composition is a tetravalent dengue vaccine composition including four live attenuated dengue virus strains representing serotype 1, serotype 2, serotype 3 and serotype 4.

3. The method of claim 2, wherein the dengue disease is due to dengue serotype 1 and/or dengue serotype 2.

4. The method of claim 2, wherein the dengue endemic region is characterized by a seroprevalence rate with respect to dengue serotype 1 and/or dengue serotype 2 of at least about 80%.

5. The method of claim 4, wherein the dengue endemic region is characterized by a seroprevalence rate with respect to dengue serotype 1 and/or dengue serotype 2 of at least about 90%.

6. The method of claim 2, wherein the dengue endemic region is in one or more of Latin America, Southeast Asia or the Pacific Islands.

7. The method of claim 2, wherein the subject or subject population is travelling to one or more of Australia, Brazil, Bangladesh, Colombia, China, the Dominican Republic, Indonesia, India, Mexico, Malaysia, Nicaragua, Nigeria, Pakistan, Panama, Philippines, Puerto Rico, Singapore, Sri Lanka, Thailand or Vietnam.

8. The method of claim 2, wherein the subject or subject population is from a dengue non-endemic region.

9. The method of claim 2, wherein the subject or subject population is traveling to the dengue endemic region within three months after administration of the one dose and wherein the administration of one dose results in a reduction of risk of dengue disease within three months after the one dose.

10. The method of claim 9, wherein the subject or subject population is traveling to the dengue endemic region within two months after administration of the one dose and wherein the administration of one dose results in a reduction of risk of dengue disease within two months after the one dose.

11. The method of claim 9, wherein the subject or subject population is traveling to the dengue endemic region within one month administration of the one dose and wherein the administration of one dose results in a reduction of risk of dengue disease within one month after the one dose.

12. The method of claim 9, wherein the subject or subject population is traveling to the dengue endemic region within two weeks after administration of the one dose and wherein the administration of one dose results in a reduction of risk of dengue disease within two weeks after the one dose.

13. The method of claim 2, wherein the administration of the one dose results in a reduction of risk of dengue disease after said one dose represented by a combined vaccine efficacy against all four dengue virus serotypes of at least 60%.

14. The method of claim 2, wherein the administration of the one dose results in a reduction of risk of dengue disease after said one dose represented by a combined vaccine efficacy against all four dengue virus serotypes of at least 70%.

15. The method of claim 2, wherein the administration of the one dose results in a reduction of risk of dengue disease after said one dose represented by a combined vaccine efficacy against all four dengue virus serotypes of at least 80%.

16. The method of claim 2, wherein the administration of the one dose results in a reduction of risk of dengue disease after said one dose represented by a combined vaccine efficacy against all four dengue virus serotypes of at least 80%, wherein the lower bound of the two-sided 95% confidence interval is more than 60%.

17. The method of claim 2, wherein each of the four live, attenuated dengue virus serotypes is selected from the following:
(i) a non-chimeric dengue virus comprising all components from the same dengue serotype, and
(ii) a chimeric dengue virus having parts from two dengue serotypes.

18. The method of claim 2, wherein the dengue vaccine composition includes a chimeric dengue serotype 2/1 strain, a non-chimeric dengue serotype 2 strain, a chimeric dengue serotype 2/3 strain and a chimeric dengue serotype 2/4 strain.

19. The method of claim 2 wherein each of the four live, attenuated dengue virus strains comprises dengue structural proteins including capsid protein (C), pre-membrane protein (prM), and envelope protein (E), and dengue non-structural proteins including NS1, NS2A, NS2B, NS3, NS4A, NS4B, and NS5.

20. The method of claim 2, wherein the dengue serotype 2 strain is a non-chimeric dengue strain derived from the wild type virus strain DEN-2 16681 and is characterized by differing in at least a) to c) from the wild type as follows:
a) 5′-noncoding region (NCR)-57
b) NS1-53 Gly-to-Asp
c) NS3-250 Glu-to-Val; and
wherein the dengue serotype 1, dengue serotype 3, and dengue serotype 4 strains are chimeric dengue strains that are derived from the serotype 2 strain by replacing the structural proteins prM and E from serotype 2 strain with the corresponding structural proteins from the other dengue serotypes, resulting in the following chimeric dengue strains:
a DENV-2/1 chimera,
a DENV-2/3 chimera and
a DENV-2/4 chimera.

21. The method of claim 2, wherein
the dengue serotype 1 is a chimeric dengue serotype 2/1 strain, the dengue serotype 2 is a non-chimeric serotype 2 strain, the dengue serotype 3 is as a chimeric dengue serotype 2/3 strain, and the dengue serotype 4 is a chimeric dengue serotype 2/4 strain,
wherein the three chimeric dengue strains are derived from the serotype 2 strain by replacing the structural proteins prM and E from the serotype 2 strain with the corresponding structural proteins from the other dengue serotypes,
wherein in the DENV-2/1 chimeric strain the structural proteins prM and E from the serotype 2 strain are replaced with the corresponding structural proteins derived from DENV-1 16007, and
wherein in the DENV-2/3 chimeric strain the structural proteins prM and E from the serotype 2 strain are replaced with the corresponding structural proteins derived from a DENV-3 16562, and
wherein in the DENV-2/4 chimeric strain the structural proteins prM and E from the serotype 2 strain are replaced with the corresponding structural proteins derived from a DENV-4 1036.

22. The method of claim 2, wherein the method does not include a step of determination whether there was a previous dengue infection in the subject or subject population, wherein the step of determination of the previous dengue infection is characterized by a laboratory confirmed history of dengue or a validated serological test.

23. The method of claim 2, wherein the baseline serostatus of the subject or subject population is unknown.

24. The method of claim 2, further comprising administering a second dose of a dengue vaccine composition to the subject or subject population.

25. The method of claim 24, further comprising administering a booster dose of the dengue vaccine composition to the subject or subject population.

26. A method to reduce the risk of severe dengue disease in a subject or subject population traveling to a dengue endemic region comprising selecting the subject or subject population based upon a plan of the subject or subject population to travel to the dengue endemic region and administering one dose of a dengue vaccine composition to the subject or subject population based upon the plan,
wherein the administration of the one dose results in a reduction of risk of dengue disease after said one dose,
wherein the dengue vaccine composition is a tetravalent dengue vaccine composition including four live attenuated dengue virus strains representing serotype 1, serotype 2, serotype 3 and serotype 4.

27. The method of claim 26, wherein the administration of the one dose results in reduction of risk of dengue hemorrhagic fever (DHF) and/or dengue shock syndrome (DSS) in the subject or subject population after said one dose.
